(19) United States
(12) Patent Application Publication
(10) Pub. No.: US 2025/0115950 A1
(43) Pub. Date: Apr. 10, 2025

(54) PROGRAMMABLE NUCLEASE-BASED ASSAY IMPROVEMENTS

(71) Applicant: Mammoth Biosciences, Inc., Brisbane, CA (US)

(72) Inventors: Sarah Jane SHAPIRO, Pacifica, CA (US); Sonal JAIN, San Mateo, CA (US); Deepika VERMA, Belmont, CA (US); James Paul BROUGHTON, South San Francisco, CA (US); Benjamin Andrew BLIZARD, Oakland, CA (US); Janice Sha CHEN, San Francisco, CA (US); Matthew VEROSLOFF, South San Francisco, CA (US); Elizabeth M. HAWKINS, Redwood City, CA (US); Nicholas John FANTIN, San Bruno, CA (US); Rachel Jordan PATRON, Oakland, CA (US); Alexander HIRSCHI, Redwood City, CA (US); Nazmiye Emel ALPAY, Mountain View, CA (US); Lisa J. KRYGSMAN, Oakland, CA (US); Xin MIAO, Mountain View, CA (US); Jesus CHING, Saratoga, CA (US); Sara ANSALONI, Mountain View, CA (US)

(21) Appl. No.: 18/741,443

(22) Filed: Jun. 12, 2024

Related U.S. Application Data
(63) Continuation of application No. PCT/US2022/081842, filed on Dec. 16, 2022.
(60) Provisional application No. 63/293,569, filed on Dec. 23, 2021.
(60) Provisional application No. 63/344,362, filed on May 20, 2022.
(60) Provisional application No. 63/376,972, filed on Sep. 23, 2022.

(51) Int. Cl. C12Q 1/6823; C12N 9/22; C12N 15/11; C12Q 1/44; C12Q 1/48; G01N 21/64

(52) U.S. Cl. CPC C12Q 1/6823; C12N 9/22; C12N 15/111; C12Q 1/44; C12Q 1/485; G01N 21/6428; C12N 2310/20; G01N 2021/6432; G01N 2333/9126; G01N 2333/9128

(57) ABSTRACT

The present disclosure describes various improvements for programmable nuclease-based detection assays. Also provided are compositions, methods, kits, systems, and devices for practicing the same. Such improvements include improved reporter designs to facilitate the cleavage of a reporter immobilized on a substrate by an activated programmable nuclease. Improved reporter designs may comprise various lengths and structures of the reporter. The substrate can also comprise immobilized guide nucleic acids and/or programmable nucleases.

### CROSS-REFERENCE

This application claims priority to U.S. Provisional Application Ser. No. 63/293,569, filed on Dec. 23, 2021, U.S. Provisional Application Ser. No. 63/344,362, filed on May 20, 2022, and U.S. Provisional Application Ser. No. 63/376,972, filed on Sep. 23, 2022 which are incorporated herein by reference in their entireties for all purposes.

### SEQUENCE LISTING

The instant application contains a Sequence Listing which has been submitted electronically in XML format and is hereby incorporated by reference in its entirety. Said XML copy, created on Dec. 29, 2022, is named 203477-749601.xml and is 213,175 bytes in size.

### BACKGROUND

Nucleic acid tests can be used to detect and identify nucleic acid sequences associated with disease-causing organisms, such as influenza. Diagnostic tests that detect the presence or absence of nucleic acids to identify pathogenic organisms often have an advantage in sensitivity and specificity over other test methods, such as those based on antigen detection of the pathogenic organism. In some cases, while antibody/antigen tests may have the ability to directly assay a subject's immune response to a pathogenic organism, these systems can have sensitivity and specificity challenges related to the response time and the variabilities of antibodies/antigens produced by the subject's body.

Programmable nucleases are effector proteins that bind and cleave nucleic acids in a sequence-specific manner. A programmable nuclease may bind a target region of a nucleic acid and cleave the nucleic acid within the target region or at a position adjacent to the target region. Such cleavage is often referred to as cis cleavage. Upon binding to the target region of the nucleic acid, some programmable nucleases may be activated to provide non-specific cleavage of non-target nucleic acids that are near the target nucleic acid. Such non-specific cleavage is often referred to as trans cleavage or transcollateral cleavage activity. A programmable nuclease, such as a CRISPR-associated (Cas) protein, may be coupled to a guide nucleic acid that imparts activity or sequence selectivity to the programmable nuclease. Cis cleavage activity is cleavage of a target nucleic acid that is hybridized to the guide nucleic acid (e.g., crRNA or sgRNA), wherein cleavage occurs within or directly adjacent to the region of the target nucleic acid that is hybridized to guide nucleic acid. Trans cleavage activity is cleavage of single stranded nucleic acids (ssDNA or ssRNA) that is near, but not hybridized to the guide nucleic acid. Trans cleavage activity is activated by the hybridization of guide nucleic acid to the target nucleic acid.

Trans cleavage activity can be used to detect the presence and/or absence of target nucleic acids. Trans-cleavage assays utilize the trans-cleavage abilities of programmable nucleases to achieve detection of a target nucleic acid in a sample. For example, following target nucleic acid extraction from a biological sample, a guide nucleic acid comprising a portion that is complementary to the target nucleic acid of interest may bind to the target nucleic acid sequence, thereby initiating trans cleavage activity of the programmable nuclease. Once the programmable nuclease is activated, it may then cleave a nearby reporter (e.g., an RNA molecule comprising a fluorophore and a fluorescence quenching moiety that may separate upon cleavage of the RNA molecule). Cleavage of the reporter may provide a detectable readout indicative of the presence of the target nucleic acid in the sample.

### SUMMARY

It would therefore be desirable to provide improved programmable nuclease-based nucleic acid assay systems, devices, methods, and compositions. Such improvements may include, but are not limited to, improved time to answer (e.g., time from sample collection to detection), improved threshold of detection, improved reagent stability, improved multiplexing capacity, improved signal to noise ratios, improved reagent designs, improved assay conditions, or the like, or any combination thereof. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to nucleic acid tests, particularly relates to programmable nuclease-based nucleic acid test compositions, devices, systems, and methods, and more particularly relates to nucleic acid in vitro diagnostic compositions, devices, systems, and methods.

An aspect of the present disclosure provides for improved compositions and systems comprising an immobilized reporter molecule, and uses thereof in a trans cleavage assay. Compositions, systems, and methods of the present disclosure leverage transcollateral cleavage activity of programmable nucleases for the detection of nucleic acids. The reporters, programmable nucleases, and/or guide nucleic acids may be immobilized on a surface or substrate. Immobilization of the reporters, programmable nucleases, and/or guide nucleic acids on the substrate may facilitate detection of multiple target nucleic simultaneously (i.e., multiplexed detection) on the same substrate. In some cases, immobilized reporters may comprise a length and structure that facilitates the cleavage of the reporter upon detection of the target nucleic acid by the programmable nuclease.

In another aspect, disclosed herein is a system for detecting a target nucleic acid, the system comprising: a detection region comprising: a guide nucleic acid complementary to the target nucleic acid, or a portion thereof: a reporter immobilized to a surface of the detection region, the reporter comprising a nucleic acid and a detection moiety, wherein the nucleic acid is at least 40 nucleotides in length; the nucleic acid comprises a double-stranded region; or a combination thereof, and wherein cleavage of the reporter by a programmable nuclease, activated upon hybridization to the target nucleic acid, releases the detection moiety from the nucleic acid, and wherein the release of the detection moiety is configured to generate a signal indicative of a presence of the target nucleic acid. In some embodiments, the system for detecting a target nucleic acid further comprises the programmable nuclease. In some embodiments, the programmable nuclease is configured to form a complex with the guide nucleic acid and to be activated through binding of the guide nucleic acid to the target nucleic acid. In some embodiments, the nucleic acid is at least 50 nucleotides in length. In some embodiments, the nucleic acid comprises a single-stranded region and the double-stranded region. In some embodiments, the single-stranded region is from about 5 to about 15 nucleotides in length. In some embodiments, the double-stranded region is from about 45 to about 55 nucleotides in length. In some embodiments, the single-stranded region is about 9 nucleotides in length, and wherein the double-stranded region is about 50 nucleotides in length. In some embodiments, the nucleic acid is single-stranded. In some embodiments, the single-stranded nucleic acid is at least about 50 nucleotides in length. In some embodiments, the single-stranded nucleic acid is from about 55 to about 65 nucleotides in length. In some embodiments, the detection moiety comprises a quencher moiety. In some embodiments, the reporter comprises a fluorophore. In some embodiments, the quencher moiety is configured to quench the fluorophore prior to the cleavage of the reporter. In some embodiments, the detection moiety comprises a fluorophore. In some embodiments, the signal is 1) a fluorescence change, 2) a color change, 3) a brightness change or 4) a combination thereof. In some embodiments, the reporter comprises a nucleic acid sequence at least 80% identical to any one of the sequences set forth in Table 1. In some embodiments, the reporter comprises a nucleic acid sequence at least 90% identical to any one of the sequences set forth in Table 1. In some embodiments, the reporter comprises a nucleic acid sequence at least 95% identical to any one of the sequences set forth in Table 1. In some embodiments, the reporter comprises any one of the sequences set forth in Table 1. In some embodiments, the programmable nuclease is a Type V Cas nuclease or a Type VI Cas nuclease. In some embodiments, the programmable nuclease comprises an amino acid sequence at least 80% identical to any one of SEQ ID NOS: 26 or 43. In some embodiments, the programmable nuclease comprises an amino acid sequence at least 90% identical to any one of SEQ ID NOS: 26 or 43. In some embodiments, the programmable nuclease comprises an amino acid sequence at least 95% identical to any one of SEQ ID NOS: 26 or 43. In some embodiments, the programmable nuclease comprises the amino acid sequence of any one of SEQ ID NOS: 26 or 43. In some embodiments, the reporter is immobilized to the surface of the detection region using NHS-amine chemistry, streptavidin-biotin chemistry, epoxy-amine chemistry, maleimide-thiol chemistry, or a combination thereof. In some embodiments, the reporter comprises an amine group and wherein the surface comprises an NHS coating. In some embodiments, the guide nucleic acid is immobilized to the surface of the detection region. In some embodiments, the programmable nuclease is immobilized to the surface of the detection region. In some embodiments, the system further comprises a polymerase, a reverse transcriptase, or both; optionally wherein: (i) the polymerase is KAPA3G DNA polymerase, RAPIDXFIRE DNA polymerase, or ACAT77 DNA polymerase; and/or (ii) the reverse transcriptase is WARMSTART reverse transcriptase, RAPIDXFIRE reverse transcriptase, ACAT138 reverse transcriptase, and ACAT141.

Another aspect of the present disclosure provides a system for the multiplexed detection of a plurality of target nucleic acids, the system comprising: a detection region comprising a plurality of detection locations, each detection location of the plurality of detection locations comprising: a guide nucleic complementary to one of the plurality of target nucleic acids, or a portion thereof; a reporter immobilized to a surface of the detection region, the reporter comprising a nucleic acid and a detection moiety, wherein the nucleic acid is at least 40 nucleotides in length; the nucleic acid comprises a double-stranded region; or a combination thereof, and wherein, at each detection location, cleavage of the reporter by a programmable nuclease activated upon hybridization to the one of the plurality of target nucleic acids releases the detection moiety from the nucleic acid, wherein the release of the detection moiety is configured to generate a signal at the detection location; and wherein the signal indicates a presence or absence of the one of the plurality of target nucleic acids at the detection location. In some embodiments, the system comprises the programmable nuclease. In some embodiments, the programmable nuclease is configured to form a complex with the guide nucleic acid and to be activated through binding of the guide nucleic acid to one of the plurality of target nucleic acids. In some embodiments, the nucleic acid is at least 50 nucleotides in length. In some embodiments, the nucleic acid comprises the single-stranded region and a double-stranded region. In some embodiments, the single-stranded region is from about 5 to about 15 nucleotides in length. In some embodiments, the double-stranded region is from about 45 to about 55 nucleotides in length. In some embodiments, the single-stranded region is about 9 nucleotides in length, and wherein the double-stranded region is about 50 nucleotides in length. In some embodiments, the nucleic acid is single-stranded. In some embodiments, the single-stranded nucleic acid is at least about 50 nucleotides in length. In some embodiments, the single-stranded nucleic acid is from about 55 to about 65 nucleotides in length. In some embodiments, the detection moiety comprises a quencher moiety. In some embodiments, the reporter comprises a fluorophore. In some embodiments, the quencher moiety is configured to quench the fluorophore prior to the cleavage of the reporter. In some embodiments, the detection moiety comprises a fluorophore. In some embodiments, the signal is 1) a fluorescence change, 2) a color change, 3) a brightness change, 4) a wavelength change of a light, or 5) a combination thereof. In some embodiments, the reporter comprises a nucleic acid sequence at least 80% identical to any one of the sequences set forth in Table 1. In some embodiments, the reporter comprises a nucleic acid sequence at least 90% identical to any one of the sequences set forth in Table 1. In some embodiments, the reporter comprises a nucleic acid sequence at least 95% identical to any one of the sequences set forth in Table 1. In some embodiments, the reporter comprises any one of the sequences set forth in Table 1. In some embodiments, the programmable nuclease is a Type V Cas nuclease or a Type VI Cas nuclease. In some embodiments, the programmable nuclease comprises an amino acid sequence at least 80% identical to any one of the sequences set forth in Table 3. In some embodiments, the programmable nuclease comprises an amino acid sequence at least 90% identical to any one of the sequences set forth in Table 3. In some embodiments, the programmable nuclease comprises an amino acid sequence at least 95% identical to any one of the sequences set forth in Table 3. In some embodiments, the reporter comprises the amino acid sequence of any one of the sequences set forth in Table 3. In some embodiments, the reporter is immobilized to the surface of the detection region using NHS-amine chemistry, streptavidin-biotin chemistry, epoxy-amine chemistry, maleimide-thiol chemistry, or a combination thereof. In some embodiments, the reporter comprises an amine group and wherein the surface comprises an NHS coating. In some embodiments, the guide nucleic acid is immobilized to the surface of the detection region. In some embodiments, the programmable nuclease is immobilized to the surface of the detection region.

Another aspect of the present disclosure provides a method for detecting a target nucleic acid, the method comprising the steps of: applying a plurality of nucleic acids to a detection region comprising a programmable nuclease; a guide nucleic acid complementary to the target nucleic acid, or a portion thereof; and a reporter immobilized to a surface of a detection region, the reporter comprising a nucleic acid and a detection moiety, wherein the nucleic acid is at least 40 nucleotides in length; the nucleic acid comprises a double-stranded region; or a combination thereof, and detecting a signal indicative of a presence or absence of a target nucleic acid in the plurality of nucleic acids, wherein the programmable nuclease is activated by binding of the target nucleic acid to the guide nucleic acid, wherein activation of the programmable nuclease cleaves the reporter, thereby releasing the detection moiety from the nucleic acid and generating the signal indicative of a presence or absence of the target nucleic acid. In some embodiments, the programmable nuclease is immobilized to the detection region. In some embodiments, the programmable nuclease is configured to form a complex with the guide nucleic acid and to be activated through binding of the guide nucleic acid to the target nucleic acid. In some embodiments, the nucleic acid is at least 50 nucleotides in length. In some embodiments, the nucleic acid comprises the single-stranded region and the double-stranded region. In some embodiments, the single-stranded region is from about 5 to about 15 nucleotides in length. In some embodiments, the double-stranded region is from about 45 to about 55 nucleotides in length. In some embodiments, the single-stranded region is about 9 nucleotides in length, and wherein the double-stranded region is about 50 nucleotides in length. In some embodiments, the nucleic acid is single-stranded. In some embodiments, the single-stranded nucleic acid is at least about 50 nucleotides in length. In some embodiments, the single-stranded nucleic acid is from about 55 to about 65 nucleotides in length. In some embodiments, the detection moiety comprises a quencher moiety. In some embodiments, the reporter comprises a fluorophore. In some embodiments, the quencher moiety is configured to quench the fluorophore prior to the cleavage of the reporter. In some embodiments, the detection moiety comprises a fluorophore. In some embodiments, the signal is 1) a fluorescence change, 2) a color change, 3) a brightness change, or 4) a combination thereof. In some embodiments, the reporter comprises a nucleic acid sequence at least 80% identical to any one of the sequences set forth in Table 1. In some embodiments, the reporter comprises a nucleic acid sequence at least 90% identical to any one of the sequences set forth in Table 1. In some embodiments, the reporter comprises a nucleic acid sequence at least 95% identical to any one of the sequences set forth in Table 1. In some embodiments, the reporter comprises any one of the sequences set forth in Table 1. In some embodiments, the programmable nuclease is a Type V Cas nuclease or a Type VI Cas nuclease. In some embodiments, the programmable nuclease comprises an amino acid sequence at least 80% identical to any one of the sequences set forth in Table 3. In some embodiments, the programmable nuclease comprises an amino acid sequence at least 90% identical to any one of the sequences set forth in Table 3. In some embodiments, the programmable nuclease comprises an amino acid sequence at least 95% identical to any one of the sequences set forth in Table 3. In some embodiments, the programmable nuclease comprises the amino acid sequence of any one of the sequences set forth in Table 3. In some embodiments, the reporter is immobilized to the surface of the detection region using NHS-amine chemistry, streptavidin-biotin chemistry, epoxy-amine chemistry, maleimide-thiol chemistry, or a combination thereof. In some embodiments, the reporter comprises an amine group and wherein the surface comprises an NHS coating. In some embodiments, the guide nucleic acid is immobilized to the surface of the detection region. In some embodiments, the programmable nuclease is immobilized to the surface of the detection region. In some embodiments, the method further comprises a polymerase, a reverse transcriptase, or both; optionally wherein: (i) the polymerase is KAPA3G DNA polymerase, RAPIDXFIRE DNA polymerase, or ACAT77 DNA polymerase; and/or (ii) the reverse transcriptase is WARMSTART reverse transcriptase, RAPIDXFIRE reverse transcriptase, ACAT138 reverse transcriptase, and ACAT141.

Another aspect of the present disclosure provides a method for detecting a plurality of target nucleic acids, the method comprising the steps of: applying a plurality of nucleic acids to the detection region comprising a plurality of detection locations, each detection location comprising a programmable nuclease; a guide nucleic complementary to one of the plurality of target nucleic acids, or a portion thereof; a reporter immobilized to a surface of a detection region, the reporter comprising a nucleic acid and a detection moiety, wherein the nucleic acid is at least 40 nucleotides in length; the nucleic acid comprises a double-stranded region; or a combination thereof, and detecting, at each detection location, a signal indicative of a presence or absence of one of a plurality of target nucleic acids in the plurality of nucleic acids, wherein, at each detection location, the programmable nuclease is activated by binding of the one of the plurality of target nucleic acids to the guide nucleic acid, wherein activation of the programmable nuclease cleaves the reporter, thereby releasing the detection moiety from the nucleic acid and generating the signal indicative of a presence or absence of the one of the plurality of target nucleic acids. In some embodiments, the programmable nuclease is immobilized to the detection location. In some embodiments, the programmable nuclease is configured to form a complex with the guide nucleic acid and to be activated through binding of the guide nucleic acid to the target nucleic acid. In some embodiments, the nucleic acid is at least 50 nucleotides in length. In some embodiments, the nucleic acid comprises the single-stranded region and a double-stranded region. In some embodiments, the single-stranded region is from about 5 to about 15 nucleotides in length. In some embodiments, the double-stranded region is from about 45 to about 55 nucleotides in length. In some embodiments, the single-stranded region is about 9 nucleotides in length, and wherein the double-stranded region is about 50 nucleotides in length. In some embodiments, the nucleic acid is single-stranded. In some embodiments, the single-stranded nucleic acid is at least about 50 nucleotides in length. In some embodiments, the single-stranded nucleic acid is from about 55 to about 65 nucleotides in length. In some embodiments, the detection moiety comprises a quencher moiety. In some embodiments, the reporter comprises a fluorophore. In some embodiments, the quencher moiety is configured to quench the fluorophore prior to the cleavage of the reporter. In some embodiments, the detection moiety comprises a fluorophore. In some embodiments, the signal is 1) a fluorescence change, 2) a color change, 3) a brightness change, or 4) a combination thereof. In some embodiments, the reporter comprises a nucleic acid sequence at least 80% identical to any one of the sequences set forth in Table 1. In some embodiments, the reporter comprises a nucleic acid sequence at least 90% identical to any one of the sequences set forth in Table 1. In some embodiments, the reporter comprises a nucleic acid sequence at least 95% identical to any one of the sequences set forth in Table 1. In some embodiments, the reporter comprises any one of the sequences set forth in Table 1. In some embodiments, the programmable nuclease is a Type V Cas nuclease or a Type VI Cas nuclease. In some embodiments, the programmable nuclease comprises an amino acid sequence at least 80% identical to any one of the sequences set forth in Table 3. In some embodiments, the programmable nuclease comprises an amino acid sequence at least 90% identical to any one of the sequences set forth in Table 3. In some embodiments, the programmable nuclease comprises an amino acid sequence at least 95% identical to any one of the sequences set forth in Table 3. In some embodiments, the programmable nuclease comprises the amino acid sequence of any one of the sequences set forth in Table 3. In some embodiments, the reporter is immobilized to the surface of the detection region using NHS-amine chemistry, streptavidin-biotin chemistry, epoxy-amine chemistry, maleimide-thiol chemistry, or a combination thereof. In some embodiments, the reporter comprises an amine group and wherein the surface comprises an NHS coating. In some embodiments, the guide nucleic acid is immobilized to the surface of the detection region. In some embodiments, the programmable nuclease is immobilized to the surface of the detection region.

Another aspect of the present disclosure provides a nucleic acid molecule comprising a nucleic acid sequence at least 80% identical to any one of the sequences set forth in Table 1. In some embodiments, the nucleic acid molecule comprises a nucleic acid sequence at least 90% identical to any one of the sequences set forth in Table 1. In some embodiments, the nucleic acid molecule comprises a nucleic acid sequence at least 95% identical to any one of the sequences set forth in Table 1. In some embodiments, the nucleic acid molecule comprises a nucleic acid sequence of any one of the sequences set forth in Table 1

Another aspect of the present disclosure provides for systems for detecting a target nucleic acid in a sample. In some embodiments, the system comprises one or more units, wherein a unit comprises: (a) a non-naturally occurring guide nucleic acid immobilized to a surface by a linkage; and b) a plurality of reporters immobilized to the surface in proximity to the non-naturally occurring guide nucleic acid. In some embodiments, the non-naturally occurring guide nucleic acid comprises (i) one or more stabilized nucleotides, (ii) a repeat region comprising a first end and a second end, and (iii) a spacer region that hybridizes to a segment of the target nucleic acid or an amplicon thereof. In some embodiments, the first end of the repeat region is joined to the linkage, and the second end of the repeat region is joined to the spacer region. In some embodiments, the one or more stabilized nucleotides (i) lack a 2′—OH group, and (ii) are located within 10 nucleotides of a terminal hairpin nucleotide in the first end of the repeat region. In some embodiments, the non-naturally occurring guide nucleic acid is effective to form a complex with a programmable nuclease that is activated upon binding the target nucleic acid or amplicon thereof. In some embodiments, the formation of the activated complex is effective to induce detectable transcollateral cleavage of the reporters. In some embodiments, the linkage comprises a tether having a length at least that of a 10-nucleotide DNA molecule, and/or the reporters are immobilized to the surface by attachment to the non-naturally occurring guide nucleic acid, the linkage, or both. In some embodiments, the one or more stabilized nucleotides comprise DNA nucleotides, modified RNA nucleotides, or both. In some embodiments, the one or more stabilized nucleotides comprise a plurality of stabilized nucleotides. In some embodiments, the one or more stabilized nucleotides are located (a) within 2 to 6 nucleotides of the terminal hairpin nucleotide, and/or (b) within the repeat region. In some embodiments, the one or more stabilized nucleotides are effective to reduce a rate of cleavage of the non-naturally occurring guide nucleic acid by the programmable nuclease, relative to that for a corresponding non-naturally occurring guide nucleic acid that consists of RNA. In some embodiments, the linkage comprises the tether, and the tether comprises a hydrocarbon chain. In some embodiments, the linkage comprises the tether, and the tether comprises a tether polynucleotide. In some embodiments, the tether polynucleotide: (a) is 10 to 200 nucleotides in length, 10 to 100 nucleotides in length, 12 to 60 nucleotides in length, or 15 to 20 nucleotides in length; (b) is single stranded; and/or (c) comprises DNA. In some embodiments, the terminal hairpin nucleotide: (a) is a 5′ terminal hairpin nucleotide located 3′ relative to the stabilized nucleotides; and/or (b) is a terminal nucleotide within a sequence of nucleotides that is complementary to a sequence of nucleotides in the second end of the repeat sequence and proximal to the linkage. In some embodiments, the reporters are immobilized to the surface by attachment to the non-naturally occurring guide nucleic acid, the linkage, or both. In some embodiments, at least one of the reporters is immobilized to the surface by attachment to an end of the non-naturally occurring guide nucleic acid that is distal to the linkage. In some embodiments, at least one of the reporters is immobilized to the surface by attachment to the linkage. In some embodiments, each of the reporters comprises a fluorophore and a quencher. In some embodiments, cleavage of the reporters is effective to produce a detectable loss of the quencher. In some embodiments, each of the reporters comprise a detection moiety. In some embodiments, cleavage of the reporters is effective to produce a detectable loss of the detection moiety. In some embodiments, the detection moiety comprises a fluorophore. In some embodiments, (a) the one or more units comprise a plurality of different units, and (b) each of the different units comprises a non-naturally occurring guide nucleic acid comprising a spacer region with a different sequence. In some embodiments, the system further comprises the programmable nuclease complexed with the non-naturally occurring guide nucleic acid. In some embodiments, the programmable nuclease comprises an RuvC catalytic domain. In some embodiments, the programmable nuclease is a type V CRISPR/Cas effector protein. In some embodiments, the type V CRISPR/Cas effector protein is a Cas 12 protein, a Cas14 protein, or a CasΦ protein. In some embodiments, the type V CRISPR/Cas effector protein is a Cas 12 protein comprising a Cas12a, a Cas12b, a Cas12c, a Cas12d, a Cas12e, a Cas12f, a Cas12g, a Cas12h, a Cas12i, a Cas12j, or a Cas12k. In some embodiments, the type V CRISPR/Cas effector protein comprises an amino acid sequence that is at least 80%, 90%, 95%, or 100% identical to SEQ ID NO: 43. In some embodiments, the programmable nuclease is a type VI CRISPR/Cas effector protein. In some embodiments, the type VI CRISPR/Cas effector protein is a Cas13 protein; optionally wherein the Cas 13 protein comprises a a Cas13a, a Cas13b, a Cas13c, a Cas13d, a Cas13e, or a Cas13f. Another aspect of the present disclosure provides for methods of assaying for one or more target nucleic acids in a sample, the method comprising: (a) contacting the system of described above with the sample; (b) cleaving the reporters in response to presence of the target nucleic acid or amplicon thereof; and (c) detecting a change in signal resulting from cleavage of the reporters. In some embodiments, the detection identifies the target nucleic acid in the sample. In some embodiments, a method further comprises amplifying the one or more target nucleic acids before or during said contacting.

Another aspect of the present disclosure provides for systems for detecting a target nucleic acid in a sample. In some embodiments, the system comprises one or more units, wherein a unit comprises: (a) a non-naturally occurring guide nucleic acid; (b) a plurality of reporters; and (c) a programmable nuclease. In some embodiments, the non-naturally occurring guide nucleic acid comprises a spacer region that hybridizes to a segment of the target nucleic acid or an amplicon thereof. In some embodiments, the segment hybridized by the spacer region is directly adjacent to a sequence that is complementary to a noncanonical PAM sequence. In some embodiments, the noncanonical PAM sequence differs from a naturally-occurring PAM sequence for a reference Cas nuclease. In some embodiments, the reference Cas nuclease is a Cas nuclease of the same type as the programmable nuclease. In some embodiments, the non-naturally occurring guide nucleic acid is effective to form a complex with the programmable nuclease that is activated upon binding the target nucleic acid or amplicon thereof. In some embodiments, formation of the activated complex is effective to induce detectable transcollateral cleavage of the reporters. In some embodiments, the programmable nuclease comprises a Type V Cas nuclease. In some embodiments, the programmable nuclease is a Cas 12 protein, a Cas14 protein, or a CasΦ protein. In some embodiments, the programmable nuclease is a Cas12 protein comprising a Cas12a, a Cas12b, a Cas12c, a Cas12d, a Cas12e, a Cas12f, a Cas12g, a Cas12h, a Cas12i, a Cas12j, or a Cas12k. In some embodiments, the programmable nuclease is a Cas12a protein. In some embodiments, the programmable nuclease comprises an amino acid sequence that is at least 80%, 90%, 95%, or 100% identical to SEQ ID NO: 43. In some embodiments, the canonical PAM sequence comprises TTTN or YYN. In some embodiments, the canonical PAM sequence comprises TTTA. In some embodiments, the noncanonical PAM sequence comprises CCGT, TCCG, CCCT, TGTC, TTGT, GGGC, TGGG, TTGG, TTTG, CTTT, CCTT, TCCT, TTCC, or CTGT. In some embodiments, the noncanonical PAM sequence comprises CCG, TTC, CCC, TGT, TTG, TCTT, TTCT, TTTT, CTTT, CCTT, TCGG, CTCG, TTTG, GGGC, TGGG, TTGG, TCCT, TTCC, or CTGT. In some embodiments, the programmable nuclease is a Type V Cas nuclease comprising an amino acid sequence that is at least 80%, 90%, 95%, or 100% identical to SEQ ID NO: 43. In some embodiments, the non-naturally occurring guide nucleic acid, the reporters, or both are immobilized to a surface. In some embodiments, (a) the one or more units comprise a plurality of different units, and (b) each of the different units comprises a non-naturally occurring guide nucleic acid comprising a spacer region with a different sequence. In some embodiments, each of the reporters comprises a fluorophore and a quencher. In some embodiments, cleavage of the reporters is effective to produce a detectable loss of the quencher. In some embodiments, each of the reporters comprises a detection moiety. In some embodiments, cleavage of the reporters is effective to produce a detectable loss of the detection moiety. In some embodiments, the detection moiety comprises a fluorophore. Another aspect of the present disclosure provides for methods of assaying for one or more target nucleic acids in a sample, the method comprising: (a) contacting the system of described above with the sample; (b) cleaving the reporters in response to presence of the target nucleic acid or amplicon thereof; and (c) detecting a change in signal resulting from cleavage of the reporters. In some embodiments, the detection identifies the target nucleic acid in the sample. In some embodiments, a method further comprises amplifying the one or more target nucleic acids before or during said contacting.

In one aspect, the present disclosure provides a method of detecting a target nucleic acids in a sample, the method comprising: (a) contacting the sample with a composition comprising primers, a polymerase, a programmable nuclease, a non-naturally occurring guide nucleic acid, and a reporter; (b) amplifying the target nucleic acid with the primers and polymerase; (c) forming an activated complex comprising (i) the programmable nuclease, and (ii) the non-naturally occurring guide nucleic acid hybridized to the target nucleic acid or an amplicon thereof; (d) cleaving the reporter with the activated complex; and (e) detecting a change in signal resulting from cleavage of the reporters, thereby detecting the target nucleic acid in the sample; wherein steps (a) through (d) are performed in a single reaction volume; and wherein steps (b) through (d) comprise (i) isothermal incubation (e.g., at a temperature of about 50° C. to about 62° C.) or (ii) incubation at a first temperature (e.g., about 62° C.) followed by incubation at a reduced second temperature (e.g., about 55° C.).

In one aspect, the present disclosure provides a method of detecting a first or second target nucleic acid in a sample, the method comprising: (a) contacting the sample with a composition comprising amplification reagents, a programmable nuclease, a first non-naturally occurring guide nucleic acid, a second non-naturally occurring guide nucleic acid, and reporters; (b) amplifying the first or second target nucleic acid; (c) forming an activated complex comprising the programmable nuclease and (i) the first non-naturally occurring guide nucleic acid hybridized to the first target nucleic acid or an amplicon thereof, or (ii) the second non-naturally occurring guide nucleic acid hybridized to the second target nucleic acid or an amplicon thereof; (d) cleaving the reporters with the activated complex; and (e) detecting a change in signal resulting from cleavage of the reporters at a first level or a second level, thereby detecting the first or second target nucleic acid in the sample; wherein steps (a) through (d) are performed in a single reaction volume; wherein the first level is above a first threshold and below a second threshold; wherein the second level is above the second threshold; wherein presence of the first target nucleic acid in the sample results in detection at the first level; and wherein presence of the second target nucleic acid in the sample results in detection at the second level.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure may be obtained by reference to the following detailed description that sets forth illustrative embodiments, in which the principles of the present disclosure are utilized, and the accompanying drawings of which:

FIGS. 1A-1E depict exemplary reporters immobilized on a substrate. Arrows indicate signal (e.g., fluorescence) change when the reporter is cleaved by a programmable nuclease.

FIG. 2 depicts an exemplary workflow of detecting nucleic acids using immobilized reporters and/or guide nucleic acids.

FIGS. 3A-3B depict images showing exemplary visually-detectable signals resulting from the cleavage of immobilized reporters comprising a fluorophore releasable by a trans-cleavage reaction in an assay having both reporters and guide nucleic acids immobilized on a substrate. FIG. 3A shows the signals from the reporter immobilized (shown as replicates on the bottom) versus a negative control (shown as replicates on the top) prior to the cleavage reaction commencing (i.e., the trans-cleavage of the reporter by the programmable nuclease being activated by the binding of the guide nucleic acid to the target nucleic acid). FIG. 3B shows the signals twenty minutes subsequent to the cleavage reaction commencing. As can be seen from FIG. 3B, cleavage of the reporter decreased the detectable signal as expected, thereby indicating the presence of the target nucleic acid.

FIG. 4 depicts an image of an exemplary substrate immobilized with guide nucleic acids and reporters. Each spot represents a discrete detection location on the substrate immobilized with at least one guide nucleic acid and at least one reporter.

FIG. 5 depicts the average percentage increase of the detectable signal resulting from the cleavage of a reporter immobilized at a discrete detection location on a substrate versus the negative control using the workflow shown in FIG. 2. A target-complementary guide nucleic acid and reporter rep203 (comprising a double-stranded region, a fluorophore, a cleavable single-stranded region, and a releasable quencher as shown in FIG. 1D) were bound to the substrate using NHS-amine chemistry. The Y-axis shows the increase in signal intensity at twenty minutes after the addition of the Cas protein and the target nucleic acid (T20) relative to before they were added (T0). Error bars depict 3 standard deviations (s.d.) of the percentage signal increase. The data represents the average of 10-12 discrete detection locations of each of 30 substrates.

FIGS. 6A-6D depict changes in detectable fluorescent signal resulting from the cleavage of various sets of exemplary improved reporters (rep136, rep204, and rep200) and 4 different guide nucleic acids (G1-G4) immobilized at a discrete detection locations on a substrate before and 60 minutes subsequent to a trans cleavage reaction commencing. The amounts of the reporters and guide nucleic acids immobilized are listed on the X-axis. Percentage change (Y-axis) represents the change of the fluorescent signal intensity at 60 minutes after the addition of the Cas protein and the target nucleic acid. Error bars depict 1 s.d. The data represents the average of 4 discrete detection locations for each immobilized reporter and guide nucleic acid. Arrows indicate expected fluorescence change when the reporter is cleaved by a programmable nuclease.

FIG. 6E depicts changes in detectable fluorescent signal resulting from the cleavage reaction of other sets of exemplary reporters (rep136, rep112, and rep135) and guide nucleic acids (G4) immobilized at a discrete detection location on a substrate in various conditions prior to the cleavage reaction has commenced versus 60 minutes subsequent to the cleavage reaction has commenced. The amounts of the reporters and guide nucleic acids immobilized are listed on the X-axis. Percentage change (Y-axis) represents the change of the fluorescent signal intensity at 60 minutes after the addition of the Cas protein and the target nucleic acid. Error bars depict 1 s.d. The data represents the average of 12 discrete detection locations for each immobilized reporter and guide nucleic acid. Arrows indicate expected fluorescence change when the reporter is cleaved by a programmable nuclease.

FIG. 7 depicts average percentage increase of a detectable fluorescent signal on various substrate replicates (X-axis) having an exemplary reporter rep203 and guide nucleic acid immobilized thereon. A negative control (rep203 without target present) is shown for comparison. The average of all thirty replicates is shown in FIG. 5. Percentage increase (Y-axis) represents the average increase of the signal intensity of the reporters on each substrate at 20 minutes after the addition of the Cas protein and the target nucleic acid (if present). Each substrate contained 10-12 discrete detection locations. Data is summarized in box and whisker plots.

FIG. 8 depicts a percentage decrease of the detectable fluorescent signal on various substrate replicates (X-axis) of another exemplary reporter rep204 and guide nucleic acid immobilized thereon. A negative control (rep204 without target) and an off-target control (rep204 with off-target guide) are shown for comparison. Percentage increase (Y-axis) represents the average decrease of the signal intensity of the reporters on each substrate at 20 minutes after the addition of the Cas protein and the target nucleic acid (if present). Each substrate contained 10-12 discrete detection locations. The data is summarized in box and whisker plots.

FIG. 9 depicts the percentage decrease (Y-axis) of the detectable signal of an exemplary reporter rep204 over time (X-axis). Each data point represents the average decrease of signal intensity of the reporter of 20 discrete detection locations after the cleavage reaction started. Error bars depict 1 s.d.

FIG. 10 depicts a substrate comprising a surface with an immobilized programmable nuclease-guide nucleic acid complex and a plurality of reporters, highlighting reporter cleavage by an activated programmable nuclease, in accordance with embodiments.

FIG. 11 depicts an exemplary device for detecting target nucleic acids using immobilized reporters, guide nucleic acids, and/or programmable nuclease using compositions and systems described herein.

FIG. 12 depicts an exemplary workflow of a system for nucleic acid analysis, in accordance with embodiments disclosed herein.

FIG. 13 depicts a graph showing reporter signals from immobilized guides after a target DNA was added (off target and on target) and when no target was added to the system. The Y-axis shows the percent signal increase and the X-axis shows results for different array slides.

FIG. 14 depicts graphs showing reporter signals from free floating chimeric guides after different concentrations of target DNA was added (0 and 5 nM) to the system. The fluorescence intensity is shown in the left Y-axis and the time is shown on the bottom X axis. Cas12 enzymes are depicted in the upper X axis and the right Y axis shows a fully-RNA guide (R777), a guide with the first 2 nucleotides being converted to DNA (F78) and a guide with the first 6 nucleotides being converted to DNA (Mod306).

FIG. 15 depicts a graph showing fold change in signal from immobilized modified guides containing DNA nucleotides in the guide RNA sequence and from a processable guide (RNA only). The fold change is shown in the left Y-axis and the reporter and guide concentrations are indicated along the X axis. Results are presented in groups of four bars, with the bars from left to right corresponding to the wells indicated in the legend from top to bottom, respectively.

FIGS. 16A-16B depict graphs showing fold change in signal from immobilized modified guides containing longer linkers between the attachment chemistry and guide sequence. The fold change is shown in the left Y-axis and the reporter and guide concentrations are indicated in the respective legends. The conditions indicated by the legends from top to bottom correspond to the results from left to right in each graph. Off target spots are shown as the two bar data points positioned in the far right of each graph.

FIG. 17A depicts graphs showing fold change of signal of modified guide nucleic acids attached to reporters (also referred to herein as “guide reporters” or “gREPs”) after being exposed to their cognate target. The fold change is shown in the left Y-axis and the reporter and guide concentrations are indicated in the respective legends. The conditions indicated by the legends from top to bottom correspond to the results from left to right in each graph.

FIG. 17B depicts an illustration of example gREPs complexed with a programmable nuclease and a target nucleic acid. Starting from the immobilized end, the attachment chemistry enables immobilization to a surface. An uncleavable carbon spacer is attached to the attachment chemistry and allows the complex room to search for its target. Additionally, the carbon spacer is attached to a chimeric guide, onto which the Cas enzyme forms an active complex. The end is comprised of a ssDNA tether holding a fluorophore. In some variations, the ssDNA tether is attached to the uncleavable carbon spacer. Upon target recognition, the ssDNA is cleaved allowing for the fluorophore to diffuse away from the spot, thereby producing a change in signal.

FIG. 18 depicts graphs showing results for experiments using the same array of spots comprised of gREPs targeting mammathus, RNAse P, or FluB. The fold change of the signal from the modified guide reporters is shown after exposure to one of the three targets. Only spots containing the cognate gREP to the introduced target presented a significant fold change. The fold change is shown in the left Y-axis and the specific guides are indicated in the legends. Guide targets indicated in the legends from top to bottom correspond to the results from left to right in each graph.

FIG. 19 depicts graphs showing single reaction volume assays with a Cas12 and a Cas14a. 1. The fluorescence is shown in the Y-axis and the time of the reaction is shown in the X-axis. Both assays targeted human RNaseP POP7 at the same target site and used the same TTTN PAM.

FIG. 20 depicts graphs showing single reaction volume assays with a thermostable Cas12. The fluorescence is shown in the Y-axis and the time of the reaction is shown in the X-axis. The assays targeted influenza B. The canonical and noncanonical PAMs tested are shown at the top of each graph.

FIG. 21 depicts graphs showing fluorescence from single reaction volume assays with a thermostable Cas12 with different concentrations of target nucleotides. The replicate number is shown in the Y-axis and the gRNA guide ID is shown on the X axis. The canonical and noncanonical PAMs tested are shown at the bottom of each graph.

FIGS. 22A-22B depict graphs showing single reaction volume assays with a thermostable Cas12. The fluorescence is shown in the Y-axis and the time of the reaction is shown in the X-axis. The assays targeted RNase P. The canonical and noncanonical PAMs tested are shown at the top of each graph. For each plot in which two curves are distinguishable, the top curve corresponds to results for the 450 pg/rxn condition.

FIGS. 23A-23D depict graphs showing illustrative results of single reaction volume assays performed under different temperature conditions (an isothermal reaction at 62° C. or a two-stage temperature reaction at 62° C. for 10 minutes followed by 55° C. for 50 minutes) with target nucleic acids at different concentrations. The fluorescence is shown in the Y-axis and the time of the reaction is shown in the X-axis. All reactions contained reagents for LAMP amplification, and some (Complex +) included reagents for programmable nuclease-based detection, while others did not (Complex −). Reactions also included a fluorescent dye (SYTO) to determine activity of the amplification reaction (FIGS. 23A-23B) and a reporter to determine activity of the programmable nuclease-based detection (FIGS. 23C-23D). Three replicates were performed per assay condition.

FIG. 24A depicts graphs showing illustrative results of single reaction volume assays combining both nucleic acid amplification and programmable nuclease-based detection performed with various polymerases, with various amounts of polymerase per reaction, and under various temperature conditions (isothermal reactions (“iso”) in comparison with a two-stage temperature conditions (“SIMMR”)). Four replicates were performed per assay condition.

FIG. 24B depicts a fluorescence intensity gradient map showing illustrative results of single reaction volume assays performed at different isothermal temperatures conditions with target nucleic acids at different concentrations. All reactions contained reagents for both LAMP amplification and programmable nuclease-based detection, a fluorescent dye (SYTO) to determine activity of the amplification reaction (top panel), and a reporter to determine activity of the programmable nuclease-based detection (bottom panel). All reactions contained a Bst 2.0 polymerase at 4U per reaction and a Cas enzyme having SEQ ID NO: 43.

FIG. 25 depicts graphs of potential fluorescence signals for single reaction volume assays performed with a programmable nuclease (e.g., Cas14a.1) to detect different target nucleic acid(s). Assay (a) includes reagents for the detection of a single target nucleic acid with a corresponding threshold for detection. Assays (b) and (c) include reagents for the multiplexed detection of any of two (assay (b)) or three (assay (c)) different target nucleic acids in a single reaction volume. The detection reaction for each different target nucleic acid saturates at a different level when the corresponding target nucleic acid is present, and is associated with a different threshold of detection. Thus, the level at which fluorescence plateaus is indicative of which target nucleic acid is present, allowing for the combination of reagents for detecting any of multiple distinct target nucleic acids in a single reaction, using the same reporter molecules. The graphs for assays (b) and (c) illustrate the various fluorescence plateaus and associated thresholds collapsed onto a single plot for the sake of comparison.

FIGS. 26A-26B depict graphs showing illustrative results of fluorescence signal detection for single volume reaction assays with a programmable nuclease (Cas14a.1) for various concentrations of different target nucleic acids. FIG. 26A shows results of an assay with reagents for the detection of RSV-A. FIG. 26B shows results of an assay with reagents for the detection of RSV-B. Each reaction contained reagents for the detection of either RSV-A or RSV-B, but not both.

FIG. 27A depicts graphs showing illustrative results of fluorescence signals for single volume reaction assays with a programmable nuclease (Cas14a.1) to detect one of two different target nucleic acids. All reactions contained reagents for the detection of both RSV-A and RSV-B, but a target nucleic acid of only one of these was added to each reaction. Each line represents a single reaction in which either RSV-B target nucleic acids (top set of lines) or RSV-A target nucleic acids (bottom set of lines) were added to the reaction. As indicated, the maximum fluorescence of the assay for reactions with RSV-B was higher than reactions with RSV-A despite both reactions having identical detection reagents.

FIG. 27B depicts graphs showing illustrative results of fluorescence signals for single volume reaction assays with a programmable nuclease (Cas14a. 1) to detect one of two different target nucleic acids. The conditions and target nucleic acids were the same as in FIG. 27A, but a higher Mg²⁺ concentration was used (6.5 mM instead of 5 mM). Each line represents a single reaction in which either RSV-B target nucleic acids (top set of lines) or RSV-A target nucleic acids (bottom set of lines) were added to the reaction.

### DETAILED DESCRIPTION

It is to be understood that both the foregoing general description and the following detailed description are exemplary and explanatory only and are not restrictive of the disclosure. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or portions of the method or process may be performed in any suitable sequence and are not necessarily limited to any particular disclosed sequence. Various operations may be described as multiple discrete operations in turn, in a manner that may be helpful to understanding certain embodiments, however, the order of the description should not be construed to imply that these operations are order dependent. Additionally, structures, systems, and/or devices described herein may be embodied as integrated components or as separate components.

For the purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages taught herein without necessarily achieving other aspects or advantages as may also be taught or suggested herein.

The present disclosure is described in relation to systems, devices, or methods for in vitro diagnostics, and in particular for detection of an ailment such as a disease, cancer, or genetic disorder. However, one of ordinary skill in the art will appreciate that this is not intended to be limiting and the devices and methods disclosed herein may be used in other nucleic acid testing including, but not limited to, detecting genetic information, such as for phenotyping, genotyping, determining ancestry, or the like.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise. Any reference to “or” herein is intended to encompass “and/or” unless otherwise stated.

As used herein, the term “about” in reference to a number or range of numbers is understood to mean the stated number and numbers +/−10% thereof, or 10% below the lower listed limit and 10% above the higher listed limit for the values listed for a range.

As used herein, the term “cleavage assay” refers to an assay designed to visualize, quantitate, or identify the trans-collateral or cis-cleavage activity of an activated programmable nuclease. As used herein, an “activated” programmable nuclease is a programmable nuclease that is activated upon hybridization of a guide nucleic acid to a target nucleic acid, thereby inducing the formation of the nuclease-guide complex effective to cleave a nucleic acid (e.g., the nucleic acid of a reporter described herein).

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the terms “individual,” “subject,” and “patient” are used interchangeably and include any member of the animal kingdom, including humans.

As used herein, the terms “percent identity” and “% identity” refer to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs may be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95).

As used herein, the term “programmable nuclease,” “Cas effector,” “effector,” “CRISPR-associated protein,” “CRISPR/Cas protein,” or “Cas protein” as used herein, refers to a polypeptide, or a fragment thereof, possessing enzymatic activity, and that is capable of binding to a target nucleic acid molecule with the support of a guide nucleic acid molecule. In some embodiments, the binding is sequence-specific. In some embodiments, the guide nucleic acid molecule comprises DNA or RNA. In different embodiments, the target nucleic acid molecule may comprise DNA or RNA. In some embodiments, the enzymatic activity may be endonuclease activity, integrase activity, nickase activity, exonuclease activity, transposase activity, and/or excision activity.

As used herein, the term “reporter” or “reporter molecule” refers generally to an non-target nucleic acid molecule that is capable of providing a detectable signal indicative of cleavage by an activated programmable nuclease. The programmable nuclease, activated upon hybridization of a guide nucleic acid to a target nucleic acid, may cleave the reporter. In some instances, the reporter comprises a moiety, chemical compound, or other component that can be used to visualize, quantitate, or identify the cleavage of the reporter. Cleaving the “reporter” may be referred to herein as cleaving the “reporter molecule,” or the “nucleic acid of the reporter.” In some instances, the reporter comprises RNA. In some instances, the reporter comprises DNA. In some instances, the reporter is single-stranded, double-stranded, or a combination thereof. In some instances, reporters comprise a protein capable of generating a signal. A signal may be a calorimetric, potentiometric, amperometric, optical (e.g., fluorescent, colorimetric, etc.), piezo-electric, or the like. In some instances, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like. A detection moiety may comprise a fluorophore and/or a quenching moiety. In some instances, the reporter comprises a cleavage site, wherein the fluorophore is located at a first site on the reporter and the quenching moiety is located at a second site on the reporter, wherein the first site and the second site are separated by the cleavage site. Sometimes the quenching moiety is a fluorescence quenching moiety. In some instances, the quenching moiety is 5′ to the cleavage site and the fluorophore is 3′ to the cleavage site. In some instances, the fluorophore is 5′ to the cleavage site and the quenching moiety is 3′ to the cleavage site. Sometimes the quenching moiety is at the 5′ terminus of the nucleic acid of a reporter. Sometimes the fluorophore is at the 3′ terminus of the nucleic acid of a reporter. In some instances, the fluorophore is at the 5′ terminus of the nucleic acid of a reporter. In some instances, the quenching moiety is at the 3′ terminus of the nucleic acid of a reporter.

The term, “detectable signal,” as used herein refers to a signal that can be detected using optical, fluorescent, chemiluminescent, electrochemical or other detection methods known in the art.

The term, “guide nucleic acid,” as used herein refers to a nucleic acid comprising: a first nucleotide sequence that hybridizes to a target nucleic acid; and a second nucleotide sequence that is capable of being non-covalently bound by an effector protein. The first sequence may be referred to herein as a spacer sequence. The second sequence may be referred to herein as a repeat sequence. In some embodiments, the first sequence is located 5′ of the second nucleotide sequence. In some embodiments, the first sequence is located 3′ of the second nucleotide sequence.

The term, “protospacer adjacent motif (PAM),” as used herein refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

As used herein, the terms “thermostable” and “thermostability” refer to the stability of a composition disclosed herein at one or more temperatures, such as an elevated operating temperature for a given reaction. Stability may be assessed by the ability of the composition to perform an activity, e.g., cleaving a target nucleic acid or reporter. Improving thermostability means improving the quantity or quality of the activity at one or more temperatures.

As used herein, a “one-pot” reaction refers to a reaction in which more than one reaction occurs in a single volume alongside a programmable nuclease-based detection (e.g., DETECTR) assay. For example, in a one-pot assay, sample preparation, reverse transcription, amplification, in vitro transcription, or any combination thereof, and programmable nuclease-based detection (e.g., DETECTR) assays (optionally including signal amplification) are carried out in a single volume. In some embodiments, amplification and detection are carried out within a same volume or region of a device (e.g., within a detection region). Readout of the detection (e.g., DETECTR) assay may occur in the single volume or in a second volume. For example, the product of the one-pot DETECTR reaction (e.g., a cleaved detection moiety comprising an enzyme) may be transferred to another volume (e.g., a volume comprising an enzyme substrate) for signal generation and indirect detection of reporter cleavage by a sensor or detector (or by eye in the case of a colorimetric signal).

### Introduction

Provided herein, in some embodiments, are methods, compositions, kits, systems, or instruments that use improved reporter designs to facilitate the detection of reporters in immobilized reporter systems. In some instances, immobilizing reporters to a surface allows for an increase in the throughput and the detection capabilities of a trans cleavage assay. For example, in some instances, immobilizing a plurality of reporters to a surface allows for multiple location-specific trans-cleavage reactions to be carried concurrently. However, surface chemistry and surface physical characteristics of a substrate may interfere with the detection of an immobilized reporter. Without being bound by any theory, in some cases, the steric effects exhibited by surfaces on immobilized reporters may have a significant influence on the rate or completion of a trans cleavage reaction. Improved reporter designs provided herein can reduce or minimize the interference of the reporter by the substrate, facilitating the detection of the reporter in a trans-cleavage assay, including multiplexed trans-cleavage assays.

Systems described herein may comprise a detection region. In some cases, the detection region may comprise a plurality of detection locations. A detection region or a detection location may comprise a guide nucleic acid complementary to the target nucleic acid, or a portion thereof, and/or a reporter immobilized to a surface of the detection region. In some cases, the reporter may comprise a nucleic acid and a detection moiety as described herein. In some cases, cleavage of the reporter by a programmable nuclease, activated upon hybridization to the target nucleic acid, may release the detection moiety from the nucleic acid of the reporter. In some instances, the reporter may be configured to generate a signal indicative of a presence or absence of the target nucleic acid. In some instances, the release of the detection moiety may be configured to generate a signal indicative of the presence of the target nucleic acid. In some instances, the reporter may be configured to generate a signal indicative of the absence of the target nucleic acid and release of the detection moiety may decrease or eliminate the signal when the target nucleic acid is present. In some instances, release of a quencher moiety may be configured to generate a signal indicative of the presence of the target nucleic acid. In some instances, methods for detecting a target nucleic acid may comprise the systems thereof. In some cases, the method may comprise (a) transferring the target nucleic acid to the detection region or the detection location, and (b) detecting a signal in the presence of the target nucleic acid.

Provided herein in some embodiments, is a system for detecting a target nucleic acid comprising a detection region. The detection region can comprise a guide nucleic acid complementary to the target nucleic acid, or a portion thereof, and a reporter immobilized to a surface of the detection region. The reporter can comprise a nucleic acid and a detection moiety, wherein a. the nucleic acid is at least 40 nucleotides in length; b. the nucleic acid comprises a double-stranded region; c. or a combination thereof. Cleavage of the reporter by a programmable nuclease, activated upon hybridization to the target nucleic acid, can release the detection moiety from the nucleic acid. The release of the detection moiety can be configured to generate or change a signal indicative of a presence of the target nucleic acid.

Also provided herein in some embodiments, is a system for multiplexed detection of a plurality of comprising a detection region. The detection region can comprise a plurality of detection locations, each detection location of the plurality of detection locations comprising: i. a guide nucleic complementary to one of the plurality of target nucleic acids, or a portion thereof; and ii. a reporter immobilized to a surface of the detection region, the reporter comprising a nucleic acid and a detection moiety, wherein a. the nucleic acid is at least 40 nucleotides in length; b. the nucleic acid comprises a double-stranded region; c. or a combination thereof. At each detection location, cleavage of the reporter by a programmable nuclease activated upon hybridization to the one of the plurality of target nucleic acids can release the detection moiety from the nucleic acid. The release of the detection moiety can be configured to change or generate a signal at the detection location. The signal can indicate a presence or absence of the one of the plurality of target nucleic acids at the detection location.

Also provided herein in some embodiments, is a method for detecting a target nucleic acid comprising a. transferring the plurality of target nucleic acid can comprise i. a programmable nuclease; ii. a guide nucleic complementary to one of the plurality of target nucleic acids, or a portion thereof; and iii. a reporter immobilized to a surface of a detection region. The reporter can comprise a nucleic acid and a detection moiety, wherein 1. the nucleic acid is at least 40 nucleotides in length; 2. the nucleic acid comprises a double-stranded region; 3, or a combination thereof, and b. detecting, at each detection location, a signal indicative of a presence or absence of one of the plurality of target nucleic acids. At each detection location, the programmable nuclease can be activated by binding of the one of the plurality of target nucleic acids to the guide nucleic acid. Activation of the programmable nuclease can cleave the reporter, thereby releasing the detection moiety from the nucleic acid and changing or generating the signal indicative of a presence or absence of the one of the plurality of target nucleic acids.

Also provided herein in some embodiments, is a method for detecting a plurality of target nucleic acids comprising the steps of: a. transferring the plurality of target nucleic acids to the detection region comprising a plurality of detection locations, each detection location comprising i. a programmable nuclease; ii. a guide nucleic complementary to one of the plurality of target nucleic acids, or a portion thereof; iii. a reporter immobilized to a surface of a detection region, the reporter comprising a nucleic acid and a detection moiety, wherein 1. the nucleic acid is at least 40 nucleotides in length; 2. the nucleic acid comprises a double-stranded region; 3, or a combination thereof, and b. detecting, at each detection location, a signal indicative of a presence or absence of one of the plurality of target nucleic acids. At each detection location, the programmable nuclease can be activated by binding of the one of the plurality of target nucleic acids to the guide nucleic acid. Activation of the programmable nuclease can cleave the reporter, thereby releasing the detection moiety from the nucleic acid and generating the signal indicative of a presence or absence of the one of the plurality of target nucleic acids.

Provided herein in some embodiments, is a composition comprising a nucleic acid molecule comprising a nucleic acid sequence at least 80% identical to any one of the sequences set forth in Table 1.

### I. Immobilized Reporter Systems

Disclosed herein are immobilized reporter systems, compositions, and methods of use thereof, e.g., for detection of a target nucleic acid or a plurality of target nucleic acids.

In some instances, systems comprise a Type V CRISPR/Cas protein and a reporter configured to undergo transcollateral cleavage by the Type V CRISPR/Cas protein. In some instances, systems comprise a Type VI CRISPR/Cas protein and a reporter configured to undergo transcollateral cleavage by the Type VI CRISPR/Cas protein. Transcollateral cleavage of the reporter may generate a signal from the reporter, alter a signal from the reporter, or trigger a downstream reaction capable of generating or changing a signal in response to cleavage of the reporter and release of a detection moiety therefrom. In some cases, the signal is an optical signal, such as a fluorescence signal or absorbance signal. Transcollateral cleavage of the reporter may alter the wavelength, intensity, and/or polarization of the optical signal. For example, the reporter may comprise a fluorophore and a quencher, such that transcollateral cleavage of the reporter separates the fluorophore and the quencher thereby increasing a fluorescence signal from the fluorophore. In some embodiments described herein is a method of assaying for a target nucleic acid in a sample comprising contacting the target nucleic acid with a programmable nuclease, a non-naturally occurring guide nucleic acid that hybridizes to a segment of the target nucleic acid, and a reporter, and assaying for a change in a signal, wherein the change in the signal is produced by cleavage of the reporter.

Also provided herein are systems for detecting a target nucleic acid in a sample which comprise an immobilized reporter system. In some cases, the system comprises one or more units. In some instances, one or more units comprise a plurality of different units. In some instances, the one or more units comprise: (a) a non-naturally occurring guide nucleic acid immobilized to a surface by a linkage; and (b) a plurality of reporters immobilized to the surface in proximity to the non-naturally occurring guide nucleic acid. In some cases, the non-naturally occurring guide nucleic acid comprises (i) one or more stabilized nucleotides, (ii) a repeat region comprising a first end and a second end, and (iii) a spacer region that hybridizes to a segment of the target nucleic acid or an amplicon thereof. In some instances, the system comprises a plurality of different units, and each of the different units comprises a non-naturally occurring guide nucleic acid comprising a spacer region with a different sequence.

In some cases, the one or more stabilized nucleotides comprise a plurality of stabilized nucleotides. In some cases, the one or more stabilized nucleotides lack a 2′—OH group. A variety of suitable nucleotides lacking a 2′-OH are available, including natural (e.g., DNA) and artificial (e.g., 2′-deoxy-2′-fluoro) nucleotides. In some cases, the one or more stabilized nucleotides comprise DNA nucleotides, modified DNA nucleotides, modified RNA nucleotides, or a mixture thereof. In some cases, one or more stabilized nucleotides comprise a synthetic nucleotide. In some cases, one or more stabilized nucleotides comprise a peptide nucleic acid (PNA), a morpholino and locked nucleic acid (LNA), a glycol nucleic acid (GNA), a threose nucleic acid (TNA), a hexitol nucleic acids (HNA), 2′-methoxyethoxy (MOE) nucleotide, a 2′-methyl-thio-ethyl nucleotide, a 2′-deoxy-2′-fluoro nucleotide, a 2′-deoxy-2′-chloro nucleotide, a 2′-azido nucleotide, a 2′-O-methyl nucleotide, or any combination thereof. In some cases, the one or more stabilized nucleotides are located within 1, 2, 3, 4, 5, 6, 7, 8, 9, 10, 11, 12, 13, 14, 15, 16, 17, 18, 19, or 20 nucleotides of a terminal hairpin nucleotide in the first end of a repeat region. In some cases, the one or more stabilized nucleotides are located within 10 nucleotides of a terminal hairpin nucleotide in the first end of a repeat region. In some cases, the one or more stabilized nucleotides are located within: 1 to 10 nucleotides; 2 to 8 nucleotides; 2 to 7 nucleotides; 2 to 6 nucleotides; 2 to 5 nucleotides; 2 to 4 nucleotides; or 2 to 3 nucleotides of a terminal hairpin nucleotide. In some embodiments, the stabilized nucleotides are located within 2 to 6 nucleotides of the terminal hairpin nucleotide. In some cases, the one or more stabilized nucleotides can be within the repeat region. In some cases, the one or more stabilized nucleotides is not within the repeat region. In some embodiments, the one or more stabilized nucleotides comprise 1, 2, 3, 4, 5, 6, 7, 8, 9, 10, 15, or more contiguous nucleotides. In some embodiments, the one or more stabilized nucleotides comprise 1-15 stabilized nucleotides, 2-10 stabilized nucleotides, or 3-6 stabilized nucleotides. In some embodiments, the one or more stabilized nucleotides comprises at least two contiguous nucleotides. In some cases, the one or more stabilized nucleotides are effective to reduce a rate of cleavage of the non-naturally occurring guide nucleic acid by the programmable nuclease, relative to that for a corresponding non-naturally occurring guide nucleic acid that consists of RNA.

In some embodiments, the non-naturally occurring guide nucleic acid is immobilized to a surface by a linkage. In some cases, the linkage can comprise a tether. In some cases, the linkage comprises a tether having a length at least that of a: 5, 10, 15, 20, 25, 30, 40, 50, 60, 70, 80, 90, 100, 110, 120, 130, 140, 150, 160, 170, 180, 190, or 200 nucleotide DNA molecule. In some cases, the linkage comprises a tether having a length at least that of a 10-nucleotide DNA molecule. In some cases, the tether has a length at least that of a DNA molecule that is 5 to 200, 10 to 150, 15 to 100, or 20 to 50 nucleotides in length. In some embodiments, the tether has a length at least that of a DNA molecule that is 10 to 100 nucleotides in length. In some embodiments, the tether has a length at least that of a DNA molecule that is at least 20 nucleotides in length. It is understood that a tether having a specified length of a DNA molecule of a certain number of nucleotides does not need to be or even comprise DNA. Rather, the specified DNA length may be used as a measure of length for the tether, while the tether need not itself comprise any DNA. In some cases, the tether comprises a hydrocarbon chain. In some cases, the tether comprises a tether polynucleotide. In some instances, a tether polynucleotide is 10 to 200 nucleotides in length, 10 to 150 nucleotides in length, 10 to 125 nucleotides in length, 10 to 100 nucleotides in length, 10 to 50 nucleotides in length, 12 to 60 nucleotides in length, or 15 to 20 nucleotides in length. In some cases, a tether polynucleotide is about or at least about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the tether polynucleotide is at least 10-100 nucleotides in length. In some embodiments, the tether polynucleotide is at least 10 nucleotides in length. In some embodiments, the tether polynucleotide is at least 20 nucleotides in length. In some cases, a polynucleotide tether is single stranded. In some cases, a polynucleotide tether is double stranded. In some cases, a polynucleotide tether comprises DNA.

In some embodiments, a repeat region comprises a first end and a second end. In some cases, the first end of the repeat region is joined to the linkage, and the second end of the repeat region is joined to the spacer region. In some cases, the terminal hairpin nucleotide of the first end of the repeat region is a 5′ terminal hairpin nucleotide located 3′ relative to a stabilized nucleotide. In some cases, the terminal hairpin nucleotide is a terminal nucleotide within a sequence of nucleotides that is complementary to a sequence of nucleotides in the second end of the repeat sequence and proximal to the linkage. In general, a “terminal hairpin nucleotide” in a repeat region of a non-naturally occurring guide nucleic acid is the last nucleotide of a hairpin structure within the repeat region that is closest to the immobilized end of the guide nucleic acid. The term “terminal hairpin nucleotide” does not require that the nucleotide is the terminal nucleotide of the guide nucleic acid. Rather, the terminal hairpin nucleotide is terminal with respect to the hairpin structure, is distal relative to the loop of the hairpin, and contributes to the secondary structure thereof by internal complementarity. Those skilled in the art are capable of determining whether a given nucleotide forms part of the hairpin structure, particularly for known repeat sequences. In some embodiments, nucleotides participating in a hairpin structure of a repeat sequence are identified using mFold with default parameters (as described by Zuker, Nucleic Acids Res. 31 (13), 3406-15, (2003)).

In some embodiments, a system comprises a plurality of reporters immobilized to the surface in proximity to a non-naturally occurring guide nucleic acid. The reporter may be any of a variety of reporters described herein, including various components thereof, such as with respect to any of the various aspects and embodiments disclosed herein. In some cases, the reporters are immobilized to the surface by attachment to the non-naturally occurring guide nucleic acid, the linkage, or both. In some instances, at least one of the reporters is immobilized to the surface by attachment to an end of the non-naturally occurring guide nucleic acid that is distal to the linkage. For example, at least one of the reporters can be immobilized by attaching to the 3′ end of the spacer region. In some cases, at least one of the reporters is immobilized to the surface by attachment to the linkage. For example, at least one of the reporters can be immobilized by attaching to a tether. In some embodiments, a plurality of reporters (e.g., at least 2, 3, 4, 5, 10, 15, or more) are attached to the linkage. In some embodiments, reporters are attached to both the linkage and an end of the guide nucleic acid. In some cases, a reporter comprises a fluorophore and a quencher. In some instances, cleavage of a reporter is effective to produce a detectable loss of the quencher. In some cases, a reporter comprises a detection moiety. In some cases, cleavage of a reporter is effective to produce a detectable loss of the detection moiety. In some cases, a detection moiety comprises a fluorophore.

In some embodiments, the non-naturally occurring guide nucleic acid is effective to form a complex with a programmable nuclease that is activated upon binding to the target nucleic acid or amplicon thereof. The programmable nuclease may be any of a variety of programmable nucleases described herein, such as with respect to any of the various aspects and embodiments disclosed herein. In some cases, the programmable nuclease is complexed with the non-naturally occurring guide nucleic acid. In some cases, the formation of the activated complex is effective to induce detectable transcollateral cleavage of the reporters. In some cases, the programmable nuclease comprises a RuvC catalytic domain. In some cases, the programmable nuclease is a type V CRISPR/Cas effector protein. In some instances, the type V CRISPR/Cas effector protein is a Cas 12 protein, a Cas14 protein, or a CasΦ protein. In some instances, the programmable nuclease is a Cas12a protein. In some instances, the type V CRISPR/Cas effector protein is a Cas 12 protein comprising a Cas12a, a Cas12b, a Cas12c, a Cas12d, a Cas12e, a Cas12f, a Cas12g, a Cas12h, a Cas12i, a Cas12j, or a Cas12k. In some instances, the programmable nuclease comprises an amino acid sequence that is at least 70%, 75%, 80%, 85%, 90%, 95%, or 100% identical to SEQ ID NO: 43. In some instances, the type V CRISPR/Cas effector protein comprises an amino acid sequence that is at least 70%, 75%, 80%, 85%, 90%, 95%, or 100% identical to SEQ ID NO: 43. In some cases, the programmable nuclease is a type VI CRISPR/Cas effector protein. In some instances, the type VI CRISPR/Cas effector protein is a Cas13 protein. In some cases the Cas 13 protein comprises a a Cas13a, a Cas13b, a Cas13c, a Cas13d, a Cas13e, or a Cas13f.

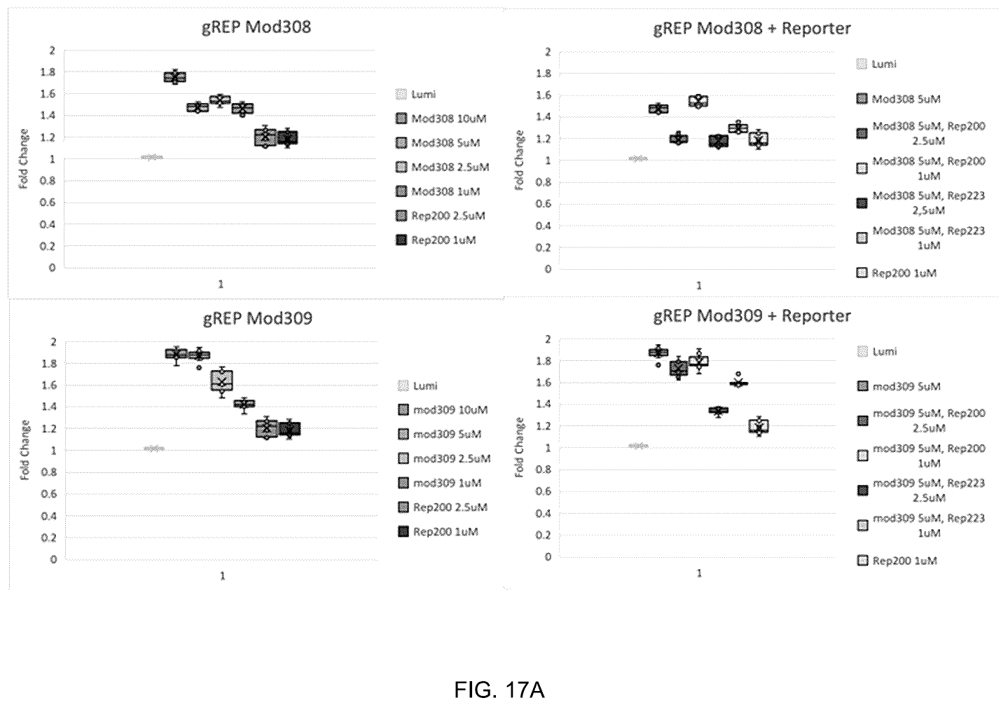

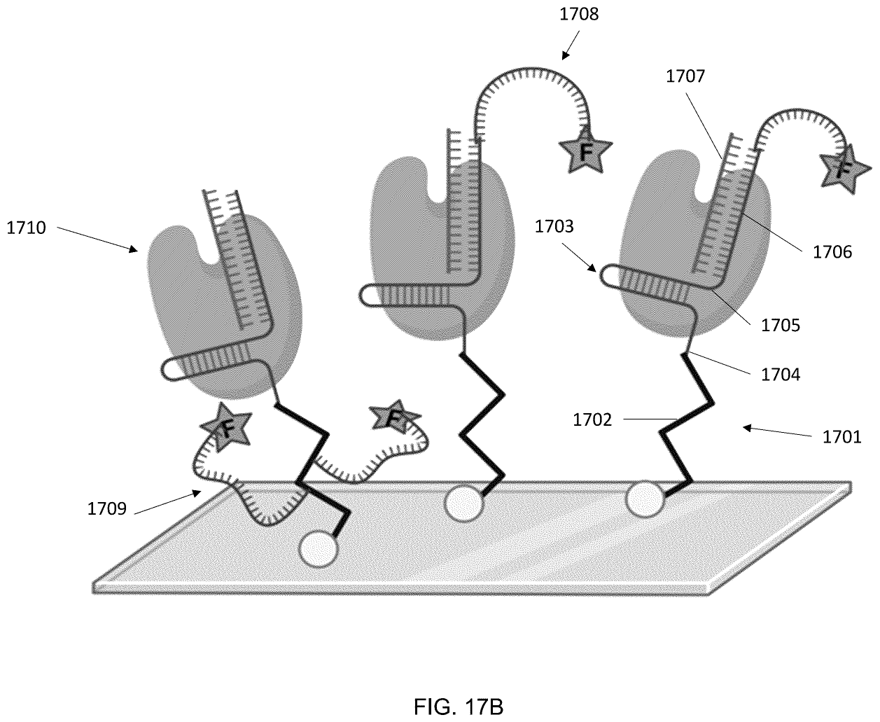

FIG. 17B provides an illustration of exemplary immobilized guide nucleic acid reporters (gREPs) in accordance with some embodiments, and complexed with a programmable nuclease and target nucleic acid. In particular, FIG. 17B illustrates a linkage (1701) comprising a tether (1702). Each illustrated tether (1702) is attached to a guide nucleic acid comprising a repeat region (1703) and a spacer region (1706). The repeat region (1703) includes a first end (1704) joined to the linkage (1701). The repeat region (1703) also includes a second end (1705) joined to the spacer region (1706). Also shown are reporters, including a reporter (1708) which is joined to the spacer region (1706) of a guide nucleic acid, and a reporter (1709) that is joined to a linkage (1701). The guide nucleic acids form complexes with a programmable nuclease (1710) and a target nucleic acid (1707) as described herein.

Also provided herein are methods of assaying for one or more target nucleic acids in a sample. In some cases, a method comprises: contacting a system described herein with a sample suspected of comprising a target nucleic acid(s); cleaving the reporters in response to presence of the target nucleic acid(s) or amplicon(s) thereof; and detecting a change in signal resulting from cleavage of the reporters. In some cases, the detection identifies the target nucleic acid in the sample. In some cases, the method further comprises amplifying one or more of the target nucleic acids before or during said contacting.

### A. Reporters

Reporter systems disclosed herein may comprise one or more reporters. Described herein are compositions and methods of use thereof comprising one or more reporter molecules. In some examples, the one or more reporter molecules comprise one or more different reporter molecules. In an example, the one or more reporter molecules comprise a first reporter molecule, a second reporter molecule, a third reporter molecule, and/or more reporter molecules or a plurality of each reporter molecule wherein each reporter molecule can be present in multiple copies (e.g., at a predefined concentration) in the composition. In some examples, the compositions and methods comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more reporter molecules or sequences.

By way of non-limiting and illustrative example, a reporter may comprise a single stranded nucleic acid and a detection moiety (e.g., a labeled single stranded RNA reporter), wherein the nucleic acid is capable of being cleaved by a programmable nuclease (e.g., a Type V or Type VI CRISPR/Cas protein as disclosed herein) or a multimeric complex thereof, releasing the detection moiety, and, generating a detectable signal. In some instances, the reporter additionally comprises a double stranded nucleic acid. As used herein, “reporter” is used interchangeably with “reporter molecule”. The programmable nucleases disclosed herein, activated upon hybridization of a guide RNA to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

### Reporter Design

In some instances, a reporter may comprise a nucleic acid. In some cases, the nucleic acid may have a polynucleotide sequence. In some cases, the polynucleotide sequence may comprise about 10 nucleotides. In some cases, the polynucleotide sequence may comprise about 11 nucleotides. In some cases, the polynucleotide sequence may comprise about 12 nucleotides. In some cases, the polynucleotide sequence may comprise about 13 nucleotides. In some cases, the polynucleotide sequence may comprise about 14 nucleotides. In some cases, the polynucleotide sequence may comprise about 15 nucleotides. In some cases, the polynucleotide sequence may comprise about 16 nucleotides. In some cases, the polynucleotide sequence may comprise about 17 nucleotides. In some cases, the polynucleotide sequence may comprise about 18 nucleotides. In some cases, the polynucleotide sequence may comprise about 19 nucleotides. In some cases, the polynucleotide sequence may comprise about 20 nucleotides. In some cases, the polynucleotide sequence may comprise about 21 nucleotides. In some cases, the polynucleotide sequence may comprise about 22 nucleotides. In some cases, the polynucleotide sequence may comprise about 23 nucleotides. In some cases, the polynucleotide sequence may comprise about 24 nucleotides. In some cases, the polynucleotide sequence may comprise about 25 nucleotides. In some cases, the polynucleotide sequence may comprise about 26 nucleotides. In some cases, the polynucleotide sequence may comprise about 27 nucleotides. In some cases, the polynucleotide sequence may comprise about 28 nucleotides. In some cases, the polynucleotide sequence may comprise about 29 nucleotides. In some cases, the polynucleotide sequence may comprise about 30 nucleotides. In some cases, the polynucleotide sequence may comprise about 31 nucleotides. In some cases, the polynucleotide sequence may comprise about 32 nucleotides. In some cases, the polynucleotide sequence may comprise about 33 nucleotides. In some cases, the polynucleotide sequence may comprise about 34 nucleotides. In some cases, the polynucleotide sequence may comprise about 35 nucleotides. In some cases, the polynucleotide sequence may comprise about 36 nucleotides. In some cases, the polynucleotide sequence may comprise about 37 nucleotides. In some cases, the polynucleotide sequence may comprise about 38 nucleotides. In some cases, the polynucleotide sequence may comprise about 39 nucleotides. In some cases, the polynucleotide sequence may comprise about 40 nucleotides. In some cases, the polynucleotide sequence may comprise about 41 nucleotides. In some cases, the polynucleotide sequence may comprise about 42 nucleotides. In some cases, the polynucleotide sequence may comprise about 43 nucleotides. In some cases, the polynucleotide sequence may comprise about 44 nucleotides. In some cases, the polynucleotide sequence may comprise about 45 nucleotides. In some cases, the polynucleotide sequence may comprise about 46 nucleotides. In some cases, the polynucleotide sequence may comprise about 47 nucleotides. In some cases, the polynucleotide sequence may comprise about 48 nucleotides. In some cases, the polynucleotide sequence may comprise about 49 nucleotides. In some cases, the polynucleotide sequence may comprise about 50 nucleotides. In some cases, the polynucleotide sequence may comprise about 51 nucleotides. In some cases, the polynucleotide sequence may comprise about 52 nucleotides. In some cases, the polynucleotide sequence may comprise about 53 nucleotides. In some cases, the polynucleotide sequence may comprise about 54 nucleotides. In some cases, the polynucleotide sequence may comprise about 55 nucleotides. In some cases, the polynucleotide sequence may comprise about 56 nucleotides. In some cases, the polynucleotide sequence may comprise about 57 nucleotides. In some cases, the polynucleotide sequence may comprise about 58 nucleotides. In some cases, the polynucleotide sequence may comprise about 59 nucleotides. In some cases, the polynucleotide sequence may comprise about 60 nucleotides. In some cases, the polynucleotide sequence may comprise about 61 nucleotides. In some cases, the polynucleotide sequence may comprise about 62 nucleotides. In some cases, the polynucleotide sequence may comprise about 63 nucleotides. In some cases, the polynucleotide sequence may comprise about 64 nucleotides. In some cases, the polynucleotide sequence may comprise about 65 nucleotides. In some cases, the polynucleotide sequence may comprise about 66 nucleotides. In some cases, the polynucleotide sequence may comprise about 67 nucleotides. In some cases, the polynucleotide sequence may comprise about 68 nucleotides. In some cases, the polynucleotide sequence may comprise about 69 nucleotides. In some cases, the polynucleotide sequence may comprise about 70 nucleotides. In some cases, the polynucleotide sequence may comprise about 71 nucleotides. In some cases, the polynucleotide sequence may comprise about 72 nucleotides. In some cases, the polynucleotide sequence may comprise about 73 nucleotides. In some cases, the polynucleotide sequence may comprise about 74 nucleotides. In some cases, the polynucleotide sequence may comprise about 75 nucleotides. In some cases, the polynucleotide sequence may comprise about 76 nucleotides. In some cases, the polynucleotide sequence may comprise about 77 nucleotides. In some cases, the polynucleotide sequence may comprise about 78 nucleotides. In some cases, the polynucleotide sequence may comprise about 79 nucleotides. In some cases, the polynucleotide sequence may comprise about 80 nucleotides. In some cases, the polynucleotide sequence may comprise about 81 nucleotides. In some cases, the polynucleotide sequence may comprise about 82 nucleotides. In some cases, the polynucleotide sequence may comprise about 83 nucleotides. In some cases, the polynucleotide sequence may comprise about 84 nucleotides. In some cases, the polynucleotide sequence may comprise about 85 nucleotides. In some cases, the polynucleotide sequence may comprise about 86 nucleotides. In some cases, the polynucleotide sequence may comprise about 87 nucleotides. In some cases, the polynucleotide sequence may comprise about 88 nucleotides. In some cases, the polynucleotide sequence may comprise about 89 nucleotides. In some cases, the polynucleotide sequence may comprise about 90 nucleotides. In some cases, the polynucleotide sequence may comprise about 91 nucleotides. In some cases, the polynucleotide sequence may comprise about 92 nucleotides. In some cases, the polynucleotide sequence may comprise about 93 nucleotides. In some cases, the polynucleotide sequence may comprise about 94 nucleotides. In some cases, the polynucleotide sequence may comprise about 95 nucleotides. In some cases, the polynucleotide sequence may comprise about 96 nucleotides. In some cases, the polynucleotide sequence may comprise about 97 nucleotides. In some cases, the polynucleotide sequence may comprise about 98 nucleotides. In some cases, the polynucleotide sequence may comprise about 99 nucleotides. In some cases, the polynucleotide sequence may comprise about 100 nucleotides. In some cases, the polynucleotide sequence may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotides. In some cases, the polynucleotide sequence may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotides. For cleavage by a programmable nuclease comprising Cas13, a reporter can comprise any numbers of nucleotides described thereof. In some cases, a reporter can be 5, 8, or 10 nucleotides in length. For cleavage by a programmable nuclease comprising Cas12, a reporter can comprise any numbers of nucleotides described thereof. In some cases, a reporter can be about 10 nucleotides in length.

Reporters may comprise RNA. Reporters may comprise DNA. Reporters may also comprise both DNA and RNA. Reporters may be double-stranded. Reporters may be single-stranded. A reporter may comprise a single-stranded region. A reporter may comprise a double-stranded region. In some cases, reporters may comprise both single-stranded and doubles-stranded regions. In some instances, cleavage of the reporter produces, changes, or reduces a signal and thereby indicate the presence of the target nucleic acid in the sample. The systems and devices disclosed herein can be used to detect these signals, which can indicate whether a target nucleic acid is present in the sample.

The reporter can comprise a single-stranded nucleic acid sequence comprising at least one deoxyribonucleotide and at least one ribonucleotide. The reporter can comprise a double-stranded nucleic acid sequence comprising at least one deoxyribonucleotide and at least one ribonucleotide. The reporter can comprise a single-stranded nucleic acid sequence and a double-stranded nucleic acid region, each comprising at least one deoxyribonucleotide and at least one ribonucleotide.

In some instances, the single-stranded region of a reporter may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotides. In some cases, the single-stranded region of the reporter may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotides. In some cases, the single-stranded region may comprise about 5 to about 15 nucleotides. In some cases, the single-stranded region may comprise about 5 to about 20 nucleotides. In some cases, the single-stranded region may comprise about 5 to about 25 nucleotides. In some cases, the single-stranded region may comprise about 5 to about 50 nucleotides. In some cases, the single-stranded region may comprise about 5 to about 100 nucleotides. In some cases, the single-stranded region may comprise about 5 to about 200 nucleotides. In some cases, the single-stranded region may comprise about 5 to about 500 nucleotides. In some cases, the single-stranded region may comprise about 4 to about 15 nucleotides. In some cases, the single-stranded region may comprise about 3 to about 15 nucleotides. In some cases, the single-stranded region may comprise about 2 to about 15 nucleotides. In some cases, the single-stranded region may comprise about 1 to about 15 nucleotides. In some cases, the single-stranded region may comprise about 1 nucleotides. In some cases, the single-stranded region may comprise about 2 nucleotides. In some cases, the single-stranded region may comprise about 3 nucleotides. In some cases, the single-stranded region may comprise about 4 nucleotides. In some cases, the single-stranded region may comprise about 5 nucleotides. In some cases, the single-stranded region may comprise about 6 nucleotides. In some cases, the single-stranded region may comprise about 7 nucleotides. In some cases, the single-stranded region may comprise about 8 nucleotides. In some cases, the single-stranded region may comprise about 9 nucleotides. In some cases, the single-stranded region may comprise about 10 nucleotides. In some cases, the single-stranded region may comprise about 11 nucleotides. In some cases, the single-stranded region may comprise about 12 nucleotides. In some cases, the single-stranded region may comprise about 13 nucleotides. In some cases, the single-stranded region may comprise about 14 nucleotides. In some cases, the single-stranded region may comprise about 15 nucleotides. In some cases, the single-stranded region may comprise about 20 nucleotides. In some cases, the single-stranded region may comprise about 30 nucleotides. In some cases, the single-stranded region may comprise about 40 nucleotides. In some cases, the single-stranded region may comprise about 50 nucleotides. In some cases, the single-stranded region may comprise about 100 nucleotides. In some cases, the single-stranded region may comprise about 150 nucleotides. In some cases, the single-stranded region may comprise about 200 nucleotides. In some cases, the single-stranded region may comprise about 500 nucleotides.

In some instances, the double-stranded region of a reporter may comprise at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, 150, 200, 250, 300, 350, 400, 450, 500 or more nucleotide pairs. In some instances, the double-stranded region of a reporter may comprise at most about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, 150, 200, 250, 300, 350, 400, 450, 500 or more nucleotide pairs.

In some cases, the double-stranded region may comprise a length of about 10 nucleotides. In some cases, the double-stranded region may comprise a length of about 15 nucleotides. In some cases, the double-stranded region may comprise a length of about 20 nucleotides. In some cases, the double-stranded region may comprise a length of about 25 nucleotides. In some cases, the double-stranded region may comprise a length of about 30 nucleotides. In some cases, the double-stranded region may comprise a length of about 35 nucleotides. In some cases, the double-stranded region may comprise a length of about 40 nucleotides. In some cases, the double-stranded region may comprise a length of about 45 nucleotides. In some cases, the double-stranded region may comprise a length of about 50 nucleotides. In some cases, the double-stranded region may comprise a length of about 55 nucleotides. In some cases, the double-stranded region may comprise a length of about 60 nucleotides. In some cases, the double-stranded region may comprise a length of about 65 nucleotides. In some cases, the double-stranded region may comprise a length of about 70 nucleotides. In some cases, the double-stranded region may comprise a length of about 75 nucleotides. In some cases, the double-stranded region may comprise a length of about 80 nucleotides. In some cases, the double-stranded region may comprise a length of about 85 nucleotides. In some cases, the double-stranded region may comprise a length of about 90 nucleotides. In some cases, the double-stranded region may comprise a length of about 95 nucleotides. In some cases, the double-stranded region may comprise a length of about 100 nucleotides. In some cases, the double-stranded region may comprise a length of about 150 nucleotides. In some cases, the double-stranded region may comprise a length of about 200 nucleotides. In some cases, the double-stranded region may comprise a length of about 300 nucleotides. In some cases, the double-stranded region may comprise a length of about 400 nucleotides. In some cases, the double-stranded region may comprise a length of about 500 nucleotides.

In some instances, the double-stranded region of a reporter may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, 500 or more nucleotide pairs. In some cases, the double-stranded region of the reporter may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 150, 200, 250, 300, 350, 400, 450, or 500 nucleotide pairs. In some cases, the double-stranded region may comprise a length of about 45 to about 55 nucleotides. In some cases, the double-stranded region may comprise a length of about 40 to about 55 nucleotides. In some cases, the double-stranded region may comprise a length of about 35 to about 55 nucleotides. In some cases, the double-stranded region may comprise a length of about 30 to about 55 nucleotides. In some cases, the double-stranded region may comprise a length of about 25 to about 55 nucleotides. In some cases, the double-stranded region may comprise a length of about 20 to about 55 nucleotides. In some cases, the double-stranded region may comprise a length of about 15 to about 55 nucleotides. In some cases, the double-stranded region may comprise a length of about 10 to about 55 nucleotides. In some cases, the double-stranded region may comprise a length of about 45 to about 60 nucleotides. In some cases, the double-stranded region may comprise a length of about 45 to about 70 nucleotides. In some cases, the double-stranded region may comprise a length of about 45 to about 80 nucleotides. In some cases, the double-stranded region may comprise a length of about 45 to about 90 nucleotides. In some cases, the double-stranded region may comprise a length of about 45 to about 100 nucleotides. In some cases, the double-stranded region may comprise a length of about 45 to about 200 nucleotides. In some cases, the double-stranded region may comprise a length of about 45 to about 500 nucleotides. In some cases, the double-stranded region may comprise a length of about 5 to about 15 nucleotides. In some cases, the double-stranded region may comprise a length of about 5 to about 20 nucleotides. In some cases, the double-stranded region may comprise a length of about 5 to about 25 nucleotides. In some cases, the double-stranded region may comprise a length of about 5 to about 50 nucleotides. In some cases, the double-stranded region may comprise a length of about 5 to about 100 nucleotides. In some cases, the double-stranded region may comprise a length of about 5 to about 200 nucleotides. In some cases, the double-stranded region may comprise a length of about 5 to about 500 nucleotides. In some cases, the double-stranded region may comprise a length of about 4 to about 15 nucleotides. In some cases, the double-stranded region may comprise a length of about 3 to about 15 nucleotides. In some cases, the double-stranded region may comprise a length of about 2 to about 15 nucleotides. In some cases, the double-stranded region may comprise a length of about 1 to about 15 nucleotides.

In some cases, the single-stranded region may comprise about 1 nucleotide, and the double-stranded region may comprise about 35 nucleotides. In some cases, the single-stranded region may comprise about 1 nucleotide, and the double-stranded region may comprise about 40 nucleotides. In some cases, the single-stranded region may comprise about 1 nucleotide, and the double-stranded region may comprise about 45 nucleotides. In some cases, the single-stranded region may comprise about 1 nucleotide, and the double-stranded region may comprise about 50 nucleotides. In some cases, the single-stranded region may comprise about 1 nucleotide, and the double-stranded region may comprise about 55 nucleotides. In some cases, the single-stranded region may comprise about 1 nucleotide, and the double-stranded region may comprise about 60 nucleotides. In some cases, the single-stranded region may comprise about 1 nucleotide, and the double-stranded region may comprise about 65 nucleotides. In some cases, the single-stranded region may comprise about 1 nucleotide, and the double-stranded region may comprise about 35 nucleotides. In some cases, the single-stranded region may comprise about 1 nucleotide, and the double-stranded region may comprise about 40 nucleotides. In some cases, the single-stranded region may comprise about 5 nucleotides, and the double-stranded region may comprise about 45 nucleotides. In some cases, the single-stranded region may comprise about 5 nucleotides, and the double-stranded region may comprise about 50 nucleotides. In some cases, the single-stranded region may comprise about 5 nucleotides, and the double-stranded region may comprise about 55 nucleotides. In some cases, the single-stranded region may comprise about 5 nucleotides, and the double-stranded region may comprise about 60 nucleotides. In some cases, the single-stranded region may comprise about 5 nucleotides, and the double-stranded region may comprise about 65 nucleotides. In some cases, the single-stranded region may comprise about 9 nucleotides, and the double-stranded region may comprise about 35 nucleotides. In some cases, the single-stranded region may comprise about 9 nucleotides, and the double-stranded region may comprise about 40 nucleotides. In some cases, the single-stranded region may comprise about 9 nucleotides, and the double-stranded region may comprise about 45 nucleotides. In some cases, the single-stranded region may comprise about 9 nucleotides, and the double-stranded region may comprise about 50 nucleotides. In some cases, the single-stranded region may comprise about 9 nucleotides, and the double-stranded region may comprise about 55 nucleotides. In some cases, the single-stranded region may comprise about 9 nucleotides, and the double-stranded region may comprise about 60 nucleotides. In some cases, the single-stranded region may comprise about 9 nucleotides, and the double-stranded region may comprise about 65 nucleotides. In some cases, the single-stranded region may comprise about 15 nucleotides, and the double-stranded region may comprise about 35 nucleotides. In some cases, the single-stranded region may comprise about 15 nucleotides, and the double-stranded region may comprise about 40 nucleotides. In some cases, the single-stranded region may comprise about 15 nucleotides, and the double-stranded region may comprise about 45 nucleotides. In some cases, the single-stranded region may comprise about 15 nucleotides, and the double-stranded region may comprise about 50 nucleotides. In some cases, the single-stranded region may comprise about 15 nucleotides, and the double-stranded region may comprise about 55 nucleotides. In some cases, the single-stranded region may comprise about 15 nucleotides, and the double-stranded region may comprise about 60 nucleotides. In some cases, the single-stranded region may comprise about 15 nucleotides, and the double-stranded region may comprise about 65 nucleotides. In some cases, the single-stranded region may comprise about 20 nucleotides, and the double-stranded region may comprise about 35 nucleotides. In some cases, the single-stranded region may comprise about 20 nucleotides, and the double-stranded region may comprise about 40 nucleotides. In some cases, the single-stranded region may comprise about 20 nucleotides, and the double-stranded region may comprise about 45 nucleotides. In some cases, the single-stranded region may comprise about 20 nucleotides, and the double-stranded region may comprise about 50 nucleotides. In some cases, the single-stranded region may comprise about 20 nucleotides, and the double-stranded region may comprise about 55 nucleotides. In some cases, the single-stranded region may comprise about 20 nucleotides, and the double-stranded region may comprise about 60 nucleotides. In some cases, the single-stranded region may comprise about 20 nucleotides, and the double-stranded region may comprise about 65 nucleotides.

In some cases, the reporter comprises a nucleic acid comprising at least one ribonucleotide residue at an internal position that functions as a cleavage site. In some cases, the reporter comprises at least 2, 3, 4, 5, 6, 7, 8, 9, or 10 ribonucleotide residues at an internal position. In some cases, the reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter comprises a nucleic acid comprising at least one deoxyribonucleotide residue at an internal position that functions as a cleavage site. In some cases, the reporter comprises at least 2, 3, 4, 5, 6, 7, 8, 9, or 10 deoxyribonucleotide residues at an internal position. In some cases, the reporter comprises from 2 to 10, from 3 to 9, from 4 to 8, or from 5 to 7 deoxyribonucleotide residues at an internal position. Sometimes the deoxyribonucleotide residues are continuous. Alternatively, the deoxyribonucleotide residues may be interspersed in between non-deoxyribonucleotide residues. In some cases, the reporter has only deoxyribonucleotide residues.

In some cases, the reporter has only deoxyribonucleotide residues. In some cases, the reporter comprises nucleotides resistant to cleavage by the programmable nuclease described herein. In some cases, the reporter comprises synthetic nucleotides. In some cases, the reporter comprises at least one ribonucleotide residue and at least one non-ribonucleotide residue. In some cases, the reporter comprises at least one uracil ribonucleotide. In some cases, the reporter comprises at least two uracil ribonucleotides. Sometimes the reporter has only uracil ribonucleotides. In some cases, the reporter comprises at least one adenine ribonucleotide. In some cases, the reporter comprises at least two adenine ribonucleotides. In some cases, the reporter has only adenine ribonucleotides. In some cases, the reporter comprises at least one cytosine ribonucleotide. In some cases, the reporter comprises at least two cytosine ribonucleotides. In some cases, the reporter comprises at least one guanine ribonucleotide. In some cases, the reporter comprises at least two guanine ribonucleotides. A reporter can comprise only unmodified ribonucleotides, only unmodified deoxyribonucleotides, or a combination thereof. A reporter can comprise a combination of modified and unmodified ribonucleotides and/or deoxyribonucleotides.

In some examples, a reporter molecule comprises a single stranded nucleic acid comprising a detection moiety, wherein the nucleic acid of the reporter molecule is capable of being cleaved by the activated programmable nuclease, thereby generating a first detectable signal. In some cases, the reporter molecule comprises a single-stranded nucleic acid sequence comprising ribonucleotides. In some cases, the reporter molecule comprises a single-stranded nucleic acid sequence comprising deoxyribonucleotides. In some cases, the reporter molecule comprises a single-stranded nucleic acid sequence comprising deoxyribonucleotides and ribonucleotides. As described herein, nucleic acid sequences can be detected using a programmable RNA nuclease, a programmable DNA nuclease, or a combination thereof, as disclosed herein. The programmable nuclease can be activated and cleave the reporter molecule upon binding of a guide nucleic acid to a target nucleic acid. Additionally, different compositions of reporter molecules can allow for multiplexing using different programmable nucleases (e.g., a programmable RNA nuclease and a programmable DNA nuclease). In some instances, the reporter may comprise any design detailed in TABLE 1 below or described herein.

TABLE 1
Exemplary Reporter Designs

| | | | | Complementary | | | |
|---|---|---|---|---|---|---|---|
| | | | | sequence | | | |
| | | | | to the that | | | |
| | | Sequence | Sequence | underlined of | Immo- | | |
| | Design | #1 (SEQ ID | #2 (SEQ | Sequence #1 | bilization | Fluoro- | |
| Reporter | (5′-3′) | NO) | ID NO) | (SEQ ID NO) | moiety* | phore* | Quencher* |
| Reporter | /5AmMC6/C | CTTACCC | TTTTTTT | GGGTTGTG | /5AmMC6/ | /i6- | /3IABKFQ/ |
| 203 | TTACCCAC | ACCTCTC | TT (SEQ | ATGTGAGT | | FAMK/ | |
| | CTCTCCCC | CCCCAAA | ID NO 6) | TAGTGGTG | | | |
| | CAAAACA | ACAAACA | | TTTGTTTTG | | | |
| | AACACCAC | CCACTAA | | GGGGAGAG | | | |
| | TAACTCAC | CTCACAT | | GTGGGTAA | | | |
| | ATCACAAC | CACAACC | | G (SEQ ID | | | |
| | CC/i6- | C (SEQ ID | | NO 7) | | | |
| | FAMK/TTT | NO 1) | | | | | |
| | TTTTTT/ | | | | | | |
| | 3IABkFQ | | | | | | |
| Reporter | /5AmMC6/C | CTTACCC | n/a | GGGTTGTG | /5AmMC6/ | /36-FAM/ | n/a |
| 204 | TTACCCAC | ACCTCTC | | ATGTGAGT | | | |
| | CTCTCCCC | CCCCAAA | | TAGTGGTG | | | |
| | CAAAACA | ACAAACA | | TTTGTTTTG | | | |
| | AACACCAC | CCACTAA | | GGGGAGAG | | | |
| | TAACTCAC | CTCACAT | | GTGGGTAA | | | |
| | ATCACAAC | CACAACC | | G (SEQ ID | | | |
| | CCTTTTTT | CTTTTTTT | | NO 8) | | | |
| | TTT/36- | TT (SEQ ID | | | | | |
| | FAM/ | NO 2) | | | | | |
| Reporter | /5AmMC6/T | TTTTTTTT | n/a | n/a | /5AmMC6/ | /36-FAM/ | n/a |
| 112 | TTTTTTTTT | TTTT (SEQ | | | | | |
| | TT/36-FAM/ | ID NO 3) | | | | | |
| Reporter | /5AmMC6// | TTTTTTTT | n/a | n/a | /5AmMC6/ | /i6- | /3IABKFQ/ |
| 136 | i6- | TTTTTTTT | | | | FAMK/ | |
| | FAMK/TTT | TTT (SEQ | | | | | |
| | TTTTTTTTT | ID NO 4) | | | | | |
| | TTTTTTT/ | | | | | | |
| | 3IABKFQ/ | | | | | | |
| Reporter | /5AmMC12/ | TTTTTTTT | n/a | n/a | /5AmMC12/ | /36-FAM/ | n/a |
| 200 | TTTTTTTTT | TTTTTTTT | | | | | |
| | TTTTTTTTT | TTTTTTTT | | | | | |
| | TTTTTTTTT | TTTTTTTT | | | | | |
| | TTTTTTTTT | TTTTTTTT | | | | | |
| | TTTTTTTTT | TTTTTTTT | | | | | |
| | TTTTTTTTT | TTTTTTTT | | | | | |
| | TTTTTT/36- | TTTT (SEQ | | | | | |
| | FAM/ | ID NO 5) | | | | | |

/36-FAM/: 3′ 6-Fluorescein (Integrated DNA Technologies)
/3IABKFQ/: 3′ Iowa Black FQ (Integrated DNA Technologies)
/i6-FAMK/: internal 6-Fluorescein (Azide) with dT base (Integrated DNA Technologies)
/5AmMC6/: 5′ amino modifier with 6 carbons (Integrated DNA Technologies)
/5AmMC12/: 5′ amino modifier with 12 carbons (Integrated DNA Technologies)
*This Table refers to the fluorophore, quencher moiety, and immobilization moiety as their tradenames and their source is identified. However, alternatives, generics, or non-tradename moieties with similar function from other sources can also be used.

In some instances, the reporter may comprise a nucleic acid sequence at least about 50%, 55%, 60%, 65%, 70%, 75%, 80%, 85%, 90%, 95%, 96%, 97%, 98%, 99%, or 100% identical to any one of SEQ IDs NO: 1-6. The reporter may comprise a nucleic acid sequence at least about 50% identical to any one of SEQ IDs NO: 1-6. The reporter may comprise a nucleic acid sequence at least about 55% identical to any one of SEQ IDs NO: 1-6. The reporter may comprise a nucleic acid sequence at least about 60% identical to any one of SEQ IDs NO: 1-6. The reporter may comprise a nucleic acid sequence at least about 65% identical to any one of SEQ IDs NO: 1-6. The reporter may comprise a nucleic acid sequence at least about 70% identical to any one of SEQ IDs NO: 1-6. The reporter may comprise a nucleic acid sequence at least about 75% identical to any one of SEQ IDs NO: 1-6. The reporter may comprise a nucleic acid sequence at least about 80% identical to any one of SEQ IDs NO: 1-6. The reporter may comprise a nucleic acid sequence at least about 85% identical to any one of SEQ IDs NO: 1-6. The reporter may comprise a nucleic acid sequence at least about 90% identical to any one of SEQ IDs NO: 1-6. The reporter may comprise a nucleic acid sequence at least about 95% identical to any one of SEQ IDs NO: 1-6. The reporter may comprise a nucleic acid sequence at least about 96% identical to any one of SEQ IDs NO: 1-6. The reporter may comprise a nucleic acid sequence at least about 97% identical to any one of SEQ IDs NO: 1-6. The reporter may comprise a nucleic acid sequence at least about 98% identical to any one of SEQ IDs NO: 1-6. The reporter may comprise a nucleic acid sequence at least about 99% identical to any one of SEQ IDs NO: 1-6. The reporter may comprise a nucleic acid sequence of any one of SEQ IDs NO: 1-6.

In some instances, the reporter may comprise at least two nucleic acid sequences at least about 50%, 55%, 60%, 65%, 70%, 75%, 80%, 85%, 90%, 95%, 96%, 97%, 98%, 99%, or 100% identical to any two of SEQ IDs NO: 1-6. The reporter may comprise at least two nucleic acid sequences at least about 50% identical to any two of SEQ IDs NO: 1-6. The reporter may comprise at least two nucleic acid sequences at least about 55% identical to any two of SEQ IDs NO: 1-6. The reporter may comprise at least two nucleic acid sequences at least about 60% identical to any two of SEQ IDs NO: 1-6. The reporter may comprise at least two nucleic acid sequences at least about 65% identical to any two of SEQ IDs NO: 1-6. The reporter may comprise at least two nucleic acid sequences at least about 70% identical to any two of SEQ IDs NO: 1-6. The reporter may comprise at least two nucleic acid sequences at least about 75% identical to any two of SEQ IDs NO: 1-6. The reporter may comprise at least two nucleic acid sequences at least about 80% identical to any two of SEQ IDs NO: 1-6. The reporter may comprise at least two nucleic acid sequences at least about 85% identical to any two of SEQ IDs NO: 1-6. The reporter may comprise at least two nucleic acid sequences at least about 90% identical to any two of SEQ IDs NO: 1-6. The reporter may comprise at least two nucleic acid sequences at least about 95% identical to any two of SEQ IDs NO: 1-6. The reporter may comprise at least two nucleic acid sequences at least about 96% identical to any two of SEQ IDs NO: 1-6. The reporter may comprise at least two nucleic acid sequences at least about 97% identical to any two of SEQ IDs NO: 1-6. The reporter may comprise at least two nucleic acid sequences at least about 98% identical to any two of SEQ IDs NO: 1-6. The reporter may comprise at least two nucleic acid sequences at least about 99% identical to any two of SEQ IDs NO: 1-6. The reporter may comprise at least two nucleic acid sequences of any two of SEQ IDs NO: 1-6.

In some instances, the reporter may comprise at least two nucleic acid sequences at least about 50%, 55%, 60%, 65%, 70%, 75%, 80%, 85%, 90%, 95%, 96%, 97%, 98%, 99%, or 100% identical to SEQ ID NOs: 1, 2, 7, and 8. The reporter may comprise at least two nucleic acid sequences at least about 50% identical to SEQ ID NOs: 1, 2, 7, and 8. The reporter may comprise at least two nucleic acid sequences at least about 55% identical to SEQ ID NOs: 1, 2, 7, and 8. The reporter may comprise at least two nucleic acid sequences at least about 60% identical to SEQ ID NOs: 1, 2, 7, and 8. The reporter may comprise at least two nucleic acid sequences at least about 65% identical to SEQ ID NOs: 1, 2, 7, and 8. The reporter may comprise at least two nucleic acid sequences at least about 70% identical to SEQ ID NOs: 1, 2, 7, and 8. The reporter may comprise at least two nucleic acid sequences at least about 75% identical to SEQ ID NOs: 1, 2, 7, and 8. The reporter may comprise at least two nucleic acid sequences at least about 80% identical to SEQ ID NOs: 1, 2, 7, and 8. The reporter may comprise at least two nucleic acid sequences at least about 85% identical to SEQ ID NOs: 1, 2, 7, and 8. The reporter may comprise at least two nucleic acid sequences at least about 90% identical to SEQ ID NOs: 1, 2, 7, and 8. The reporter may comprise at least two nucleic acid sequences at least about 95% identical to SEQ ID NOs: 1, 2, 7, and 8. The reporter may comprise at least two nucleic acid sequences at least about 96% identical to SEQ ID NOs: 1, 2, 7, and 8. The reporter may comprise at least two nucleic acid sequences at least about 97% identical to SEQ ID NOs: 1, 2, 7, and 8. The reporter may comprise at least two nucleic acid sequences at least about 98% identical to SEQ ID NOs: 1, 2, 7, and 8. The reporter may comprise at least two nucleic acid sequences at least about 99% identical to SEQ ID NOs: 1, 2, 7, and 8. The reporter may comprise at least two nucleic acid sequences of SEQ ID NOs: 1, 2, 7, and 8.

### Reporter Immobilization

In some instances, a reporter may be immobilized on a substrate. In some cases, a reporter may be immobilized to a surface of the substrate. In some cases, the reporter may be immobilized to a detection location of a substrate. In some instances, the reporter may be immobilized on the substrate. The reporter can be attached to a solid support. The solid support, for example, is a surface. A surface can be an electrode. Sometimes the solid support is a bead. In some cases, the bead is a magnetic bead. The surface can also be an array or a slide.

The reporter comprising a nucleic acid, in some cases, may be immobilized at the 5′end of the nucleic acid. In some cases, the reporter may be immobilized at the 3′ end of the nucleic acid. In some cases, the reporter may be immobilized at the 5′ and 3′ end of the nucleic acid.

In some instances, a reporter may be immobilized to a substrate via covalent bonding. In some cases, the reporter may comprise a thiol or an amine group for immobilization.

In some embodiments, the one or more detection reagents can be immobilized in discrete detection locations using NHS-amine chemistry as described herein. For example, a primary amine-modified guide nucleic acid and a primary amine-modified reporter may be conjugated to an NHS-coated surface of the detection region. In some cases, the amine may form an amide bond with the substrate. For example, the substrate may comprise graphene oxide. NHS-amine, in some cases, may have a structure of

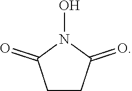

In some cases, the immobilization moiety of a reporter may comprise a thiol group. The thiol group may form an Au—S bond with the substrate. The substrate may comprise gold. In some embodiments, the one or more detection reagents may be immobilized using maleimide-thiol chemistry as described herein. For example, a thiol-modified guide nucleic acid and a thiol-modified reporter may be conjugated to a maleimide-coated surface of the detection region. Thiol group, in some cases, may have a structure of S—H, wherein S is sulfur.

In some cases, a reporter may be immobilized to a substrate via non-covalent bonding. In some embodiments, the one or more detection reagents may be immobilized using avidin/streptavidin-biotin chemistry as described herein. For example, a biotinylated reporter and a biotinylated guide nucleic acid may be immobilized to a streptavidin-coated surface of the detection region.

In some instances, the immobilization of the reporter on a substrate may comprise an immobilization moiety. In some cases, the reporter may comprise an amino group moiety, a peptide moiety, a polypeptide moiety, or a protein moiety. The amino group moiety, peptide moiety, polypeptide moiety, or protein moiety may be the immobilization moiety. The immobilization moiety may comprise an amino modifier. The immobilization moiety may be 5′ or 3′ of the nucleic acid of the reporter.

In some instances, a reporter may be immobilized by surface adsorption. In some cases, the reporter may be immobilized on the surface via electrostatic interaction between the reporter and the surface. For example, a reporter may comprise a negative charge and a surface may comprise a positive charge. In some cases, the surface of a substrate may be coated with a material. The coated material may comprise polyamine, poly-L-lysine, polypyrrole, polyaniline, polyethyleneimine, or a combination thereof.

In some instances, the immobilization moiety may also be used to immobilize a guide nucleic acid. In some cases, the immobilization moiety can be located at an end of a guide nucleic acid. In some cases, the immobilization moiety can be located at the 5′ end of a guide nucleic acid. In some cases, the immobilization moiety can be located at the 3′ end of a guide nucleic acid. In some cases, the immobilization moiety can be located at the 5′ and 3′ end of a guide nucleic acid.

In some embodiments, the programmable nuclease, guide nucleic acid, reporter, or a combination thereof can be immobilized to a device surface (e.g., by a linkage). In some embodiments, the linkage comprises a covalent bond, a non-covalent bond, an electrostatic bond, an interaction (e.g., a covalent or noncovalent bond) between members of a binding pair (e.g., streptavidin and biotin), an amide bond, or any combination thereof. In embodiments where more than one element is immobilized to the surface (e.g., reporters and guide nucleic acid, programmable nuclease and reporters, or all three), the linkage may be the same or different for each species. For example, the guide nucleic acid may be immobilized to the surface by a single-stranded linker polynucleotide, and the reporters may be immobilized by the interaction between a first member of a binding pair on the reporters and a second member of a binding pair on the surface. In general, the term “binding pair” refers to a first and a second moiety that have a specific binding affinity for each other. In some embodiments, a binding pair has a dissociation constant Kd of less than or equal to about: 10-8 mol/L, 10-9 mol/L, 10-10 mol/L, 10-11 mol/L, 10-12 mol/L, 10-13 mol/L, 10-14 mol/L, 10-15 mol/L, or ranges including two of these values as endpoints. Non limiting examples of binding pairs include an antibody or an antigen-binding portion thereof and an antigen (e.g., fluorescein, digoxin, digoxigenin); a biotin (bio) moiety and an avidin (or streptavidin) moiety; a dinitrophenol (DNP) and an anti-DNP antibody; a hapten and an anti hapten; folate and a folate binding protein; vitamin B12 and an intrinsic factor; a carbohydrate and a lectin or carbohydrate receptor; a polysaccharide and a polysaccharide binding moiety; a lectin and a receptor; a ligand and a receptor; a drug and a drug receptor; complementary chemical reactive groups (e.g., sulfhydryl/maleimide, thiol/maleimide, sulfhydryl/haloacetyl derivative, amine/epoxy, amine/isotriocyanate, amine/succinimidyl ester, and amine/sulfonyl halides); an antibody (e.g., IgG) and protein A or protein G; a toxin and a toxin receptor; and an enzyme substrate and an enzyme. In some embodiments, the binding pair comprises biotin and either of avidin or streptavidin.

In some embodiments, the linkage utilizes non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, the programmable nuclease, guide nucleic acid, and/or the reporter are immobilized to or within a polymer matrix. The polymer matrix may comprise a hydrogel. Co-polymerization of the programmable nuclease, guide nucleic acid, or the reporter into the polymer matrix may result in a higher density of reporter/unit volume or reporter/unit area than other immobilization methods utilizing surface immobilization (e.g., onto beads, after matrix polymerization, etc.). Co-polymerization of the programmable nuclease, guide nucleic acid, or the reporter into the polymer matrix may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the polymer matrix as a co-polymer and fewer “free” reporter molecules retained on the hydrogel via non-covalent interactions or non-specific binding interactions.

In some embodiments, a plurality of oligomers and a plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for free-floating programmable nucleases to diffuse into the hydrogel and access immobilized internal reporter molecules. The relative percentages and/or molecular weights of the oligomers may be varied to vary the pore size of the hydrogel. For example, pore size may be tailored to increase the diffusion coefficient of the programmable nucleases.

In some embodiments, the functional groups attached to the reporters and/or guide nucleic acids may be selected to preferentially incorporate the reporters and/or guide nucleic acids into the polymer matrix via covalent binding at the functional group versus other locations along the nucleic acid backbone of the reporter and/or guide nucleic acid. In some embodiments, the functional groups attached to the reporters and/or guide nucleic acids may be selected to favorably transfer free radicals from the functionalized ends of polymerizable oligomers to the functional group on the end of the reporter and/or guide nucleic acid (e.g., 5′ end), thereby forming a covalent bond and immobilizing the reporter and/or guide nucleic acid rather than destroying other parts of the reporter and/or guide nucleic acid molecules, respectively. In some embodiments, the functional group may comprise a single stranded nucleic acid, a double stranded nucleic acid, an acrydite group, a 5′ thiol modifier, a 3′ thiol modifier, an amine group, a I-Linker™ group, methacryl group, or any combination thereof. One of ordinary skill in the art will recognize that a variety of functional groups may be used depending on the desired properties of the immobilized components.

### Reporter Moiety

In some embodiments, the reporter molecule comprises a detection moiety capable of generating a detectable signal. A signal can be a calorimetric, potentiometric, amperometric, optical (e.g., fluorescent, colorimetric, etc.), or piezo-electric. Suitable detectable labels and/or moieties that may provide a signal include, but are not limited to, an enzyme, a radioisotope, a member of a specific binding pair, a fluorophore, a fluorescent protein, a quantum dot, and the like.

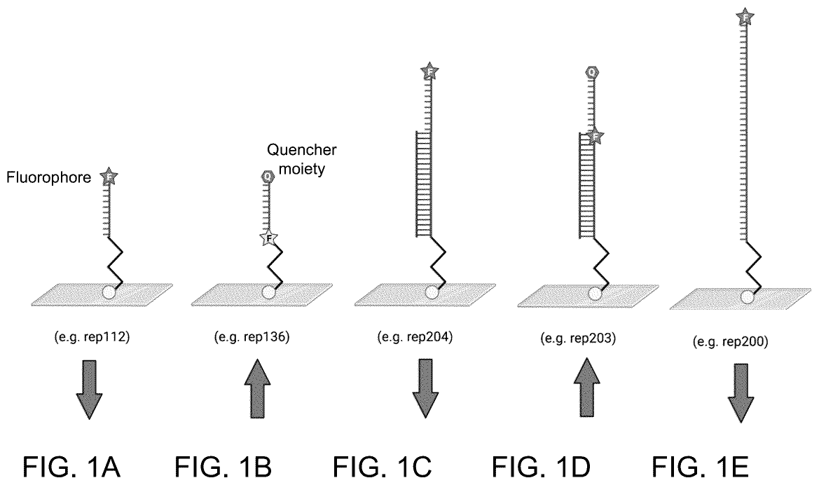

In some instances, a detection moiety can be located at an end of a reporter. In some cases, a detection moiety can be located at an end of a nucleic acid of a reporter. Cleavage of the nucleic acid can release the detection moiety, thereby decreasing the signal a reporter. In some cases, a detection moiety can be located at the 3′ end of a nucleic acid of a reporter. For example, a fluorophore can be located at the 3′ end of a reporter, as shown in FIG. 1A, 1C, or 1E. In other cases, the fluorophore can be at the 5′ end of a reporter. Optionally, a quenching moiety is on the other side of the cleavage site. Sometimes the quenching moiety is a fluorescence quenching moiety. For example, from 5′ to 3′, the reporter may comprise an immobilization moiety, a first nucleic acid, a fluorophore, a second nucleic acid, and a quencher moiety, as shown in FIG. 1B or 1D. The quencher moiety may quench the signal of the fluorophore. Cleavage of the second nucleic acid may release the quencher moiety, thereby increasing the signal of the reporter.

In some cases, the quenching moiety is 5′ to the cleavage site and the fluorophore is 3′ to the cleavage site. In some cases, the fluorophore is 5′ to the cleavage site and the quenching moiety is 3′ to the cleavage site. Sometimes the quenching moiety is at the 5′ terminus of the reporter molecule. Sometimes the fluorophore is at the 3′ terminus of the reporter molecule. In some cases, the fluorophore is at the 5′ terminus of the reporter molecule. In some cases, the quenching moiety is at the 3′ terminus of the reporter molecule.

TABLE 2 provides a list of exemplary detection moieties.

TABLE 2
Exemplary Fluorescent Reporter Molecules

| Detection moiety | Types |
|---|---|
| /56-FAM/ | Fluorophore |
| /36-FAM/ | Fluorophore |
| /i6-FAMK/ | Fluorophore |
| /3IABkFQ/ | Quenching moiety |

/56-FAM/: 5′ 6-Fluorescein (Integrated DNA Technologies)
/36-FAM/: 3′ 6-Fluorescein (Integrated DNA Technologies)
/3IABkFQ/: 3′ Iowa Black FQ (Integrated DNA Technologies)
/i6-FAMK/: internal 6-Fluorescein (Azide) with dT base (Integrated DNA Technologies)
*This Table refers to the detection moiety and quencher moiety as their tradenames and their source is identified. However, alternatives, generics, or non-tradename moieties with similar function from other sources can also be used.

A detection moiety can be a fluorophore. The fluorophore of a reporter that emits fluorescence in the visible spectrum. In some embodiments, the fluorophore of a reporter emits fluorescence in the visible spectrum. In some embodiments, fluorophore of a reporter can be a fluorophore that emits fluorescence in the near-IR spectrum. In some embodiments, fluorophore of a reporter can be a fluorophore that emits fluorescence in the IR spectrum. A detection moiety can be a fluorophore that emits fluorescence in the range of from 500 nm and 720 nm. A detection moiety can be a fluorophore that emits fluorescence in the range of from 500 nm and 720 nm. In some cases, fluorophore of a reporter emits fluorescence at a wavelength of 700 nm or higher. In other cases, fluorophore of a reporter emits fluorescence at about 660 nm or about 670 nm. In some cases, fluorophore of a reporter emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, fluorophore of a reporter emits fluorescence in the range from 450 nm to 750 nm, from 500 nm to 650 nm, or from 550 to 650 nm. A detection moiety can be a fluorophore that emits a fluorescence in the same range as 6-Fluorescein, IRDye 700, TYE 665, Alex Fluor, or ATTO™ 633 (NHS Ester). A detection moiety can be fluorescein amidite, 6-Fluorescein, IRDye 700, TYE 665, Alex Fluor 594, or ATTO™ 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). Any of the detection moieties described herein can be from any commercially available source, can be an alternative with a similar function, a generic, or a non-tradename of the detection moieties listed.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, (E≤-glucuronidase, invertase, Xanthine Oxidase, firefly luciferase, and glucose oxidase (GO).

A detection moiety can be chosen for use based on the type of sample to be tested. For example, a detection moiety that is an infrared fluorophore is used with a urine sample. As another example, a fluorophore that emits a fluorescence around 520 nm is used for testing in non-urine samples, and a fluorophore that emits a fluorescence around 700 nm is used for testing in urine samples.

A quenching moiety can be chosen based on its ability to quench fluorophore of a reporter. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO™ 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A quenching moiety can be Iowa Black RQ (Integrated DNA Technologies), Iowa Black FQ (Integrated DNA Technologies), Black Hole Quencher (Sigma Aldrich), or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of fluorophore of a reporter indicates that cleavage by the programmable nuclease has occurred and that the sample contains the target nucleic acid. In addition, in some examples, a catalytic oligonucleotide can be activated by the programmable nuclease upon its hybridization to the target nucleic acid molecule. In some instances, a catalytic oligonucleotide can be used to further intensify the detectable signal. This can decrease the detection threshold. For examples, analytes (e.g., target nucleic acid molecules) at lower concentrations can be detected using the assay as the assay sensitivity can be increased using a catalytic oligonucleotide as described herein.

In some cases, fluorophore of a reporter comprises a fluorescent dye. In some examples, fluorophore of a reporter comprises a fluorescence resonance energy transfer (FRET) pair. In some cases, fluorophore of a reporter comprises an infrared (IR) dye. In some cases, fluorophore of a reporter comprises an ultraviolet (UV) dye. Alternatively, or in combination, fluorophore of a reporter comprises a polypeptide. Sometimes fluorophore of a reporter comprises a biotin. Sometimes fluorophore of a reporter comprises at least one of avidin or streptavidin. In some instances, fluorophore of a reporter comprises a polysaccharide, a polymer, or a nanoparticle. In some instances, fluorophore of a reporter comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A reporter molecule, sometimes, is protein-nucleic acid that is capable of generating a calorimetric, potentiometric, amperometric, optical (e.g., fluorescent, colorometric, etc.), or piezo-electric signal upon cleavage of the nucleic acid. In some cases, a calorimetric signal is heat produced after cleavage of the reporter molecules. Sometimes, a calorimetric signal is heat absorbed after cleavage of the reporter molecules. A potentiometric signal, for example, is electrical potential produced after cleavage of the reporter molecules. An amperometric signal can be movement of electrons produced after the cleavage of reporter molecule. The signal is an optical signal, such as a colorometric signal or a fluorescence signal. An optical signal is, for example, a light output produced after the cleavage of the reporter molecules. Sometimes, an optical signal is a change in light absorbance between before and after the cleavage of reporter molecules. In some cases, a piezo-electric signal is a change in mass between before and after the cleavage of the reporter molecule. Other methods of detection can also be used, such as optical imaging, surface plasmon resonance (SPR), and/or interferometric sensing.

In some embodiments, the reporter comprises a nucleic acid conjugated to an affinity molecule which is in turn conjugated to the fluorophore (e.g., nucleic acid-affinity molecule-fluorophore) or the nucleic acid conjugated to the fluorophore which is in turn conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicates that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore-no intervening moiety is present between the nucleic acid and the affinity molecule and no intervening moiety is present between the affinity molecule and the fluorophore. The affinity molecule can be biotin, avidin, streptavidin, or any similar molecule.

In some instances, the reporter is a protein-nucleic acid. A protein-nucleic acid may comprise a protein or a peptide (or polypeptide) moiety attached to a nucleic acid. In some instances, a method of assaying for a target nucleic acid in a sample comprises contacting the sample to a complex comprising a) a guide nucleic acid comprising a segment that is reverse complementary to a segment of the target nucleic acid and b) a programmable nuclease that exhibits sequence independent cleavage upon forming an activated complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The protein-nucleic acid is an enzyme-nucleic acid or an enzyme substrate-nucleic acid. Sometimes, the protein-nucleic acid is attached to a solid support. The nucleic acid can be DNA, RNA, or a DNA/RNA hybrid.

In some instances, the protein moiety of a reporter can be at the 5′ of a nucleic acid. In some cases, the protein moiety can be at the 3′ of a nucleic acid. In some instances, the protein moiety can be at the 5′ and/or 3′ of a nucleic acid. In some cases, the protein moiety can be in-between two nucleic acids.

Cleavage of the protein-nucleic acid produces a signal. The systems and devices disclosed herein can be used to detect these signals, which indicate whether a target nucleic acid is present in the sample.

In some cases, the protein-nucleic acid reporter is an enzyme-nucleic acid. The enzyme can be sterically hindered when present as in the enzyme-nucleic acid, but then functional upon cleavage from the nucleic acid. The enzyme is an enzyme that produces a reaction with a substrate. An enzyme can be invertase. The substrate of invertase is sucrose and DNS reagent. In some cases, it is preferred that the nucleic acid and invertase are conjugated using a heterobifunctional linker via sulfo-SMCC chemistry.

In some examples, the protein-nucleic acid is a substrate-nucleic acid. The substrate is a substrate that produces a reaction with an enzyme. Release of the substrate upon cleavage by the programmable nuclease may free the substrate to react with the enzyme.

A protein-nucleic acid or other reporter molecule can be attached to a solid support. The solid support, for example, is a surface. A surface can be an electrode. Sometimes the solid support is a bead. In some cases, the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to a device. Thus, the detecting steps disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the nucleic acid of the reporter by the programmable nuclease and/or a signal amplifier. In other embodiments, the signal changes upon cleavage of the reporter by the programmable nuclease and/or the signal amplifier. In other embodiments, a signal can be present in the absence of reporter cleavage and disappear upon cleavage of the target nucleic acid by the programmable nuclease and/or the signal amplifier. For example, a signal can be produced in a microfluidic device or lateral flow device after contacting a sample with a composition comprising a programmable nuclease and a signal amplifier as described herein.

In some instances, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of fluorophore of a reporter to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of reporter molecule. In some cases, the detectable signal is generated directly by the cleavage event. Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized on the surface of a chamber in a device as disclosed herein. In some cases, the reporter is immobilized on beads, such as magnetic beads, in a chamber of a device as disclosed herein where they can be held in position by a magnet placed below the chamber.

In the presence of a large amount of non-target nucleic acids, an activity of a programmable nuclease (e.g., a Type V or Type VI CRISPR/Cas protein as disclosed herein) may be inhibited. If total nucleic acids are present in large amounts, they may outcompete reporters for the programmable nucleases. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Multiplex Reporter Systems

In some instances, systems and methods may be used for multiple detection of target nucleic acids. In some cases, a substrate and/or detection region may be immobilized with multiple reporters, multiple guide nucleic acids, or a combination thereof. Localizing the guide nucleic acids and reporter may localize the detectable signal for each target nucleic acid to the detection spot, thus enabling the spatial multiplexing. For example, in some embodiments, the detection region may comprise an array of detection spots at discrete locations. Each detection spot of the array may comprise an immobilized reporter and a different immobilized guide nucleic acid which is complementary to a different target nucleic acid of a plurality of target nucleic acids. In some embodiments, at each detection spot of the array, upon addition of a programmable nuclease, the immobilized reporter is cleaved by a complex comprising the programmable nuclease and the different immobilized guide nucleic acid to generate a different signal of a plurality of signals. Each different signal may therefore be indicative of the presence or absence of a different target nucleic acid. The target nucleic acids may be freely available within the fluid volume of the detection region. In some embodiments, the array may comprise a number of spots within a range of about 1 to about 200, within a range of about 3 to about 200, or within a range of about 10 to about 200. In some embodiments, the array may comprise at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more spots. In some embodiments, multiple guide nucleic acids for a single target nucleic acid may be combined within a single detection spot in order to increase a rate of reaction.

In some instances, a substrate may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 same or different reporters and/or guide nucleic acids immobilized thereto. In some cases, a detection region may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 same or different reporters and/or guide nucleic acids immobilized thereto. In some cases, each detection location may comprise 1, 2, 3, 4, 5, 6, 7, 8, 9, 10 or more reporters and/or guide nucleic acids immobilized thereto.

### B. Programmable Nucleases

Disclosed herein are programmable nucleases and uses thereof, e.g., detection and editing of target nucleic acids. In some cases, a programmable nuclease is capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and can non-specifically degrade a non-target nucleic acid in its environment. The programmable nuclease has trans cleavage activity once activated. A programmable nuclease can be a Cas protein (also referred to, interchangeably, as a Cas nuclease or Cas effector protein). A guide nucleic acid (e.g., crRNA) and Cas protein can form a CRISPR enzyme (also referred to herein as a programmable nuclease complex or probe).

In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. The programmable nuclease can be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the programmable nuclease can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The nucleic acids described and referred to herein can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine, Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5hmC), 5-formylcytosine (5fC), or 5-carboxylcytosine (5caC).

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of reporter nucleic acids comprising a detection moiety. Once the reporter is cleaved by the activated programmable nuclease, the detection moiety can be released or separated from the reporter and can directly or indirectly generate a detectable signal. The reporter and/or the detection moiety can be immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The systems and methods of the present disclosure can be implemented using a device that is compatible with any type of programmable nuclease that is human-engineered or naturally occurring. The programmable nuclease can comprise a nuclease that is capable of being activated when complexed with a guide nucleic acid and a target nucleic acid segment or a portion thereof. A programmable nuclease can become activated when complexed with a guide nucleic acid and a target sequence of a target gene of interest. The programmable nuclease can be activated upon binding of a guide nucleic acid to a target nucleic acid and can exhibit or enable trans cleavage activity once activated. In any instances or embodiments where a CRISPR-based programmable nuclease is described or used, it is recognized herein that any other type of programmable nuclease can be used in addition to or in substitution of such CRISPR-based programmable nuclease.

The systems and methods of the present disclosure can be implemented using a device that is compatible with a plurality of programmable nucleases. The device can comprise a plurality of programmable nuclease probes (also referred to herein as programmable nuclease complexes) comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpf1. Programmable nucleases can also include, for example, PfAgo and/or NgAgo.

ZFNs can cut genetic material in a sequence-specific matter and can be designed, or programmed, to target specific viral targets. A ZFN is composed of two domains: a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets. TALENs contain the Fokl nuclease domain at their carboxyl termini and a class of DNA binding domains known as transcription activator-like effectors (TALEs). TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

The programmable nuclease can comprise any type of engineered enzyme. Alternatively, the programmable nuclease can comprise CRISPR enzymes derived from naturally occurring bacteria or phage. A programmable nuclease can be a Cas effector protein (also referred to, interchangeably, as a Cas nuclease). A guide nucleic acid (e.g., a crRNA) and Cas effector protein can form a CRISPR enzyme. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The programmable nuclease can comprise one or more amino acid modifications. The programmable nuclease can be a nuclease derived from a CRISPR-Cas system. The programmable nuclease can be a nuclease derived from recombineering. In some embodiments, the programmable nuclease further comprises a Cas enzyme. In some embodiments, the Cas enzyme is selected from the group consisting of Cas12, Cas13, Cas14, Cas14a, Cas14al, and CasPhi.

In some cases, the programmable nuclease is Cas13. Sometimes the Cas13 is Cas13a, Cas13b, Cas13c, Cas13d, Cas13e, or Cas13f. In some cases, the programmable nuclease is Mad7 or Mad2. In some cases, the programmable nuclease is Cas12. Sometimes the Cas12 is Cas12a, Cas12b, Cas12c, Cas12d, Cas12e, Cas12f, Cas12g, Cas12h, Cas12i, Cas12j, or Cas12k. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas14f, Cas14g, or Cas14h. Sometimes, the programmable nuclease is a type V CRISPR-Cas system. In some cases, the programmable nuclease is a type VI CRISPR-Cas system. Sometimes the programmable nuclease is a type III CRISPR-Cas system. In some cases, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum ((am), Prevotella sp. (Psm), Capnocytophaga canimorsus (Cca Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus ((′ca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

Disclosed herein are programmable nucleases and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide nucleic acids of Type V CRISPR/Cas proteins hybridize to ssDNA or dsDNA. However, the trans cleavage activity of Type V CRISPR/Cas protein is typically directed towards ssDNA. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease.

In some instances, the Type V Cas protein is a CasΦ protein. A CasΦ protein can function as an endonuclease that catalyzes cleavage at a specific sequence in a target nucleic acid. A programmable CasΦ nuclease may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

In some instances, the programmable nuclease is a Type VI Cas protein. In some embodiments, the Type VI Cas protein is a programmable Cas13 nuclease. The general architecture of a Cas13 protein includes an N-terminal domain and two HEPN (higher eukaryotes and prokaryotes nucleotide-binding) domains separated by two helical domains. The HEPN domains each comprise aR-X₄-H motif. Shared features across Cas13 proteins include that upon binding of the crRNA of the guide nucleic acid to a target nucleic acid, the protein undergoes a conformational change to bring together the HEPN domains and form a catalytically active RNase. Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic components. In some instances, the Cas effector is a Cas 13 effector. In some instances, the Cas13 effector is a Cas13a, a Cas13b, a Cas 13c, a Cas 13d, or a Cas 13e effector protein.

In some embodiments, the programmable nuclease comprises a Cas12 protein, wherein the Cas12 enzyme binds and cleaves double stranded DNA and single stranded DNA. In some embodiments, programmable nuclease comprises a Cas13 protein, wherein the Cas13 enzyme binds and cleaves single stranded RNA. In some embodiments, programmable nuclease comprises a Cas14 protein, wherein the Cas14 enzyme binds and cleaves both double stranded DNA and single stranded DNA.

Table 3 provides illustrative amino acid sequences of programmable nucleases having trans-cleavage activity. The programmable nuclease may comprise an amino acid sequence that is at least 65%, at least 70%, at least 75%, at least 80%, at least 85%, at least 90%, at least 95%, at least 98%, at least 99%, or 100% identical to any one of SEQ ID Nos: 9-69 or 81-88. The programmable nuclease may consist of an amino acid sequence that is at least 65%, at least 70%, at least 75%, at least 80%, at least 85%, at least 90%, at least 95%, at least 98%, at least 99%, or 100% identical to any one of SEQ ID Nos: 9-69 or 81-88. The programmable nuclease may comprise at least about 50, at least about 100, at least about 150, at least about 200, at least about 250, at least about 300, at least about 350, at least about 400, at least about 450, at least about 500 consecutive amino acids of any one of SEQ ID Nos: 9-69 or 81-88.

TABLE 3
Amino Acid Sequences of Exemplary Programmable Nucleases

| SEQ ID NO: | Programmable Nuclease Amino Acid Sequence |
|---|---|
| SEQ ID NO: | MADLSQFTHKYQVPKTLRFELIPQGKTLENLSAYGMVADDKQRSENYKKLKPVIDRIYKYFI |
| 9 | EESLKNTNLDWNPLYEAIREYRKEKTTATITNLKEQQDICRRAIASRFEGKVPDKGDKSVKD |
| | FNKKQSKLFKELFGKELFTDSVLEQLPGVSLSDEDKALLKSFDKFTTYFVGFYDNRKNVFSS |
| | DDISTGIPHRLVQENFPKFIDNCDDYKRLVLVAPELKEKLEKAAEATKIFEDVSLDEIFSIKFY |
| | NRLLQQNQIDQFNQLLGGIAGAPGTPKIQGLNETLNLSMQQDKTLEQKLKSVPHRFSPLYKQ |
| | ILSDRSSLSFIPESFSCDAEVLLAVQEYLDNLKTEHVIEDLKEVFNRLTTLDLKHIYVNSTKVT |
| | AFSQALFGDWNLCREQLRVYKMSNGNEKITKKALGELESWLKNSDIAFTELQEALADEALP |
| | AKVNLKVQEAISGLNEQMAKSLPKELKIPEEKEELKALLDAIQEVYHTLEWFIVSDDVETDT |
| | DFYVPLKETLQIIQPIIPLYNKVRNFATQKPYSVEKFKLNFANPTLADGWDENKEQQNCAVL |
| | FQKGNNYYLGILNPKNKPDFDNVDTEKQGNCYQKMVYKQFPDFSKMMPKCTTQLKEVKQ |
| | HFEGKDSDYILNNKNFIKPLTITREVYDLNNVLYDGKKKFQIDYLRKTKDEDGYYTALHTWI |
| | DFAKKFVASYKSTSIYDTSTILPPEKYEKLNEFYGALDNLFYQIKFENIPEEIIDTYVEDGKLFL |
| | FQIYNKDFAAGATGAPNLHTIYWKAVFDPENVKDVVVKLNGQAELFYRPKSNMDVIRHKV |
| | GEKLVNRTLKDGSILTDELHKELYLYANGSLKKGLSEDAKIILDKNLAVIYDVHHEIVKDRR |
| | FTTDKFFFHVPLTLNYKCDKNPVKFNAEVQEYLKENPDTYVIGIDRGERNLIYAVVIDPKGRI |
| | VEQKSFNVINGFDYHGKLDQREKERVKARQAWTAVGKIKELKQGYLSLVVHEISKMMVRY |
| | QAVVVLENLNVGFKRVRSGIAEKAVYQQFEKMLINKLNYLMFKDAGGTEPGSVLNAYQLT |
| | DRFESFAKMGLQTGFLFYIPAAFTSKIDPATGFVDPFRWGAIKTLADKREFLSGFESLKFDSTT |
| | GNFILHFDVSKNKNFQKKLEGFVPDWDIIIEANKMKTGKGATYIAGKRIEFVRDNNSQGHYE |
| | DYLPCNALAETLRQCDIPYEEGKDILPLILEKNDSKLLHSVFKVVRLTLQMRNSNAETGEDYI |
| | SSPVEDVSGSCFDSRMENEKLPKDADANGAYHIALKGMLALERLRKDEKMAISNNDWLNYI |
| | QEKRA* |
| SEQ ID NO: | MAGKKKDKDVINKTLSVRIIRPRYSDDIEKEISDEKAKRKQDGKTGELDRAFFSELKSRNPDII |
| 10 | TNDELFPLFTEIQKNLTEIYNKSISLLYMKLIVEEEGGSTASALSAGPYKECKARFNSYISLGL |
| | RQKIQSNFRRKELKGFQVSLPTAKSDRFPIPFCHQVENGKGGFKVYETGDDFIFEVPLIKYTA |
| | TNKKSTSGKNYTKVQLNNPPVPMNVPLLLSTMRRRQTKKGMQWNKDEGTNAELRRVMSG |
| | EYKVSYAEIIRRTRFGKHDDWFVNFSIKFKNKTDELNQNVRGGIDIGVSNPLVCAVINGLDR |
| | YIVANNDIMAFNERAMARRRTLLRKNRFKRSGHGAKNKLEPITVLTEKNERFRKSILQRWA |
| | REVAEFFKRTSASVVNMEDLSGITEREDFFSTKLRTTWNYRLMQTTIENKLKEYGIAVNYISP |
| | KYTSQTCHSCGKRNDYFTFSYRSENNYPPFECKECNKVKCNADFNAAKNIALKVVL |
| SEQ ID NO: | MAKNTITKTLKLRIVRPYNSAEVEKIVADEKNNREKIALEKNKDKVKEACSKHLKVAAYCT |
| 11 | TQVERNACLFCKARKLDDKFYQKLRGQFPDAVFWQEISEIFRQLQKQAAEIYNQSLIELYYEI |
| | FIKGKGIANASSVEHYLSDVCYTRAAELFKNAAIASGLRSKIKSNFRLKELKNMKSGLPTTKS |
| | DNFPIPLVKQKGGQYTGFEISNHNSDFIIKIPFGRWQVKKEIDKYRPWEKFDFEQVQKSPKPIS |
| | LLLSTQRRKRNKGWSKDEGTEAEIKKVMNGDYQTSYIEVKRGSKIGEKSAWMLNLSIDVPK |
| | IDKGVDPSIIGGIDVGVKSPLVCAINNAFSRYSISDNDLFHFNKKMFARRRILLKKNRHKRAG |
| | HGAKNKLKPITILTEKSERFRKKLIERWACEIADFFIKNKVGTVQMENLESMKRKEDSYFNIR |
| | LRGFWPYAEMQNKIEFKLKQYGIEIRKVAPNNTSKTCSKCGHLNNYFNFEYRKKNKFPHFK |
| | CEKCNFKENADYNAALNISNPKLKSTKEEP |
| SEQ ID NO: | MATLVSFTKQYQVQKTLRFELIPQGKTQANIDAKGFINDDLKRDENYMKVKGVIDELHKNFI |
| 12 | EQTLVNVDYDWRSLATAIKNYRKDRSDTNKKNLEKTQEAARKEIIAWFEGKRGNSAFKNN |
| | QKSFYGKLFKKELFSEILRSDDLEYDEETQDAIACFDKFTTYFVGFHENRKNMYSTEAKSTS |
| | VAYRVVNENFSKFLSNCEAFSVLEAVCPNVLVEAEQELHLHKAFSDLKLSDVFKVEAYNKY |
| | LSQTGIDYYNQIIGGISSAEGVRKIRGVNEVVNNAIQQNDELKVALRNKQFTMVQLFKQILSD |
| | RSTLSFVSEQFTSDQEVITVVKQFNDDIVNNKVLAVVKTLFENFNSYDLEKIYINSKELASVS |
| | NALLKDWSKIRNAVLENKIIELGANPPKTKISAVEKEVKNKDFSIAELASYNDKYLDKEGND |
| | KEICSIANVVLEAVGALEIMLAESLPADLKTLENKNKVKGILDAYENLLHLLNYFKVSAVND |
| | VDLAFYGAFEKVYVDISGVMPLYNKVRNYATKKPYSVEKFKLNFAMPTLADGWDKNKER |
| | DNGSIILLKDGQYYLGVMNPQNKPVIDNAVCNDAKGYQKMVYKMFPEISKMVTKCSTQLN |
| | AVKAHFEDNTNDFVLDDTDKFISDLTITKEIYDLNNVLYDGKKKFQIDYLRNTGDFAGYHK |
| | ALETWIDFVKEFLSKYRSTAIYDLTTLLPTNYYEKLDVFYSDVNNLCYKIDYENISVEQVNE |
| | WVEEGNLYLFKIYNKDFATGSTGKPNLHTMYWNAVFAEENLHDVVVKLNGGAELFYRPKS |
| | NMPKVEHRVGEKLVNRKNVNGEPIADSVHKEIYAYANGKISKSELSENAQEELPLAIIKDVK |
| | HNITKDKRYLSDKYFFHVPITLNYKANGNPSAFNTKVQAFLKNNPDVNIIGIDRGERNLLYV |
| | VVIDQQGNIIDKKQVSYNKVNGYDYYEKLNQREKERIEARQSWGAVGKIKELKEGYLSLVV |
| | REIADMMVKYNAIVVMENLNAGFKRVRGGIAEKAVYQKFEKMLIDKLNYLVFKDVEAKEA |
| | GGVLNAYQLTDKFDSFEKMGNQSGFLFYVPAAYTSKIDPVTGFANVFSTKHITNTEAKKEFI |
| | CSFNSLRYDEAKDKFVLECDLNKFKIVANSHIKNWKFIIGGKRIVYNSKNKTYMEKYPCEDL |
| | KATLNASGIDFSSSEIINLLKNVPANREYGKLFDETYWAIMNTLQMRNSNALTGEDYIISAVA |
| | DDNEKVFDSRTCGAELPKDADANGAYHIALKGLYLLQRIDISEEGEKVDLSIKNEEWFKFVQ |
| | QKEYAR* |
| SEQ ID NO: | MCMKITKIDGISHKKYKEKGKLIKNNDTAKDIIEERFNDIEKKTKELFQKTLDFYVKNYEKC |
| 13 | KEQNKERREKAKNYFSKVKILVDNKKITICNENTEKMEIEDFNEYDVRSGKYFNVLNKILNG |
| | ENYTEEDLEVFENDLQKRTGRIKSIKNSLEENKAHFKKESINNNIIYDRVKGNNKKSLFYEYY |
| | RISSKHQEYVNNIFEAFDKLYSNSHEAMNNLFSEITKDSKDRNIRKIREAYHEILNKNKTEFGE |
| | ELYKKIQDNRNNFDKLLEIEPEIKELTKSQIFYKYYIDKVNLDETSIKHCFCHLVEIEVNQLLK |
| | NYVYSKRNINKEKLENIFEYCKLKNLIKNKLVNKLNNYIRNCGKYNAYISNNDVVVNSEKIS |
| | EIRTKEAFLRSIIGVSSSAYFSLRNILNTDNTQDITNKVDKEVDKLYQENKKIELEERLKLFFG |
| | NYFDINNQQEIKVFLMNIDKIISSIRHEIIHFKMETNAQNIFDENNVNLGNTAKNIFSNEINEEKI |
| | KFKIFKQLNSANVFDYLSNKDITEYMDKVVFSFTNRNVSFVPSFTKIYNRVQDLANSLEIKK |
| | WKIPDKSEGKDAQIYLLKNIYYGKFLDEFLNEENGIFISIKDKIIELNRNQNKRTGFYKLEKFE |
| | KIEETNPKKYLEIIQSLYMINIEEIDSEGKNIFLDFIQKIFLKGFFEFIKNNYNYLLELKKIQDKK |
| | NIFDSEMSEYIAGEKTLEDIGEINEIIQDIKITEIDKILNQTDKINCFYLLLKLLNYKEITELKGNL |
| | EKYQILSKTNVYEKELMLLNIVNLDNNKVKIENFKILAEEIGKFIEKINIEEINKNKKIKTFEEL |
| | RNFEKGENTGEYYNIYSDDKNIKNIRNLYNIKKYGMLDLLEKISEKTNYCIKKKDLEEYSELR |
| | KQLEDEKTNFYKIQEYLHSKYQQKPKKILLKNNKNDYEKYKKSIENIEKYVHLKNKIEFNEL |
| | NLLQSLLLKILHRLVGFTSIWERDLRFRLIGEFPDELDVEDIFDHRKRYKGTGKGICKKYDRFI |
| | NTHTEYKNNNKMENVKFADNNPVRNYIAHFNYLPNPKYSILKMMEKLRKLLDYDRKLKNA |
| | VMKSIKDILEEYGFKAEFIINSDKEIILNLVKSVEIIHLGKEDLKSRRNSEDLCKLVKAMLEYS |
| | K* |
| SEQ ID NO: | MEDKQFLERYKEFIGLNSLSKTLRNSLIPVGSTLKHIQEYGILEEDSLRAQKREELKGIMDDY |
| 14 | YRNYIEMHLRDVHDIDWNELFEALTEVKKNQTDDAKKRLEKIQEKKRKEIYQYLSDDAVFS |
| | EMFKEKMISGILPDFIRCNEGYSEEEKEEKLKTVALFHRFTSSFNDFFLNRKNVFTKEAIVTAI |
| | GYRVVHENAEIFLENMVAFQNIQKSAESQISIIERKNEHYFMEWKLSHIFTADYYMMLMTQK |
| | AIEHYNEMCGVVNQQMREYCQKEKKNWNLYRMKRLHKQILSNASTSFKIPEKYENDAEVY |
| | ESVNSFLQNVMEKTVMERIAVLKNSTDNFDLSKIYITAPYYEKISNYLCGSWNTITDCLTHY |
| | YEQQIAGKGARKDQKVKAAVKADKWKSLSEIEQLLKEYARAEEVKRKPEEYIAEIENIVSLK |
| | EAHLLEYHPEVNLIENEKYATEIKDVLDNYMELFHWMKWFYIEEAVEKEVNFYGELDDLYE |
| | EIKDIVPLYNKVRNYVTQKPYSDTKIKLNFGTPTLANGWSKSKEYDYNAILLQKDGKYYMG |
| | IFNPIQKPEKEIIEGHSQPLEGNEYKKMVYYYLPSANKMLPKVLLSKKGMEIYQPSEYIINGY |
| | KERRHIKSEEKFDLQFCHDLIDYFKSGIERNSDWKVFGFDFSDTDTYQDISGFYREVEDQGY |
| | KIDWTYIKEADIDRLNEEGKLYLFQIYNKDFSEKSTGRENLHTMYLKNLFSEENVREQVLKL |
| | NGEAEIFFRKSSVKKPIIHKKGTMLVNRTYMEEVNGNSVRRNIPEKEYQEIYNYKNHRLKGE |
| | LSTEAKKYLEKAVCHETKKDIVKDYRYSVDKFFIHLPITINYRASGKETLNSVAQRYIAHQN |
| | DMHVIGIDRGERNLIYVSVINMQGEIKEQKSFNIINEFNYKEKLKEREQSRGAARRNWKEIGQ |
| | IKDLKEGYLSGVIHEIAKMMIKYHAIIAMEDLNYGFKRGRFKVERQVYQKFENMLIQKLNYL |
| | VFKDRPADEDGGVLRGYQLAYIPDSVKKMGRQCGMIFYVPAAFTSKIDPTTGFVDIFKHKV |
| | YTTEQAKREFILSFDEICYDVERQLFRFTFDYANFVTQNVTLARNNWTIYTNGTRAQKEFGN |
| | GRMRDKEDYNPKDKMVELLESEGIEFKSGKNLLPALKKVSNAKVFEELQKIVRFTVQLRNS |
| | KSEENDVDYDHVISPVLNEEGNFFDSSKYKNKEEKKESLLPVDADANGAYCIALKGLYIMQ |
| | AIQKNWSEEKALSPDVLRLNNNDWFDYIQNKRYR* |
| SEQ ID NO: | MEEKKMSKIEKFIGKYKISKTLRFRAVPVGKTQDNIEKKGILEKDKKRSEDYEKVKAYLDSL |
| 15 | HRDFIENTLKKVKLNELNEYACLFFSGTKDDGDKKKMEKLEEKMRKTISNEFCNDEMYKKI |
| | FSEKILSENNEEDVSDIVSSYKGFFTSLNGYVNNRKNLYVSDAKPTSIAYRCINENLPKFLRN |
| | VECYKKVVQVIPKEQIEYMSNNLNLSPYRIEDCFNIDFFEFCLSQGGIDLYNTFIGGYSKKDG |
| | TKVQGINEIVNLYNQKNKKDKEKYKLPQFTPLFKQILSDRDTKSFSIEKLENIYEVVELVKKS |
| | YSDEMFDDIETVFSNLNYYDASGIYVKNGPAITHISMNLTKDWATIRNNWNYEYDEKHSTK |
| | KNKNIEKYEDTRNTMYKKIDSFTLEYISRLVGKDIDELVKYFENEVANFVMDIKKTYSKLTP |
| | LFDRCQKENFDISEDEVNDIKGYLDNVKLLESFMKSFTINGKENNIDYVFYGKFTDDYDKLH |
| | EFDHIYNKVRNYITTSRKPYKLDKYKLYFDNPQLLGGWDINKEKDYRTVMLTKDGKYYFAI |
| | IDKGEHPFDNIPKDYFDNNGYYKKIIYRQIPNAAKYLSSKQIVPQNPPEEVKRILDKKKADSK |
| | SLTEEEKNIFIDYIKSDFLKNYKLLFDKNNNPYFNFAFRESSTYESLNEFFEDVERQAYSVRYE |
| | NLPADYIDNLVNEGKIYLFEIYSKDFSEYSKGTNNLHTMYFKALFDNDNLKNTVFKLSGNAE |
| | LFIRPASIKKDELVIHPKNQLLQNKNPLNPKKQSIFDYDLVKDKRFFENQYMLHISIEINKNER |
| | DAKKIKNINEMVRKELKDSDDNYIIGIDRGERNLLYVCVINSAGKIVEQMSLNEIINEYNGIK |
| | HTVDYQGLLDKCEKERNAQRQSWKSIENIKELKDGYISQVVHKLCQLVEKYDAIIAMENLN |
| | GGFKRGRTKFEKQVYQKFENKLINKMEYMADKKRKTTENGGILRGYQLTNGCINNSYQNG |
| | FIFYVPAWLTSKIDPTTGFVDLLKPKYTNVEEAHLWINKFNSITYDKKLDMFAFNINYSQFPR |
| | ADIDYRKIWTFYTNGYRIETFRNSEKNNEFDWKEVHLTSVIKKLLEEYQINYISGKNIIDDLIQ |
| | IKDKPFWNSFIKYIRLTLQMRNSITGRTDVDYIISPVINNEGTFYDSRKDLDEITLPQDADANG |
| | AYNIARKALWIIEKLKESPDEELNKVKLAITQREWLEYAQINI* |
| SEQ ID NO: | MEKIKKPSNRNSIPSIIISDYDANKIKEIKVKYLKLARLDKITIQDMEIVDNIVEFKKILLNGVE |
| 16 | HTIIDNQKIEFDNYEITGCIKPSNKRRDGRISQAKYVVTITDKYLRENEKEKRFKSTERELPNN |
| | TLLSRYKQISGFDTLTSKDIYKIKRYIDFKNEMLFYFQFIEEFFNPLLPKGKNFYDLNIEQNKD |
| | KVAKFIVYRLNDDFKNKSLNSYITDTCMIINDFKKIQKILSDFRHALAHFDFDFIQKFFDDQLD |
| | KNKFDINTISLIETLLDQKEEKNYQEKNNYIDDNDILTIFDEKGSKFSKLHNFYTKISQKKPAF |
| | NKLINSFLSQDGVPNEEFKSYLVTKKLDFFEDIHSNKEYKKIYIQHKNLVIKKQKEESQEKPD |
| | GQKLKNYNDELQKLKDEMNTITKQNSLNRLEVKLRLAFGFIANEYNYNFKNFNDEFTNDVK |
| | NEQKIKAFKNSSNEKLKEYFESTFIEKRFFHFSVNFFNKKTKKEETKQKNIFNSIENETLEELV |
| | KESPLLQIITLLYLFIPRELQGEFVGFILKIYHHTKNITSDTKEDEISIEDAQNSFSLKFKILAKNL |
| | RGLQLFHYSLSHNTLYNNKQCFFYEKGNRWQSVYKSFQISHNQDEFDIHLVIPVIKYYINLN |
| | KLMGDFEIYALLKYADKNSITVKLSDITSRDDLKYNGHYNFATLLFKTFGIDTNYKQNKVSI |
| | QNIKKTRNNLAHQNIENMLKAFENSEIFAQREEIVNYLQTEHRMQEVLHYNPINDFTMKTVQ |
| | YLKSLSVHSQKEGKIADIHKKESLVPNDYYLIYKLKAIELLKQKVIEVIGESEDEKKIKNAIAK |
| | EEQIKKGNN |
| SEQ ID NO: | MEKSLNDFIGLYSVSKTLRFELKPVSETLENIKKFHFLEEDKKKANDYKDVKKIIDNYHKYFI |
| 17 | DDVLKNASFNWKKLEEAIREYNKNKSDDSALVAEQKKLGDAILKLFTSDKRYKALTAATPK |
| | ELFESILPDWFGEQCNQDLNKAALKTFQKFTSYFTGFQENRKNVYSAEAIPTAVPYRIVNDN |
| | FPKFLQNVLIFKTIQEKCPQIIDEVEKELSSYLGKEKLAGIFTLESFNKYLGQGGKENQRGIDF |
| | YNQIIGGVVEKEGGINLRGVNQFLNLYWQQHPDFTKEDRRIKMVPLYKQILSDRSSLSFKIES |
| | IENDEELKNALLECADKLELKNDEKKSIFEEVCDLFSSVKNLDLSGIYINRKDINSVSRILTGD |
| | WSWLQSRMNVYAEEKFTTKAEKARWQKSLDDEGENKSKGFYSLTDLNEVLEYSSENVAET |
| | DIRITDYFEHRCRYYVDKETEMFVQGSELVALSLQEMCDDILKKRKAMNTVLENLSSENKL |
| | REKTDDVAVIKEYLDAVQELLHRIKPLKVNGVGDSTFYSVYDSIYSALSEVISVYNKTRNYIT |
| | KKAASPEKYKLNFDNPTLADGWDLNKEQANTSVILRKDGMFYLGIMNPKNKPKFAEKYDC |
| | GNESCYEKMIYKQFDATKQIPKCSTQKKEVQKYFLSGATEPYILNDKKSFKSELIITKDIWFM |
| | NNHVWDGEKFVPKRDNETRPKKFQIGYFKQTGDFDGYKNALSNWISFCKNFLQSYLSATVY |
| | DYNFKNSEEYEGLDEFYNYLNATCYKLNFINIPETEINKMVSEGKLYLFQIYNKDFASGSTG |
| | MPNMHTLYWKNLFSDENLKNVCLKLNGEAELFYRPAGIKEPVIHKEGSYLVNRTTEDGESIP |
| | EKIYFEIYKNANGKLEKLSDEAQNYISNHEVVIKKAGHEIIKDRHYTEPKFLFHVPLTINFKAS |
| | GNSYSINENVRKFLKNNPDVNIIGLDRGERHLIYLSLINQKGEIIKQFTFNEVERNKNGRTIKV |
| | NYHEKLDQREKERDAARKSWQAIGKIAELKEGYLSAVIHQLTKLMVEYNAVVVMEDLNFG |
| | FKRGRFHVEKQVYQKFEHILIDKSNYLVFKDRGLNEPGGVLNGYQIAGQFESFQKLGKQSG |
| | MLFYVPAGYTSKIDPKTGFVSMMNFKDLTNVHKKRDFFSKFDNIHYDEANGSFVFTFDYKK |
| | FDGKAKEEMKLTKWSVYSRDKRIVYFAKTKSYEDVLPTEKLQKIFESNGIDYKSGNNIQDSV |
| | MAIGADLKEGAKPSKEISDFWDGLLSNFKLILQMRNSNARTGEDYIISPVMADDGTFFDSRE |
| | EFKKGEDAKLPLDADANGAYHIALKGLSLINKINLSKDEELKKFDMKISNADWFKFAQEKN |
| | YAK* |
| SEQ ID NO: | MENYGGFTGLYPLQKTLKFELRPQGRTMEHLVSSNFFEEDRDRAEKYKIVKKVIDNYHKDFI |
| 18 | NECLSKRSFDWTPLMKTSEKYYASKEKNGKKKQDLDQKIIPTIENLSEKDRKELELEQKRMR |
| | KEIVSVFKEDKRFKYLFSEKLFSILLKDEDYSKEKLTEKEILALKSFNKFSGYFIGLHKNRANF |
| | YSEGDESTAIAYRIVNENFPKFLSNLKKYREVCEKYPEIIQDAEQSLAGLNIKMDDIFPMENF |
| | NKVMTQDGIDLYNLAIGGKAQALGEKQKGLNEFLNEVNQSYKKGNDRIRMTPLFKQILSER |
| | TSYSYILDAFDDNSQLITSINGFFTEVEKDKEGNTFDRAVGLIASYMKYDLSRVYIRKADLNK |
| | VSMEIFGSWERLGGLLRIFKSELYGDVNAEKTSKKVDKWLNSGEFSLSDVINAIAGSKSAETF |
| | DEYILKMRVARGEIDNALEKIKCINGNFSEDENSKMIIKAILDSVQRLFHLFSSFQVRADFSQD |
| | GDFYAEYNEIYEKLFAIVPLYNRVRNYLTKNNLSMKKIKLNFKNPALANGWDLNKEYDNTA |
| | VIFLREGKYYLGIMNPSKKKNIKFEEGSGTGPFYKKMAYKLLPDPNKMLPKVFFAKKNINYY |
| | NPSDEIVKGYKAGKYKKGENFDIDFCHKLIDFFKESIQKNEDWRAFNYLFSATESYKDISDFY |
| | SEVEDQGYRMYFLNVPVANIDEYVEKGDLFLFQIYNKDFASGAKGNKDMHTIYWNAAFSD |
| | ENLRNVVVKLNGEAELFYRDKSIIEPICHKKGEMLVNRTCFDKTPVPDKIHKELFDYHNGRA |
| | KTLSIEAKGYLDRVGVFQASYEIIKDRRYSENKMYFHVPLKLNFKADGKKNLNKMVIEKFLS |
| | DKDVHIIGIDRGERNLLYYSVIDRRGNIIDQDSLNIIDGFDYQKKLGQREIERREARQSWNSIG |
| | KIKDLKEGYLSKAVHKVSKMVLEYNAIVVLEDLNFGFKRGRFKVEKQVYQKFEKMLIDKL |
| | NYLVFKEVLDSRDAGGVLNAYQLTTQLESFNKLGKQSGILFYVPAAYTSKIDPTTGFVSLFN |
| | TSRIESDSEKKDFLSGFDSIVYSAKDGGIFAFKFDYRNRNFQREKTDHKNIWTVYTNGDRIKY |
| | KGRMKGYEITSPTKRIKDVLSSSGIRYDDGQELRDSIIQSGNKVLINEVYNSFIDTLQMRNSD |
| | GEQDYIISPVKNRNGEFFRTDPDRRELPVDADANGAYHIALRGELLMQKIAEDFDPKSDKFT |
| | MPKMEHKDWFEFMQTRGD* |
| SEQ ID NO: | MEVQKTVMKTLSLRILRPLYSQEIEKEIKEEKERRKQAGGTGELDGGFYKKLEKKHSEMFSF |
| 19 | DRLNLLLNQLQREIAKVYNHAISELYIATIAQGNKSNKHYISSIVYNRAYGYFYNAYIALGIC |
| | SKVEANFRSNELLTQQSALPTAKSDNFPIVLHKQKGAEGEDGGFRISTEGSDLIFEIPIPFYEYN |
| | GENRKEPYKWVKKGGQKPVLKLILSTFRRQRNKGWAKDEGTDAEIRKVTEGKYQVSQIEIN |
| | RGKKLGEHQKWFANFSIEQPIYERKPNRSIVGGLDVGIRSPLVCAINNSFSRYSVDSNDVFKF |
| | SKQVFAFRRRLLSKNSLKRKGHGAAHKLEPITEMTEKNDKFRKKIIERWAKEVTNFFVKNQ |
| | VGIVQIEDLSTMKDREDHFFNQYLRGFWPYYQMQTLIENKLKEYGIEVKRVQAKYTSQLCS |
| | NPNCRYWNNYFNFEYRKVNKFPKFKCEKCNLEISADYNAARNLSTPDIEKFVAKATKGINLP |
| | EK* |
| SEQ ID NO: | MIIHNCYIGGSFMKKIDSFTNCYSLSKTLRFKLIPIGATQSNFDLNKMLDEDKKRAENYSKAK |
| 20 | SIIDKYHRFFIDKVLSSVTENKAFDSFLEDVRAYAELYYRSNKDDSDKASMKTLESKMRKFI |
| | ALALQSDEGFKDLFGQNLIKKTLPEFLESDTDKEIIAEFDGFSTYFTGFFNNRKNMYSADDQP |
| | TAISYRCINDNLPKFLDNVRTFKNSDVASILNDNLKILNEDFDGIYGTSAEDVFNVDYFPFVLS |
| | QKGIEAYNSILGGYTNSDGSKIKGLNEYINLYNQKNENIHRIPKMKQLFKQILSERESVSFIPE |
| | KFDSDDDVLSSINDYYLERDGGKVLSIEKTVEKIEKLFSAVTDYSTDGIFVKNAAELTAVCSG |
| | AFGYWGTVQNAWNNEYDALNGYKETEKYIDKRKKAYKSIESFSLADIQKYADVSESSETNA |
| | EVTEWLRNEIKEKCNLAVQGYESSKDLISKPYTESKKLFNNDNAVELIKNALDSVKELENVL |
| | RLLLGTGKEESKDENFYGEFLPCYERICEVDSLYDKVRNYMTQKLYKTDKIKLNFQNPQFLG |
| | GWDRNKEADYSAVLLRRNSLYYIAIMPSGYKRVFEKIPAPKADETVYEKVIYKLLPGPNKM |
| | LPKVFFSKKGIETFNPPKEILEKYELGTHKTGDGFNLDDCHALIDYFKSALDVHSDWSNFGFR |
| | FSDTSTYKNIADFYNEVKNQGYKITFCDVPQSYINELVDEGKLYLFQLYNKDFSEHSKGTPN |
| | LHTLYFKMLFDERNLENVVFKLNGEAEMFYREASISKDDMIVHPKNQPIKNKNEQNSRKQS |
| | TFEYDIVKDRRYTVDQFMLHIPITLNFTANGGTNINNEVRKALKDCDKNYVIGIDRGERNLL |
| | YICVVDSEGRIIEQYSLNEIINEYNGNTYSTDYHALLDKKEKERLESRKAWKTVENIKELKEG |
| | YISQVVHKICELVEKYDAVIVMEDLNLGFKQGRSGKFEKSVYQKFEKMLIDKLNYFADKKK |
| | SPEEIGSVLNAYQLTNAFESFEKMGKQNGFIFYVPAYLTSKIDPTTGFADLLHPSSKQSKESM |
| | RDFVGRFDSITFNKTENYFEFELDYNKFPRCNTDYRKKWTVCTYGSRIKTFRNPEKNSEWDN |
| | KTVELTPAFMALFEKYSIDVNGDIKAQIMSVDKKDFFVELIGLLRLTLQMRNSETGKVDRDY |
| | LISPVKNSEGVFYNSDDYKGIENASLPKDADANGAYNIARKGLWIIEQIKACENDAELNKIRL |
| | AISNAEWLEYAQKK* |
| SEQ ID NO: | MKDYIRKTLSLRILRPYYGEEIEKEIAAAKKKSQAEGGDGALDNKFWDRLKAEHPEIISSREF |
| 21 | YDLLDAIQRETTLYYNRAISKLYHSLIVEREQVSTAKALSAGPYHEFREKFNAYISLGLREKI |
| | QSNFRRKELARYQVALPTAKSDTFPIPIYKGFDKNGKGGFKVREIENGDFVIDLPLMAYHRV |
| | GGKAGREYIELDRPPAVLNVPVILSTSRRRANKTWFRDEGTDAEIRRVMAGEYKVSWVEIL |
| | QRKRFGKPYGGWYVNFTIKYQPRDYGLDPKVKGGIDIGLSSPLVCAVTNSLARLTIRDNDLV |
| | AFNRKAMARRRTLLRQNRYKRSGHGSANKLKPIEALTEKNELYRKAIMRRWAREAADFFR |
| | QHRAATVNMEDLTGIKDREDYFSQMLRCYWNYSQLQTMLENKLKEYGIAVKYIEPKDTSK |
| | TCHSCGHVNEYFDFNYRSAHKFPMFKCEKCGVECGADYNAARNIAQA |
| SEQ ID NO: | MKEQFINRYPLSKTLRFSLIPVGETENNFNKNLLLKKDKQRAENYEKVKCYIDRFHKEYIESV |
| 22 | LSKARIEKVNEYANLYWKSNKDDSDIKAMESLENDMRKQISKQLTSTEIYKKRLFGKELICE |
| | DLPSFLTDKDERETVECFRSFTTYFKGFNTNRENMYSSDGKSTAIAYRCINDNLPRFLDNVKS |
| | FQKVFDNLSDETITKLNTDLYNIFGRNIEDIFSVDYFEFVLTQSGIEIYNSMIGGYTCSDKTKIQ |
| | GLNECINLYNQQVAKNEKSKKLPLMKPLYKQILSEKDSVSFIPEKFNSDNEVLHAIDDYYTG |
| | HIGDFDLLTELLQSLNTYNANGIFVKSGVAITDISNGAFNSWNVLRSAWNEKYEALHPVTSK |
| | TKIDKYIEKQDKIYKAIKSFSLFELQSLGNENGNEITDWYISSINESNSKIKEAYLQAQKLLNS |
| | DYEKSYNKRLYKNEKATELVKNLLDAIKEFQKLIKPLNGTGKEENKDELFYGKFTSYYDSIA |
| | DIDRLYDKVRNYITQKPYSKDKIKLNFDNPQLLGGWDKNKESDYRTVLLHKDGLYYLAVM |
| | DKSHSKAFVDAPEITSDDKDYYEKMEYKLLPGPNKMLPKVFFASKNIDTFQPSDRILDIRKRE |
| | SFKKGATFNKAECHEFIDYFKDSIKKHDDWSQFGFKFSPTESYNDISEFYREISDQGYSVRFN |
| | KISKNYIDGLVNNGYIYLFQIYNKDFSKYSKGTPNLHTLYFKMLFDERNLSNVVYKLNGEAE |
| | MFYREASIGDKEKITHYANQPIKNKNPDNEKKESVFEYDIVKDKRFTKRQFSLHLPITINFKA |
| | HGQEFLNYDVRKAVKYKDDNYVIGIDRGERNLIYISVINSNGEIVEQMSLNEIISDNGHKVDY |
| | QKLLDTKEKERDKARKNWTSVENIKELKEGYISQVVHKICELVIKYDAVIAMEDLNFGFKR |
| | GRFPVEKQVYQKFENMLISKLNLLIDKKAEPTEDGGLLRAYQLTNKFDGVNKAKQNGIIFYV |
| | PAWDTSKIDPATGFVNLLKPKCNTSVPEAKKLFETIDDIKYNANTDMFEFYIDYSKFPRCNSD |
| | FKKSWTVCTNSSRILTFRNKEKNNKWDNKQIVLTDEFKSLFNEFGIDYKGNLKDSILSISNAD |
| | FYRRLIKLLSLTLQMRNSITGSTLPEDDYLISPVANKSGEFYDSRNYKGTNAALPCDADANG |
| | AYNIARKALWAINVLKDTPDDMLNKAKLSITNAEWLEYTQK* |
| SEQ ID NO: | MKEQFVNQYPISKTLRFSLIPIGKTEENFNKNLLLKEDEKKAEEYQKVKGYIDRYHKFFIETA |
| 23 | LCNINFEGFEEYSLLYYKCSKDDNDLKTMEDIEIKLRKQISKTMTSHKLYKDLFGENMIKTIL |
| | PNFLDSDEEKNSLEMFRGFYTYFSGFNTNRKNMYTEEAKSTSIAYRCINDNLPKFLDNSKSFE |
| | KIKCALNKEELKAKNEEFYEIFQIYATDIFNIDFFNFVLTQPGIDKYNGIIGGYTCSDGTKVQG |
| | LNEIINLYNQQIAKDDKSKRLPLLKMLYKQILSDRETVSFIPEKFSSDNEVLESINNYFSKNVS |
| | NAIKSLKELFQGFEAYNMNGIFISSGVAITDLSNAVFGDWNAISTAWEKAYFETNPPKKNKS |
| | QEKYEEELKANYKKIKSFSLDEIQRLGSIAKSPDSIGSVAEYYKITVTEKIDNITELYDGSKELL |
| | NCNYSESYDKKLIKNDTVIEKVKTLLDAVKSLEKLIKPLVGTGKEDKDELFYGTFLPLYTSLS |
| | AVDRLYDKVRNYATQKPYSKDKIKLNFNCSSFLSGWATDYSSNGGLIFEKDGLYYLGIVNK |
| | KFTTEEIDYLQQNADENPAQRIVYDFQKPDNKNTPRLFIRSKGTNYSPSVKEYNLPVEEIVEL |
| | YDKRYFTTEYRNKNPELYKASLVKLIDYFKLGFTRHESYRHYDFKWKKSEEYNDISEFYKD |
| | VEISCYSLKQEKINYNTLLNFVAENRIYLFQIYNKDFSKYSKGTPNLHTRYFKALFDENNLSD |
| | VVFKLNGGSEMFFRKASIKDNEKVVHPANQPIDNKNPDNSKKQSTFDYELIKDKRFTKHQFS |
| | IHIPITMNFKARGRDFINNDIRKAIKSEYKPYVIGIDRGERNLIYISVINNNGEIVEQMSLNDIIS |
| | DNGYKVDYQRLLDRKEKERDNARKSWGTIENIKELKEGYISQVIHKICELVIKYDAVIAMED |
| | LNFGFKRGRFNVEKQVYQKFENMLISKLNYLCDKKSEANSEGGLLKAYQLTNKFDGVNKG |
| | KQNGIIFYVPAWLTSKIDPVTGFVDLLHPKYISVEETHSLFEKLDDIRYNFEKDMFEFDIDYSK |
| | LPKCNADFKQKWTVCTNADRIMTFRNSEKNNEWDNKRILLSDEFKRLFEEFGIDYCHNLKN |
| | KILSISNKDFCYRFIKLFALTMQMRNSITGSTNPEDDYLISPVRDENGVFYDSRNFIGSKAGLPI |
| | DADANGAYNIARKGLWAINAIKSTADDMLDKVDLSISNAKWLEYVQK* |
| SEQ ID NO: | MKITKIDGILHKKYIKEGKLVKSTSEENKTDERLSELLTIRLDTYIKNPDNASEEENRIRRETL |
| 24 | KEFFSNKVLYLKDSILYLKDRREKNQLQNKNYSEEDISEYDLKNKNSFLVLKKILLNEDINSE |
| | ELEIFRNDFEKKLDKINSLKYSLEENKANYQKINENNIKKVEGKSKRNIFYNYYKDSAKRND |
| | YINNIQEAFDKLYKKEDIENLFFLIENSKKHEKYKIRECYHKIIGRKNDKENFATIIYEEIQNVN |
| | NMKELIEKVPNVSELKKSQVFYKYYLNKEKLNDENIKYVFCHFVEIEMSKLLKNYVYKKPS |
| | NISNDKVKRIFEYQSLKKLIENKLLNKLDTYVRNCGKYSFYLQDGEIATSDFIVGNRQNEAFL |
| | RNIIGVSSTAYFSLRNILETENENDITGRIKGKTVKNKKGEEKYISGEIDKLYDNNKQNEVKK |
| | NLKMFYSYDFNMNRKKEIEDFFSNIDEAISSIRHGIVHFNLELEGKDIFTFKNIVPSQISKKMFQ |
| | NEINEKKLKLKIFRQLNSANVFRYLEKYKILNYLNRTRFEFVNKNIPFVPSFTKLYSRIDDLKN |
| | SLCIYWKIPKANDNNKTKEITDAQIYLLKNIYYGEFLNYFMSNNGNFFEIIKEIIELNKNDKRN |
| | LKTGFYKLQKFENLQEKTPKEYLANIQSFYMIDAGNKDEEEKDAYIDFIQKIFLKGFMTYLA |
| | NNGRLSLMYIGNDEQINTSLAGKKQEFDKFLKKYEQNNNIEIPHEINEFVREIKLGKILKYTES |
| | LNMFYLILKLLNHKELTNLKGSLEKYQSANKEEAFSDQLELINLLNLDNNRVTEDFELEADEI |
| | GKFLDFNGNKVKDNKELKKFDTNKIYFDGENIIKHRAFYNIKKYGILNLLEKISDEAKYKISIE |
| | ELKNYSNKKIEIEKNHTTQENLHRKYARPRKDEKFNDEDYKKYEKTIRNIQQYTHLKNKVEF |
| | NELNLLQSLLLRILHRLVGYTSIWERDLRFRLKGEFPENQYIEEIFNFDNSKNVKYKNGQIVE |
| | KYISFYKELYKDDMEKISIYSDKKVKELKKEKKDLYIRNYIAHFNYIPNAEVSLLEVLENLRK |
| | LLSYDRKLKNAIMKSIVDILKEYGFVVTFKIEKDKKIRIESLKSEEVVHLKKLKLKDNDKKKE |
| | PIKTYRNSKELCKLVKVMFEYKMKEKKSEN* |
| SEQ ID NO: | MKITKIDGISHKKYIKEGKLVKSTSEENKTDERLSELLTIRLDTYIKNPDNASEEENRIRRENL |
| 25 | KEFFSNKVLYLKDGILYLKDRREKNQLQNKNYSEEDISEYDLKNKNSFLVLKKILLNEDINSE |
| | ELEIFRKDVEAKLNKINSLKYSFEENKANYQKINENNVEKVGGKSKRNIIYDYYRESAKRND |
| | YINNVQEAFDKLYKKEDIEKLFFLIENSKKHEKYKIRECYHKIIGRKNDKENFAKIIYEEIQNV |
| | NNIKELIEKVPDMSELKKSQVFYKYYLDKEELNDKNIKYAFCHFVEIEMSQLLKNYVYKRLS |
| | NISNDKIKRIFEYQNLKKLIENKLLNKLDTYVRNCGKYNYYLQDGEIATSDFIAGNRQNEAFL |
| | RNIIGVSSVAYFSLRNILETENKDDITGKMRGKTRIDSKTGEEKYIPGEVDQIYYENKQNEVK |
| | NKLKMFYGYDFDMDNKKEIEDFFANIDEAISSIRHGIVHFNLDLDGKDIFAFKNIVPSEISKK |
| | MFQNEINEKKLKLKIFRQLNSANVFRYLEKYKILNYLKRTRFEFVNKNIPFVPSFTKLYSRIDD |
| | LKNSLGIYWKTPKTNDDNKTKEIIDAQIYLLKNIYYGEFLNYFMSNNGNFFEISREIIELNKND |
| | KRNLKTGFYKLQKFEDIQEKTPKKYLANIQSLYMINAGNQDEEEKDTYIDFIQKIFLKGFMTY |
| | LANNGRLSLMYIGNDEQINTSLAGKKQEFDKFLKKYEQNNNIEIPHEINEFLREIKLGKILKYT |
| | ESLNMFYLILKLLNHKELTNLKGSLEKYQSANKEETFSDELELINLLNLDNNRVTEDFELEAN |
| | EIGKFLDFNGNKIKDRKELKKFDTKKIYFDGENIIKHRAFYNIKKYGMLNLLEKIADKAKYKI |
| | SLKELKEYSNKKNEIEKNYTMQQNLHRKYARPKKDEKFNDEDYKEYEKAIGNIQKYTHLKN |
| | KVEFNELNLLQGLLLKILHRLVGYTSIWERDLRFRLKGEFPENQYIEEIFNFDNSKNVKYKSG |
| | QIVEKYINFYKELYKDNVEKRSIYSDKKVKKLKQEKKDLYIRNYIAHFNYIPHAEISLLEVLE |
| | NLRKLLSYDRKLKNAIMKSVVDILKEYGFVATFKIGADKKIGIQTLESEKIVHLKNLKKKKL |
| | MTDRNSEELCKLVKVMFEYKMEEKNLKTKKCKVI* |
| SEQ ID NO: | MKKIDNFVGCYPVSKTLRFKAIPIGKTQENIEKKRLVEEDEVRAKDYKAVKKLIDRYHREFIE |
| 26 | GVLDNVKLDGLEEYYMLFNKSDREESDNKKIEIMEERFRRVISKSFKNNEEYKKIFSKKIIEEI |
| | LPNYIKDEEEKELVKGFKGFYTAFVGYAQNRENMYSDEKKSTAISYRIVNENMPRFITNIKVF |
| | EKAKSILDVDKINEINEYILNNDYYVDDFFNIDFFNYVLNQKGIDIYNAIIGGIVTGDGRKIQG |
| | LNECINLYNQENKKIRLPQFKPLYKQILSESESMSFYIDEIESDDMLIDMLKESLQIDSTINNAI |
| | DDLKVLFNNIFDYDLSGIFINNGLPITTISNDVYGQWSTISDGWNERYDVLSNAKDKESEKYF |
| | EKRRKEYKKVKSFSISDLQELGGKDLSICKKINEIISEMIDDYKSKIEEIQYLFDIKELEKPLVT |
| | DLNKIELIKNSLDGLKRIERYVIPFLGTGKEQNRDEVFYGYFIKCIDAIKEIDGVYNKTRNYLT |
| | KKPYSKDKFKLYFENPQLMGGWDRNKESDYRSTLLRKNGKYYVAIIDKSSSNCMMNIEEDE |
| | NDNYEKINYKLLPGPNKMLPKVFFSKKNREYFAPSKEIERIYSTGTFKKDTNFVKKDCENLIT |
| | FYKDSLDRHEDWSKSFDFSFKESSAYRDISEFYRDVEKQGYRVSFDLLSSNAVNTLVEEGKL |
| | YLFQLYNKDFSEKSHGIPNLHTMYFRSLFDDNNKGNIRLNGGAEMFMRRASLNKQDVTVH |
| | KANQPIKNKNLLNPKKTTTLPYDVYKDKRFTEDQYEVHIPITMNKVPNNPYKINHMVREQL |
| | VKDDNPYVIGIDRGERNLIYVVVVDGQGHIVEQLSLNEIINENNGISIRTDYHTLLDAKERER |
| | DESRKQWKQIENIKELKEGYISQVVHKICELVEKYDAVIALEDLNSGFKNSRVKVEKQVYQK |
| | FEKMLITKLNYMVDKKKDYNKPGGVLNGYQLTTQFESFSKMGTQNGIMFYIPAWLTSKMD |
| | PTTGFVDLLKPKYKNKADAQKFFSQFDSIRYDNQEDAFVFKVNYTKFPRTDADYNKEWEIY |
| | TNGERIRVFRNPKKNNEYDYETVNVSERMKELFDSYDLLYDKGELKETICEMEESKFFEELI |
| | KLFRLTLQMRNSISGRTDVDYLISPVKNSNGYFYNSNDYKKEGAKYPKDADANGAYNIARK |
| | VLWAIEQFKMADEDKLDKTKISIKNQEWLEYAQTHCE |
| SEQ ID NO: | MKKIDSFVNYYPLSKTLRFSLIPVGKTEDNFNAKLLLEEDEKRAIEYEKVKRYIDRYHKHFIE |
| 27 | TVLANFHLDDVNEYAELYYKAGKDDKDLKYMEKLEGKMRKSISAAFTKDKKYKEIFGQEII |
| | KNILPEFLENEDEKESVKMFQGFFTYFTGFNDNRKNMYTHEAQTTAISYRCINENLPKFLDN |
| | VQSFAKIKESISSDIMNKLDEVCMDLYGVYAQDMFCTDYFSFVLSQSGIDRYNNIIGGYVDD |
| | KGVKIQGINEYINLYNQQVDEKNKRLPLMKKLYKQILIEKESISFIPEKFESDNIVINAISDYYH |
| | NNVENLFDDFNKLFNEFSEYDDNGIFVTSGLAVTDISNAVFGSWNIISDSWNEEYKDSHPMK |
| | KTTNAEKYYEDMKKEYKKNLSFTIAELQRLGEAGCNDECKGDIKEYYKTTVAEKIENIKNA |
| | YEISKDLLASDYEKSNDKKLCKNDSAISLLKNLLDSIKDLEKTIKPLLGTGKEENKDDVFYGK |
| | FTNLYEMISEIDRLYDKVRNYVTQKPYSKDKIKLNFENPQHLGGWDKNKERDYRSVLLKKE |
| | DKYYLAIMDKSNNKAFIDFPDDGECYEKIEYKLLPGPNKMLPKVFFASSNIEYFAPSKKILEIR |
| | SRESFKKGDMFNLKDCHEFIDFFKESIKKHEDWSQFGFEFSPTEKYNDISEFYNEVKIQGYSL |
| | KYKNVSKKYIDELIECGQLYLFQIYNKDFSVYAKGNPNLHTMYFKMLFDERNLANVVYQLN |
| | GGAEMFYRKASIKDSEKIVHHANQPIKNKNADNVKKESVFEYDIIKDKRFTKRQFSIHIPITLN |
| | FKAKGQNFINNDVRMALKKADENYVIGIDRGERNLLYICVINSKGEIVEQKSLNEIIGDNGYR |
| | VDYHKLLDKKEAERDEARKSWGTIENIKELKEGYLSQIVHEISKLVIKYDAVIAIEDLNSGFK |
| | KGRFKVEKQVYQKFENMLCTKLNYLVDKNADANECGGLLKAYQLTNKEDGANRGRQNGI |
| | IFSVPAWLTSKIDPVTGFADLLRPKYKSVSESVEFISKIDNIRYNSKEDYFEFDIDYSKFPNSTA |
| | SYKKKWTVCTYGERIINVRNKEKNNMWDNKTIVLTDEFKKLFADFGVDVSKNIKESVLAID |
| | SKDFYYRFINLLANTLQLRNSEVGNVDVDYLISPVKGVDGSFYDSRLVKEKTLPENADANG |
| | AYNIARKALWAIDVLKQTKDEELKNANLSIKNAEWLEYVQK* |
| SEQ ID NO: | MKNQNTLPSNPTDILKDKPFWAAFFNLARHNVYLTVNHINKLLDLEKLYNKDKHKEIFEHE |
| 28 | DIFNISDDVMNDVNSNGKKRKLDIKKIWANLDTDLTRKYQLRELILKHFPFIQPAIIGAQTKE |
| | RTTIDKDKRSTSTSNDSLKPTGEGDINDPLSLSNVKSIFFRLLQMLEQLRNYYSHVKHSKSAT |
| | MPNFDEGLLKSMYNIFIDSVNKVKEDYSSNSVIDPNTSFSHLISKDEQGEIKPCRYSFTSKDGS |
| | INASGLLFFVSLFLEKQDSIWMQKKIPGFKKTSENYMKMTNEVFCRNHILLPKMRLETVYDK |
| | DWMLLDMLNEVVRCPLSLYKRLAPADQNKFKVPEKSSDNANRQEDDNPFSRILVRHQNRFP |
| | YFALRFFDLNEVFTTLRFQINLGCYHFAICKKQIGDKKEVHHLTRTLYGFSRLQNFTQNTRPE |
| | EWNTLVKTTEPSSGNDGKTVQGVPLPYISYTIPHYQIENEKIGIKIFDGDTAVDTDIWPSVSTE |
| | KQLNKPDKYTLTPGFKADVFLSVHELLPMMFYYQLLLCEGMLKTDAGNAVEKVLIDTRNAI |
| | FNLYDAFVQEKINTITDLENYLQDKPILIGHLPKQMIDLLKGHQRDMLKAVEQKKAMLIKDT |
| | ERRLERLNKQPEQKPNVAAKNTGTLLRNGQIADWLVKDMMRFQPVKRDKEGNPINCSKAN |
| | STEYQMLQRAFAFYTTDSYRLPRYFEQLHLINCDNSHLFLSRFEYDKQPNLIAFYAAYLEAK |
| | LEFLNELQPQNWASDNYFLLLRAPKNDRQKLAEGWKNGFNLPRGLFTEKIKTWFNEHKTIV |
| | DISDCDIFKNRVGQVARLIPVFFDKKFKDHSQPFYTYNFNVGNVSKITEANYLSKEKRENLFK |
| | SYQNKFKNNIPAEKTKEYREYKNFSSWKKFERELRLIKNQDILTWLMCKNLFDEKIKPKKDI |
| | LEPRIAVSYIKLDSLQTNTSTAGSLNALAKVVPMTLAIHIDSPKPKGKAGNNEKENKEFTVYI |
| | KEEGTKLLKWGNFKTLLADRRIKGLFSYIEHDDINLEKYPLTKYQVDSELDLYQKYRIDIFKQ |
| | TLDLEAQLLDKYSDLNTDNFNQMLSGWSEKEGIPRNIKQDVAFLIGVRNGFSHNQYPDSKRI |
| | AFSRIKKFNPKTSSLQESEGLNIAKQMYEEAQQVVNKIKNIESFD* |
| SEQ ID NO: | MKVTKIDGISHKKFEDEGKLVKFTGHFNIKNEMKERLEKLKELKLSNYIKNPENVKNKDKN |
| 29 | KEKETKSRRENLKKYFSEIILRKKEEKYLLKKTRKFKNITEEINYDDIKKRENQQKIFDVLKEL |
| | LEQRINENDKEEILNFDSVKLKEAFEEDFIKKELKIKAIEESLEKNRADYRKDYVELENEKYE |
| | DVKGQNKRSLVFEYYKNPENREKFKENIKYAFENLYTEENIKNLYSEIKEIFEKVHLKSKVR |
| | YFYQNEIIGESEFSEKDEEGISILYKQIINSVEKKEKFIEFLQKVKIKDLTRSQIFYKYFLENEEL |
| | NDENIKYVFSYFVEIEVNKLLKENVYKTKKFNEGNKYRVKNIFNYDKLKNLVVYKLENKLN |
| | NYVRNCGKYNYHMENGDIATSDINMKNRQTEAFLRSILGVSSFGYFSLRNILGVNDDDFYKI |
| | EKDERKNENFILKKAKEDFTSKNIFEKVVDKSFEKKGIYQIKENLKMFYGNSFDKVDKDELK |
| | KFFVNMLEAITSVRHRIVHYNINTNSENIFDFSNIEVSKLLKNIFEKEIDTRELKLKIFRQLNSA |
| | GVFDYWESWVIKKYLENVKFEFVNKNVPFVPSFKKLYDRIDNLKGWNALKLGNNINIPKRK |
| | EAKDSQIYLLKNIYYGEFVEKFVNDNKNFEKIVKEIIEINRGAGTNKKTGFYKLEKFETLKAN |
| | TPTKYLEKLQSLHKISYDKEKIEEDKDVYVDFVQKIFLKGFVNYLKKLDSLKSLNLLNLRKD |
| | ETITDKKSVHDEKLKLWENSGSNLSKMPEEIYEYVKKIKISNINYNDRMSIFYLLLKLIDYREL |
| | TNLRGNLEKYESMNKNKIYSEELTIINLVNLDNNKVRTNFSLEAEDIGKFLKSSITIKNIAQLN |
| | NFSKIFADGENVIKHRSFYNIKKYGILDLLEKIVAKADLKITKEEIKKYENLQNELKRNDFYKI |
| | QEQIHRNYNQKPFSIKKIENKKDFEKYKKVIEKIQDYTQLKNKIEFNDLNLLQSLIFRILHRLA |
| | GYTSLWERDLQFKLKGEFPEDKYIDEIFNSDGNNNQKYKHGGIADKYANFLIEKKEEKSGEI |
| | LNKKQRKKKIKEDLEIRNYIAHFNYLPNAEKSILEILEELRELLKHDRKLKNAVMKSIKDIFRE |
| | YGFIVEFTISHTKNGKKIKVCSVKSEKIKHLKNNELITTRNSEDLCELVKIMLEHKELQK* |
| SEQ ID NO: | MKVTKVGGISHKKYTSEGRLVKSESEENRTDERLSALLNMRLDMYIKNPSSTETKENQKRIG |
| 30 | KLKKFFSNKMVYLKDNTLSLKNGKKENIDREYSETDILESDVRDKKNFAVLKKIYLNENVN |
| | SEELEVFRNDIKKKLNKINSLKYSFEKNKANYQKINENNIEKVEGKSKRNIIYDYYRESAKRD |
| | AYVSNVKEAFDKLYKEEDIAKLVLEIENLTKLEKYKIREFYHEIIGRKNDKENFAKIIYEEIQN |
| | VNNMKELIEKVPDMSELKKSQVFYKYYLDKEELNDKNIKYAFCHFVEIEMSQLLKNYVYKR |
| | LSNISNDKIKRIFEYQNLKKLIENKLLNKLDTYVRNCGKYNYYLQDGEIATSDFIARNRQNEA |
| | FLRNIIGVSSVAYFSLRNILETENENDITGRMRGKTVKNNKGEEKYVSGEVDKIYNENKKNE |
| | VKENLKMFYSYDFNMDNKNEIEDFFANIDEAISSIRHGIVHFNLELEGKDIFAFKNIAPSEISK |
| | KMFQNEINEKKLKLKIFRQLNSANVFRYLEKYKILNYLKRTRFEFVNKNIPFVPSFTKLYSRID |
| | DLKNSLGIYWKTPKTNDDNKTKEIIDAQIYLLKNIYYGEFLNYFMSNNGNFFEISKEIIELNKN |
| | DKRNLKTGFYKLQKFEDIQEKIPKEYLANIQSLYMINAGNQDEEEKDTYIDFIQKIFLKGFMT |
| | YLANNGRLSLIYIGSDEETNTSLAEKKQEFDKFLKKYEQNNNIKIPYEINEFLREIKLGNILKY |
| | TERLNMFYLILKLLNHKELTNLKGSLEKYQSANKEEAFSDQLELINLLNLDNNRVTEDFELE |
| | ADEIGKFLDFNGNKVKDNKELKKFDTNKIYFDGENIIKHRAFYNIKKYGMLNLLEKIADKAG |
| | YKISIEELKKYSNKKNEIEKNHKMQENLHRKYARPRKDEKFTDEDYESYKQAIENIEEYTHL |
| | KNKVEFNELNLLQGLLLRILHRLVGYTSIWERDLRFRLKGEFPENQYIEEIFNFENKKNVKYK |
| | GGQIVEKYIKFYKELHQNDEVKINKYSSANIKVLKQEKKDLYIRNYIAHFNYIPHAEISLLEVL |
| | ENLRKLLSYDRKLKNAVMKSVVDILKEYGFVATFKIGADKKIGIQTLESEKIVHLKNLKKKK |
| | LMTDRNSEELCKLVKIMFEYKMEEKKSEN |
| SEQ ID NO: | MNELVKNRCKQTKTICQKLIPIGKTRETIEKYNLMEIDRKIAANKELMNKLFSLIAGKHINDT |
| 31 | LSKCTDLDFEPLLTSLSSLNNAKENDRDNLREYYDSVFEEKKTLAEEISSRLTAVKFAGKDFF |
| | TKNIPDFLETYEGDDKNEMSELVSLVIENTVTAGYVKKLEKIDRSMEYRLVSGTVVKRVLTD |
| | NADIYEKNIEKAKDFDYGVLNIDEASQFTTLVAKDYANYLTADGIAIYNVGIGKINLALNEY |
| | CQKNKEYSYNKLALLPLQKMLYGEKLSLFEKLEDFTSDEELINSYNKFAKTVNESGLAEIIKK |
| | AVPSYDEIVIKPNKISNYSNSITGHWSLVNRIMKDYLENNGIKNADKYMEKGLTLSEIGDALE |
| | NKNIKHSDFISNLINDLGHTYTEIKENKESLKKDESVNALIIKKELDMLLSILQNLKVEDIDNE |
| | MFDTGFGIEVSKAIEILGYGVPLYNKIRNYITKKPDPKKKFMTKFGSATIGTGITTSVEGSKKA |
| | TFLKDGDAVFLLLYNTAGCKANNVSVSNLADLINSSLEIENSGKCYQKMIYQTPGDIKKQIPR |
| | VFVYKSEDDDLIKDFKAGLHKTDLSFLNGRLIPYLKEAFATHETYKNYTFSYRNSYESYDEF |
| | CEHMSEQAYILEWKWIDKKLIDDLVEDGSLLMFRVWNRFMKKKEGKISKHAKIVNELFSDE |
| | NASNAAIKLLSVFDIFYRDKQIDNPIVHKAGTTLYNKRTKDGEVIVDYTTMVKNKEKRPNVY |
| | TTTKKYDIIKDRRYTEEQFEIHLHVNIGKEENKEKLETSKVINEKKNTLVVTRSNEHLLYVVIF |
| | DENDNILLKKSLNTVKGMNFKSKLEVVEIQKKENMQSWKTVGSNQALMEGYLSFAIKEIAD |
| | LVKEYDAILVLEQNSVGKNILNERVYTRFKEMLITNLSLDVDYENKDFYSYTELGGKVASW |
| | RDCVTNGICIQVPSAYKYKDPTTSFSTISMYAKTTAEKSKKLKQIKSFKYNRERGLFELVIAK |
| | GVGLENNIVCDSFGSRSIIENDISKEVSCTLKIEKYLIDAGIEYNDEKEVLKDLDTAAKTDAVH |
| | KAVTLLLKCFNESPDGRYYISPCGEHFTLCDAPEVLSAINYYIRSRYIREQIVEGVKKMEYKK |
| | TILLAK* |
| SEQ ID NO: | MNGNRIIVYREFVGVTPVAKTLRNELRPIGHTQEHIIHNGLIQEDELRQEKSTELKNIMDDYY |
| 32 | REYIDKSLSGVTDLDFTLLFELMNLVQSSPSKDNKKALEKEQSKMREQICTHMQSDSNYKNI |
| | FNAKFLKEILPDFIKNYNQYDAKDKAGKLETLALFNGFSTYFTDFFEKRKNVFTKEAVSTSIA |
| | YRIVHENSLTFLANMTSYKKISEKALDEIEVIEKNNQDKMGDWELNQIFNPDFYNMVLIQSGI |
| | DFYNEICGVVNAHMNLYCQQTKNNYNLFKMRKLHKQILAYTSTSFEVPKMFEDDMSVYNA |
| | VNAFIDETEKGNIIGKLKDIVNKYDELDEKRIYISKDFYETLSCFMSGNWNLITGCVENFYDE |
| | NIHAKGKSKEEKVKKAVKEDKYKSINDVNDLVEKYIDEKERNEFKNSNAKQYIREISNIITDT |
| | ETAHLEYDEHISLIESEEKADEMKKRLDMYMNMYHWAKAFIVDEVLDRDEMFYSDIDDIYN |
| | ILENIVPLYNRVRNYVTQKPYNSKKIKLNFQSPTLANGWSQSKEFDNNAIILIRDNKYYLAIF |
| | NAKNKPDKKIIQGNSDKKNDNDYKKMVYNLLPGANKMLPKVFLSKKGIETFKPSDYIISGY |
| | NAHKHIKTSENFDISFCRDLIDYFKNSIEKHAEWRKYEFKFSATDSYNDISEFYREVEMQGYR |
| | IDWTYISEADINKLDEEGKIYLFQIYNKDFAENSTGKENLHTMYFKNIFSEENLKDIIIKLNGQ |
| | AELFYRRASVKNPVKHKKDSVLVNKTYKNQLDNGDVVRIPIPDDIYNEIYKMYNGYIKEND |
| | LSEAAKEYLDKVEVRTAQKDIVKDYRYTVDKYFIHTPITINYKVTARNNVNDMAVKYIAQN |
| | DDIHVIGIDRGERNLIYISVIDSHGNIVKQKSYNILNNYDYKKKLVEKEKTREYARKNWKSIG |
| | NIKELKEGYISGVVHEIAMLMVEYNAIIAMEDLNYGFKRGRFKVERQVYQKFESMLINKLN |
| | YFASKGKSVDEPGGLLKGYQLTYVPDNIKNLGKQCGVIFYVPAAFTSKIDPSTGFISAFNFKSI |
| | STNASRKQFFMQFDEIRYCAEKDMFSFGFDYNNFDTYNITMSKTQWTVYTNGERLQSEFNN |
| | ARRTGKTKSINLTETIKLLLEDNEINYADGHDVRIDMEKMDEDKNSEFFAQLLSLYKLTVQM |
| | RNSYTEAEEQEKGISYDKIISPVINDEGEFFDSDNYKESDDKECKMPKDADANGAYCIALKG |
| | LYEVLKIKSEWTEDGFDRNCLKLPHAEWLDFIQNKRYE* |
| SEQ ID NO: | MNKDIRKNFTDFVGISEIQKTLRFILIPIGKTAQNIDKYNMFEDDEIRHEYYPILKEACDDFYR |
| 33 | NHIDQQFENLELDWSKLDEALASEDRDLINETRATYRQVLFNRLKNSVDIKGDSKKNKTLSL |
| | ESSDKNLGKKKTKNTFQYNFNDLFKAKLIKAILPLYIEYIYEGEKLENAKKALKMYNRFTSR |
| | LSNFWQARANIFTDDEISTGSPYRLVNDNFTIFRINNSIYTKNKPFIEEDILEFEKKLKSKKIIKD |
| | FESVDDYFTVNAFNKLCTQNGIDKYNSILGGFTTKEREKVKGLNELFNLAQQSINKGKKGEY |
| | RKNIRLGKLTKLKKQILAISDSTSFLIEQIEDDQDLYNKIKDFFELLLKEEIENENIFTQYANLQ |
| | KLIEQADLSKIYINAKHLNKISHQVTGKWDSLNKGIALLLENININEESKEKSEVISNGQTKDI |
| | SSEAYKRYLQIQSEEKDIERLRTQIYFSLEDLEKALDLVLIDENMDRSDKSILSYVQSPDLNVN |
| | FERDLTDLYSRIMKLEENNEKLLANHSAIDLIKEFLDLIMLRYSRWQILFCDSNYELDQTFYPI |
| | YDAVMEILSNIIRLYNLARNYLSRKPDRMKKKKINFNNPTLADGWSESKIPDNSSMLFIKDG |
| | MYYLGIIKNRAAYSELLEAESLQSSEKKKSENSSYERMNYHFLPDAFRSIPKSSIAMKAVKEH |
| | FEINQKTADLLLDTDKFSKPLRITKEIFDMQYVDLHKNKKKYQVDYLRDTGDKKGYRKALN |
| | TWLNFCKDFISKYKGRNLFDYSKIKDADHYETVNEFYNDVDKYSYHIFFTSVAETTVEKFISE |
| | GKLYLFQLYNKDFSPHSTGKPNLHTIYWRALFSEENLTSKNIKLNGQAEIFFRPKQIETPFTHK |
| | KGSILVNRFDVNGNPIPINVYQEIKGFKNNVIKWDDLNKTTQEGLENDQYLYFESEFEIIKDR |
| | RYTEDQLFFHVPISFNWDIGSNPKINDLATQYIVNSNDIHIIGIDRGENHLIYYSVIDLQGAIVE |
| | QGSLNTITEYTENKFLNNKTNNLRKIPYKDILQQREDERADARIKWHAIDKIKDLKDGYLGQI |
| | VHFLAKLIIKYNAIVILEDLNYGFKRGRFKVERQVYQKFEMALMKKLNVLVFKDYDIDEIGG |
| | PLKPWQLTRPIDSYERMGRQNGILFYVPAAYTSAVDPVTGFANLFYLNNVKNSEKFHFFSKF |
| | ESIKYHSDQDMFSFAFDYNNFGTTTRINDLSKSKWQVFTNHERSVWNNKEKNYVTQNLTDL |
| | IKKLLRTYNIEFKNNQNVLDSILKIENNTDKENFARELFRLFRLTIQLRNTTVNENNTEITENE |
| | LDYIISPVKDKNGNFFDSRDELKNLPDNGDANGAYNIARKGLLYIEQLQESIKTGKLPTLSIST |
| | LDWFNYIMK* |
| SEQ ID NO: | MNKGGYVIMEKMTEKNRWENQFRITKTIKEEIIPTGYTKVNLQRVNMLKREMERNEDLKK |
| 34 | MKEICDEYYRNMIDVSLRLEQVRTLGWESLIHKYRMLNKDEKEIKALEKEQEDLRKKISKGF |
| | GEKKAWTGEQFIKKILPQYLMDHYTGEELEEKLRIVKKFKGCTMFLSTFFKNRENIFTDKPIH |
| | TAVGHRITSENAMLFAANINTYEKMESNVTLEIERLQREFWRRGINISEIFTDAYYVNVLTQK |
| | QIEAYNKICGDINQHMNEYCQKQKLKFSEFRMRELKKQILAVVGEHFEIPEKIESTKEVYREL |
| | NEYYESLKELHGQFEELKSVQLKYSQIYVQKKGYDRISRYIGGQWDLIQECMKKDCASGMK |
| | GTKKNHDAKIEEEVAKVKYQSIEHIQKLVCTYEEDRGHKVTDYVDEFIVSVCDLLGADHIIT |
| | RDGERIELPLQYEPGTDLLKNDTINQRRLSDIKTILDWHMDMLEWLKTFLVNDLVIKDEEFY |
| | MAIEELNERMQCVISVYNRIRNYVTQKGYEPEKIRICFDKGTILTGWTTGDNYQYSGFLLMR |
| | NDKYYLGIINTNEKSVRKILDGNEECKDENDYIRVGYHLINDASKQLPRIFVMPKAGKKSEIL |
| | MKDEQCDYIWDGYCHNKHNESKEFMRELIDYYKRSIMNYDKWEGYCFKFSSTESYDNMQ |
| | DFYKEVREQSYNISFSYINENVLEQLDKDGKIYLFQVYNKDFAAGSTGTPNLHTMYLQNLFS |
| | SQNLELKRLRLGGNAELFYRPGTEKDVTHRKGSILVDRTYVREEKDGIEVRDTVPEKEYLEI |
| | YRYLNGKQKGDLSESAKQWLDKVHYREAPCDIIKDKRYAQEKYFLHFSVEINPNAEGQTAL |
| | NDNVRRWLSEEEDIHVIGIDRGERNLIYVSLMDGKGRIKDQKSYNIVNSGNKEPVDYLAKLK |
| | VREKERDEARRNWKAIGKIKDIKTGYLSYVVHEIVEMAVREKAIIVMEDLNYGFKRGRFKV |
| | ERQVYQKFEEMLINKLNYVVDKQLSVDEPGGLLRGYQLAFIPKDKKSSMRQNGIVFYVPAG |
| | YTSKIDPTTGFVNIFKFPQFGKGDDDGNGKDYDKIRAFFGKFDEIRYECDEKVTADNTREVK |
| | ERYRFDFDYSKFETHLVHMKKTKWTVYAEGERIKRKKVGNYWTSEVISDIALRMSNTLNIA |
| | GIEYKDGHNLVNEICALRGKQAGIILNELLEIVRLTVQLRNSTTEGDVDERDEIISPVLNEKYG |
| | CFYHSTEYKQQNGDVLPKDADANGAYCIGLKGIYEIRQIKNKWKEDMTKGEGKALNEGMR |
| | ISHDQWFEFIQNMNKGE* |
| SEQ ID NO: | MNNPRGAFGGFTNLYSLSKTLRFELKPYLEIPEGEKGKLFGDDKEYYKNCKTYTEYYLKKA |
| 35 | NKEYYDNEKVKNTDLQLVNFLHDERIEDAYQVLKPVFDTLHEEFITDSLESAEAKKIDFGNY |
| | YGLYEKQKSEQNKDEKKKIDKPLETERGKLRKAFTPIYEAEGKNLKNKAGKEKKDKDILKE |
| | SGFKVLIEAGILKYIKNNIDEFADKKLKNNEGKEITKKDIETALGAENIEGIFDGFFTYFSGFN |
| | QNRENYYSTEEKATAVASRIVDENLSKFCDNILLYRKNENDYLKIFNFLKNKGKDLKLKNSK |
| | FGKENEPEFIPAYDMKNDEKSFSVADFVNCLSQGEIEKYNAKIANANYLINLYNQNKDGNSS |
| | KLSMFKILYKQIGCGEKKDFIKTIKDNAELKQILEKACEAGKKYFIRGKSEDGGVSNIFDFTD |
| | YIQSHENYKGVYWSDKAINTISGKYFANWDTLKNKLGDAKVFNKNTGEDKADVKYKVPQ |
| | AVMLSELFAVLDDNAGEDWREKGIFFKASLFEGDQNKSEIIKNANRPSQALLKMICDDMESL |
| | AKNFIDSGDKILKISDRDYQKDENKQKIKNWLDNALWINQILKYFKVKANKIKGDSIDARID |
| | SGLDMLVFSSDNPAEDYDMIRNYLTQKPQDEINKLKLNFENSSLAGGWDENKEKDNSCIILK |
| | DEQDKQYLAVMKYENTKVFEQKNSQLYIADNAAWKKMIYKLVPGASKTLPKVFFSKKWT |
| | ANRPTPSDIVEIYQKGSFKKENVDFNDKKEKDESRKEKNREKIIAELQKTCWMDIRYNIDGKI |
| | ESAKYVNKEKLAKLIDFYKENLKKYPSEEESWDRLFAFGFSDTKSYKSIDQFYIEVDKQGYK |
| | LEFVTINKARLDEYVRDGKIYLFEIRSRDNNLVNGEEKTSAKNLQTIYWNAAFGGDDNKPKL |
| | NGEAEIFYRPAIAENKLNKKKDKNGKEIIDGYRFSKEKFIFHCPITLNFCLKETKINDKLNAAL |
| | AKPENGQGVYFLGIDRGEKHLAYYSLVNQKGEILEQGTLNLPFLDKNGKSRSIKVEKKSFEK |
| | DSNGGIIKDKDGNDKIKIEFVECWNYNDLLDARAGDRDYARKNWTTIGTIKELKDGYISQV |
| | VRKIVDLSIYKNTETKEFREMPAFIVLEDLNIGFKRGRQKIEKQVYQKLELALAKKLNFLVDK |
| | KADIGEIGSVTKAIQLTPPVNNFGDMENRKQFGNMLYIRADYTSQTDPATGWRKSIYLKSGS |
| | ESNVKEQIEKSFFDIRYESGDYCFEYRDRHGKMWQLYSSKNGVSLDRFHGERNNSKNVWES |
| | EKQPLNEMLDILFDEKRFDKSKSLYEQMFKGGVALTRLPKEINKKDKPAWESLRFVIILIQQI |
| | RNTGKNGDDRNGDFIQSPVRDEKTGEHFDSRIYLDKEQKGEKADLPTSGDANGAYNIARKG |
| | IVVAEHIKRGFDKLYISDEEWDTWLAGDEIWDKWLKENRESLTKTRK* |
| SEQ ID NO: | MNYKTGLEDFIGKESLSKTLRNALIPTESTKIHMEEMGVIRDDELRAEKQQELKEIMDDYYR |
| 36 | TFIEEKLGQIQGIQWNSLFQKMEETMEDISVRKDLDKIQNEKRKEICCYFTSDKRFKDLFNAK |
| | LITDILPNFIKDNKEYTEEEKAEKEQTRVLFQRFATAFTNYFNQRRNNFSEDNISTAISFRIVNE |
| | NSEIHLQNMRAFQRIEQQYPEEVCGMEEEYKDMLQEWQMKHIYSVDFYDRELTQPGIEYYN |
| | GICGKINEHMNQFCQKNRINKNDFRMKKLHKQILCKKSSYYEIPFRFESDQEVYDALNEFIKT |
| | MKKKEIIRRCVHLGQECDDYDLGKIYISSNKYEQISNALYGSWDTIRKCIKEEYMDALPGKG |
| | EKKEEKAEAAAKKEEYRSIADIDKIISLYGSEMDRTISAKKCITEICDMAGQISIDPLVCNSDIK |
| | LLQNKEKTTEIKTILDSFLHVYQWGQTFIVSDIIEKDSYFYSELEDVLEDFEGITTLYNHVRSY |
| | VTQKPYSTVKFKLHFGSPTLANGWSQSKEYDNNAILLMRDQKFYLGIFNVRNKPDKQIIKGH |
| | EKEEKGDYKKMIYNLLPGPSKMLPKVFITSRSGQETYKPSKHILDGYNEKRHIKSSPKFDLGY |
| | CWDLIDYYKECIHKHPDWKNYDFHFSDTKDYEDISGFYREVEMQGYQIKWTYISADEIQKL |
| | DEKGQIFLFQIYNKDFSVHSTGKDNLHTMYLKNLFSEENLKDIVLKLNGEAELFFRKASIKTP |
| | IVHKKGSVLVNRSYTQTVGNKEIRVSIPEEYYTEIYNYLNHIGKGKLSSEAQRYLDEGKIKSF |
| | TATKDIVKNYRYCCDHYFLHLPITINFKAKSDVAVNERTLAYIAKKEDIHIIGIDRGERNLLYI |
| | SVVDVHGNIREQRSFNIVNGYDYQQKLKDREKSRDAARKNWEEIEKIKELKEGYLSMVIHYI |
| | AQLVVKYNAVVAMEDLNYGFKTGRFKVERQVYQKFETMLIEKLHYLVFKDREVCEEGGVL |
| | RGYQLTYIPESLKKVGKQCGFIFYVPAGYTSKIDPTTGFVNLFSFKNLTNRESRQDFVGKFDE |
| | IRYDRDKKMFEFSFDYNNYIKKGTILASTKWKVYTNGTRLKKIVVNGKYTSQSMEVELTDA |
| | MEKMLQRAGIEYHDGKDLKGQIVEKGIEAEIIDIFRLTVQMRNSRSESEDREYDRLISPVLND |
| | KGEFFDTATADKTLPQDADANGAYCIALKGLYEVKQIKENWKENEQFPRNKLVQDNKTWF |
| | DFMQKKRYL* |
| SEQ ID NO: | MRISKTLSLRIVRPFYTPEVEAGIKAEKDKREAQGQTRSLDAKFFNELKKKHSEIILSSEFYSL |
| 37 | LSEVQRQLTSIYNHAMSNLYHKIIVEGEKTSTSKALSNIGYDECKAIFPSYMALGLRQKIQSN |
| | FRRRDLKNFRMAVPTAKSDKFPIPIYRQVDGSKGGFKISENDGKDFIVELPLVDYVAEEVKT |
| | AKGRFTKINISKPPKIKNIPVILSTLRRRQSGQWFSDDGTNAEIRRVISGEYKVSWIEIVRRTRF |
| | GKHDDWFVNMVIKYDKPEEGLDSKVVGGIDVGVSSPLVCALNNSLDRYFVKSSDIIAFNKR |
| | AMARRRTLLRQNKYKRSGHGSKNKLEPITVLTEKNERFKKSIMQRWAKEVAEFFRGKGASV |
| | VRMEELSGLKEKDNFFSSYLRMYWNYGQLQQIIENKLKEYGIKVNYVSPKDTSKKCHSCTHI |
| | NEFFTFEYRQKNNFPLFKCEKCGVECSADYNAAKNMAIA |
| SEQ ID NO: | MRTMVTFEDFTKQYQVSKTLRFELIPQGKTLENMKRDGIISVDRQRNEDYQKAKGILDKLY |
| 38 | KYILDFTMETVVIDWEALATATEEFRKSKDKKTYEKVQSKIRTALLEHVKKQKVGTEDLFK |
| | GMFSSKIITGEVLAAFPEIRLSDEENLILEKFKDFTTYFTGFFENRKNVFTDEALSTSFTYRLVN |
| | DNFIKFFDNCIVFKNVVNISPHMAKSLETCASDLGIFPGVSLEEVFSISFYNRLLTQTGIDQFNQ |
| | LLGGISGKEGEHKQQGLNEIINLAMQQNLEVKEVLKNKAHRFTPLFKQILSDRSTMSFIPDAF |
| | ADDDEVLSAVDAYRKYLSEKNIGDRAFQLISDMEAYSPELMRIGGKYVSVLSQLLFYSWSEI |
| | RDGVKAYKESLITGKKTKKELENIDKEIKYGVTLQEIKEALPKKDIYEEVKKYAMSVVKDY |
| | HAGLAEPLPEKIETDDERASIKHIMDSMLGLYRFLEYFSHDSIEDTDPVFGECLDTILDDMNE |
| | TVPLYNKVRNFSTRKVYSTEKFKLNFNNSSLANGWDKNKEQANGAILLRKEGEYFLGIFNS |
| | KNKPKLVSDGGAGIGYEKMIYKQFPDFKKMLPKCTISLKDTKAHFQKSDEDFTLQTDKFEKS |
| | IVITKQIYDLGTQTVNGKKKFQVDYPRLTGDMEGYRAALKEWIDFGKEFIQAYTSTAIYDTS |
| | LFRDSSDYPDLPSFYKDVDNICYKLTFEWIPDAVIDDCIDDGSLYLFKLHNKDFSSGSIGKPNL |
| | HTLYWKALFEEENLSDVVVKLNGQAELFYRPKSLTRPVVHEEGEVIINKTTSTGLPVPDDVY |
| | VELSKFVRNGKKGNLTDKAKNWLDKVTVRKMPHAITKDRRFTVDKFFFHVPITLNYKADSS |
| | PYRFNDFVRQYIKDCSDVKIIGIDRGERNLIYAVVIDGKGNIIEQRSFNTVGTYNYQEKLEQK |
| | EKERQTARQDWATVTKIKDLKKGYLSAVVHELSKMIVKYKAIVALENLNVGFKRMRGGIA |
| | ERSVYQQFEKALIDKLNYLVFKDEEQSGYGGVLNAYQLTDKFESFSKMGQQTGFLFYVPAA |
| | YTSKIDPLTGFINPFSWKHVKNREDRRNFLNLFSKLYYDVNTHDFVLAYHHSNKDSKYTIKG |
| | NWEIADWDILIQENKEVFGKTGTPYCVGKRIVYMDDSTTGHNRMCAYYPHTELKKLLSEYG |
| | IEYTSGQDLLKIIQEFDDDKLVKGLFYIIKAALQMRNSNSETGEDYISSPIEGRPGICFDSRAEA |
| | DTLPYDADANGAFHIAMKGLLLTERIRNDDKLAISNEEWLNYIQEMRG* |
| SEQ ID NO: | MSKLEKFTNCYSLSKTLRFKAIPVGKTQENIDNKRLLVEDEKRAEDYKGVKKLLDRYYLSFI |
| 39 | NDVLHSIKLKNLNNYISLFRKKTRTEKENKELENLEINLRKEIAKAFKGNEGYKSLFKKDIIET |
| | ILPEFLDDKDEIALVNSFNGFTTAFTGFFDNRENMFSEEAKSTSIAFRCINENLTRYISNMDIFE |
| | KVDAIFDKHEVQEIKEKILNSDYDVEDFFEGEFFNFVLTQEGIDVYNAIIGGFVTESGEKIKGL |
| | NEYINLYNQKTKQKLPKFKPLYKQVLSDRESLSFYGEGYTSDEEVLEVFRNTLNKNSEIFSSI |
| | KKLEKLFKNFDEYSSAGIFVKNGPAISTISKDIFGEWNVIRDKWNAEYDDIHLKKKAVVTEK |
| | YEDDRRKSFKKIGSFSLEQLQEYADADLSVVEKLKEIIIQKVDEIYKVYGSSEKLFDADFVLE |
| | KSLKKNDAVVAIMKDLLDSVKSFENYIKAFFGEGKETNRDESFYGDFVLAYDILLKVDHIYD |
| | AIRNYVTQKPYSKDKFKLYFQNPQFMGGWDKDKETDYRATILRYGSKYYLAIMDKKYAKC |
| | LQKIDKDDVNGNYEKINYKLLPGPNKMLPKVFFSKKWMAYYNPSEDIQKIYKNGTFKKGD |
| | MFNLNDCHKLIDFFKDSISRYPKWSNAYDFNFSETEKYKDIAGFYREVEEQGYKVSFESASK |
| | KEVDKLVEEGKLYMFQIYNKDFSDKSHGTPNLHTMYFKLLFDENNHGQIRLSGGAELFMRR |
| | ASLKKEELVVHPANSPIANKNPDNPKKTTTLSYDVYKDKRFSEDQYELHIPIAINKCPKNIFKI |
| | NTEVRVLLKHDDNPYVIGIDRGERNLLYIVVVDGKGNIVEQYSLNEIINNFNGIRIKTDYHSL |
| | LDKKEKERFEARQNWTSIENIKELKAGYISQVVHKICELVEKYDAVIALEDLNSGFKNSRVK |
| | VEKQVYQKFEKMLIDKLNYMVDKKSNPCATGGALKGYQITNKFESFKSMSTQNGFIFYIPA |
| | WLTSKIDPSTGFVNLLKTKYTSIADSKKFISSFDRIMYVPEEDLFEFALDYKNFSRTDADYIKK |
| | WKLYSYGNRIRIFRNPKKNNVFDWEEVCLTSAYKELFNKYGINYQQGDIRALLCEQSDKAF |
| | YSSFMALMSLMLQMRNSITGRTDVDFLISPVKNSDGIFYDSRNYEAQENAILPKNADANGAY |
| | NIARKVLWAIGQFKKAEDEKLDKVKIAISNKEWLEYAQTSVKH |
| SEQ ID NO: | MTNFDNFTKKYVNSKTIRLEAIPVGKTLKNIEKMGFIAADRQRDEDYQKAKSVIDHIYKAFM |
| 40 | DDCLKDLFLDWDPLYEAVVACWRERSPEGRQALQIMQADYRKKIADRFRNHELYGSLFTK |
| | KIFDGSVAQRLPDLEQSAEEKSLLSNFNKFTSYFRDFFDKRKRLFSDDEKHSAIAYRLINENFL |
| | KFVANCEAFRRMTERVPELREKLQNTGSLQVYNGLALDEVFSADFYNQLIVQKQIDLYNQLI |
| | GGIAGEPGTPNIQGLNATINLALQGDSSLHEKLAGIPHRFNPLYKQILSDVSTLSFVPSAFQSD |
| | GEMLAAVRGFKVQLESGRVLQNVRRLFNGLETEADLSRVYVNNSKLAAFSSMFFGRWNLC |
| | SDALFAWKKGKQKKITNKKLTEIKKWLKNSDIAIAEIQEAFGEDFPRGKINEKIQAQADALH |
| | SQLALPIPENLKALCAKDGLKSMLDTVLGLYRMLQWFIVGDDNEKDSDFYFGLGKILGSLDP |
| | VLVLYNRVRNYITKKPYSLTKFRLNFDNSQLLNGWDENNLDTNCASIFIKDGKYYLGISNKN |
| | NRPQFDTVATSGKSGYQRMVYKQFANWGRDLPHSTTQMKKVKKHFSASDADYVLDGDKF |
| | IRPLIITKEIFDLNNVKFNGKKKLQVDYLRNTGDREGYTHALHTWINFAKDFCACYKSTSIYD |
| | ISCLRPTDQYDNLMDFYADLGNLSHRIVWQTIPEEAIDNYVEQGQLFLFQLYNKDFAPGADG |
| | KPNLHTLYWKAVFNPENLEDVVVKLNGKAELFYRPRSNMDVVRHKVGEKLVNRKLKNGL |
| | TLPSRLHEEIYRYVNGTLNKDLSADARSVLPLAVVRDVQHEIIKDRRFTADKFFFHASLTFNF |
| | KSSDKPVGFNEDVREYLREHPDTYVVGVDRGERNLIYIVVIDPQGNIVEQRSFNMINGIDYW |
| | SLLDQKEKERVEAKQAWETVGKIKDLKCGYLSFLIHEITKIIIKYHAVVILENLSLGFKRVRT |
| | GIAEKAVYQQFERMLVTKLGYVVFKDRAGKAPGGVLNAYQLTDNTRTAENTGIQNGFLFY |
| | VPAAFTSRVDPATGFFDFYDWGKIKTATDKKNFIAGFNSVRYERSTGDFIVHVGAKNLAVR |
| | RVAEDVRTEWDIVIEANVRKMGIDGNSYISGKRIRYRSGEQGHGQYENHLPCQELIRALQQY |
| | GIQYETGKDILPAILQQDDAKLTDTVFDVFRLALQMRNTSAETGEDYFNSVVRDRSGRCFDT |
| | RRAEAAMPKEADANDAYHIALKGLFVLEKLRKGESIGIKNTEWLRYVQQRHS* |
| SEQ ID NO: | MTPIFCNFVVYQIMLFNNNININVKTMNKKHLSDFTNLFPVSKTLRFRLEPQGKTMENIVKA |
| 41 | QTIETDEERSHDYEKTKEYIDDYHRQFIDDTLDKFAFKVESTGNNDSLQDYLDAYLSANDNR |
| | TKQTEEIQTNLRKAIVSAFKMQPQFNLLFKKEMVKHLLPQFVDTDDKKRIVAKFNDFTTYFT |
| | GFFTNRENMYSDEAKSTSIAYRIVNQNLIKFVENMLTFKSHILPILPQEQLATLYDDFKEYLN |
| | VASIAEMFELDHFSIVLTQRQIEVYNSVIGGRKDENNKQIKPGLNQYINQHNQAVKDKSARL |
| | PLLKPLFNQILSEKAGVSFLPKQFKSASEVVKSLNEAYAELSPVLAAIQDVVTNITDYDCNGIF |
| | IKNDLGLTDIAQRFYGNYDAVKRGLRNQYELETPMHNGQKAEKYEEQVAKHLKSIESVSLA |
| | QINQVVTDGGDICDYFKAFGATDDGDIQRENLLASINNAHTAISPVLNKENANDNELRKNT |
| | MLIKDLLDAIKRLQWFAKPLLGAGDETNKDQVFYGKFEPLYNQLDETISPLYDKVRSYLTK |
| | KPYSLDKFKINFEKSNLLGGWDPGADRKYQYNAVILRKDNDFYLGIMRDEATSKRKCIQVL |
| | DCNDEGLDENFEKVEYKQIKPSQNMPRCAFAKKECEENADIMELKRKKNAKSYNTNKDDK |
| | NALIRHYQRYLDRTYPEFGFVYKDADEYDTVKAFTDSMDSQDYKLSFLQVSETGLNKLVDE |
| | GDLYLFKITNKDFSSYAKGRPNLHTIYWRMLFDPKNLANVVYKLEGKAEVFFRRKSLASTT |
| | THKAKQAIKNKSRYNEAVKPQSTFDYDIIKDRRFTADKFEFHVPIKMNFKAAGWNSTRLTNE |
| | VREFIKSQGVRHIIGIDRGERHLLYLTMIDMDGNIVKQCSLNAPAQDNARASEVDYHQLLDS |
| | KEADRLAARRNWGTIENIKELKQGYLSQVVHLLATMMVDNDAILVLENLNAGFMRGRQKV |
| | EKSVYQKFEKMLIDKLNYIVDKGQSPDKPTGALHAVQLTGLYSDFNKSNMKRANVRQCGF |
| | VFYIPAWNTSKIDPVTGFVNLFDTHLSSMGEIKAFFSKFDSIRYNQDKGWFEFKFDYSRFTTR |
| | AEGCRTQWTVCTYGERIWTHRSKNQNNQFVNDTVNVTQQMLQLLQDCGIDPNGNLKEAIA |
| | NIDSKKSLETLLHLFKLTVQMRNSVTGSEVDYMISPVADERGHFFDSRESDEHLPANADANG |
| | AFNIARKGLMVVRQIMATDDVSKIKFAVSNKDWLRFAQHIDD* |
| SEQ ID NO: | VKISKTLSLRIIRPYYTPEVESAIKAEKDKREAQGQTRNLDAKFFNELKKKHPQIILSGEFYSL |
| 42 | LFEMQRQLTSIYNRAMSSLYHKIIVEGEKTSTSKALSDIGYDECKSVFPSYIALGLRQKIQSNF |
| | RRKELKGFRMAVPTAKSDKFPIPIYKQVDDGKGGFKISENKEGDFIVELPLVEYTAEDVKTA |
| | KGKFTKINISKPPKIKNIPVILSTLRRKQSGQWFSDEGTNAEIRRVISGEYKVSWIEVVRRTRF |
| | GKHDDWFLNIVIKYDKTEDGLDPEVVGGIDVGVSTPLVCAVNNSLDRYFVKSSDIIAFKKRA |
| | MARRRTLLRQNRFKRSGHGSKSKLEPITILTEKNERFKKSIMQRWAKEVAEFFKGERASVVQ |
| | MEELSGLKEKDNFFGSYLRMYWNYGQLQQIIENKLKEYGIKVNYVSPKDTSKKCHSCGYIN |
| | EFFTFEFRQKNNFPLFKCKKCGVECNADYNAAKNIAIA |
| SEQ ID NO: | MLKSYDYFTKLYSLQKTLRFELKPIGKTLEHIKNSGIIESDETLEEQYAIVKNIIDKLHRKHIDE |
| 43 | ALSLVDFTKHLDTLKTFQELYLKRGKTDKEKEELEKLSADLRKLIVSYLKGNVKEKTQHNL |
| | NPIKERFEILFGKELFTNEEFFLLAENEKEKKAIQAFKGFTTYFKGFQENRKNMYSEEGNSTSI |
| | AYRIINENLPLFIENIARFQKVMSTIEKTTIKKLEQNLKTELKKHNLPGIFTIEYFNNVLTQEGIS |
| | RYNTIIGGKTTHEGVKIQGLNEIINLYNQQSKDVKLPILKPLHKQILSEEYSTSFKIKAFENDNE |
| | VLKAIDTFWNEHIEKSIHPVTGNKFNILSKIENLCDQLQKYKDKDLEKLFIERKNLSTVSHQV |
| | YGQWNIIRDALRMHLEMNNKNIKEKDIDKYLDNDAFSWKEIKDSIKIYKEHVEDAKELNEN |
| | GIIKYFSAMSINEEDDEKEYSISLIKNINEKYNNVKSILQEDRTGKSDLHQDKEKVGIIKEFLDS |
| | LKQLQWFLRLLYVTVPLDEKDYEFYNELEVYYEALLPLNSLYNKVRNYMTRKPYSVEKFKL |
| | NFNSPTLLDGWDKNKETANLSIILRKNGKYYLGIMNKENNTIFEYYPGTKSNDYYEKMIYKL |
| | LPGPNKMLPKVFFSKKGLEYYNPPKEILNIYEKGEFKKDKSGNFKKESLHTLIDFYKEAIAKN |
| | EDWEVFNFKFKNTKEYEDISQFYRDVEEQGYLITFEKVDANYVDKLVKEGKLYLFQIYNKD |
| | FSENKKSKGNPNLHTIYWKGLYDSENLKNVVYKLNGEAEVFYRKKSIDYPEEIYNHGHHKE |
| | ELLGKFNYPIIKDRRYTQDKFLFHVPITMNFISKEEKRVNQLACEYLSATKEDVHIIGIDRGER |
| | HLLYLSLIDKEGNIKKQLSLNTIKNENYDKEIDYRVKLDEKEKKRDEARKNWDVIENIKELK |
| | EGYMSQVIHIIAKMMVEEKAILIMEDLNIGFKRGRFKVEKQVYQKFEKMLIDKLNYLVFKNK |
| | NPLEPGGSLNAYQLTSKFDSFKKLGKQSGFIFYVPSAYTSKIDPTTGFYNFIQVDVPNLEKGK |
| | EFFSKFEKIIYNTKEDYFEFHCKYGKFVSEPKNKDNDRKTKESLTYYNAIKDTVWVVCSTNH |
| | ERYKIVRNKAGYYESHPVDVTKNLKDIFSQANINYNEGKDIKPIIIESNNAKLLKSIAEQLKLI |
| | LAMRYNNGKHGDDEKDYILSPVKNKQGKFFCTLDGNQTLPINADANGAYNIALKGLLLIEKI |
| | KKQQGKIKDLYISNLEWFMFMMSR |
| SEQ ID NO: | MIKNPSNRHSLPKVIISEVDHEKILEFKIKYEKLARLDRFEVKAMHYEGKEIVFDEVLVNGGL |
| 44 | IEVEYQDDNKTLFVKVGEKSYSIRGKKVGGKQRLLEDRVSKTKVQLELSDGVVDNKGNLR |
| | KSRTERELIVADNIKLYSQIVGREVTTTKEIYLVKRFLAYRSDLLFYYSFVDNFFKVAGNEKE |
| | LWKINFDDATSAQFMGYIPFMVNDNLKNDNAYLKDYVRNDVQIKDDLKKVQTIFSALRHT |
| | LLHFNYEFFEKLFNGEDVGFDFDIGFLNLLIENIDKLNIDAKKEFIDNEKIRLFGENLSLAKVY |
| | RLYSDICVNRVGFNKFINSMLIKDGVENQVLKAEFNRKFGGNAYTIDIHSNQEYKRIYNEHK |
| | KLVIKVSTLKDGQAIRRGNKKISELKEQMKSMTKKNSLARLECKMRLAFGFLYGEYNNYKA |
| | FKNNFDTNIKNSQFDVNDVEKSKAYFLSTYERRKPRTREKLEKVAKDIESLELKTVIANDTLL |
| | KFILLMFVFMPQELKGDFLGFVKKYYHDVHSIDDDTKEQEEDVVEAMSTSLKLKILGRNIRS |
| | LTLFKYALSSQVNYNSTDNIFYVEGNRYGKIYKKLGISHNQEEFDKTLVVPLLRYYSSLFKL |
| | MNDFEIYSLAKANPTAVSLQELVDDETSPYKQGNYFNFNKMLRDIYGLTSDEIKSGQVVFM |
| | RNKIAHFDTEVLLSKPLLGQTKMNLQRKDIVSFIEARGDIKELLGYDAINDFRMKVIHLRTK |
| | MRVYSDKLQTMMDLLRNAKTPNDFYNVYKVKGVESINKHLLEVLAQTAEERTVEKQIRDG |
| | NEKYDL |
| SEQ ID NO: | LNSIEKIKKPSNRNSIPSIIISDYDENKIKEIKVKYLKLARLDKITIQDMEIRDNIVEFKKILLNGI |
| 45 | EHTIKDNQKIEFDNYEITAYVRASKQRRDGKITQAKYVVTITDKYLRDNEKEKRFKSTERELP |
| | NDTLLMRYKQISGFDTLTSKDIYKIKRYIDFKNEMLFYFQFIEEFFSPLLPKGTNFYSLNIEQN |
| | KDKVVKYIVYRLNDDFKNQSLNQFIKKTDTIKYDFLKIQKILSDFRHALAHFDFDFIQKFFDD |
| | ELDKNRFDISTISLIKTMLQEKEEKYYQEKNNYIEDSDTLTLFDEKESNFSKIHNFYIKISQKKP |
| | AFNKLINSFLSKDGVPNEELKSYLATKKIDFFEDIHSNKEYKKIYIKHKNLVVEKQKEESQEK |
| | PNGQKLKNYNDELQKLKDEMNKITKQNSLNRLEVKLRLAFGFIANEYNYNFKNFNDKFTLD |
| | VKKEQKIKVFKNSSNEKLKEYFESTFIEKRFFHFCVKFFNKKTKKEETKQKNIFNLIENETLEE |
| | LVKESPLLQIITLLYLFIPKELQGEFVGFILKIYHHTKNITNDTKEDEKSIEDTQNSFSLKLKILA |
| | KNLRGLQLFNYSLSHNTLYNTKEHFFYEKGNRWQSVYKSLEISHNQDEFDIHLVIPVIKYYIN |
| | LNKLIGDFEIYALLTYADKNSITEKLSDITKRDDLKFRGYYNFSTLLFKTFMINTNYEQNQKS |
| | TQYIKQTRNDIAHQNIENMLKAFENNEIFAQREEIVNYLQKEHKMQEILHYNPINDFTMKTV |
| | QYLKSLNIHSQKESKIADIHKKESLVPNDYYLIYKLKVIELLKQKVIEAIGETKDEEKIKNAIA |
| | KEEQIKKGYNK |
| SEQ ID NO: | MLKHKRKNKNSLARVVLSNYDSNNIYEIKIKYEKLAKLDKINIIEMDYDADNNVMFKKVLF |
| 46 | NNKEIDLSHKDKTKINIELDNKKYNISAKKQIGKTHLVVRNKQTSKISRIKKIQDTYYRGKDV |
| | FILDNNIEILDKKQTKDKFIVTLNDITNNKTTSTEAELIDDTKDIFKKISAKKDLKSSDIYKIKRF |
| | ISIRSNFSFYYTFVDNYFKIFHAKKDKNKEELYKIKFKDEINIKPYLENILDNMKNKNGILYNY |
| | ANDRKKVLNDLRNIQYVFKEFRHKLAHFDYNFLDNFFSNSVEEKYKQKVNEIKLLDILLDNI |
| | DSLNVVPKQNYIEDETISVFDAKDIKLKRLYTYYIKLTINYPGFKKLINSFFIQDGIENQELKEY |
| | INNKEKDTQVLKELDNKAYYMDISQYRKYKNIYNKHKELVSEKELSSDGKKINSLNQKINK |
| | LKIDMKNITKPNALNRLIYRLRVAFGFIYKEYATINNFNKSFLQDTKTKRFENISQQDIKSYLD |
| | ISYQDKGKFFVKSKKTFKNKTTVKYTFEDLDLTLNEIITQDDIFVKVIFLFSIFMPKELNGDFF |
| | GFINMYYHKMKNISYDTKDIDMLDTISQNMKLKILEQNIKKTYVFKYYLDLDSSIYSKLVQN |
| | IKITEDIDSKKYLYAKIFKYYQHLYKLISDVEIYLLYKYNSKENLSITIDKDELKHRGYYNFQS |
| | LLIKNNINKDDAYWSIVNMRNNLSHQNIDELVGHFCKGCLRKSTTDIAELWLRKDILTITNEII |
| | NKIESFKDIKITLGYDCVNDFTQKVKQYKQKLKASNERLAKKIEEKQNQVVDEKNKEELEK |
| | NILNMKNIQKINRYILDIL |
| SEQ ID NO: | MLKHKRKNKNSLARVVLSNYDSNNIYEIKIKYEKLAKLDKINIIEMDYDADNNVMFKKVLF |
| 47 | NNKEIDLSHKDKTKINIELDNKKYNISAKKQIGKTHLVVRDKQTSKISRIKKIQDTYYRGKDV |
| | FILDNNIEILDKKQTKDKFIVTLNDITNDKTTSTEAELIDDTKDIFKKISAKKDLKSSDIYKIKRF |
| | ISIRSNFSFYYTFVDNYFKIFHAKKDKNKEELYKIKFKDEINIKPYLENILDNMKNKNGILYDY |
| | ADDREKVLNDLKNIQYVFTEFRHKLAHFDYNFLDNFFSNSVTDQYKQKVNEIKLLDILLDNI |
| | DSLNVVPKQNYIEDETISVFDAKDIKLKRLYTYYIKLTINYPGFKKLINSFFIQDGIENQELKEY |
| | INNKEKDTQVLKELDNKAYYMDISQYRKYKNIYNKHKELVSEKELSSDGQKINSLNQKINK |
| | LKIEMKNITKPNALNRLIYRLRVAFGFIYKEYATINNFNKSFLQDTKIKRFENISQQDIKNYLDI |
| | SYQDKGKFFVKSKKTFKNKTTIKYTFEDLDLTLNEIITQDDIFVKVIFLFSIFMPKELNGDFFGF |
| | INMYYHKMKNISYDTKDIDMLDTISQNMKLKILEQNIKKTYVFKYYLDLDSSIYSKLVQNIKI |
| | TEDIDSKKYLYAKIFKYYQHLYKLISDVEIYLLYKYNSKENLSITIDKDELKHRGYYNFQSLLI |
| | KNNINKDDAYWSIVNMRNNLSHQNIDELVGHFCKGCLRKSTTDIAELWLRKDILTITNEIINK |
| | IESFKDIKITLGYDCVNDFTQKVKQYKQKLKASNERLAKKIEEKQNQVVDEKNKEELEKKIL |
| | NMKNIQKINRYILDIL |
| SEQ ID NO: | MSQLKNPSNKNSLPRIIISDFNEIKINEIKIKYHKLDRLDKIIVKEMEIINNKIFFKKILFNNQIKD |
| 48 | INSENIELENYILAGEVKPSNTKIILNRDGKEKSFIVYDGFTFKYKPNDKRISETKTNAKYILTI |
| | KDKTRHRESSTQRDILKSSIIETYKQISGFENITSKDIYTIKRYIDFKNEMMFYYTFIDDFFFPIT |
| | GKNKQDKKNNFYNYKIKENAKKFISLINYRINDDFKNKNGILYDYLSNKEEIIINDFIHIQTILK |
| | DVRHAIAHFNFDFIQKLFDNEQAFNSKFDGIEILNILFNQKQEKYFEAQTNYIEEETIKILDEKE |
| | LSFKKLHSFYSQICQKKPAFNKLINSFIIQDGIENKELKDYISQKYNSKFDYYLDIHTCKIYKDI |
| | YNQHKKFVADKQFLENQKTDGQKIKKLNDQINQLKTKMNNLTKKNSLKRLEIKFRLAFGFI |
| | FTEYQTFKNFNERFIEDIKANKYSTKIELLDYGKIKEYISITHEEKRFFNYKTFNKKTNKNINK |
| | TIFQSLEKETFENLVKNDNLIKMMFLFQLLLPRELKGEFLGFILKIYHDLKNIDNDTKPDEKSL |
| | SELNISTALKLKILVKNIRQINLFNYTISNNTKYEEKEKRFYEEGNQWKDIYKKLYISHDFDIF |
| | DIHLIIPIIKYNINLYKLIGDFEVYLLLKYLERNTNYKTLDKLIEAEELKYKGYYNFTTLLSKAI |
| | NIALNDKEYHNITHLRNNTSHQDIQNIISSFKNNKLLEQRENIIELISKESLKKKLHFDPINDFT |
| | MKTLQLLKSLEVHSDKSEKIENLLKKEPLLPNDVYLLYKLKGIEFIKKELISNIGITKYEEKIQE |
| | KIAKGVEK |
| SEQ ID NO: | MVKNPANRHALPKVIISEVDNNNILEFKIKYEKLARLDKVEVKSMHFDNNKQVVFDEVVIN |
| 49 | GGLIEPTYEDKHKKLVVTAGEKSYSIVGQKVGGKPRLLEDRVSKTKVQLELTNYVEDKEGK |
| | KRVSKTERELIVADNIELYSQIVGREVKTTKEIYLIKRFLEYRSDLLFYYGFVDNFFKVAGNG |
| | KELWKIDFTNSDSLHLIEYFKFSINDNLKNDENYLKNYVSDNTKIENDLVKCQNNFNSLRHA |
| | LMHFDYDFFEKLFNGEDVGFDFDIEFLNIMIDKVDKLNIDTKKEFIDDEEVTLFGEALSLKKL |
| | YGLFSHIAINRVAFNKLINSFIIEDGIENKELKDFFNNKKESQAYEIDIHSNAEYKALYVQHKK |
| | LVMATSAMTDGDEIAKKNQEISDLKEKMKVITKENSLARLEHKLRLAFGFIYTEYKDYKTFK |
| | KHFDQDIKGAKYKGLNVEKLKEYYETTLKNSKPKTDEKLEDVAKKIDKLSLKELIDDDTLL |
| | KFVLLLFIFMPQELKGDFLGFIKKYYHDKKHIDQDTKDKDTEIEELSTGLKLKVLDKNIRSLSI |
| | LKHSFSFQVKYNRKDKNFYEDGNLHGKFYKKLSISHNQEEFNKSVYAPLFRYYSALYKLIND |
| | FEIYALAQHVENHETLADQVNKSQFIQKSYFNFRKLLDNTDSISQSSSYNTLIVMRNDISHLS |
| | YEPLFNYPLDERKSYKKKTQKGVKTFHVELLYISRAKIIELISLQTDMKKLLGYDAVNDFNM |
| | KVVHLRKRLSVYANKEESIRKMQADAKTPNDFYNIYKVKGVESINQHLLKVIGVTEAEKSIE |
| | KQINEGNKKHNT |
| SEQ ID NO: | MTKKPSNRNSLPKVIINKVDESSILEFKIKYEKLARLDRFEVRSMRYDGDGRIIFDEVVANAG |
| 50 | LLDVDYEDDNRTIVVKIENKAYNIYGKKVGGEKRLNGKISKAKVQLILTDSIRKNANDTHRH |
| | SLTERELINKNEVDLYSKIAEREISTTKDIYLVKRFLAYRSDLLLYYAFINHYVRVNGNKKEF |
| | WKTEIDDKIIDYFIYTINDTLKNKEGYLEKYIVDRDQIKKDLEKIKQIFSHLRHKLMHYDFRFF |
| | TDLFDGKDVDIKVDNSIQKISELLDIEFLNIVIDKLEKLNIDAKKEFIDDEKITLFGQEIELKKL |
| | YSLYAHTSINRVAFNKLINSFLIKDGVENKELKEYFNAHNQGKESYYIDIHQNQEYKKLYIEH |
| | KNLVAKLSATTDGKEIAKINRELADKKEQMKQITKANSLKRLEYKLRLAFGFIYTEYKDYER |
| | FKNSFDTDTKKKKFDAIDNAKIIEYFEATNKAKKIEKLEEILKGIDKLSLKTLIQDDILLKFLLL |
| | FFTFLPQEIKGEFLGFIKKYYHDITSLDEDTKDKDDEITELPRSLKLKIFSKNIRKLSILKHSLSY |
| | QIKYNKKESSYYEAGNVFNKMFKKQAISHNLEEFGKSIYLPMLKYYSALYKLINDFEIYALY |
| | KDMDTSETLSQQVDKQEYKRNEYFNFETLLRKKFGNDIEKVLVTYRNKIAHLDFNFLYDKPI |
| | NKFISLYKSREKIVNYIKNHDIQAVLKYDAVNDFVMKVIQLRTKLKVYADKEQTIESMIQNT |
| | QNPNGFYNIYKVKAVENINRHLLKVIGYTESEKAVEEKIRAGNTSKS |
| SEQ ID NO: | MIKNPSNRYALPKVIISKIDNQNILEFKIKYKKLSKLDIVKVKSMHYDDRAIIFDEVIVNDGLI |
| 51 | DVEYRDNHKTIFVKVGNKSYSISGQKVGGKERLLENRVSKTKVQLELKDKATNRVSKTERE |
| | LIVDDNIKIYSQIVGRDVKTTKDIYLIKRFLAYRSDLLFYYGFVNNFFHVANNRSEFWKIDFN |
| | DSNNSKLIEYFKFTINDHLKNDENYLKDYISDNEKLKNDLIKVKNSFEKIRHALMHFDYDFF |
| | VKLFNGEDVGLELDIEFLDIMIDKLDKLNIDTKKEFIDDEKITIFGEELSLAKLYRFYAHTAIN |
| | RVAFNKLINSFIIENGVENQSLKEYFNQQAGGIAYEIDIHQNREYKNLYNEHKKLVSRVLSIS |
| | DGQEIAILNQKIAKLKDQMKQITKANSIKRLEYKLRLALGFIYTEYENYEEFKNNFDTDIKNG |
| | RFTPKDNDGNKRAFDSRELEQLKGYYEATIQTQKPKTDEKIEEVSKKIDRLSLKSLIADDILL |
| | KFILLMFTFMPQELKGEFLGFIKKYYHDTKHIDQDTISDSDDTIETLSIGLKLKILDKNIRSLSIL |
| | KHSLSFQTKYNKKDRNYYEDGNIHGKFFKKLGISHNQEEFNKSVYAPLFRYYSALYKLINDF |
| | EIYTLSLHIVGSETLTDQVNKSQFLSGRYFNFRKLLTQSYHINNNSTHSTIFNAVINMRNDISH |
| | LSYEPLFDCPLNGKKSYKRKIRNQFKTINIKPLVESRKIIIDFITLQTDMQKVLGYDAVNDFTM |
| | KIVQLRTRLKAYANKEQTIQKMITEAKTPNDFYNIYKVQGVEEINKYLLEVIGETQAEKEIRE |
| | KIERGNIANF |
| SEQ ID NO: | MKKSIFDQFVNQYALSKTLRFELKPVGETGRMLEEAKVFAKDETIKKKYEATKPFFNKLHRE |
| 52 | FVEEALNEVELAGLPEYFEIFKYWKRYKKKFEKDLQKKEKELRKSVVGFFNAQAKEWAKK |
| | YETLGVKKKDVGLLFEENVFAILKERYGNEEGSQIVDESTGKDVSIFDSWKGFTGYFIKFQET |
| | RKNFYKDDGTATALATRIIDQNLKRFCDNLLIFESIRDKIDFSEVEQTMGNSIDKVFSVIFYSS |
| | CLLQEGIDFYNCVLGGETLPNGEKRQGINELINLYRQKTSEKVPFLKLLDKQILSEKEKFMDE |
| | IENDEALLDTLKIFRKSAEEKTTLLKNIFGDFVMNQGKYDLAQIYISRESLNTISRKWTSETDI |
| | FEDSLYEVLKKSKIVSASVKKKDGGYAFPEFIALIYVKSALEQIPTEKFWKERYYKNIGDVLN |
| | KGFLNGKEGVWLQFLLIFDFEFNSLFEREIIDENGDKKVAGYNLFAKGFDDLLNNFKYDQKA |
| | KVVIKDFADEVLHIYQMGKYFAIEKKRSWLADYDIDSFYTDPEKGYLKFYENAYEEIIQVYN |
| | KLRNYLTKKPYSEDKWKLNFENPTLADGWDKNKEADNSTVILKKDGRYYLGLMARGRNK |
| | LFDDRNLPKILEGVENGKYEKVVYKYFPDQAKMFPKVCFSTKGLEFFQPSEEVITIYKNSEFK |
| | KGYTFNVRSMQRLIDFYKDCLVRYEGWQCYDFRNLRKTEDYRKNIEEFFSDVAMDGYKISF |
| | QDVSESYIKEKNQNGDLYLFEIKNKDWNEGANGKKNLHTIYFESLFSADNIAMNFPVKLNG |
| | QAEIFYRPRTEGLEKERIITKKGNVLEKGDKAFHKRRYTENKVFFHVPITLNRTKKNPFQFNA |
| | KINDFLAKNSDINVIGVDRGEKQLAYFSVISQRGKILDRGSLNVINGVNYAEKLEEKARGRE |
| | QARKDWQQIEGIKDLKKGYISQVVRKLADLAIQYNAIIVFEDLNMRFKQIRGGIEKSVYQQL |
| | EKALIDKLTFLVEKEEKDVEKAGHLLKAYQLAAPFETFQKMGKQTGIVFYTQAAYTSRIDPV |
| | TGWRPHLYLKYSSAEKAKADLLKFKKIKFVDGRFEFTYDIKSFREQKEHPKATVWTVCSCV |
| | ERFRWNRYLNSNKGGYDHYSDVTKFLVELFQEYGIDFERGDIVGQIEVLETKGNEKFFKNFV |
| | FFFNLICQIRNTNASELAKKDGKDDFILSPVEPFFDSRNSEKFGEDLPKNGDDNGAFNIARKG |
| | LVIMDKITKFADENGGCEKMKWGDLYVSNVEWDNFVANK |
| SEQ ID NO: | MFNNFIKKYSLQKTLRFELKPVGETADYIEDFKSEYLKDTVLKDEQRAKDYQEIKTLIDDYH |
| 53 | REYIEECLREPVDKKTGEILDFTQDLEDAFSYYQKLKENPTENRVGWEKEQESLRKKLVTSF |
| | VGNDGLFKKEFITRDLPEWLQKKGLWGEYKDTVENFKKFTTYFSGFHENRKNMYTAEAQS |
| | TAIANRLMNDNLPKFFNNYLAYQTIKEKHPDLVFRLDDALLQAAGVEHLDEAFQPRYFSRLF |
| | AQSGITAFNELIGGRTTENGEKIQGLNEQINLYRQQNPEKAKGFPRFMPLFKQILSDRETHSFL |
| | PDAFENDKELLQALRDYVDAATSEEGMISQLNKAMNQFVTADLKRVYIKSAALTSLSQELF |
| | HFFGVISDAIAWYAEKRLSPKKAQESFLKQEVYAIEELNQAVVGYIDQLEDQSELQQLLVDL |
| | PDPQKPVSSFILTHWQKSQEPLQAVIAKVEPLFELEELSKNKRAPKHDKDQGGEGFQQVDAI |
| | KNMLDAFMEVSHAIKPLYLVKGRKAIDMPDVDTGFYADFAEAYSAYEQVTVSLYNKTRNH |
| | LSKKPFSKDKIKINFDAPTLLNGWDLNKESDNKSIILRKDGNFYLAIMHPKHTKVFDCYSASE |
| | AAGKCYEKMNYKLLSGANKMLPKVFFSKKGIETFSPPQEILDLYKNNEHKKGATFKLESCH |
| | KLIDFFKRNIPKYKVHPTDNFGWDVFGFHFSPTSSYGDLSGFYREVEAQGYKLWFSDVSEAY |
| | INKCVEEGKLFLFQIYNKDFSPNSTGKPNLHTLYWKGLFEPENLKDVVLKLNGEAEIFYRKH |
| | SIKHEDKTIHRAKDPIANKNADNPKKQSVFDYDIIKDKRYTQDKFFFHVPISLNFKSQGVVRF |
| | NDKINGLLAAQDDVHVIGIDRGERHLLYYTVVNGKGEVVEQGSLNQVATDQGYVVDYQQ |
| | KLHAKEKERDQARKNWSTIENIKELKAGYLSQVVHKLAQLIVKHNAIVCLEDLNFGFKRGR |
| | FKVEKQVYQKFEKALIDKLNYLVFKERGATQAGGYLNAYQLAAPFESFEKLGKQTGILYYV |
| | RSDYTSKIDPATGFVDFLKPKYESMAKSKVFFESFERIQWNQAKGYFEFEFDYKKMCPSRKF |
| | GDYRTRWVVCTFGDTRYQNRRNKSSGQWETETIDVTAQLKALFAAYGITYNQEDNIKDAIA |
| | AVKYTKFYKQLYWLLRLTLSLRHSVTGTDEDFILSPVADENGVFFDSRKATDKQPKDADAN |
| | GAYHIALKGLWNLQQIRQHDWNVEKPKKLNLAMKNEEWFGFAQKKKFRA |
| SEQ ID NO: | MIKNPSNRYALPKVIISKIDNQNILEFKIKYKKLSKLDIVKVKSMHYDDRAIIFDEVIVNDGLI |
| 54 | DVEYRDNHKTIFVKVGNKSYSISGQKVGGKERLLENRVSKTKVQLELKDKATNRVSKTERE |
| | LIVDDNIKIYSQIVGRDVKTTKDIYLIKRFLAYRSDLLFYYGFVNNFFHVANNRSEFWKIDFN |
| | DSNNSKLIEYFKFTINDHLKNDENYLKDYISDNEKLKNDLIKVKNSFEKIRHALMHFDYDFF |
| | VKLFNGEDVGLELDIEFLDIMIDKLDKLNIDTKKEFIDDEKITIFGEELSLAKLYRFYAHTAIN |
| | RVAFNKLINSFIIENGVENQSLKEYFNQQAGGIAYEIDIHQNREYKNLYNEHKKLVSRVLSIS |
| | DGQEIAILNQKIAKLKDQMKQITKANSIKRLEYKLRLALGFIYTEYENYEEFKNNFDTDIKNG |
| | RFTPKDNDGNKRAFDSRELEQLKGYYEATIQTQKPKTDEKIEEVSKKIDRLSLKSLIADDILL |
| | KFILLMFTFMPQELKGEFLGFIKKYYHDTKHIDQDTISDSDDTIETLSIGLKLKILDKNIRSLSIL |
| | KHSLSFQTKYNKKDRNYYEDGNIHGKFFKKLGISHNQEEFNKSVYAPLFRYYSALYKLINDF |
| | EIYTLSLHIVGSETLTDQVNKSQFLSGRYFNFRKLLTQSYHINNNSTHSTIFNAVINMRNDISH |
| | LSYEPLFDCPLNGKKSYKRKIRNQFKTINIKPLVESRKIIIDFITLQTDMQKVLGYDAVNDFTM |
| | KIVQLRTRLKAYANKEQTIQKMITEAKTPNDFYNIYKVQGVEEINKYLLEVIGETQAEKEIRE |
| | KIERGNIANF |
| SEQ ID NO: | MIKNPSNRHSLPKVIISEVDHEKILEFKIKYEKLARLDRFEVKAMHYEGKEIVFDEVLVNGGL |
| 55 | IEVEYQDDNKTLFVKVGEKSYSIRGKKVGGKQRLLEDRVSKTKVQLELSDGVVDNKGNLR |
| | KSRTERELIVADNIKLYSQIVGREVTTTKEIYLVKRFLAYRSDLLFYYSFVDNFFKVAGNEKE |
| | LWKINFDDATSAQFMGYIPFMVNDNLKNDNAYLKDYVRNDVQIKDDLKKVQTIFSALRHT |
| | LLHFNYEFFEKLFNGEDVGFDFDIGFLNLLIENIDKLNIDAKKEFIDNEKIRLFGENLSLAKVY |
| | RLYSDICVNRVGFNKFINSMLIKDGVENQVLKAEFNRKFGGNAYTIDIHSNQEYKRIYNEHK |
| | KLVIKVSTLKDGQAIRRGNKKISELKEQMKSMTKKNSLARLECKMRLAFGFLYGEYNNYKA |
| | FKNNFDTNIKNSQFDVNDVEKSKAYFLSTYERRKPRTREKLEKVAKDIESLELKTVIANDTLL |
| | KFILLMFVFMPQELKGDFLGFVKKYYHDVHSIDDDTKEQEEDVVEAMSTSLKLKILGRNIRS |
| | LTLFKYALSSQVNYNSTDNIFYVEGNRYGKIYKKLGISHNQEEFDKTLVVPLLRYYSSLFKL |
| | MNDFEIYSLAKANPTAVSLQELVDDETSPYKQGNYFNFNKMLRDIYGLTSDEIKSGQVVFM |
| | RNKIAHFDTEVLLSKPLLGQTKMNLQRKDIVSFIEARGDIKELLGYDAINDFRMKVIHLRTK |
| | MRVYSDKLQTMMDLLRNAKTPNDFYNVYKVKGVESINKHLLEVLAQTAEERTVEKQIRDG |
| | NEKYDL |
| SEQ ID NO: | MEEKMLKSYDYFTKLYSLQKTLRFELKPIGKTLEHIKNSGIIESDETLEEQYAIVKNIIDKLHR |
| 56 | KHIDEALSLVDFTKHLDTLKTFQELYLKRGKTDKEKEELEKLSADLRKLIVSYLKGNVKEKT |
| | QHNLNPIKERFEILFGKELFTNEEFFLLAENEKEKKAIQAFKGFTTYFKGFQENRKNMYSEEG |
| | NSTSIAYRIINENLPLFIENIARFQKVMSTIEKTTIKKLEQNLKTELKKHNLPGIFTIEYFNNVLT |
| | QEGISRYNTIIGGKTTHEGVKIQGLNEIINLYNQQSKDVKLPILKPLHKQILSEEYSTSFKIKAF |
| | ENDNEVLKAIDTFWNEHIEKSIHPVTGNKFNILSKIENLCDQLQKYKDKDLEKLFIERKNLST |
| | VSHQVYGQWNIIRDALRMHLEMNNKNIKEKDIDKYLDNDAFSWKEIKDSIKIYKEHVEDAK |
| | ELNENGIIKYFSAMSINEEDDEKEYSISLIKNINEKYNNVKSILQEDRTGKSDLHQDKEKVGII |
| | KEFLDSLKQLQWFLRLLYVTVPLDEKDYEFYNELEVYYEALLPLNSLYNKVRNYMTRKPYS |
| | VEKFKLNFNSPTLLDGWDKNKETANLSIILRKNGKYYLGIMNKENNTIFEYYPGTKSNDYYE |
| | KMIYKLLPGPNKMLPKVFFSKKGLEYYNPPKEILNIYEKGEFKKDKSGNFKKESLHTLIDFYK |
| | EAIAKNEDWEVFNFKFKNTKEYEDISQFYRDVEEQGYLITFEKVDANYVDKLVKEGKLYLF |
| | QIYNKDFSENKKSKGNPNLHTIYWKGLYDSENLKNVVYKLNGEAEVFYRKKSIDYPEEIYN |
| | HGHHKEELLGKFNYPIIKDRRYTQDKFLFHVPITMNFISKEEKRVNQLACEYLSATKEDVHII |
| | GIDRGERHLLYLSLIDKEGNIKKQLSLNTIKNENYDKEIDYRVKLDEKEKKRDEARKNWDVI |
| | ENIKELKEGYMSQVIHIIAKMMVEEKAILIMEDLNIGFKRGRFKVEKQVYQKFEKMLIDKLN |
| | YLVFKNKNPLEPGGSLNAYQLTSKFDSFKKLGKQSGFIFYVPSAYTSKIDPTTGFYNFIQVDV |
| | PNLEKGKEFFSKFEKIIYNTKEDYFEFHCKYGKFVSEPKNKDNDRKTKESLTYYNAIKDTVW |
| | VVCSTNHERYKIVRNKAGYYESHPVDVTKNLKDIFSQANINYNEGKDIKPIIIESNNAKLLKSI |
| | AEQLKLILAMRYNNGKHGDDEKDYILSPVKNKQGKFFCTLDGNQTLPINADANGAYNIALK |
| | GLLLIEKIKKQQGKIKDLYISNLEWFMFMMSR |
| SEQ ID NO: | MEKSLNDFIGLYSVSKTLRFELKPVSETLENIKKFHFLEEDKKKANDYKDVKKIIDNYHKYFI |
| 57 | DDVLKNASFNWKKLEEAIREYNKNKSDDSALVAEQKKLGDAILKLFTSDKRYKALTAATPK |
| | ELFESILPDWFGEQCNQDLNKAALKTFQKFTSYFTGFQENRKNVYSAEAIPTAVPYRIVNDN |
| | FPKFLQNVLIFKTIQEKCPQIIDEVEKELSSYLGKEKLAGIFTLESFNKYLGQGGKENQRGIDF |
| | YNQIIGGVVEKEGGINLRGVNQFLNLYWQQHPDFTKEDRRIKMVPLYKQILSDRSSLSFKIES |
| | IENDEELKNALLECADKLELKNDEKKSIFEEVCDLFSSVKNLDLSGIYINRKDINSVSRILTGD |
| | WSWLQSRMNVYAEEKFTTKAEKARWQKSLDDEGENKSKGFYSLTDLNEVLEYSSENVAET |
| | DIRITDYFEHRCRYYVDKETEMFVQGSELVALSLQEMCDDILKKRKAMNTVLENLSSENKL |
| | REKTDDVAVIKEYLDAVQELLHRIKPLKVNGVGDSTFYSVYDSIYSALSEVISVYNKTRNYIT |
| | KKAASPEKYKLNFDNPTLADGWDLNKEQANTSVILRKDGMFYLGIMNPKNKPKFAEKYDC |
| | GNESCYEKMIYKQFDATKQIPKCSTQKKEVQKYFLSGATEPYILNDKKSFKSELIITKDIWFM |
| | NNHVWDGEKFVPKRDNETRPKKFQIGYFKQTGDFDGYKNALSNWISFCKNFLQSYLSATVY |
| | DYNFKNSEEYEGLDEFYNYLNATCYKLNFINIPETEINKMVSEGKLYLFQIYNKDFASGSTG |
| | MPNMHTLYWKNLFSDENLKNVCLKLNGEAELFYRPAGIKEPVIHKEGSYLVNRTTEDGESIP |
| | EKIYFEIYKNANGKLEKLSDEAQNYISNHEVVIKKAGHEIIKDRHYTEPKFLFHVPLTINFKAS |
| | GNSYSINENVRKFLKNNPDVNIIGLDRGERHLIYLSLINQKGEIIKQFTFNEVERNKNGRTIKV |
| | NYHEKLDQREKERDAARKSWQAIGKIAELKEGYLSAVIHQLTKLMVEYNAVVVMEDLNFG |
| | FKRGRFHVEKQVYQKFEHILIDKSNYLVFKDRGLNEPGGVLNGYQIAGQFESFQKLGKQSG |
| | MLFYVPAGYTSKIDPKTGFVSMMNFKDLTNVHKKRDFFSKFDNIHYDEANGSFVFTFDYKK |
| | FDGKAKEEMKLTKWSVYSRDKRIVYFAKTKSYEDVLPTEKLQKIFESNGIDYKSGNNIQDSV |
| | MAIGADLKEGAKPSKEISDFWDGLLSNFKLILQMRNSNARTGEDYIISPVMADDGTFFDSRE |
| | EFKKGEDAKLPLDADANGAYHIALKGLSLINKINLSKDEELKKFDMKISNADWFKFAQEKN |
| | YAK |
| SEQ ID NO: | MNTQKKEFNPKSFKDFTNLYSLNKTLRFSLTPNKKTAEILEFNKQKEVKCFSNDRKIAGAYQ |
| 58 | EIKKYLNKLHQEFIQEAMKFFAFSEEELKGFEKEYLNLLNFTDKDNFKKKNKIRNEYEQERKI |
| | LTIKIATYFSKFKSEKYQSFNLANITGKKVFSILEQKYKEDKKTLKIIHIFKYKPTKDEKKEGE |
| | AVNFSTYLTGFNENRKNFYKSEDKAGQFATRTIDNLAQFIKNKKLFEDKYQKNYSKIGILDE |
| | QIKIFNLDYFNNLFLQEGLDEYNGILGNNKGEENKSNEGINQKINIFKQKEKARLKKEKENFN |
| | KSDFPLFKELYKQIGSIRKENDVYVEIKTDKELVEELNNFPKNVENYLKDIQSFYKTFFEKLQ |
| | NEEYELDKIYLPKSVGTYFSYIAFSDWNKLAFIYNKRYKNEKIKIVEGGDVNVQYRSLEVLK |
| | NRIDELKDEDNLNFNKFFIDKLKFNEAKKENNWQNFWFCIEYYINSQFIGGEKNILNKEKNE |
| | YEILPFGSLKELKEKYFEAVKKYKEKMVDTESGLTDDEEKEIKETLKNYLDRIKEIERIAKYF |
| | DLKKSFEEIKQEDLDSNFYGEYQKVVDKTNELKIYQYYSEFRNYLTQNNSVEEKIKLNFNSG |
| | LLLDGWDLNKEKVKFSIIFQENGKYYLGIINKEKDKTILDKDKHPEIFTKNSDFRKMEYKLFP |
| | SPSKMLPKISFSETAKKGDEDVGWSEEIQKIKDEFAEFQEYKKKSKDNWKDEFNRGKLNKLI |
| | DYYKQVLEKHSEGYMNTYNFELKDSSKYKNLGEFNDDIARQNYKVKFVGIDKNYIDEKVA |
| | NGELFLFQIYNKDFSEDKKEGSTNNLETIYFKELFSKENLENPVFKLSGGAEMFFRNKIEKKK |
| | EKKKLDKDGKPMISKKGEKVVDKRRFSENKILFHLPIEINYGKGKMPNFNKKINEYISKNPEN |
| | IKIIGIDRGEKHLLYYSIIDQNGNNIESMSLNAVDEFGNFVNPEKLEEYEIDNNGKKERRWKYI |
| | VNDKEIKVTNYQRKLDELEKERQKSRQSWQNINKIKNLKKGYISFVVKKIVDLAIENNAIIIL |
| | EDLNFGFKSFRQKIEKNVYQQFEKALIDKLGFVVDKQKQNQRFAPQLSAPFESFQKIGKQTGI |
| | VYYVLANNTSKVCPSCQWIKNFYLKYEKKNTIFNLQKNQKLKVFFEQEKNRFRFEYQMSKE |
| | YISVYSDVDRQRYDKTKNQNKGGYLEYKNSNQKEIIDKDGVIQKQSITLQLKELFKENHIDL |
| | EKEILKQLDNKKEKNSGYTGVYNKFIYLFNLILQIRNAISFREKDYIQCPSCHFDTRKENYLKI |
| | NDGDGNGAYNIALRGLYLLKGKNGIINNLEKIKLIFSNNDYFQWAKKLKNKK |
| SEQ ID NO: | MQNKQSFADFTNLYSLSKTLRFELKPIGQTQAMLDENKIFEVDENRKKAYDKTKPYFDRLH |
| 59 | REFINESLSNAQLKGISEYFETFKQFRSNQNNKDLKELINKQQKFLRHQIVTLFDENGKHWAT |
| | TKYAHLKIKKKNLDILFDEQVFYILKERYGSEKETQLVDKETGAVTSIFDNWKGFTGYFTKF |
| | FETRKNFYKSDGTSTALATRIIDQNLNRFFDNLETFHKIKDKIDVKEVEIFFKLKADNVFSIDF |
| | YNQCLLQNGIDKYNDFLGGQTLENGEKQKGINEIINKYRQDNKDQKLPFLKKLDKQILSEKD |
| | RFINEIESKEEFFQVLTEFYQSATVKVTIIKTLLNDFVHNTDKYKLEKIYLTKEAFNTIANKWT |
| | DETQIFEDNLDLVLKNKKITAKQDFIPLAYIKEALEVIEKDRKFFKDRYYNDPQIGFFPDQSY |
| | WEQFLAILNFEFMTHFQRVAKDKITGKKIELGYFVFEKRIKELLDSDPSLNSQSKIIIKEFADE |
| | VLHIFQMAKYFALEKKREWKGDYYQLDDQFYNHIDYGFKDQFYENAYEKIVQPYNKIRNY |
| | LTKKPYSDVKWKLNFGNPTLANGWDKNKEADNTAVILKKDGNYYLGVMKKGKNKIFSDQ |
| | NKEKYKAYNSAYYEKLVYKLFPDPSKMFPKVCFSKKGLNFFQPSEEILRIYKNNEFKKGNTF |
| | SISSMQKLIAFYIDCLGLYEGWKHYEFKNIKDVRQYKENIGEFYADVAESGYKLWFEKISEE |
| | YITQKNQLGELFLFQIYNKDFAKKTTGRKNLHTIYFEELFSQTNIDNNFPFKLNGQAELFYRP |
| | KSLEKIEEKRNFKRSIVNKKRYTQNKIFFHVPITLNRTSENIGRFNVRVNNFLANNSNVNIVG |
| | VDRGEKNLAYYSIIKQNGEVLKSGSLNIINGVDYHALLTDRAQRREQERRNWQDVESIKDLK |
| | RGYISQVVHELVSLAIKYNAIIVMEDLNMRFKQIRGGIEKSTYQQLEKALIEKLNFLVNKEET |
| | DSNQAGNLLNAYQLTAPFKTFKDMGKQTGIIFYTQASYTSKIDPLTGWRPNIYLRYSNAKQA |
| | KADILMFTNIYFSEKKDRFEFTYDLEKIDDKRKDLPIKTEWTVCSNVERFSWEKSLNNNKGG |
| | YVHYPIQDSNGEESITSKLKKLFMDFGIDLTDIKTQIESLDTNKKDNANFFRKFIFYFQLICQIR |
| | NTQVNKSDDGNDFIFSPVEPFFDSRFADKFRKNLPKNGDENGAYNIARKGLIILHKISDYFVK |
| | EGSTDKISWKDLSISQTEWDNFTTDK |
| SEQ ID NO: | MKKEKEFKSFGDFTNLYEISKTLRFELKPVENTQTMLDEADVFGKDKVIKDKYTKTKPFIDK |
| 60 | LHREFVDESLKDVSLSGLKKYSEVLENWKKNKKDKDIVKELKKEEERLRKEVVEFFDNTAK |
| | KWANEKYKELGLKKKDIGILFEESVFDLLKEKYGEEQDSFLKEEKGDFLKNEKGEKVSIFDE |
| | WKGFVGYFTKFQETRKNFYKNDGTETALATRIIDQNLKRFCDNIDDFKKIKNKIDFSEVEKN |
| | FNKTADVFSLDFYNQCLLQKGIDSYNEFIGGKTLENGKKLKGVNELVNEYRQKNKNEKVSF |
| | LKLLDKQILSEKEKLSFGIENDEQLLVVLNSFYETAEEKTKILRTLFGDFVEHNENYDLDKTY |
| | ISKVAFNTISHKWTNETHKFEELLYGAMKEDKPIGLNYDKKEDSYKFPDFIALGYLKKCLNN |
| | LDCDTKFWKEKYYENNADKKDKDKGFLTGGQNAWDQFLQIFIFEFNQLFNSEAFDNKGKEI |
| | KIGYDNFRKDFEEIINQKDFKNDENLKIAIKNFADSVLWIYQMAKYFAIEKKRGWDDDFELS |
| | EFYTNPSNGYSLFYDRAYEEIVQKYNDLRNYLTKKPYKEDKWKLNFENPTLANGFDKNKES |
| | DNSTVILRKKRKYYLGLMKKGNNKIFEDRNKAEFIRNIESGAYEKMAYKYLPDVAKMIPKC |
| | STQLNEAKNHFRNSADDLEIKKSFSNPLKITKRIFDLNNIQYDKTNVSKKISGDNKGIKIFQKE |
| | YYKISGDFDVYKSALNDWIDFCKDFLSKYDSTKDFDFSILRKTKDYKSLDEFYVDVAKITYK |
| | ISFTPVSESYIDQKNKNGELYLFEIYNQDFAKGKMGAKNLHTLYFENVFSPENISKNFPIKLN |
| | GNAELFFRPKSIESKKEKRNFVREIVNKKRYSEDKIFFHCPITLNRETGSIYRFNNYVNNFLSE |
| | NNINIIGVDRGEKHLAYYSVIDKNGVKIGGGSFNEINKVDYAKKLEERAGEREQSRKDWQV |
| | VEGIKDLKKGYISQVVRELADLAIKHNAIIVLEDLNMRFKQIRGGIEKSIYQQLEKALIDKLSF |
| | LVEKGEKDPNQAGHILKAYQLAAPFTSFKDMGKQTGIVFYTQASYTSKTCPNCGFRKNNNK |
| | FYFENNIGKAQDALKKLKTFEYDSENKCFGLSYCLSDFANKEEVEKNKNKKRNNAPYSDIE |
| | KKDCFELSTKDAVRYRWHDKNTERGKTFFEGESVYEEKEEKEIGQTKRGLVKEYDISKCLIG |
| | LFEKTGLDYKQNLLDKINSGKFDGTFYKNLFNYLNLLFEIRNSISGTEIDYISCPECQFHTDKS |
| | KTIKNGDDNGSYNIARKGMIILDKIKQFKKENGSLDKMGWGELFIDLEEWDKFAQKKNNNII |
| | DK |
| SEQ ID NO: | MKYTDFTGIYSVSKTLRFELIPQGSTVENMKREGILNNDMHRADSYKEMKKLIDEYHKAFIE |
| 61 | RCLSDFSLKYDDTGKHDSLEEYFFYYEQKRNDKTKKIFEDIQVALRKQISKRFTGDTAFKRLF |
| | KKELIKEDLPSFVKNDPVKTELIKEFSDFTTYFQEFHKNRKNMYTSDAKSTAIAYRIINENLPK |
| | FIDNINAFDIVAKVPEMQEHFKTIADELRSHLQVGNDIDKMFNLQFFNKVLTQSQLDVYNAV |
| | IGGKSEGNKKIQGINEYINLYNQQHKKARLPMLKLLYKQILSDRVAISWLQDEFDNDQDML |
| | DTIEAFYNKLNSNETGVLGEGKLKQILMGLDGYNLDGVFMRNDLQLSEVSQRLCGGWNIIK |
| | DAMTSDLKRSVQKKKKETDADFEERVSKLFSAQNSFSIAYINQCLGQAGIRCKIQDYFACLG |
| | AKEGENEAETTPDIFDQIAEAYHGAAPILNARPSSHNLAQDIEKVKAIKALLDALKRLQRFVK |
| | PLLGRGDEGDKDNFFYGDFMPIWEVLDQLTPLYNKVRNRMTRKPYSQEKIKLNFENSTLLN |
| | GWDLNKEHDNTSVILRREGLYYLGIMNKNYNKIFDANNVETIGDCYEKMIYKLLPGPNKML |
| | PKVFFSKSRVQEFSPSKKILEIWESKSFKKGDNFNLDDCHALIDFYKDSIAKHPDWNKFNFKF |
| | SDTQSYTNISDFYRDVNQQGYSLSFTKVSVDYVNRMVDEGKLYLFQIYNKDFSPQSKGTPN |
| | MHTLYWRMLFDERNLHNVIYKLNGEAEVFYRKASLRCDRPTHPAHQPITCKNENDSKRVC |
| | VFDYDIIKNRRYTVDKFMFHVPITINYKCTGSDNINQQVCDYLRSAGDDTHIIGIDRGERNLL |
| | YLVIIDQHGTIKEQFSLNEIVNEYKGNTYCTNYHSLLEEKEAGNKKARQDWQTIESIKELKEG |
| | YLSQVIHKISMLMQRYHAIVVLEDLNGSFMRSRQKVEKQVYQKFEHMLINKLNYLVNKQY |
| | DATEPGGLLHALQLTSRMDSFKKLGKQSGFLFYIPAWNTSKIDPVTGFVNLFDTRYCNEAKA |
| | KEFFEKFDDISYNDERDWFEFSFDYRHFTNKPTGTRTQWTLCTQGTRVRTFRNPEKSNHWD |
| | NEEFDLTQAFKDLFNKYGIDIASGLKARIVNGQLTKETSAVKDFYESLLKLLKLTLQMRNSV |
| | TGTDIDYLVSPVADKDGIFFDSRTCGSLLPANADANGAFNIARKGLMLLRQIQQSSIDAEKIQ |
| | LAPIKNEDWLEFAQEKPYL |
| SED ID NO: | MEKEITELTKIRREFPNKKFSSTDMKKAGKLLKAEGPDAVRDFLNSCQEIIGDFKPPVKTNIV |
| 62 | SISRPFEEWPVSMVGRAIQEYYFSLTKEELESVHPGTSSEDHKSFFNITGLSNYNYTSVQGLNL |
| | IFKNAKAIYDGTLVKANNKNKKLEKKFNEINHKRSLEGLPIITPDFEEPFDENGHLNNPPGINR |
| | NIYGYQGCAAKVFVPSKHKMVSLPKEYEGYNRDPNLSLAGFRNRLEIPEGEPGHVPWFQRM |
| | DIPEGQIGHVNKIQRFNFVHGKNSGKVKFSDKTGRVKRYHHSKYKDATKPYKFLEESKKVS |
| | ALDSILAHITIGDDWVVFDIRGLYRNVFYRELAQKGLTAVQLLDLFTGDPVIDPKKGVVTFSY |
| | KEGVVPVFSQKIVPRFKSRDTLEKLTSQGPVALLSVDLGQNEPVAARVCSLKNINDKITLDNS |
| | CRISFLDDYKKQIKDYRDSLDELEIKIRLEAINSLETNQQVEIRDLDVFSADRAKANTVDMFDI |
| | DPNLISWDSMSDARVSTQISDLYLKNGGDESRVYFEINNKRIKRSDYNISQLVRPKLSDSTRK |
| | NLNDSIWKLKRTSEEYLKLSKRKLELSRAVVNYTIRQSKLLSGINDIVIILEDLDVKKKFNGR |
| | GIRDIGWDNFFSSRKENRWFIPAFHKAFSELSSNRGLCVIEVNPAWTSATCPDCGFCSKENRD |
| | GINFTCRKCGVSYHADIDVATLNIARVAVLGKPMSGPADRERLGDTKKPRVARSRKTMKRK |
| | DISNSTVEAMVTA |
| SED ID NO: | MIKPTVSQFLTPGFKLIRNHSRTAGLKLKNEGEEACKKFVRENEIPKDECPNFQGGPAIANIIA |
| 63 | KSREFTEWEIYQSSLAIQEVIFTLPKDKLPEPILKEEWRAQWLSEHGLDTVPYKEAAGLNLIIK |
| | NAVNTYKGVQVKVDNKNKNNLAKINRKNEIAKLNGEQEISFEEIKAFDDKGYLLQKPSPNK |
| | SIYCYQSVSPKPFITSKYHNVNLPEEYIGYYRKSNEPIVSPYQFDRLRIPIGEPGYVPKWQYTF |
| | LSKKENKRRKLSKRIKNVSPILGIICIKKDWCVFDMRGLLRTNHWKKYHKPTDSINDLFDYF |
| | TGDPVIDTKANVVRFRYKMENGIVNYKPVREKKGKELLENICDQNGSCKLATVDVGQNNP |
| | VAIGLFELKKVNGELTKTLISRHPTPIDFCNKITAYRERYDKLESSIKLDAIKQLTSEQKIEVDN |
| | YNNNFTPQNTKQIVCSKLNINPNDLPWDKMISGTHFISEKAQVSNKSEIYFTSTDKGKTKDV |
| | MKSDYKWFQDYKPKLSKEVRDALSDIEWRLRRESLEFNKLSKSREQDARQLANWISSMCD |
| | VIGIENLVKKNNFFGGSGKREPGWDNFYKPKKENRWWINAIHKALTELSQNKGKRVILLPA |
| | MRTSITCPKCKYCDSKNRNGEKFNCLKCGIELNADIDVATENLATVAITAQSMPKPTCERSG |
| | DAKKPVRARKAKAPEFHDKLAPSYTVVLREAV |
| SED ID NO: | MEKEITELTKIRREFPNKKFSSTDMKKAGKLLKAEGPDAVRDFLNSCQEIIGDFKPPVKTNIV |
| 64 | SISRPFEEWPVSMVGRAIQEYYFSLTKEELESVHPGTSSEDHKSFFNITGLSNYNYTSVQGLNL |
| | IFKNAKAIYDGTLVKANNKNKKLEKKFNEINHKRSLEGLPIITPDFEEPFDENGHLNNPPGINR |
| | NIYGYQGCAAKVFVPSKHKMVSLPKEYEGYNRDPNLSLAGFRNRLEIPEGEPGHVPWFQRM |
| | DIPEGQIGHVNKIQRFNFVHGKNSGKVKFSDKTGRVKRYHHSKYKDATKPYKFLEESKKVS |
| | ALDSILAIITIGDDWVVFDIRGLYRNVFYRELAQKGLTAVQLLDLFTGDPVIDPKKGVVTFSY |
| | KEGVVPVFSQKIVPRFKSRDTLEKLTSQGPVALLSVDLGQNEPVAARVCSLKNINDKITLDNS |
| | CRISFLDDYKKQIKDYRDSLDELEIKIRLEAINSLETNQQVEIRDLDVFSADRAKANTVDMFDI |
| | DPNLISWDSMSDARVSTQISDLYLKNGGDESRVYFEINNKRIKRSDYNISQLVRPKLSDSTRK |
| | NLNDSIWKLKRTSEEYLKLSKRKLELSRAVVNYTIRQSKLLSGINDIVIILEDLDVKKKFNGR |
| | GIRDIGWDNFFSSRKENRWFIPAFHKTFSELSSNRGLCVIEVNPAWTSATCPDCGFCSKENRD |
| | GINFTCRKCGVSYHADIDVATLNIARVAVLGKPMSGPADRERLGDTKKPRVARSRKTMKRK |
| | DISNSTVEAMVTA |
| SED ID NO: | VPDKKETPLVALCKKSFPGLRFKKHDSRQAGRILKSKGEGAAVAFLEGKGGTTQPNFKPPV |
| 65 | KCNIVAMSRPLEEWPIYKASVVIQKYVYAQSYEEFKATDPGKSEAGLRAWLKATRVDTDGY |
| | FNVQGLNLIFQNARATYEGVLKKVENRNSKKVAKIEQRNEHRAERGLPLLTLDEPETALDET |
| | GHLRHRPGINCSVFGYQHMKLKPYVPGSIPGVTGYSRDPSTPIAACGVDRLEIPEGQPGYVPP |
| | WDRENLSVKKHRRKRASWARSRGGAIDDNMLLAVVRVADDWALLDLRGLLRNTQYRKLL |
| | DRSVPVTIESLLNLVINDPTLSVVKKPGKPVRYTATLIYKQGVVPVVKAKVVKGSYVSKML |
| | DDTTETFSLVGVDLGVNNLIAANALRIRPGKCVERLQAFTLPEQTVEDFFRFRKAYDKHQEN |
| | LRLAAVRSLTAEQQAEVLALDTFGPEQAKMQVCGHLGLSVDEVPWDKVNSRSSILSDLAKE |
| | RGVDDTLYMFPFFKGKGKKRKTEIRKRWDVNWAQHFRPQLTSETRKALNEAKWEAERNSS |
| | KYHQLSIRKKELSRHCVNYVIRTAEKRAQCGKVIVAVEDLHHSFRRGGKGSRKSGWGGFFA |
| | AKQEGRWLMDALFGAFCDLAVHRGYRVIKVDPYNTSRTCPECGHCDKANRDRVNREAFIC |
| | VCCGYRGNADIDVAAYNIAMVAITGVSLRKAARASVASTPLESLAAE |
| SEQ ID NO: | MPKPAVESEFSKVLKKHFPGERFRSSYMKRGGKILAAQGEEAVVAYLQGKSEEEPPNFQPPA |
| 66 | KCHVVTKSRDFAEWPIMKASEAIQRYIYALSTTERAACKPGKSSESHAAWFAATGVSNHGY |
| | SHVQGLNLIFDHTLGRYDGVLKKVQLRNEKARARLESINASRADEGLPEIKAEEEEVATNET |
| | GHLLQPPGINPSFYVYQTISPQAYRPRDEIVLPPEYAGYVRDPNAPIPLGVVRNRCDIQKGCP |
| | GYIPEWQREAGTAISPKTGKAVTVPGLSPKKNKRMRRYWRSEKEKAQDALLVTVRIGTDW |
| | VVIDVRGLLRNARWRTIAPKDISLNALLDLFTGDPVIDVRRNIVTFTYTLDACGTYARKWTL |
| | KGKQTKATLDKLTATQTVALVAIDLGQTNPISAGISRVTQENGALQ |
| | CEPLDRFTLPDDLLKDISAYRIAWDRNEEELRARSVEALPEAQQAEVRALDGVSKETARTQL |
| | CADFGLDPKRLPWDKMSSNTTFISEALLSNSVSRDQVFFTPAPKKGAKKKAPVEVMRKDRT |
| | WARAYKPRLSVEAQKLKNEALWALKRTSPEYLKLSRRKEELCRRSINYVIEKTRRRTQCQIV |
| | IPVIEDLNVRFFHGSGKRLPGWDNFFTAKKENRWFIQGLHKAFSDLRTHRSFYVFEVRPERTS |
| | ITCPKCGHCEVGNRDGEAFQCLSCGKTCNADLDVATHNLTQVALTGKTMPKREEPRDAQG |
| | TAPARKTKKASKSKAPPAEREDQTPAQEPSQTS |
| SEQ ID NO: | MSNKTTPPSPLSLLLRAHFPGLKFESQDYKIAGKKLRDGGPEAVISYLTGKGQAKLKDVKPP |
| 67 | AKAFVIAQSRPFIEWDLVRVSRQIQEKIFGIPATKGRPKQDGLSETAFNEAVASLEVDGKSKL |
| | NEETRAAFYEVLGLDAPSLHAQAQNALIKSAISIREGVLKKVENRNEKNLSKTKRRKEAGEE |
| | ATFVEEKAHDERGYLIHPPGVNQTIPGYQAVVIKSCPSDFIGLPSGCLAKESAEALTDYLPHD |
| | RMTIPKGQPGYVPEWQHPLLNRRKNRRRRDWYSASLNKPKATCSKRSGTPNRKNSRTDQIQ |
| | SGRFKGAIPVLMRFQDEWVIIDIRGLLRNARYRKLLKEKSTIPDLLSLFTGDPSIDMRQGVCTF |
| | IYKAGQACSAKMVKTKNAPEILSELTKSGPVVLVSIDLGQTNPIAAKVSRVTQLSDGQLSHE |
| | TLLRELLSNDSSDGKEIARYRVASDRLRDKLANLAVERLSPEHKSEILRAKNDTPALCKARV |
| | CAALGLNPEMIAWDKMTPYTEFLATAYLEKGGDRKVATLKPKNRPEMLRRDIKFKGTEGV |
| | RIEVSPEAAEAYREAQWDLQRTSPEYLRLSTWKQELTKRILNQLRHKAAKSSQCEVVVMAF |
| | EDLNIKMMHGNGKWADGGWDAFFIKKRENRWFMQAFHKSLTELGAHKGVPTIEVTPHRTS |
| | ITCTKCGHCDKANRDGERFACQKCGFVAHADLEIATDNIERVALTGKPMPKPESERSGDAK |
| | KSVGARKAAFKPEEDAEAAE |
| SEQ ID NO: | MSKTKELNDYQEALARRLPGVRHQKSVRRAARLVYDRQGEDAMVAFLDGKEVDEPYTLQ |
| 68 | PPAKCHILAVSRPIEEWPIARVTMAVQEHVYALPVHEVEKSRPETTEGSRSAWFKNSGVSNH |
| | GVTHAQTLNAILKNAYNVYNGVIKKVENRNAKKRDSLAAKNKSRERKGLPHFKADPPELA |
| | TDEQGYLLQPPSPNSSVYLVQQHLRTPQIDLPSGYTGPVVDPRSPIPSLIPIDRLAIPPGQPGYV |
| | PLHDREKLTSNKHRRMKLPKSLRAQGALPVCFRVFDDWAVVDGRGLLRHAQYRRLAPKNV |
| | SIAELLELYTGDPVIDIKRNLMTFRFAEAVVEVTARKIVEKYHNKYLLKLTEPKGKPVREIGL |
| | VSIDLNVQRLIALAIYRVHQTGESQLALSPCLHREILPAKGLGDFDKYKSKFNQLTEEILTAA |
| | VQTLTSAQQEEYQRYVEESSHEAKADLCLKYSITPHELAWDKMTSSTQYISRWLRDHGWN |
| | ASDFTQITKGRKKVERLWSDSRWAQELKPKLSNETRRKLEDAKHDLQRANPEWQRLAKRK |
| | QEYSRHLANTVLSMAREYTACETVVIAIENLPMKGGFVDGNGSRESGWDNFFTHKKENRW |
| | MIKDIHKALSDLAPNRGVHVLEVNPQYTSQTCPECGHRDKANRDPIQRERFCCTHCGAQRH |
| | ADLEVATHNIAMVATTGKSLTGKSLAPQRLQEAAE |
| SEQ ID NO: | VAFLDGKEVDEPYTLQPPAKCHILAVSRPIEEWPIARVTMAVQEHVYALPVHEVEKSRPETT |
| 69 | EGSRSAWFKNSGVSNHGVTHAQTLNAILKNAYNVYNGVIKKVENRNAKKRDSLAAKNKSR |
| | ERKGLPHFKADPPELATDEQGYLLQPPSPNSSVYLVQQHLRTPQIDLPSGYTGPVVDPRSPIPS |
| | LIPIDRLAIPPGQPGYVPLHDREKLTSNKHRRMKLPKSLRAQGALPVCFRVFDDWAVVDGRG |
| | LLRHAQYRRLAPKNVSIAELLELYTGDPVIDIKRNLMTFRFAEAVVEVTARKIVEKYHNKYL |
| | LKLTEPKGKPVREIGLVSIDLNVQRLIALAIYRVHQTGESQLALSPCLHREILPAKGLGDFDK |
| | YKSKFNQLTEEILTAAVQTLTSAQQEEYQRYVEESSHEAKADLCLKYSITPHELAWDKMTSS |
| | TQYISRWLRDHGWNASDFTQITKGRKKVERLWSDSRWAQELKPKLSNETRRKLEDAKHDL |
| | QRANPEWQRLAKRKQEYSRHLANTVLSMAREYTACETVVIAIENLPMKGGFVDGNGSRES |
| | GWDNFFTHKKENRWMIKDIHKALSDLAPNRGVHVLEVNPQYTSQTCPECGHRDKANRDPI |
| | QRERFCCTHCGAQRHADLEVATHNIAMVATTGKSLTGKSLAPQRLQ |
| SEQ ID NO: | MRSIKYELKDSYGIAGLRNRIADATISDNKWLYGNINLNDYLEWRSSKTDKQIEDGDRESSL |
| 81 | LGFWLEALRLGFVFSKQSHAPNDFNETALQDLFETLDDDLKHVLDRKKWCDFIKIGTPKTN |
| | DQGRLKKQIKNLLKGNKREEIEKTLNESDDELKEKINRIADVFAKNKSDKYTIFKLDKPNTE |
| | KYPRINDVQVAFFCHPDFEEITERDRTKTLDLIINRFNKRYEITENKKDDKTSNRMALYSLNQ |
| | GYIPRVLNDLFLFVKDNEDDFSQFLSDLENFFSFSNEQIKIIKERLKKLKKYAEPIPGKPQLAD |
| | KWDDYASDFGGKLESWYSNRIEKLKKIPESVSDLRNNLEKIRNVLKKQNNASKILELSQKIIE |
| | YIRDYGVSFEKPEIIKFSWINKTKDGQKKVFYVAKMADREFIEKLDLWMADLRSQLNEYNQ |
| | DNKVSFKKKGKKIEELGVLDFALNKAKKNKSTKNENGWQQKLSESIQSAPLFFGEGNRVRN |
| | EEVYNLKDLLFSEIKNVENILMSSEAEDLKNIKIEYKEDGAKKGNYVLNVLARFYARFNEDG |
| | YGGWNKVKTVLENIAREAGTDFSKYGNNNNRNAGRFYLNGRERQVFTLIKFEKSITVEKILE |
| | LVKLPSLLDEAYRDLVNENKNHKLRDVIQLSKTIMALVLSHSDKEKQIGGNYIHSKLSGYNA |
| | LISKRDFISRYSVQTTNGTQCKLAIGKGKSKKGNEIDRYFYAFQFFKNDDSKINLKVIKNNSH |
| | KNIDFNDNENKINALQVYSSNYQIQFLDWFFEKHQGKKTSLEVGGSFTIAEKSLTIDWSGSNP |
| | RVGFKRSDTEEKRVFVSQPFTLIPDDEDKERRKERMIKTKNRFIGIDIGEYGLAWSLIEVDNG |
| | DKNNRGIRQLESGFITDNQQQVLKKNVKSWRQNQIRQTFTSPDTKIARLRESLIGSYKNQLES |
| | LMVAKKANLSFEYEVSGFEVGGKRVAKIYDSIKRGSVRKKDNNSQNDQSWGKKGINEWSF |
| | ETTAAGTSQFCTHCKRWSSLAIVDIEEYELKDYNDNLFKVKINDGEVRLLGKKGWRSGEKIK |
| | GKELFGPVKDAMRPNVDGLGMKIVKRKYLKLDLRDWVSRYGNMAIFICPYVDCHHISHAD |
| | KQAAFNIAVRGYLKSVNPDRAIKHGDKGLSRDFLCQEEGKLNFEQIGLL |
| SEQ ID NO: | MIKKPSNRHALPKVIISKVDNQNILEFKIKYKKLSRLDRVEIKTMHYDDRAIVFDEVIINGGLI |
| 82 | DVEYRDNHKTIFVKVGDKSYSISGQKVGGKERLLENRISQTKVQLELKDEATNRVSKTEREL |
| | IVDDNIKLYSQIVGRDVKTTKDIYLIKRFLGYRSDLLFYYGFVNNFFHVANNRPEFWKIDFND |
| | NRNSKLIEYFIFTINDHLKNDENYLKDYISDRGQIVDDLENIKHIFSALRHGLMHFDYDFFEAL |
| | FNGEDIDIKMDNQGNTQPLSSLNIKFLDIMIDKLDKLNIDTKKEFIDAEKITIFGEELSLAKLYR |
| | FYAHTAINRVAFNKLINSFIIENGVENQSLKEYFNQQAGGIAYEIDIHQNREYKNLYNEHKKL |
| | VSRVLSISDGQEIATLNQKIVELKEQMKQITKINSIKRLEYKLRLAFGFIYTEYKNYEEFKNSF |
| | DTDIKNGRFTPKDEDGNKRAFDSRELEHLKGYYKATLQTQKPQTDEKMEEVSKRVDRLSLK |
| | SLIGDDTLLKFILLMFTFMPQELKGEFLGFIKKYYHDTKHIDQDTISDSDDTIEEGLSIGLKLKI |
| | LDKNIRSLSILKHSLSFQTKYNKKDRSYYEDGNIHGKFFKKLGISHNQEEFNKSVYAPLFRYY |
| | SALYKLINDFEIYTLSLHIVGNETLSDQVNKPQFLSGRYFNFRKLLTQSYNISNNSTHSVIFNA |
| | VINMRNDISHLSYEPLLDCPLNGKKSYKRKIRNQFRTINIKPLVESRKMIIDFITLQTDMQKVL |
| | GCDAVNDFTMKIVQLRTRLKAYANKEQTIEKMITEAKTPNDFYNIYKVKGVEAINKYLLEVI |
| | GETQVEKEIREEIERGNIANS |
| SEQ ID NO: | MKSIFDNFTGLYSLSKTLRFELRPVGQTLENIKNGHFLESDKKMADDYQDVKKIIDNYHKFFI |
| 83 | DDVLKGASFDWALLEKELTDFNKNKTDDSKVEAEQKKLREQIAKTLAGDKRFKSLTASTPN |
| | DLFNKDKDFIGWLEQSSVKEIRKDALDTFKKFSSYFKGFQENRKNVYSADDIPTAVPYRIVN |
| | DNFPKFLQNISIFKTIQEKCPQVIADVENELASYLGKEKLADIFTVQAFNKYLCQGGKENQRG |
| | IDFYNQVIGGIAEKEGGVNLRGINQFLNLYWQQHPDFAKENRRIKMVPLYKQILSDRSSLSFK |
| | IETIDTDEELKTAISEYADKLESKSNDEKKSVLDVCVELFDSIKEQNLQEIYVNRKDINNISRIL |
| | TGDWSWLQSRMNLYADEVFTTKAEKTRWQKSVDGDEGENKSKGAYSLAELNRVLEYASE |
| | NVAETDIRITDYFCHRNRFYYEKESGLFKQGEELVALSIKESCEDILSKRKAMNEAFANISES |
| | NSLRDNSEDIAKTKTYLDSVQDLLHRIKPLKVNGLGDPSFYAVFDSIYSALSEVISIYNKTRN |
| | YITRKAESPEKYKLNFDNPTLANGWDLNKEKDNTCVLLRKNGMYYLGIMNPKDKPKFAEK |
| | YDCGTESCYEKMIYKLLPGPNKMLPKVFFSTKGKKQYNPPENILHGYEQGKHKKGVAFDIN |
| | FCHELIDWFKSAINQHEDWKKFGFKFSDTKSYKDISDFYREVTEQGYKLTFINIPESEISKMVS |
| | EGKLYLFQIYNMDFAPGANGMPNMHTLYWKNLFSEENLKDVVLKLNGEAELFYRPAGIKE |
| | PVVHAKGSYLVNRITKDGEPIPEKIHDEIYRNANGNLNNLSKEASEYKESHKVVIKQASHEII |
| | KDRHYTEPKFLFHIPLTINFKAPSMKPTVCTAIQNENVRRFLKNNPDVNIIGLDRGERNLIYLS |
| | LINQKGEIIKQFSFNDVEREQNGQTVKVNYHEKLDQREKERDAARKSWQAIGKIAELKEGYL |
| | SAVIHQLTKLMVEYNAIVVMEDLNFGFKRGRFHVEKQVYQKFEHMLIDKLNYLVFKDRGL |
| | NEPGGALNGYQLAGQFESFQKLGKQSGMLFYVPAAYTSKIDPKTGFVSMMNFKDLTNVHK |
| | KRDFFSKFEDIHFDEATCSFVFTFDYKNINGKAKEEMKQTKWAVYSREKRIVYFSKTKSSED |
| | IMPTEKLRALFESDGIEYKSGNNIHDSVMAVGADLKEGAKPSKEIADFWDGLLYNFKLILQM |
| | RNSNAKTGEDYIISPVMASDGTFFDSRVEAKKGKDAKLPLDADANGAYHIALKGLSLINKIN |
| | LAGEDELKKFDMKISNEDWFNFAQEKKYAE |
| SEQ ID NO: | MTKQNKSVTQNTKRKNFGEFTNLYSLSKTLRFELKPVKATKTILEVERKDRENFKKDRKIAK |
| 84 | NYQKLKGILNELHQEFIQDVMREFSFQEKEIKEFEERYLEALNFKEKDNYKKRTQLKNAYEK |
| | VAKKLAGKIATAFGKYNQEKYGVKFTKKNLTGENVFDILEGKYKGDKKILGIIHTFKFKPTK |
| | EEKKQGKEAVNFSTYLGGFNQNRENFYKGEMKAGQFATRTIENLIQFLKNKKLFIDKYKDN |
| | YQKIGFSQEQVEIFNLNYFNNLFLQEGLDVYNGILGAKKGEKNTENDGLNQKINLFKQKEKT |
| | RCKANGEKFNKSDYPIFKELYKQIGSIKKDNDVYVEIKSDEELVNVLQSLPEKTANTLREVQ |
| | KFYENFFDKIFNDEFDLDKIYLPKSVGTHFSHLAFSDWSKLAFVFNKRWRNEKVKIKEGEDV |
| | NVQSRSLADIKKRMEEILEMDGGVSFGKTYCQKVGLEKEARTIEDVWSGFWKIIQYHINSQF |
| | IGGEKEVFDKEKKDDKTEKIQTIDDLQEEYLQATEMYRERMVESEEGLNDGEEKEIKTKLKN |
| | YLDRIKDIERIARYFDLRKHFDDIDEASKDGDFYFIYQELLQDISEAKINDHYNEIRNYLTKAN |
| | VVDDKFKLNFNDGQTLSGWDLNKETEKFSLIFKRKVDGGVEYYLGIINKEKNKTIFDKKKHP |
| | EIFTENSEFEKMEYKLFPSPSKMLPKIAFTKNKEGERIKPVFLDENAGKEIAQIKKEFALFQDA |
| | KKEDKNKWSDEFDRKKLNKLIDYYKLVLEKHPEKYMQTFNFVFKSSAKYKNLGEFNDDVA |
| | RQNYVTKFVSVDKDYIDQKVESGELYLFKIHNKDWNLTKAGDTKKQSKKNLHTIYFEELFS |
| | EKNIAEPVFKLSGGAEVFFRDAIEKKKQKKKKDKKGKEILEKFRFTKNKILFHVPITINYGKPS |
| | INQGQFNQKINEFIADNSRSVNILGIDRGEKHLLYYSLVDNNGKIIKSGSLNEINGVDYHEKLD |
| | KAEKERQEARKSWQKINQIKNLKAGYISQVIKKIVDLAIENNAIIVLEDLNFGFKSFRQKIEKN |
| | VYQQFEKALIDKLGFVTDKEKLNHRQAPQLSAPFESFEKMGKQTGIVFYVLATNTSKVCPQC |
| | QWKKNIFPHYSTKKSIAENLQKQYKMKMYWRENENRFEFEYKGDGDKEFSSIFSNVDRVRY |
| | DKRANNNQGGYVIYQIDSTTKEKDGRNIKEKSITNLLKELLLEKFEIDNLEGELLVKLSEKSP |
| | DVSKETIKDFFGLLNSILNIRNSMTDTEEDYIQCPACGFDTRKENKIGIKNGDDNGAYNIALR |
| | GRFLIERIKKAKKEDKKPNLTFSNNDYFQWVREFVK |
| SEQ ID NO: | MIKSYDDFTKLYSLQKTLRFELKPIGKTLEHIKKSEIIESDETLEEQYAIVKNIIDRFHRKHIDE |
| 85 | ALSLVDFTKHLDTFKTIQELYLKRGKTDREKKELEELSADLRKLIVSYLKGNVKQKTQHNLN |
| | PIKERFEILFGKELFTNEEFFTLAENKEEKKAIQAFKGFTTYFKGFQENRKNMYSEEDKSTAIA |
| | YRIINENMPLFIENIARFQKVLDVIEKTKLTELKQNLKTELKGHSVSDIFRIEYFNNVLTQEGIS |
| | RYNTIIGGKTTTEGVKIQGLNEIINLHNQQSKDVKLPILKPLHKQILSEEYSTSFKIKAFENDNE |
| | VLKAIDTFWNEHIEKSIHPVTGKRFNILLKIENLCKKLEKYKDKEIEKLFIERKNLSTVSHQVY |
| | GQWNIIRDALRMHLEMNNKNIKEKDIDKYLDNDAFAWKEIKDSIKIYKEHVEDAKELDENG |
| | IVKYFSSMSINEEDDEKEYSISLIKNINEKYNNVKSILEEDRTGKSDLHQDKEKVAIIKEFLDSL |
| | KQLQWFLKLLYVTVPLDEKDYEFYNELEVYYEALLPLNSLYNKVRNYMTRKPYSVEKFKL |
| | NFYSPTLLDGWDKNKETANLSIILKKNGKYYLGIMNKENNTIFENFPKSKSNDYYEKMIYKL |
| | LPGPNKMLPKVFFSKKGLEYYKPSKEILRIYEKGEFKKDKSGNFKKESLHTLIDFYKEAIAKN |
| | EDWKIFKFKFKNTREYEDISQFYRDVEEQGYLIIFEKVDANYVDKLVEEGELYLFQIYNKDFS |
| | ENKKSKGNPNLHTIYWESLFDNQNLKDVVYKLNGEAEVFYRKKSIDYPEEIYNNGHHKEEL |
| | NGKFNYPIIKDRRYTQDKFLFHVPITMNFISKEEKRVNQLACEYLSTTKEDVHIIGIDRGERHL |
| | LYLSLIDKEGNIKKQLSLNTIKNENYDKEIDYRVKLDEKEKKRDEARKNWDVIENIKELKEG |
| | YMSQVIHIIAKMMAEEKAILIMEDLNIGFKRGRFKVEKQVYQKFEKMMIDKLNYLVFKNKE |
| | PLEPGGSLNAYQLTSKFDSFKKLGKQSGFIFYVPSAYTSKIDPTTGFYNFIRVDVPNLEKGKEF |
| | FSKFEKIIYNTKEDYFEFHCKYGKFVPEPKNKDNDRKTKESLTYYNAIKDTVWVVCSTHHER |
| | YKIVRNKAGYYESQPVDVTKNLRDIFSEANINYSDGKDIKPIIIESNNAKLLKSIAEQLKLILA |
| | MRYNNGKHDDDEKDYILSPVKNKQGKFFCSLDGDQSLPINADANGAYNIALKGLLLIEKIKK |
| | QQGKAKDLYISNLEWFMFMMSR |
| SEQ ID NO: | MRTTTSLDAFTNRYALSKTLRFELKPIGNTQMMLEQNNVFAKDRAIREKYEKTKPWIDLLH |
| 86 | REFVAESLQNAQLGNLDDYYAALQNVQKITKDTNAEDKKRWKKGFEKQEKRLRKEVVALF |
| | DKAAHIWATQRYPQLKKKTKDFLFEEGVFEHVLFARYGSAPDTTVKIVTSNPETGEVIDERE |
| | ESIFKGWKGFTGYFDKFFETRKNFYKDNGTATAIATRAINQNLRRFAENMQKLIDIKNNYPE |
| | LLAHTDFGDFDIAHAQSLDFYARTCLLQEGIDAYNKKFVGVLKSAINEYQQKNKGVRISYPK |
| | TLDNQILGERERRLFDVIEDDRELHDVFRAFVDDGTVFAAEMRQLAQAFSAQNGTYDYTQI |
| | YISKKGFETISRKYTHDTRAWHDALADVFKAKAKKRIATTASGEKKFPAYIPVAYITQALTL |
| | VQESEDTECTWKERYASITENKTLEEGFFAIFADEFERLFVHMEATVQDTDYVVAEDKAKK |
| | LLSDGQITKNEQTTQIIKEYADALLRIYQMAKYFAVEKKSMWDDAVAIDDTFYETFKEIYGN |
| | THSTIVASYNLLRNYLTKKPWEDVQKWKLNFENPTLLDGWDKNKEAANFGVILRDGDKFY |
| | LGIMRKGHNNIFANQHHSNFEGQGLQKMVYKFFPDPKKMFPKVCFSAKGMEFFAPSEEIVRI |
| | YKNAEFKSGDTFNVESMQKLIDFYKNALQKYDGWKIYDFKHLKDTAQYTSNIGEFYDDVA |
| | KGGYQLGWQNISKEYVEEKNANGELYLFQIKNKDWNDGATGRKNLHTLYFEYLFSEKNAA |
| | ADFVFRLNGGAEVFYRPAAIESKTERRGNREVAAKKRYTQDKVFLHVPITLNRTAGDVKTS |
| | AFNDAVNRFLAGNPDINIMGIDRGEKHLAYYSIIDQNGNRIVSGSFNTIGSKDYHALLTERQG |
| | AREEARKNWQRVEQIKDLKKGYISLVVREIADLAIKHNAIIVLENLNMRFKQIRGGIEKSVYQ |
| | QLEKALIEKLNFLVNKGEVDATKAGHLLRAYQLAAPFETFEKMGNQTGIIFYTTASYTSQVD |
| | PVTGWRPHVYLKYRNAQKTKEDILRIFDDIVENDEKQRFEFAYRHNGVSWTVCSSVERHRW |
| | NRSNNAGKGGYDVFPVEGEGSITQRLQEACAQRGIDTTRNILAQIDELDESASATVSFLRDLC |
| | FYFRLICQIRNTDDGADDINAQDFLMSPVEPFEDTRNAQEQYPQNGDENGAYNIARKGIIILQ |
| | KITAWGRSQDTQRRYPDTFVSQDEWDTFLTQHTT |
| SEQ ID NO: | MLKEKQFKTFGDFTNLYELSKTLRFELRPTPETKDLLDKNKIIQTDKKIAENYQEIKKYFDKL |
| 87 | HKKFIKEALSNTQIDFSDFCKLWEQNSKDSGKIKDLSRKLRKSIKQAFDKKGADWHKRYLE |
| | KGIKLKKKNLDILFEERVLDILKEEFKDDVDVKLFESFKGFSTYFTNFHESRKNFYKDDGTAS |
| | AIATRIIDENLKRFCDNIKVKKHSKKLISELNEREAKIFEADFYNRCLLQQGIDDYNQVIGDIN |
| | KKINNLRQNKIENTPTLKILYKQILGDVRRQETEQDAFIEIKNNEEVFDFLQDFIKHSDENNKY |
| | FKNLFYKFIEGKHSLDKIFLAKRFVNTISGKWFASWEVFGAELIKKFGNKKDLPDFIPFAAVK |
| | DVLQNCNIPANELFKEKIKNDEDKNIYDIFINLWKEEFDSNLKKVEESKKEVENMIAEDKVY |
| | SNKKEKRKNDNGEEIEIEIQKEKIKNYADAAMNIFRMMKYFLLEKNGKTVEGMGEDNNFYN |
| | ELNIVFKGGEIDGKVYEGVKTYLYYNEFRNYLTKKPFNEEKTKLNFDCGQILSGWDKNKES |
| | EKLGVILRKDNKYYLAIINKKHINKIFDVKKNSYAYIVGDNFYEKMEYKLFPDAKRMIPKIAF |
| | AKNNKEKFGWTDEIQKIKNEYAEFQEGKKNDKNLWKDKFNKNKMEKLITYYQNCLEKGG |
| | YKDIYNFRWKSPDKYSGIGEFNDEIDRQSYCLKFVKVDFNYVFEKVKSGELYLFQIYNKDFS |
| | DKADRAQKENIHTEYFKLLFDQRNLDNVVLKLSGGAEIFYRPKTEGLPKKKDNKGNEVVRH |
| | RRYADDKYFLHLPIQLNFGRGNLSGGEFNSKINQYLSEQREIKIIGIDRGEKHLAYYSVINQD |
| | GKIEEIESLNTVNGIDYRKKLDELEKKREQERKSWQSISKIKDLKKGYISHVIKKICDLAIEHN |
| | AIIVFEDLSGGFKNSRKKIEKQIYQNLELALATKLNYLTFKDKNFGESGHYLNAYQLAPKIDN |
| | YQDIKMQTGIVFYTPAGYTSSTCPQCGFRKTLKFDYTATISKAEDLIRGSKLNIVFEKEKNRF |
| | KINYLFNPIEKKKKKIKENELFADAGAKNEFTIYSDVKRIKWHNTGTKRLEEAEGERLLENK |
| | NSRGRDKEYDINKCLTRLFRENKIDVNGDIIGQITKIKSLKLYQDLFYYLFLATLIRNNVSGSD |
| | IDYIQCPSCHFHSDGGFQKQKENGDANGAYNIARKGILILKKIKQFAAQDKDMKNFGWKHL |
| | TVDINEWDKFTQK |
| SEQ ID NO: | MNKNFSNFTELYTLSKTLRFELKPVAQTKENIKKGKFLESDKKKADDYKDVKKIIDNYHKFF |
| 88 | IDDVLKNASFDWTVLEKEMSDFNKSKADDSKVEAEQKKLRDQIAKKLTSDKRFKALTASTP |
| | SDLFNKDKDFIDWFTQNSTKDINKEALETFKRFSSYFKGFQENRKNVYSAEPIPTAVPYRLVN |
| | DNFPKFLQNIALFKIIQEKCPQVISDVEKELASYLGKEKLADIFTVQAFNKYLCQGGKENQRG |
| | IDFYNNILGGIAEKEGGINLRGINQFLNLYWQQHQDFAKQNKRIKMIPLFKQILSDRSSLSFKI |
| | ESINTDQELLTSITEYADKLETKSNDEKKSVLDICSDLFASIKAQNLQEIYVNRKDINSISRILT |
| | GDWSWLQSRMNVYADEVFTTKAEKTRWQKSIDGDEGENKSKGVFSLAELNSVLEYSSENV |
| | SETDVRITDFFDHRNRFYYEKESGLFKQGDELVALSIRESCEDILAKRKAMDEAFANVSENN |
| | SLRDNSEDVAKIKIYLDCVQELLHRIKPLKVNGLGDPAFYAVFDTVYNSLSEVISLYNKTRN |
| | YITKKAANPEKYKLNFDNPTLADGWDLNKEQANTSVLMRKDGMYYLGIMNPKDKPKFAE |
| | KYECGNEACYEKMIYKQFDATKQIPKCSTQVKEVKKHFQSGATDSIILNDKSKFKLDLVITK |
| | EIWFLNNHVWNGEKFVPKRESNETRPKKFQIGYYKQTGDLGGYKEALNIWISFCKTFLQSYI |
| | SSSIYDYDFKESSNYDSLDEFYNYLNATCYKLSFINIPEATISQMVSEGKLYLFQIYNKDFAPG |
| | ASGMPNMHTLYWKNLFSEENLKNVVLKLNGEAELFYRPAGIKEPVIHAKGSYLVNRITKDG |
| | EPIPEKIHDEIYRNANGKLESLSKEATEYKASHKVVIKEAKHDIIKDRHYTEPKFLFHVPLTIN |
| | FKASGNSYINENVRRFLKNNPDVNVIGLDRGERHLIYLSLINQKGEIIKQFTFNEVERNKNGQ |
| | VIKVNYHEKLDQREKVRGAARKSWQAIGKIAELKEGYLSAVIHQLTKLMVEYNAIVVMEDL |
| | NFGFKRGRFHVEKQVYQKFEHMLIDKLNYLVFKDRGLTEAGGVLNGYQLAGQFESFQKLG |
| | KQSGMLFYVPAGYTSKIDPKTGFASMSNFKDLTNVHKKRAFFSKFDDIHFDDATGSFVFTFD |
| | YKNFDGKAKEEMKRTKWSVYSKDKRIVYLSKTKSYEDVQPTEKIKASLESVGIEYMSGNNL |
| | IDSIMVIGAELKDGAKPSKEIADFWDRLLYNFKLILQMRNSNAKTGEDYIISPVMADDGTFFD |
| | SREEFKKGENAKMPVDADANGAYHIALKGLSLLKRFDAASENELKKFDMKISNVDWFKFA |
| | QEKSYAE |

Other Exemplary protein sequences are described in the following applications: PCT/US21/33271; PCT/US21/35031, and U.S. Provisional Patent Application No. 63/187,298, all of which are herein incorporated by reference in their entirety.

In some cases, the effector proteins comprise a RuvC domain (e.g., a partial RuvC domain). In some instances, the RuvC domain may be defined by a single, contiguous sequence, or a set of partial RuvC domains that are not contiguous with respect to the primary amino acid sequence of the protein. An effector protein of the present disclosure may include multiple partial RuvC domains, which may combine to generate a RuvC domain with substrate binding or catalytic activity. For example, an effector protein may include three partial RuvC domains (RuvC-I, RuvC-II, and RuvC-III, also referred to herein as subdomains) that are not contiguous with respect to the primary amino acid sequence of the effector protein, but form a RuvC domain once the protein is produced and folds. In some cases, effector proteins comprise a recognition domain with a binding affinity for a guide nucleic acid or for a guide nucleic acid-target nucleic acid heteroduplex. In some instances, the effector protein does not comprise a zinc finger domain. In some instances, the effector protein does not comprise an HNH domain.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide nucleic acid (e.g., a dual gRNA or a sgRNA), wherein cleavage occurs within or directly adjacent to the region of the target nucleic acid that is hybridized to guide nucleic acid. Trans cleavage activity (also referred to as transcollateral cleavage) is cleavage of ssDNA or ssRNA that is near, but not hybridized to the guide nucleic acid. Trans cleavage activity is triggered by the hybridization of guide nucleic acid to the target nucleic acid. Nickase activity is a selective cleavage of one strand of a dsDNA.

Effector proteins of the present disclosure, dimers thereof, and multimeric complexes thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region.

In some instances, the Type V CRISPR/Cas protein has been modified (also referred to as an engineered protein). For example, a Type V CRISPR/Cas protein disclosed herein or a variant thereof may comprise a nuclear localization signal (NLS). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

Several programmable nucleases are consistent with the methods and devices of the present disclosure. For example, Cas proteins are programmable nucleases used in the methods and systems disclosed herein. Cas proteins can include any of the known Classes and Types of CRISPR/Cas enzymes. Programmable nucleases disclosed herein include Class 1 Cas proteins, such as the Type I, Type IV, or Type III Cas proteins. Programmable nucleases disclosed herein also include the Class 2 Cas proteins, such as the Type II, Type V, and Type VI Cas proteins. Programmable nucleases included in the devices disclosed herein and methods of use thereof include a Type V or Type VI Cas proteins.

In some instances, the programmable nuclease is a Type V Cas protein. In general, a Type V Cas effector protein comprises a RuvC domain, but lacks an HNH domain. In most instances, the RuvC domain of the Type V Cas effector protein comprises three patrial RuvC domains (RuvC-I, RuvC-II, and RuvC-III, also referred to herein as subdomains). In some instances, the three RuvC subdomains are located within the C-terminal half of the Type V Cas effector protein. In some instances, none of the RuvC subdomains are located at the N terminus of the protein. In some instances, the RuvC subdomains are contiguous. In some instances, the RuvC subdomains are not contiguous with respect to the primary amino acid sequence of the Type V Cas protein, but form a ruvC domain once the protein is produced and folds. In some instances, there are zero to about 50 amino acids between the first and second RuvC subdomains. In some instances, there are zero to about 50 amino acids between the second and third RuvC subdomains. In some instances, the Cas effector is a Cas14 effector. In some instances, the Cas14 effector is a Cas14a, Cas14al, Cas14b, Cas14c, Cas14d, Cas14e, Cas14f, Cas14g, Cas14h, or Cas14u effector. In some instances, the Cas effector is a CasPhi effector. In some instances, the Cas effector is a Cas12 effector. In some instances, the Cas12 effector is a Cas12a, Cas12b, Cas12c, Cas12d, Cas12e, or Cas12j effector.

In some instances, the Type V CRISPR/Cas protein comprises a Cas14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas 14 proteins may comprise a RuvC domain or a partial RuvC domain. The RuvC domain may be defined by a single, contiguous sequence, or a set of partial RuvC domains that are not contiguous with respect to the primary amino acid sequence of the Cas 14 protein. In some instances, a partial RuvC domain does not have any substrate binding activity or catalytic activity on its own. A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas 14 protein, but form a RuvC domain once the protein is produced and folds. A Cas14 protein may comprise a linker loop connecting a carboxy terminal domain of the Cas14 protein with the amino terminal domain of the Cas 14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas 14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas14 proteins may be relatively small compared to many other Cas proteins, making them suitable for nucleic acid detection or gene editing. For instance, a Cas14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas14 protein is 400 to 800 amino acid residues long, 400 to 600 amino acid residues long, 440 to 580 amino acid residues long, 460 to 560 amino acid residues long, 460 to 540 amino acid residues long, 460 to 500 amino acid residues long, 400 to 500 amino acid residues long, or 500 to 600 amino acid residues long. In some cases, a Cas14 protein is less than about 550 amino acid residues long. In some cases, a Cas 14 protein is less than about 500 amino acid residues long.

In some instances, a Cas14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas 14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of reporters with a detection moiety.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acid via a single catalytic RuvC domain. The RuvC domain is within a nuclease, or “NUC” lobe of the protein, and the Cas 12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas12 nuclease can be a Cas12a protein, a Cas12b protein, Cas12c protein, Cas12d protein, or a Cas12e protein.

In some embodiments, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the Cas12 can be Cas12M08, which is a specific protein variant within the Cas12 protein family/classification). In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Ish), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum ((′am), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus ((ca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR/Cas enzyme (e.g., a Cas13 nuclease). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. An RNA reporter can be an RNA-based reporter. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA reporters by target DNA. Thus, Type VI CRISPR/Cas enzyme (e.g., a Cas13 nuclease, such as Cas13a) can be DNA-activated programmable RNA nucleases, and therefore can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly taken from organisms or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

Other Exemplary protein sequences are described in the following applications: PCT/US21/33271; PCT/US21/35031, and U.S. Provisional Patent Application No. 63/187,298, all of which are herein incorporated by reference in their entirety.

In some embodiments a programmable nuclease is referred to as an effector protein. In some instances, an effector protein disclosed herein is an engineered protein. The engineered protein is not identical to a naturally-occurring protein. The engineered protein may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise. Compositions and systems may comprise an engineered protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wildtype counterpart protein. The modified form of the wildtype counterpart may comprise an amino acid change (e.g., deletion, insertion, or substitution) that reduces the nucleic acid-cleaving activity of the programmable nuclease. For example, a nuclease domain (e.g., RuvC domain) of a Type V CRISPR/Cas protein may be deleted or mutated so that it is no longer functional or comprises reduced nuclease activity. The modified form of the programmable nuclease may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g. inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with an engineered guide nucleic acid to activate or repress transcription of a target nucleic acid sequence. In some embodiments, the enzymatically inactive protein is fused with a protein comprising recombinase activity.

In some instances, a programmable nuclease is a fusion protein, wherein the fusion protein comprises a protein comprising the amino acid sequence of any one of SEQ ID NOs: 9-69 or 81-88. In some instances, the fusion protein comprises a programmable nuclease and a fusion partner protein.

A fusion partner protein is also simply referred to herein as a fusion partner. In some cases, the fusion partner promotes the formation of a multimeric complex of the programmable nuclease. In some cases, the fusion partner is an additional programmable nuclease. In some cases, the multimeric complex comprising the programmable nuclease and the additional programmable nuclease binds a guide nucleic acid. The programmable nucleases of the multimeric complex may bind the guide nucleic acid in an asymmetric fashion. In some cases, one programmable nuclease of the multimeric complex interacts more strongly with the guide nucleic acid than the additional programmable nuclease of the multimeric complex. In some cases, a programmable nuclease interacts more strongly with a target nucleic acid when it is complexed with the guide nucleic acid relative to when the programmable nuclease or the multimeric complex is not complexed with the guide nucleic acid.

In some cases, the fusion partner has enzymatic activity in the presence of its enzyme substrate. For example, the fusion partner may comprise an enzyme such as horse radish peroxidase (HRP) which can catalyze a detectable color change reaction in the presence of its substrate (e.g., TMB).

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some cases, a terminus of the programmable nuclease is linked to a terminus of the fusion partner through an amide bond. In some cases, a programmable nuclease is coupled to a fusion partner via a linker protein. In some cases, a programmable nuclease is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. These linkers may be produced by using synthetic, linker-encoding oligonucleotides to couple the proteins, or may be encoded by a nucleic acid sequence encoding a fusion protein (e.g., an programmable nuclease coupled to a fusion partner). Examples of linker proteins include glycine polymers (G) n (SEQ ID NO: 70), glycine-serine polymers (including, for example, (GS)n (SEQ ID NO: 71), GSGGSn (SEQ ID NO: 72), GGSGGSn (SEQ ID NO: 73), and GGGSn (SEQ ID NO: 74), where n is an integer of at least one), glycine-alanine polymers, and alanine-serine polymers. Exemplary linkers may comprise amino acid sequences including, but not limited to, GGSG (SEQ ID NO: 75), GGSGG (SEQ ID NO: 76), GSGSG (SEQ ID NO: 77), GSGGG (SEQ ID NO: 78), GGGSG (SEQ ID NO: 79), and GSSSG (SEQ ID NO: 80).

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, compositions and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid may comprise a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid may comprise two naturally occurring sequences arranged in an order or proximity that is not observed in nature. In some instances, compositions and systems comprise a ribonucleotide complex comprising a programmable nuclease and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

Described herein are various embodiments of thermostable programmable nucleases. In some embodiments, a programmable nuclease is referred to as a programmable nuclease. A programmable nuclease may be thermostable. In some instances, known programmable nucleases (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of a programmable nuclease in a trans cleavage assay at 40° C. may be at least 50% of that at 37° C. In some instances, the trans cleavage activity of a programmable nuclease in a trans cleavage assay at 40° C. may be at least 55% of that at 37° C. In some instances, the trans cleavage activity of a programmable nuclease in a trans cleavage assay at 40° C. may be at least 60% of that at 37° C. In some instances, the trans cleavage activity of a programmable nuclease in a trans cleavage assay at 40° C. may be at least 65% of that at 37° C. In some instances, the trans cleavage activity of a programmable nuclease in a trans cleavage assay at 40° C. may be at least 70% of that at 37° C. In some instances, the trans cleavage activity of a programmable nuclease in a trans cleavage assay at 40° C. may be at least 75% of that at 37° C. In some instances, the trans cleavage activity of a programmable nuclease in a trans cleavage assay at 40° C. may be at least 80% of that at 37° C. In some instances, the trans cleavage activity of a programmable nuclease in a trans cleavage assay at 40° C. may be at least 85% of that at 37° C. In some instances, the trans cleavage activity of a programmable nuclease in a trans cleavage assay at 40° C. may be at least 90% of that at 37° C. In some instances, the trans cleavage activity of a programmable nuclease in a trans cleavage assay at 40° C. may be at least 95% of that at 37° C. In some instances, the trans cleavage activity of a programmable nuclease in a trans cleavage assay at 40° C. may be at least 100% of that at 37° C. In some instances, the trans cleavage activity of a programmable nuclease in a trans cleavage assay at 40° C. may be at least 1-fold of that at 37° C. In some instances, the trans cleavage activity of a programmable nuclease in a trans cleavage assay at 40° C. may be at least 2-fold of that at 37° C. In some instances, the trans cleavage activity of a programmable nuclease in a trans cleavage assay at 40° C. may be at least 3-fold of that at 37° C. In some instances, the trans cleavage activity of a programmable nuclease in a trans cleavage assay at 40° C. may be at least 4-fold of that at 37° C. In some instances, the trans cleavage activity of a programmable nuclease in a trans cleavage assay at 40° C. may be at least 5-fold of that at 37° C. In some instances, the trans cleavage activity of a programmable nuclease in a trans cleavage assay at 40° C. may be at least 6-fold of that at 37° C. In some instances, the trans cleavage activity of a programmable nuclease in a trans cleavage assay at 40° C. may be at least 7-fold of that at 37° C. In some instances, the trans cleavage activity of a programmable nuclease in a trans cleavage assay at 40° C. may be at least 8-fold of that at 37° C. In some instances, the trans cleavage activity of a programmable nuclease in a trans cleavage assay at 40° C. may be at least 9-fold of that at 37° C. In some instances, the trans cleavage activity of a programmable nuclease in a trans cleavage assay at 40° C. may be at least 10-fold of that at 37° C. In some instances, the trans cleavage activity of a programmable nuclease in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 45° C. may be at least 50% of that at 37° C. In some instances, the trans cleavage activity of a programmable nuclease in a trans cleavage assay at 45° C. may be at least 55% of that at 37° C. In some instances, the trans cleavage activity of a programmable nuclease in a trans cleavage assay at 45° C. may be at least 60% of that at 37° C. In some instances, the trans cleavage activity of a programmable nuclease in a trans cleavage assay at 45° C. may be at least 65% of that at 37° C. In some instances, the trans cleavage activity of a programmable nuclease in a trans cleavage assay at 45° C. may be at least 70% of that at 37° C. In some instances, the trans cleavage activity of a programmable nuclease in a trans cleavage assay at 45° C. may be at least 75% of that at 37° C. In some instances, the trans cleavage activity of a programmable nuclease in a trans cleavage assay at 45° C. may be at least 80% of that at 37° C. In some instances, the trans cleavage activity of a programmable nuclease in a trans cleavage assay at 45° C. may be at least 85% of that at 37° C. In some instances, the trans cleavage activity of a programmable nuclease in a trans cleavage assay at 45° C. may be at least 90% of that at 37° C. In some instances, the trans cleavage activity of a programmable nuclease in a trans cleavage assay at 45° C. may be at least 95% of that at 37° C. In some instances, the trans cleavage activity of a programmable nuclease in a trans cleavage assay at 45° C. may be at least 100% of that at 37° C. In some instances, the trans cleavage activity of a programmable nuclease in a trans cleavage assay at 45° C. may be at least 1-fold of that at 37° C. In some instances, the trans cleavage activity of a programmable nuclease in a trans cleavage assay at 45° C. may be at least 2-fold of that at 37° C. In some instances, the trans cleavage activity of a programmable nuclease in a trans cleavage assay at 45° C. may be at least 3-fold of that at 37° C. In some instances, the trans cleavage activity of a programmable nuclease in a trans cleavage assay at 45° C. may be at least 4-fold of that at 37° C. In some instances, the trans cleavage activity of a programmable nuclease in a trans cleavage assay at 45° C. may be at least 5-fold of that at 37° C. In some instances, the trans cleavage activity of a programmable nuclease in a trans cleavage assay at 45° C. may be at least 6-fold of that at 37° C. In some instances, the trans cleavage activity of a programmable nuclease in a trans cleavage assay at 45° C. may be at least 7-fold of that at 37° C. In some instances, the trans cleavage activity of a programmable nuclease in a trans cleavage assay at 45° C. may be at least 8-fold of that at 37° C. In some instances, the trans cleavage activity of a programmable nuclease in a trans cleavage assay at 45° C. may be at least 9-fold of that at 37° C. In some instances, the trans cleavage activity of a programmable nuclease in a trans cleavage assay at 45° C. may be at least 10-fold of that at 37° C. In some instances, the trans cleavage activity of a programmable nuclease in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 50° C. may be at least 50% of that at 37° C. In some instances, the trans cleavage activity of a programmable nuclease in a trans cleavage assay at 50° C. may be at least 55% of that at 37° C. In some instances, the trans cleavage activity of a programmable nuclease in a trans cleavage assay at 50° C. may be at least 60% of that at 37° C. In some instances, the trans cleavage activity of a programmable nuclease in a trans cleavage assay at 50° C. may be at least 65% of that at 37° C. In some instances, the trans cleavage activity of a programmable nuclease in a trans cleavage assay at 50° C. may be at least 70% of that at 37° C. In some instances, the trans cleavage activity of a programmable nuclease in a trans cleavage assay at 50° C. may be at least 75% of that at 37° C. In some instances, the trans cleavage activity of a programmable nuclease in a trans cleavage assay at 50° C. may be at least 80% of that at 37° C. In some instances, the trans cleavage activity of a programmable nuclease in a trans cleavage assay at 50° C. may be at least 85% of that at 37° C. In some instances, the trans cleavage activity of a programmable nuclease in a trans cleavage assay at 50° C. may be at least 90% of that at 37° C. In some instances, the trans cleavage activity of a programmable nuclease in a trans cleavage assay at 50° C. may be at least 95% of that at 37° C. In some instances, the trans cleavage activity of a programmable nuclease in a trans cleavage assay at 50° C. may be at least 100% of that at 37° C. In some instances, the trans cleavage activity of a programmable nuclease in a trans cleavage assay at 50° C. may be at least 1-fold of that at 37° C. In some instances, the trans cleavage activity of a programmable nuclease in a trans cleavage assay at 50° C. may be at least 2-fold of that at 37° C. In some instances, the trans cleavage activity of a programmable nuclease in a trans cleavage assay at 50° C. may be at least 3-fold of that at 37° C. In some instances, the trans cleavage activity of a programmable nuclease in a trans cleavage assay at 50° C. may be at least 4-fold of that at 37° C. In some instances, the trans cleavage activity of a programmable nuclease in a trans cleavage assay at 50° C. may be at least 5-fold of that at 37° C. In some instances, the trans cleavage activity of a programmable nuclease in a trans cleavage assay at 50° C. may be at least 6-fold of that at 37° C. In some instances, the trans cleavage activity of a programmable nuclease in a trans cleavage assay at 50° C. may be at least 7-fold of that at 37° C. In some instances, the trans cleavage activity of a programmable nuclease in a trans cleavage assay at 50° C. may be at least 8-fold of that at 37° C. In some instances, the trans cleavage activity of a programmable nuclease in a trans cleavage assay at 50° C. may be at least 9-fold of that at 37° C. In some instances, the trans cleavage activity of a programmable nuclease in a trans cleavage assay at 50° C. may be at least 10-fold of that at 37° C. In some instances, the trans cleavage activity of a programmable nuclease in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 55° C. may be at least 50% of that at 37° C. In some instances, the trans cleavage activity of a programmable nuclease in a trans cleavage assay at 55° C. may be at least 55% of that at 37° C. In some instances, the trans cleavage activity of a programmable nuclease in a trans cleavage assay at 55° C. may be at least 60% of that at 37° C. In some instances, the trans cleavage activity of a programmable nuclease in a trans cleavage assay at 55° C. may be at least 65% of that at 37° C. In some instances, the trans cleavage activity of a programmable nuclease in a trans cleavage assay at 55° C. may be at least 70% of that at 37° C. In some instances, the trans cleavage activity of a programmable nuclease in a trans cleavage assay at 55° C. may be at least 75% of that at 37° C. In some instances, the trans cleavage activity of a programmable nuclease in a trans cleavage assay at 55° C. may be at least 80% of that at 37° C. In some instances, the trans cleavage activity of a programmable nuclease in a trans cleavage assay at 55° C. may be at least 85% of that at 37° C. In some instances, the trans cleavage activity of a programmable nuclease in a trans cleavage assay at 55° C. may be at least 90% of that at 37° C. In some instances, the trans cleavage activity of a programmable nuclease in a trans cleavage assay at 55° C. may be at least 95% of that at 37° C. In some instances, the trans cleavage activity of a programmable nuclease in a trans cleavage assay at 55° C. may be at least 100% of that at 37° C. In some instances, the trans cleavage activity of a programmable nuclease in a trans cleavage assay at 55° C. may be at least 1-fold of that at 37° C. In some instances, the trans cleavage activity of a programmable nuclease in a trans cleavage assay at 55° C. may be at least 2-fold of that at 37° C. In some instances, the trans cleavage activity of a programmable nuclease in a trans cleavage assay at 55° C. may be at least 3-fold of that at 37° C. In some instances, the trans cleavage activity of a programmable nuclease in a trans cleavage assay at 55° C. may be at least 4-fold of that at 37° C. In some instances, the trans cleavage activity of a programmable nuclease in a trans cleavage assay at 55° C. may be at least 5-fold of that at 37° C. In some instances, the trans cleavage activity of a programmable nuclease in a trans cleavage assay at 55° C. may be at least 6-fold of that at 37° C. In some instances, the trans cleavage activity of a programmable nuclease in a trans cleavage assay at 55° C. may be at least 7-fold of that at 37° C. In some instances, the trans cleavage activity of a programmable nuclease in a trans cleavage assay at 55° C. may be at least 8-fold of that at 37° C. In some instances, the trans cleavage activity of a programmable nuclease in a trans cleavage assay at 55° C. may be at least 9-fold of that at 37° C. In some instances, the trans cleavage activity of a programmable nuclease in a trans cleavage assay at 55° C. may be at least 10-fold of that at 37° C. In some instances, the trans cleavage activity of a programmable nuclease in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 60° C. may be at least 50% of that at 37° C. In some instances, the trans cleavage activity of a programmable nuclease in a trans cleavage assay at 60° C. may be at least 55% of that at 37° C. In some instances, the trans cleavage activity of a programmable nuclease in a trans cleavage assay at 60° C. may be at least 60% of that at 37° C. In some instances, the trans cleavage activity of a programmable nuclease in a trans cleavage assay at 60° C. may be at least 65% of that at 37° C. In some instances, the trans cleavage activity of a programmable nuclease in a trans cleavage assay at 60° C. may be at least 70% of that at 37° C. In some instances, the trans cleavage activity of a programmable nuclease in a trans cleavage assay at 60° C. may be at least 75% of that at 37° C. In some instances, the trans cleavage activity of a programmable nuclease in a trans cleavage assay at 60° C. may be at least 80% of that at 37° C. In some instances, the trans cleavage activity of a programmable nuclease in a trans cleavage assay at 60° C. may be at least 85% of that at 37° C. In some instances, the trans cleavage activity of a programmable nuclease in a trans cleavage assay at 60° C. may be at least 90% of that at 37° C. In some instances, the trans cleavage activity of a programmable nuclease in a trans cleavage assay at 60° C. may be at least 95% of that at 37° C. In some instances, the trans cleavage activity of a programmable nuclease in a trans cleavage assay at 60° C. may be at least 100% of that at 37° C. In some instances, the trans cleavage activity of a programmable nuclease in a trans cleavage assay at 60° C. may be at least 1-fold of that at 37° C. In some instances, the trans cleavage activity of a programmable nuclease in a trans cleavage assay at 60° C. may be at least 2-fold of that at 37° C. In some instances, the trans cleavage activity of a programmable nuclease in a trans cleavage assay at 60° C. may be at least 3-fold of that at 37° C. In some instances, the trans cleavage activity of a programmable nuclease in a trans cleavage assay at 60° C. may be at least 4-fold of that at 37° C. In some instances, the trans cleavage activity of a programmable nuclease in a trans cleavage assay at 60° C. may be at least 5-fold of that at 37° C. In some instances, the trans cleavage activity of a programmable nuclease in a trans cleavage assay at 60° C. may be at least 6-fold of that at 37° C. In some instances, the trans cleavage activity of a programmable nuclease in a trans cleavage assay at 60° C. may be at least 7-fold of that at 37° C. In some instances, the trans cleavage activity of a programmable nuclease in a trans cleavage assay at 60° C. may be at least 8-fold of that at 37° C. In some instances, the trans cleavage activity of a programmable nuclease in a trans cleavage assay at 60° C. may be at least 9-fold of that at 37° C. In some instances, the trans cleavage activity of a programmable nuclease in a trans cleavage assay at 60° C. may be at least 10-fold of that at 37° C. In some instances, the trans cleavage activity of a programmable nuclease in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of a programmable nuclease in a trans cleavage assay at 65° C. may be at least 50% of that at 37° C. In some instances, the trans cleavage activity of a programmable nuclease in a trans cleavage assay at 65° C. may be at least 55% of that at 37° C. In some instances, the trans cleavage activity of a programmable nuclease in a trans cleavage assay at 65° C. may be at least 60% of that at 37° C. In some instances, the trans cleavage activity of a programmable nuclease in a trans cleavage assay at 65° C. may be at least 65% of that at 37° C. In some instances, the trans cleavage activity of a programmable nuclease in a trans cleavage assay at 65° C. may be at least 70% of that at 37° C. In some instances, the trans cleavage activity of a programmable nuclease in a trans cleavage assay at 65° C. may be at least 75% of that at 37° C. In some instances, the trans cleavage activity of a programmable nuclease in a trans cleavage assay at 65° C. may be at least 80% of that at 37° C. In some instances, the trans cleavage activity of a programmable nuclease in a trans cleavage assay at 65° C. may be at least 85% of that at 37° C. In some instances, the trans cleavage activity of a programmable nuclease in a trans cleavage assay at 65° C. may be at least 90% of that at 37° C. In some instances, the trans cleavage activity of a programmable nuclease in a trans cleavage assay at 65° C. may be at least 95% of that at 37° C. In some instances, the trans cleavage activity of a programmable nuclease in a trans cleavage assay at 65° C. may be at least 100% of that at 37° C. In some instances, the trans cleavage activity of a programmable nuclease in a trans cleavage assay at 65° C. may be at least 1-fold of that at 37° C. In some instances, the trans cleavage activity of a programmable nuclease in a trans cleavage assay at 65° C. may be at least 2-fold of that at 37° C. In some instances, the trans cleavage activity of a programmable nuclease in a trans cleavage assay at 65° C. may be at least 3-fold of that at 37° C. In some instances, the trans cleavage activity of a programmable nuclease in a trans cleavage assay at 65° C. may be at least 4-fold of that at 37° C. In some instances, the trans cleavage activity of a programmable nuclease in a trans cleavage assay at 65° C. may be at least 5-fold of that at 37° C. In some instances, the trans cleavage activity of a programmable nuclease in a trans cleavage assay at 65° C. may be at least 6-fold of that at 37° C. In some instances, the trans cleavage activity of a programmable nuclease in a trans cleavage assay at 65° C. may be at least 7-fold of that at 37° C. In some instances, the trans cleavage activity of a programmable nuclease in a trans cleavage assay at 65° C. may be at least 8-fold of that at 37° C. In some instances, the trans cleavage activity of a programmable nuclease in a trans cleavage assay at 65° C. may be at least 9-fold of that at 37° C. In some instances, the trans cleavage activity of a programmable nuclease in a trans cleavage assay at 65° C. may be at least 10-fold of that at 37° C. In some instances, the trans cleavage activity of a programmable nuclease in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of a programmable nuclease in a trans cleavage assay at 70° C., 75° C. 80° C., or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity may be measured against a negative control in a trans cleavage assay. In some instances, the trans cleavage activity of a programmable nuclease against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of a programmable nuclease against a nucleic acid in a trans cleavage assay at 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of a programmable nuclease against a nucleic acid in a trans cleavage assay at 70° C., 75° C., 80° C., or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

The reporters described herein can be RNA reporters. The RNA reporters can comprise at least one ribonucleic acid and a detectable moiety. In some embodiments, a programmable nuclease probe or a CRISPR probe comprising a programmable nuclease can recognize and detect ssDNA and, further, can specifically trans-cleave RNA reporters. The detection of the target nucleic acid in the sample can indicate the presence of the disease (or disease-causing agent) in the sample and can provide information for taking action to reduce the transmission of the disease to individuals in the disease-affected environment or near the disease-carrying individual.

Cleavage of a reporter (i.e., a protein-nucleic acid or detector nucleic acid) can produce a signal. The signal can indicate a presence of the target nucleic acid in the sample, and an absence of the signal can indicate an absence of the target nucleic acid in the sample. In some cases, cleavage of the reporter can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors and detectors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor or detector suitable for detecting calorimetric signals, potentiometric signals, amperometric signals, optical signals, or piezo-electric signals.

In an aspect, the present disclosure provides a method for target detection. The method can comprise sample collection. The method can further comprise sample preparation. The method can further comprise detection of one or more target molecules in the collected and prepared sample. In some embodiments, sample preparation can include nucleic acid amplification and the target molecules can include target amplicons.

In another aspect, the present disclosure provides a detection device for target detection. The detection device can be configured for multiplexed target detection. The detection device can be used to collect one or more samples, prepare or process the one or more samples for detection, and optionally divide the one or more samples into a plurality of droplets, aliquots, volumes, or subsamples for amplification of one or more target sequences or target nucleic acids. The target sequences may comprise, for example, a biological sequence. The biological sequence can comprise a nucleic acid sequence or an amino acid sequence. In some embodiments, the target sequences are associated with an organism of interest, a disease of interest, a disease state of interest, a phenotype of interest, a genotype of interest, or a gene of interest.

The detection device can be configured to amplify target nucleic acids contained within the plurality of droplets, aliquots, or subsamples. The detection device can be configured to amplify the target sequences or target nucleic acids contained within the plurality of droplets or volumes by individually processing each of the plurality of droplets or volumes (e.g., by using a thermocycling process or any other suitable amplification process as described in greater detail below). In some cases, the plurality of droplets or volumes can undergo separate thermocycling processes. In some cases, the thermocycling processes can occur simultaneously. In other cases, the thermocycling processes can occur at different times for each droplet or volume.

The detection device can be further configured to remix the droplets, aliquots, volumes, or subsamples after the target nucleic acids in each of the droplets undergo amplification. The detection device can be configured to provide the remixed sample comprising the droplets, aliquots, volumes, or subsamples to a detection chamber of the device. The detection chamber can be configured to direct the remixed droplets, aliquots, volumes, or subsamples to a plurality of programmable nuclease probes. The detection chamber can be configured to direct the remixed droplets, aliquots, volumes, or subsamples along one or more fluid flow paths such that the remixed droplets, aliquots, volumes, or subsamples are positioned adjacent to and/or in contact with the one or more programmable nuclease probes. In some cases, the detection chamber can be configured to recirculate or recycle the remixed droplets, aliquots, volumes, or subsamples such that the remixed droplets, aliquots, volumes, or subsamples are repeatedly placed in contact with one or more programmable nuclease probes over a predetermined period of time.

The instrument and/or detection device can comprise one or more sensors or detectors. The one or more sensors or detectors of the instrument and/or detection device can be configured to detect one or more signals that are generated after one or more programmable nucleases of the one or more programmable nuclease probes become activated due to a binding of a guide nucleic acid of the programmable nuclease probes with a target nucleic acid present in the sample or amplicon thereof. As described elsewhere herein, the activated programmable nuclease can bind or cleave the target nucleic acid, which can result in a trans cleavage activity. Trans cleavage activity can be a non-specific cleavage of nearby single-stranded nucleic acids by the activated programmable nuclease, such as trans cleavage of reporter nucleic acids with a detection moiety. Once the reporter nucleic acids are cleaved by the activated programmable nucleases, the detection moiety can be released or separated from the reporter, thereby generating one or more detectable signals. The one or more sensors or detectors of the instrument or detection device can be configured to register and/or process the one or more detectable signals to confirm a presence and/or an absence of a particular target (e.g., a target nucleic acid) in a sample.

The one or more programmable nuclease probes of the detection device can be configured for multiplexed detection. In some cases, each programmable nuclease probe can be configured to detect a particular target. In other cases, each programmable nuclease probe can be configured to detect a plurality of targets. In some cases, a first programmable nuclease probe can be configured to detect a first target or a first set of targets, and a second programmable nuclease probe can be configured to detect a second target or a second set of targets. In other cases, a first programmable nuclease probe can be configured to detect a first set of targets, and a second programmable nuclease probe can be configured to detect a second set of targets. The programmable nuclease probes of the present disclosure can be used to detect a plurality of different target sequences or target nucleic acids. In any of the embodiments described herein, the sample provided to the detection device can comprise a plurality of target sequences or target nucleic acids. In any of the embodiments described herein, the sample provided to the detection device can comprise multiple classes of target sequences or target nucleic acids. Each class of target sequences or class of target nucleic acids can comprise a plurality of target sequences or target nucleic acids associated with a particular organism, disease state, phenotype, or genotype present within the sample. In some cases, each programmable nuclease probe can be used to detect a particular class of target sequences, or a particular class of target nucleic acids associated with a particular organism, disease state, phenotype, or genotype present within the sample. In some cases, two or more programmable nuclease probes can be used to detect different classes of target sequences or different classes of target nucleic acids. In such cases, the two or more programmable nuclease probes can comprise different sets or classes of guide nucleic acids complexed to the programmable nucleases of the probes.

### C. Guide Nucleic Acids

Provided herein are compositions comprising an effector protein and an engineered guide nucleic acid. In general, a guide nucleic acid is a nucleic acid molecule that binds to an effector protein (e.g., a Cas effector protein), thereby forming a ribonucleoprotein complex (RNP). In some instances, the engineered guide nucleic acid imparts activity or sequence selectivity to the effector protein. In general, the engineered guide nucleic acid may comprise a CRISPR RNA (crRNA) that is at least partially complementary to a target nucleic acid. In some instances, the engineered guide RNA comprises a trans-activating crRNA (tracrRNA), at least a portion of which interacts with the effector protein. The tracrRNA may hybridize to a portion of the guide RNA that does not hybridize to the target nucleic acid. In some instances, the crRNA and tracrRNA are provided as a single guide RNA (sgRNA). In some instances, compositions comprise a crRNA and tracrRNA that function together as two separate, unlinked molecules. Guide nucleic acids can be referred to as “guide RNA.” However, a guide nucleic acid may comprise deoxyribonucleotides. In some instances, at least one of the crRNA and tracrRNA is an engineered guide nucleic acid. The term “guide RNA,” as well as crRNA and tracrRNA, includes guide nucleic acids comprising DNA bases and RNA bases.

Table 4 shows a list of exemplary gRNAs that can be used with the immobilized reporters with the designs described thereof.

TABLE 4
Guides for Surface-Based Trans-Cleavage Assays

| Guide Description | Guide Sequence |
|---|---|
| G1 | 3′ no spacer R777 |
| G2 | 3′ C6 spacer R777 |
| G3 | 3′ C12 spacer R777 |
| G4 | 3′ C6 spacer R1763 |

In general, the crRNA comprises a spacer region that hybridizes to a target sequence of a target nucleic acid, and a repeat region that interacts with the effector protein. The repeat region may also be referred to as a “protein-binding segment.” Typically, the repeat region is adjacent to the spacer region. For example, a guide RNA that interacts with the effector protein comprises a repeat region that is 5′ of the spacer region. The spacer region of the guide RNA may comprise complementarity with (e.g., hybridize to) a target sequence of a target nucleic acid. In some cases, the spacer region is 15-28 linked nucleosides in length. In some cases, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some cases, the spacer region is 18-24 linked nucleosides in length. In some cases, the spacer region is at least 15 linked nucleosides in length. In some cases, the spacer region is at least 16, 18, 20, or 22 linked nucleosides in length. In some cases, the spacer region comprises at least 10, 11, 12, 13, 14, 15, 16, 17, 18, 19, 20, 21, 22, 23, 24, 25, 26, 27, 28, 29, or 30 nucleotides. In some cases, the spacer region is at least 17 linked nucleosides in length. In some cases, the spacer region is at least 18 linked nucleosides in length. In some cases, the spacer region is at least 20 linked nucleosides in length. In some cases, the spacer region is at least 80%, at least 85%, at least 90%, at least 95% or 100% complementary to a target sequence of the target nucleic acid. In some cases, the spacer region is 100% complementary to the target sequence of the target target nucleic acid. In some cases, the spacer region comprises at least 15 contiguous nucleobases that are complementary to the target nucleic acid.

The guide RNA may bind to a target nucleic acid (e.g., a single strand of a target nucleic acid) or a portion thereof. The guide nucleic acid may bind to a target nucleic acid such as a nucleic acid from a bacterium, a virus, a parasite, a protozoa, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment. The guide nucleic acid may bind to a target nucleic acid, such as DNA or RNA, from a cancer gene or gene associated with a genetic disorder, or an amplicon thereof, as described herein. The guide nucleic acid may comprise a first region complementary to a target nucleic acid (FR1) and a second region that is not complementary to the target nucleic acid (FR2). In some cases, FR1 is located 5′ to FR2 (FR1-FR2). In some cases, FR2 is located 5′ to FR1 (FR2-FR1).

In some cases, the guide comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some instances, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

### Pooling Guide Nucleic Acids

In some instances, compositions, systems or methods provided herein comprise a pool of guide nucleic acids. In some instances, the pool of guide nucleic acids were tiled against a target nucleic acid, e.g., the genomic locus of interest or uses thereof. In some instances, a guide nucleic acid is selected from a group of guide nucleic acids that have been tiled against a nucleic acid sequence of a genomic locus of interest. The genomic locus of interest may belong to a viral genome, a bacterial genome, or a mammalian genome. Non-limiting examples of viral genomes are an HPV genome, an HIV genome, an influenza genome, or a coronavirus genome. These guide nucleic acids are pooled for detecting a target nucleic acid in a single assay. Pooling of guide nucleic acids may ensure broad spectrum identification, or broad coverage, of a target species within a single reaction. This may be particularly helpful in diseases or indications, like sepsis, that may be caused by multiple organisms. The pool of guide nucleic acids may enhance the detection of a target nucleic using systems of methods described herein relative to detection with a single guide nucleic acid. The pool of guide nucleic acids may ensure broad coverage of the target nucleic acid within a single reaction using the methods described herein. In some instances, the pool of guide nucleic acids are collectively complementary to at least 50%, at least 55%, at least 60%, at least 65%, at least 70%, at least 75%, at least 80%, at least 85%, at least 90%, at least 95%, at least 97%, or 100% of the target nucleic acid. In some instances, at least a portion of the guide nucleic acids of the pool overlap in sequence. In some instances, at least a portion of the guide nucleic acids of the pool do not overlap in sequence. In some cases, the pool of guide nucleic acids comprises at least 2, at least 3, at least 4, at least 5, or at least 6 guide nucleic acids targeting different sequences of a target nucleic acid.

### Intermediary Nucleic Acids

A guide nucleic acid may comprise or be coupled to an intermediary nucleic acid. The intermediary nucleic acid may also be referred to as an intermediary RNA, although it may comprise deoxyribonucleosides in addition to ribonucleosides. The intermediary RNA may be separate from, but form a complex with a crRNA to form a discrete gRNA system. The intermediary RNA may be linked to a crRNA to form a composite gRNA. A programmable nuclease may bind a crRNA and an intermediary RNA. In some cases, the crRNA and the intermediary RNA are provided as a single nucleic acid (e.g., covalently linked). In some embodiments, the crRNA and the intermediary RNA are separate polynucleotides (e.g., a discrete gRNA system). An intermediary RNA may comprise a repeat hybridization region and a hairpin region. The repeat hybridization region may hybridize to all or part of the sequence of the repeat of a crRNA. The repeat hybridization region may be positioned 3′ of the hairpin region. The hairpin region may comprise a first sequence, a second sequence that is reverse complementary to the first sequence, and a stem-loop linking the first sequence and the second sequence.

The CRISPR/Cas ribonucleoprotein (RNP) complex may comprise a Cas protein complexed with a guide nucleic acid (e.g., a crRNA) and an intermediary RNA. Sometimes, a guide nucleic acid comprises a crRNA and an intermediary RNA (e.g., the crRNA and intermediary RNA are provided as a single nucleic acid molecule). A composition may comprise a crRNA, an intermediary RNA, a Cas protein, and a detector nucleic acid.

In some instances, the length of intermediary RNAs is not greater than 50, 56, 68, 71, 73, 95, or 105 linked nucleosides. In some embodiments, the length of an intermediary RNA is about 30 to about 120 linked nucleosides. In some embodiments, the length of an intermediary RNA is about 50 to about 105, about 50 to about 95, about 50 to about 73, about 50 to about 71, about 50 to about 68, or about 50 to about 56 linked nucleosides. In some embodiments, the length of an intermediary RNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of an intermediary RNA is 40 to 60 nucleotides. In some embodiments, the length of the intermediary RNA is 50, 56, 68, 71, 73, 95, or 105 linked nucleosides. In some embodiments, the length of the intermediary RNA is 50 nucleotides.

An exemplary intermediary RNA may comprise, from 5′ to 3′, a 5′ region, a hairpin region, a repeat hybridization region, and a 3′ region. In some cases, the 5′ region may hybridize to the 3′ region. In some embodiments, the 5′ region does not hybridize to the 3′ region. In some cases, the 3′ region is covalently linked to the crRNA (e.g., through a phosphodiester bond). In some embodiments, an intermediary RNA may comprise an unhybridized region at the 3′ end of the intermediary RNA. The unhybridized region may have a length of about 1, about 2, about 3, about 4, about 5, about 6, about 7, about 8, about 9, about 10, about 12, about 14, about 16, about 18, or about 20 linked nucleosides. In some embodiments, the length of the un-hybridized region is 0 to 20 linked nucleosides.

### II. Noncanonical PAM Sequences

In one aspect, the present disclosure provides systems for detecting a target nucleic acid in a sample using non-canonical PAM sequences. In some cases, the system comprises one or more units. In some instances, the one or more units comprise a plurality of different units. In some cases, a unit of the one or more units comprises: (a) a non-naturally occurring guide nucleic acid; (b) a plurality of reporters; and (c) a programmable nuclease. In some cases, the non-naturally occurring guide nucleic acid comprises a spacer region that hybridizes to a segment of the target nucleic acid or an amplicon thereof. In some cases, the segment hybridized by the spacer region is directly adjacent to a sequence that is complementary to a noncanonical PAM sequence. In some cases, the non-naturally occurring guide nucleic acid, the reporters, or both are immobilized to a surface, such as in the case of any of the immobilized reporter systems described herein. In some instances, each of the different units comprise a non-naturally occurring guide nucleic acid comprising a spacer region with a different sequence. In some cases, the non-naturally occurring guide nucleic acid is effective to form a complex with the programmable nuclease that is activated upon binding the target nucleic acid or amplicon thereof. In some cases, the formation of the activated complex is effective to induce detectable transcollateral cleavage of the reporters.

In some cases, the noncanonical PAM sequence can differ from a naturally-occurring PAM sequence for a reference Cas nuclease. In some cases, the reference Cas nuclease is a Cas nuclease of the same type. In some embodiments, the type of Cas is considered the same if it is the same general class of Cas nuclease (e.g., Type V or Type VI), same family (e.g., Cas12 or Cas14), same sub-family (e.g., Cas12a or Cas12b), or the same Cas nuclease (i.e., identical polypeptide sequence). In some embodiments, the Cas nuclease is a Cas12 protein, and the reference Cas is a Cas12 protein of the same or different type (e.g., one is a Cas12a variant and the other is a naturally occurring Cas12a). In some embodiments, the Cas nuclease is a Cas14 protein, and the reference Cas is a Cas14 protein of the same or different type (e.g., one is a Cas14a variant and the other is a naturally occurring Cas14a). In some embodiments, the Cas nuclease is a CasΦ protein, and the reference Cas is a CasΦ protein of the same or different type. In some embodiments, the Cas nuclease and the reference Cas nuclease are the same.

In some embodiments, a canonical PAM sequence is the consensus PAM sequence among targets of the reference Cas nuclease. In some embodiments, a canonical PAM sequence is the PAM sequence that results in the most efficient cleavage by the reference Cas nuclease. In general, the noncanonical PAM differs in sequence from the canonical PAM, and for an otherwise identical target sequence results in reduced cleavage activity by the programmable nuclease as compared to a target with the canonical PAM. In some cases, the canonical PAM sequence comprises TTTN or YYN. In some cases, the canonical PAM sequence comprises TTTA. In some instances, the noncanonical PAM sequence comprises CCGT, TCCG, CCCT, TGTC, TTGT, GGGC, TGGG, TTGG, TTTG, CTTT, CCTT, TCCT, TTCC, or CTGT. In some instances, the noncanonical PAM sequence comprises CCG, TTC, CCC, TGT, TTG, TCTT, TTCT, TTTT, CTTT, CCTT, TCGG, CTCG, TTTG, GGGC, TGGG, TTGG, TCCT, TTCC, or CTGT. As used herein when describing a PAM sequence, N is an adenine (A), a guanine (G), a cytosine (C), or a thymine (T); Y is a C or a T; S is a G or a C; K is a G or a T; V is an A, a C, or a G; B is a C, G, or a T; R is an A or a G.

Table 5 below provides illustrative examples of Cas enzymes and corresponding canonical PAM sequences. In some embodiments, the reference Cas has a sequence that is at least 80%, 90%, or 95% identical to one of SEQ ID NOs: 19, 26, 43, 52, 53, 62, 63, 65-69, 81, and 83-88 and has the corresponding canonical PAM sequence indicated in Table 5. In some embodiments, the reference Cas has as a sequence of any one of SEQ ID NOs: 19, 26, 43, 52, 53, 62, 63, 65-69, 81, and 83-88, and has the corresponding canonical PAM sequence indicated in Table 5.

TABLE 5
Cas Enzymes and Associated Canonical PAMs

| | Canonical |
|---|---|
| Cas Enzymes | PAM |
| MKKIDNFVGCYPVSKTLRFKAIPIGKTQENIEKKRLVEEDEVRAKDYKAVKKLIDRY | YYN |
| HREFIEGVLDNVKLDGLEEYYMLFNKSDREESDNKKIEIMEERFRRVISKSFKNNEEY | |
| KKIFSKKIIEEILPNYIKDEEEKELVKGFKGFYTAFVGYAQNRENMYSDEKKSTAISYR | |
| IVNENMPRFITNIKVFEKAKSILDVDKINEINEYILNNDYYVDDFFNIDFFNYVLNQKG | |
| IDIYNAIIGGIVTGDGRKIQGLNECINLYNQENKKIRLPQFKPLYKQILSESESMSFYIDE | |
| IESDDMLIDMLKESLQIDSTINNAIDDLKVLFNNIFDYDLSGIFINNGLPITTISNDVYG | |
| QWSTISDGWNERYDVLSNAKDKESEKYFEKRRKEYKKVKSFSISDLQELGGKDLSIC | |
| KKINEIISEMIDDYKSKIEEIQYLFDIKELEKPLVTDLNKIELIKNSLDGLKRIERYVIPFL | |
| GTGKEQNRDEVFYGYFIKCIDAIKEIDGVYNKTRNYLTKKPYSKDKFKLYFENPQLM | |
| GGWDRNKESDYRSTLLRKNGKYYVAIIDKSSSNCMMNIEEDENDNYEKINYKLLPG | |
| PNKMLPKVFFSKKNREYFAPSKEIERIYSTGTFKKDTNFVKKDCENLITFYKDSLDRH | |
| EDWSKSFDFSFKESSAYRDISEFYRDVEKQGYRVSFDLLSSNAVNTLVEEGKLYLFQL | |
| YNKDFSEKSHGIPNLHTMYFRSLFDDNNKGNIRLNGGAEMFMRRASLNKQDVTVHK | |
| ANQPIKNKNLLNPKKTTTLPYDVYKDKRFTEDQYEVHIPITMNKVPNNPYKINHMVR | |
| EQLVKDDNPYVIGIDRGERNLIYVVVVDGQGHIVEQLSLNEIINENNGISIRTDYHTLL | |
| DAKERERDESRKQWKQIENIKELKEGYISQVVHKICELVEKYDAVIALEDLNSGFKN | |
| SRVKVEKQVYQKFEKMLITKLNYMVDKKKDYNKPGGVLNGYQLTTQFESFSKMGT | |
| QNGIMFYIPAWLTSKMDPTTGFVDLLKPKYKNKADAQKFFSQFDSIRYDNQEDAFVF | |
| KVNYTKFPRTDADYNKEWEIYTNGERIRVFRNPKKNNEYDYETVNVSERMKELFDS | |
| YDLLYDKGELKETICEMEESKFFEELIKLFRLTLQMRNSISGRTDVDYLISPVKNSNG | |
| YFYNSNDYKKEGAKYPKDADANGAYNIARKVLWAIEQFKMADEDKLDKTKISIKN | |
| QEWLEYAQTHCE (SEQ ID NO: 26) | |
| MEVQKTVMKTLSLRILRPLYSQEIEKEIKEEKERRKQAGGTGELDGGFYKKLEKKHS | TTTN |
| EMFSFDRLNLLLNQLQREIAKVYNHAISELYIATIAQGNKSNKHYISSIVYNRAYGYF | |
| YNAYIALGICSKVEANFRSNELLTQQSALPTAKSDNFPIVLHKQKGAEGEDGGFRIST | |
| EGSDLIFEIPIPFYEYNGENRKEPYKWVKKGGQKPVLKLILSTFRRQRNKGWAKDEG | |
| TDAEIRKVTEGKYQVSQIEINRGKKLGEHQKWFANFSIEQPIYERKPNRSIVGGLDVGI | |
| RSPLVCAINNSFSRYSVDSNDVFKFSKQVFAFRRRLLSKNSLKRKGHGAAHKLEPITE | |
| MTEKNDKFRKKIIERWAKEVTNFFVKNQVGIVQIEDLSTMKDREDHFFNQYLRGFW | |
| PYYQMQTLIENKLKEYGIEVKRVQAKYTSQLCSNPNCRYWNNYFNFEYRKVNKFPK | |
| FKCEKCNLEISADYNAARNLSTPDIEKFVAKATKGINLPEK (SEQ ID NO: 19) | |
| MLKSYDYFTKLYSLQKTLRFELKPIGKTLEHIKNSGIIESDETLEEQYAIVKNIIDKLHR | YYN |
| KHIDEALSLVDFTKHLDTLKTFQELYLKRGKTDKEKEELEKLSADLRKLIVSYLKGN | |
| VKEKTQHNLNPIKERFEILFGKELFTNEEFFLLAENEKEKKAIQAFKGFTTYFKGFQEN | |
| RKNMYSEEGNSTSIAYRIINENLPLFIENIARFQKVMSTIEKTTIKKLEQNLKTELKKH | |
| NLPGIFTIEYFNNVLTQEGISRYNTIIGGKTTHEGVKIQGLNEIINLYNQQSKDVKLPIL | |
| KPLHKQILSEEYSTSFKIKAFENDNEVLKAIDTFWNEHIEKSIHPVTGNKFNILSKIENL | |
| CDQLQKYKDKDLEKLFIERKNLSTVSHQVYGQWNIIRDALRMHLEMNNKNIKEKDI | |
| DKYLDNDAFSWKEIKDSIKIYKEHVEDAKELNENGIIKYFSAMSINEEDDEKEYSISLI | |
| KNINEKYNNVKSILQEDRTGKSDLHQDKEKVGIIKEFLDSLKQLQWFLRLLYVTVPL | |
| DEKDYEFYNELEVYYEALLPLNSLYNKVRNYMTRKPYSVEKFKLNFNSPTLLDGWD | |
| KNKETANLSIILRKNGKYYLGIMNKENNTIFEYYPGTKSNDYYEKMIYKLLPGPNKM | |
| LPKVFFSKKGLEYYNPPKEILNIYEKGEFKKDKSGNFKKESLHTLIDFYKEAIAKNED | |
| WEVFNFKFKNTKEYEDISQFYRDVEEQGYLITFEKVDANYVDKLVKEGKLYLFQIYN | |
| KDFSENKKSKGNPNLHTIYWKGLYDSENLKNVVYKLNGEAEVFYRKKSIDYPEEIYN | |
| HGHHKEELLGKFNYPIIKDRRYTQDKFLFHVPITMNFISKEEKRVNQLACEYLSATKE | |
| DVHIIGIDRGERHLLYLSLIDKEGNIKKQLSLNTIKNENYDKEIDYRVKLDEKEKKRD | |
| EARKNWDVIENIKELKEGYMSQVIHIIAKMMVEEKAILIMEDLNIGFKRGRFKVEKQ | |
| VYQKFEKMLIDKLNYLVFKNKNPLEPGGSLNAYQLTSKFDSFKKLGKQSGFIFYVPS | |
| AYTSKIDPTTGFYNFIQVDVPNLEKGKEFFSKFEKIIYNTKEDYFEFHCKYGKFVSEPK | |
| NKDNDRKTKESLTYYNAIKDTVWVVCSTNHERYKIVRNKAGYYESHPVDVTKNLK | |
| DIFSQANINYNEGKDIKPIIIESNNAKLLKSIAEQLKLILAMRYNNGKHGDDEKDYILS | |
| PVKNKQGKFFCTLDGNQTLPINADANGAYNIALKGLLLIEKIKKQQGKIKDLYISNLE | |
| WFMFMMSR (SEQ ID NO: 43) | |
| MIKPTVSQFLTPGFKLIRNHSRTAGLKLKNEGEEACKKFVRENEIPKDECPNFQGGPA | TTTS, |
| IANIIAKSREFTEWEIYQSSLAIQEVIFTLPKDKLPEPILKEEWRAQWLSEHGLDTVPYK | NTTN |
| EAAGLNLIIKNAVNTYKGVQVKVDNKNKNNLAKINRKNEIAKLNGEQEISFEEIKAF | |
| DDKGYLLQKPSPNKSIYCYQSVSPKPFITSKYHNVNLPEEYIGYYRKSNEPIVSPYQFD | |
| RLRIPIGEPGYVPKWQYTFLSKKENKRRKLSKRIKNVSPILGIICIKKDWCVFDMRGLL | |
| RTNHWKKYHKPTDSINDLFDYFTGDPVIDTKANVVRFRYKMENGIVNYKPVREKKG | |
| KELLENICDQNGSCKLATVDVGQNNPVAIGLFELKKVNGELTKTLISRHPTPIDFCNKI | |
| TAYRERYDKLESSIKLDAIKQLTSEQKIEVDNYNNNFTPQNTKQIVCSKLNINPNDLP | |
| WDKMISGTHFISEKAQVSNKSEIYFTSTDKGKTKDVMKSDYKWFQDYKPKLSKEVR | |
| DALSDIEWRLRRESLEFNKLSKSREQDARQLANWISSMCDVIGIENLVKKNNFFGGS | |
| GKREPGWDNFYKPKKENRWWINAIHKALTELSQNKGKRVILLPAMRTSITCPKCKY | |
| CDSKNRNGEKFNCLKCGIELNADIDVATENLATVAITAQSMPKPTCERSGDAKKPVR | |
| ARKAKAPEFHDKLAPSYTVVLREAV (SEQ ID NO: 63) | |
| MEKEITELTKIRREFPNKKFSSTDMKKAGKLLKAEGPDAVRDFLNSCQEIIGDFKPPV | VTTK, |
| KTNIVSISRPFEEWPVSMVGRAIQEYYFSLTKEELESVHPGTSSEDHKSFFNITGLSNY | NTTN |
| NYTSVQGLNLIFKNAKAIYDGTLVKANNKNKKLEKKFNEINHKRSLEGLPIITPDFEE | |
| PFDENGHLNNPPGINRNIYGYQGCAAKVFVPSKHKMVSLPKEYEGYNRDPNLSLAGF | |
| RNRLEIPEGEPGHVPWFQRMDIPEGQIGHVNKIQRFNFVHGKNSGKVKFSDKTGRVK | |
| RYHHSKYKDATKPYKFLEESKKVSALDSILAIITIGDDWVVFDIRGLYRNVFYRELAQ | |
| KGLTAVQLLDLFTGDPVIDPKKGVVTFSYKEGVVPVFSQKIVPRFKSRDTLEKLTSQG | |
| PVALLSVDLGQNEPVAARVCSLKNINDKITLDNSCRISFLDDYKKQIKDYRDSLDELE | |
| IKIRLEAINSLETNQQVEIRDLDVFSADRAKANTVDMFDIDPNLISWDSMSDARVSTQI | |
| SDLYLKNGGDESRVYFEINNKRIKRSDYNISQLVRPKLSDSTRKNLNDSIWKLKRTSE | |
| EYLKLSKRKLELSRAVVNYTIRQSKLLSGINDIVIILEDLDVKKKFNGRGIRDIGWDNF | |
| FSSRKENRWFIPAFHKAFSELSSNRGLCVIEVNPAWTSATCPDCGFCSKENRDGINFT | |
| CRKCGVSYHADIDVATLNIARVAVLGKPMSGPADRERLGDTKKPRVARSRKTMKR | |
| KDISNSTVEAMVTA (SEQ ID NO: 62) | |
| VPDKKETPLVALCKKSFPGLRFKKHDSRQAGRILKSKGEGAAVAFLEGKGGTTQPNF | GTTB |
| KPPVKCNIVAMSRPLEEWPIYKASVVIQKYVYAQSYEEFKATDPGKSEAGLRAWLK | |
| ATRVDTDGYFNVQGLNLIFQNARATYEGVLKKVENRNSKKVAKIEQRNEHRAERGL | |
| PLLTLDEPETALDETGHLRHRPGINCSVFGYQHMKLKPYVPGSIPGVTGYSRDPSTPI | |
| AACGVDRLEIPEGQPGYVPPWDRENLSVKKHRRKRASWARSRGGAIDDNMLLAVV | |
| RVADDWALLDLRGLLRNTQYRKLLDRSVPVTIESLLNLVTNDPTLSVVKKPGKPVR | |
| YTATLIYKQGVVPVVKAKVVKGSYVSKMLDDTTETFSLVGVDLGVNNLIAANALRI | |
| RPGKCVERLQAFTLPEQTVEDFFRFRKAYDKHQENLRLAAVRSLTAEQQAEVLALD | |
| TFGPEQAKMQVCGHLGLSVDEVPWDKVNSRSSILSDLAKERGVDDTLYMFPFFKGK | |
| GKKRKTEIRKRWDVNWAQHFRPQLTSETRKALNEAKWEAERNSSKYHQLSIRKKEL | |
| SRHCVNYVIRTAEKRAQCGKVIVAVEDLHHSFRRGGKGSRKSGWGGFFAAKQEGR | |
| WLMDALFGAFCDLAVHRGYRVIKVDPYNTSRTCPECGHCDKANRDRVNREAFICVC | |
| CGYRGNADIDVAAYNIAMVAITGVSLRKAARASVASTPLESLAAE (SEQ ID NO: 65) | |
| MSKTKELNDYQEALARRLPGVRHQKSVRRAARLVYDRQGEDAMVAFLDGKEVDEP | RTTN |
| YTLQPPAKCHILAVSRPIEEWPIARVTMAVQEHVYALPVHEVEKSRPETTEGSRSAW | |
| FKNSGVSNHGVTHAQTLNAILKNAYNVYNGVIKKVENRNAKKRDSLAAKNKSRER | |
| KGLPHFKADPPELATDEQGYLLQPPSPNSSVYLVQQHLRTPQIDLPSGYTGPVVDPRS | |
| PIPSLIPIDRLAIPPGQPGYVPLHDREKLTSNKHRRMKLPKSLRAQGALPVCFRVFDD | |
| WAVVDGRGLLRHAQYRRLAPKNVSIAELLELYTGDPVIDIKRNLMTFRFAEAVVEV | |
| TARKIVEKYHNKYLLKLTEPKGKPVREIGLVSIDLNVQRLIALAIYRVHQTGESQLAL | |
| SPCLHREILPAKGLGDFDKYKSKFNQLTEEILTAAVQTLTSAQQEEYQRYVEESSHEA | |
| KADLCLKYSITPHELAWDKMTSSTQYISRWLRDHGWNASDFTQITKGRKKVERLWS | |
| DSRWAQELKPKLSNETRRKLEDAKHDLQRANPEWQRLAKRKQEYSRHLANTVLSM | |
| AREYTACETVVIAIENLPMKGGFVDGNGSRESGWDNFFTHKKENRWMIKDIHKALS | |
| DLAPNRGVHVLEVNPQYTSQTCPECGHRDKANRDPIQRERFCCTHCGAQRHADLEV | |
| ATHNIAMVATTGKSLTGKSLAPQRLQEAAE (SEQ ID NO: 68) | |
| VAFLDGKEVDEPYTLQPPAKCHILAVSRPIEEWPIARVTMAVQEHVYALPVHEVEKS | NTTB |
| RPETTEGSRSAWFKNSGVSNHGVTHAQTLNAILKNAYNVYNGVIKKVENRNAKKR | |
| DSLAAKNKSRERKGLPHFKADPPELATDEQGYLLQPPSPNSSVYLVQQHLRTPQIDLP | |
| SGYTGPVVDPRSPIPSLIPIDRLAIPPGQPGYVPLHDREKLTSNKHRRMKLPKSLRAQG | |
| ALPVCFRVFDDWAVVDGRGLLRHAQYRRLAPKNVSIAELLELYTGDPVIDIKRNLM | |
| TFRFAEAVVEVTARKIVEKYHNKYLLKLTEPKGKPVREIGLVSIDLNVQRLIALAIYR | |
| VHQTGESQLALSPCLHREILPAKGLGDFDKYKSKFNQLTEEILTAAVQTLTSAQQEEY | |
| QRYVEESSHEAKADLCLKYSITPHELAWDKMTSSTQYISRWLRDHGWNASDFTQIT | |
| KGRKKVERLWSDSRWAQELKPKLSNETRRKLEDAKHDLQRANPEWQRLAKRKQE | |
| YSRHLANTVLSMAREYTACETVVIAIENLPMKGGFVDGNGSRESGWDNFFTHKKEN | |
| RWMIKDIHKALSDLAPNRGVHVLEVNPQYTSQTCPECGHRDKANRDPIQRERFCCT | |
| HCGAQRHADLEVATHNIAMVATTGKSLTGKSLAPQRLQ (SEQ ID NO: 69) | |
| MPKPAVESEFSKVLKKHFPGERFRSSYMKRGGKILAAQGEEAVVAYLQGKSEEEPPN | GTTK, |
| FQPPAKCHVVTKSRDFAEWPIMKASEAIQRYIYALSTTERAACKPGKSSESHAAWFA | NTTN |
| ATGVSNHGYSHVQGLNLIFDHTLGRYDGVLKKVQLRNEKARARLESINASRADEGL | |
| PEIKAEEEEVATNETGHLLQPPGINPSFYVYQTISPQAYRPRDEIVLPPEYAGYVRDPN | |
| APIPLGVVRNRCDIQKGCPGYIPEWQREAGTAISPKTGKAVTVPGLSPKKNKRMRRY | |
| WRSEKEKAQDALLVTVRIGTDWVVIDVRGLLRNARWRTIAPKDISLNALLDLFTGDP | |
| VIDVRRNIVTFTYTLDACGTYARKWTLKGKQTKATLDKLTATQTVALVAIDLGQTN | |
| PISAGISRVTQENGALQCEPLDRFTLPDDLLKDISAYRIAWDRNEEELRARSVEALPEA | |
| QQAEVRALDGVSKETARTQLCADFGLDPKRLPWDKMSSNTTFISEALLSNSVSRDQV | |
| FFTPAPKKGAKKKAPVEVMRKDRTWARAYKPRLSVEAQKLKNEALWALKRTSPEY | |
| LKLSRRKEELCRRSINYVIEKTRRRTQCQIVIPVIEDLNVRFFHGSGKRLPGWDNFFTA | |
| KKENRWFIQGLHKAFSDLRTHRSFYVFEVRPERTSITCPKCGHCEVGNRDGEAFQCL | |
| SCGKTCNADLDVATHNLTQVALTGKTMPKREEPRDAQGTAPARKTKKASKSKAPP | |
| AEREDQTPAQEPSQTS (SEQ ID NO: 66) | |
| MSNKTTPPSPLSLLLRAHFPGLKFESQDYKIAGKKLRDGGPEAVISYLTGKGQAKLK | VTTS, |
| DVKPPAKAFVIAQSRPFIEWDLVRVSRQIQEKIFGIPATKGRPKQDGLSETAFNEAVAS | NTTN |
| LEVDGKSKLNEETRAAFYEVLGLDAPSLHAQAQNALIKSAISIREGVLKKVENRNEK | |
| NLSKTKRRKEAGEEATFVEEKAHDERGYLIHPPGVNQTIPGYQAVVIKSCPSDFIGLP | |
| SGCLAKESAEALTDYLPHDRMTIPKGQPGYVPEWQHPLLNRRKNRRRRDWYSASLN | |
| KPKATCSKRSGTPNRKNSRTDQIQSGRFKGAIPVLMRFQDEWVIIDIRGLLRNARYRK | |
| LLKEKSTIPDLLSLFTGDPSIDMRQGVCTFIYKAGQACSAKMVKTKNAPEILSELTKS | |
| GPVVLVSIDLGQTNPIAAKVSRVTQLSDGQLSHETLLRELLSNDSSDGKEIARYRVAS | |
| DRLRDKLANLAVERLSPEHKSEILRAKNDTPALCKARVCAALGLNPEMIAWDKMTP | |
| YTEFLATAYLEKGGDRKVATLKPKNRPEMLRRDIKFKGTEGVRIEVSPEAAEAYREA | |
| QWDLQRTSPEYLRLSTWKQELTKRILNQLRHKAAKSSQCEVVVMAFEDLNIKMMH | |
| GNGKWADGGWDAFFIKKRENRWFMQAFHKSLTELGAHKGVPTIEVTPHRTSITCTK | |
| CGHCDKANRDGERFACQKCGFVAHADLEIATDNIERVALTGKPMPKPESERSGDAK | |
| KSVGARKAAFKPEEDAEAAE (SEQ ID NO: 67) | |
| MRSIKYELKDSYGIAGLRNRIADATISDNKWLYGNINLNDYLEWRSSKTDKQIEDGD | TR |
| RESSLLGFWLEALRLGFVFSKQSHAPNDFNETALQDLFETLDDDLKHVLDRKKWCD | |
| FIKIGTPKTNDQGRLKKQIKNLLKGNKREEIEKTLNESDDELKEKINRIADVFAKNKS | |
| DKYTIFKLDKPNTEKYPRINDVQVAFFCHPDFEEITERDRTKTLDLIINRFNKRYEITE | |
| NKKDDKTSNRMALYSLNQGYIPRVLNDLFLFVKDNEDDFSQFLSDLENFFSFSNEQIK | |
| IIKERLKKLKKYAEPIPGKPQLADKWDDYASDFGGKLESWYSNRIEKLKKIPESVSDL | |
| RNNLEKIRNVLKKONNASKILELSQKIIEYIRDYGVSFEKPEIIKFSWINKTKDGQKKV | |
| FYVAKMADREFIEKLDLWMADLRSQLNEYNQDNKVSFKKKGKKIEELGVLDFALN | |
| KAKKNKSTKNENGWQQKLSESIQSAPLFFGEGNRVRNEEVYNLKDLLFSEIKNVENI | |
| LMSSEAEDLKNIKIEYKEDGAKKGNYVLNVLARFYARFNEDGYGGWNKVKTVLENI | |
| AREAGTDFSKYGNNNNRNAGRFYLNGRERQVFTLIKFEKSITVEKILELVKLPSLLDE | |
| AYRDLVNENKNHKLRDVIQLSKTIMALVLSHSDKEKQIGGNYIHSKLSGYNALISKR | |
| DFISRYSVQTTNGTQCKLAIGKGKSKKGNEIDRYFYAFQFFKNDDSKINLKVIKNNSH | |
| KNIDENDNENKINALQVYSSNYQIQFLDWFFEKHQGKKTSLEVGGSFTIAEKSLTIDW | |
| SGSNPRVGFKRSDTEEKRVFVSQPFTLIPDDEDKERRKERMIKTKNRFIGIDIGEYGLA | |
| WSLIEVDNGDKNNRGIRQLESGFITDNQQQVLKKNVKSWRQNQIRQTFTSPDTKIAR | |
| LRESLIGSYKNQLESLMVAKKANLSFEYEVSGFEVGGKRVAKIYDSIKRGSVRKKDN | |
| NSQNDQSWGKKGINEWSFETTAAGTSQFCTHCKRWSSLAIVDIEEYELKDYNDNLF | |
| KVKINDGEVRLLGKKGWRSGEKIKGKELFGPVKDAMRPNVDGLGMKIVKRKYLKL | |
| DLRDWVSRYGNMAIFICPYVDCHHISHADKQAAFNIAVRGYLKSVNPDRAIKHGDK | |
| GLSRDFLCQEEGKLNFEQIGLL (SEQ ID NO: 81) | |
| MKSIFDNFTGLYSLSKTLRFELRPVGQTLENIKNGHFLESDKKMADDYQDVKKIIDN | YTTN, |
| YHKFFIDDVLKGASFDWALLEKELTDFNKNKTDDSKVEAEQKKLREQIAKTLAGDK | TTYN |
| RFKSLTASTPNDLFNKDKDFIGWLEQSSVKEIRKDALDTFKKFSSYFKGFQENRKNV | |
| YSADDIPTAVPYRIVNDNFPKFLQNISIFKTIQEKCPQVIADVENELASYLGKEKLADIF | |
| TVQAFNKYLCQGGKENQRGIDFYNQVIGGIAEKEGGVNLRGINQFLNLYWQQHPDF | |
| AKENRRIKMVPLYKQILSDRSSLSFKIETIDTDEELKTAISEYADKLESKSNDEKKSVL | |
| DVCVELFDSIKEQNLQEIYVNRKDINNISRILTGDWSWLQSRMNLYADEVFTTKAEK | |
| TRWQKSVDGDEGENKSKGAYSLAELNRVLEYASENVAETDIRITDYFCHRNRFYYE | |
| KESGLFKQGEELVALSIKESCEDILSKRKAMNEAFANISESNSLRDNSEDIAKTKTYLD | |
| SVQDLLHRIKPLKVNGLGDPSFYAVFDSIYSALSEVISIYNKTRNYITRKAESPEKYKL | |
| NFDNPTLANGWDLNKEKDNTCVLLRKNGMYYLGIMNPKDKPKFAEKYDCGTESCY | |
| EKMIYKLLPGPNKMLPKVFFSTKGKKQYNPPENILHGYEQGKHKKGVAFDINFCHEL | |
| IDWFKSAINQHEDWKKFGFKFSDTKSYKDISDFYREVTEQGYKLTFINIPESEISKMVS | |
| EGKLYLFQIYNMDFAPGANGMPNMHTLYWKNLFSEENLKDVVLKLNGEAELFYRP | |
| AGIKEPVVHAKGSYLVNRITKDGEPIPEKIHDEIYRNANGNLNNLSKEASEYKESHKV | |
| VIKQASHEIIKDRHYTEPKFLFHIPLTINFKAPSMKPTVCTAIQNENVRRFLKNNPDVNI | |
| IGLDRGERNLIYLSLINQKGEIIKQFSFNDVEREQNGQTVKVNYHEKLDQREKERDAA | |
| RKSWQAIGKIAELKEGYLSAVIHQLTKLMVEYNAIVVMEDLNFGFKRGRFHVEKQV | |
| YQKFEHMLIDKLNYLVFKDRGLNEPGGALNGYQLAGQFESFQKLGKQSGMLFYVPA | |
| AYTSKIDPKTGFVSMMNFKDLTNVHKKRDFFSKFEDIHFDEATCSFVFTFDYKNING | |
| KAKEEMKQTKWAVYSREKRIVYFSKTKSSEDIMPTEKLRALFESDGIEYKSGNNIHD | |
| SVMAVGADLKEGAKPSKEIADFWDGLLYNFKLILQMRNSNAKTGEDYIISPVMASD | |
| GTFFDSRVEAKKGKDAKLPLDADANGAYHIALKGLSLINKINLAGEDELKKFDMKIS | |
| NEDWFNFAQEKKYAE (SEQ ID NO: 83) | |
| MTKQNKSVTQNTKRKNFGEFTNLYSLSKTLRFELKPVKATKTILEVERKDRENFKKD | TTTN |
| RKIAKNYQKLKGILNELHQEFIQDVMREFSFQEKEIKEFEERYLEALNFKEKDNYKKR | |
| TQLKNAYEKVAKKLAGKIATAFGKYNQEKYGVKFTKKNLTGENVFDILEGKYKGD | |
| KKILGIIHTFKFKPTKEEKKQGKEAVNFSTYLGGFNONRENFYKGEMKAGQFATRTIE | |
| NLIQFLKNKKLFIDKYKDNYQKIGFSQEQVEIFNLNYFNNLFLQEGLDVYNGILGAKK | |
| GEKNTENDGLNQKINLFKQKEKTRCKANGEKFNKSDYPIFKELYKQIGSIKKDNDVY | |
| VEIKSDEELVNVLQSLPEKTANTLREVQKFYENFFDKIFNDEFDLDKIYLPKSVGTHFS | |
| HLAFSDWSKLAFVFNKRWRNEKVKIKEGEDVNVQSRSLADIKKRMEEILEMDGGVS | |
| FGKTYCQKVGLEKEARTIEDVWSGFWKIIQYHINSQFIGGEKEVFDKEKKDDKTEKI | |
| QTIDDLQEEYLQATEMYRERMVESEEGLNDGEEKEIKTKLKNYLDRIKDIERIARYFD | |
| LRKHFDDIDEASKDGDFYFIYQELLQDISEAKINDHYNEIRNYLTKANVVDDKFKLNF | |
| NDGQTLSGWDLNKETEKFSLIFKRKVDGGVEYYLGIINKEKNKTIFDKKKHPEIFTEN | |
| SEFEKMEYKLFPSPSKMLPKIAFTKNKEGERIKPVFLDENAGKEIAQIKKEFALFQDA | |
| KKEDKNKWSDEFDRKKLNKLIDYYKLVLEKHPEKYMQTFNFVFKSSAKYKNLGEF | |
| NDDVARQNYVTKFVSVDKDYIDQKVESGELYLFKIHNKDWNLTKAGDTKKQSKKN | |
| LHTIYFEELFSEKNIAEPVFKLSGGAEVFFRDAIEKKKQKKKKDKKGKEILEKFRFTK | |
| NKILFHVPITINYGKPSINQGQFNQKINEFIADNSRSVNILGIDRGEKHLLYYSLVDNN | |
| GKIIKSGSLNEINGVDYHEKLDKAEKERQEARKSWQKINQIKNLKAGYISQVIKKIVD | |
| LAIENNAIIVLEDLNFGFKSFRQKIEKNVYQQFEKALIDKLGFVTDKEKLNHRQAPQL | |
| SAPFESFEKMGKQTGIVFYVLATNTSKVCPQCQWKKNIFPHYSTKKSIAENLQKQYK | |
| MKMYWRENENRFEFEYKGDGDKEFSSIFSNVDRVRYDKRANNNQGGYVIYQIDSTT | |
| KEKDGRNIKEKSITNLLKELLLEKFEIDNLEGELLVKLSEKSPDVSKETIKDFFGLLNSI | |
| LNIRNSMTDTEEDYIQCPACGFDTRKENKIGIKNGDDNGAYNIALRGRFLIERIKKAK | |
| KEDKKPNLTFSNNDYFQWVREFVK (SEQ ID NO: 84) | |
| MKKSIFDQFVNQYALSKTLRFELKPVGETGRMLEEAKVFAKDETIKKKYEATKPFFN | TTTN |
| KLHREFVEEALNEVELAGLPEYFEIFKYWKRYKKKFEKDLQKKEKELRKSVVGFFN | |
| AQAKEWAKKYETLGVKKKDVGLLFEENVFAILKERYGNEEGSQIVDESTGKDVSIFD | |
| SWKGFTGYFIKFQETRKNFYKDDGTATALATRIIDQNLKRFCDNLLIFESIRDKIDFSE | |
| VEQTMGNSIDKVFSVIFYSSCLLQEGIDFYNCVLGGETLPNGEKRQGINELINLYRQK | |
| TSEKVPFLKLLDKQILSEKEKFMDEIENDEALLDTLKIFRKSAEEKTTLLKNIFGDFVM | |
| NQGKYDLAQIYISRESLNTISRKWTSETDIFEDSLYEVLKKSKIVSASVKKKDGGYAF | |
| PEFIALIYVKSALEQIPTEKFWKERYYKNIGDVLNKGFLNGKEGVWLQFLLIFDFEFN | |
| SLFEREIIDENGDKKVAGYNLFAKGFDDLLNNFKYDQKAKVVIKDFADEVLHIYQM | |
| GKYFAIEKKRSWLADYDIDSFYTDPEKGYLKFYENAYEEIIQVYNKLRNYLTKKPYS | |
| EDKWKLNFENPTLADGWDKNKEADNSTVILKKDGRYYLGLMARGRNKLFDDRNLP | |
| KILEGVENGKYEKVVYKYFPDQAKMFPKVCFSTKGLEFFQPSEEVITIYKNSEFKKGY | |
| TFNVRSMQRLIDFYKDCLVRYEGWQCYDFRNLRKTEDYRKNIEEFFSDVAMDGYKI | |
| SFQDVSESYIKEKNQNGDLYLFEIKNKDWNEGANGKKNLHTIYFESLFSADNIAMNF | |
| PVKLNGQAEIFYRPRTEGLEKERIITKKGNVLEKGDKAFHKRRYTENKVFFHVPITLN | |
| RTKKNPFQFNAKINDFLAKNSDINVIGVDRGEKQLAYFSVISQRGKILDRGSLNVING | |
| VNYAEKLEEKARGREQARKDWQQIEGIKDLKKGYISQVVRKLADLAIQYNAIIVFED | |
| LNMRFKQIRGGIEKSVYQQLEKALIDKLTFLVEKEEKDVEKAGHLLKAYQLAAPFET | |
| FQKMGKQTGIVFYTQAAYTSRIDPVTGWRPHLYLKYSSAEKAKADLLKFKKIKFVD | |
| GRFEFTYDIKSFREQKEHPKATVWTVCSCVERFRWNRYLNSNKGGYDHYSDVTKFL | |
| VELFQEYGIDFERGDIVGQIEVLETKGNEKFFKNFVFFFNLICQIRNTNASELAKKDGK | |
| DDFILSPVEPFFDSRNSEKFGEDLPKNGDDNGAFNIARKGLVIMDKITKFADENGGCE | |
| KMKWGDLYVSNVEWDNFVANK (SEQ ID NO: 52) | |
| MIKSYDDFTKLYSLQKTLRFELKPIGKTLEHIKKSEIIESDETLEEQYAIVKNIIDRFHR | TTTN |
| KHIDEALSLVDFTKHLDTFKTIQELYLKRGKTDREKKELEELSADLRKLIVSYLKGNV | |
| KOKTQHNLNPIKERFEILFGKELFTNEEFFTLAENKEEKKAIQAFKGFTTYFKGFQEN | |
| RKNMYSEEDKSTAIAYRIINENMPLFIENIARFQKVLDVIEKTKLTELKQNLKTELKG | |
| HSVSDIFRIEYFNNVLTQEGISRYNTIIGGKTTTEGVKIQGLNEIINLHNQQSKDVKLPI | |
| LKPLHKQILSEEYSTSFKIKAFENDNEVLKAIDTFWNEHIEKSIHPVTGKRFNILLKIEN | |
| LCKKLEKYKDKEIEKLFIERKNLSTVSHQVYGQWNIIRDALRMHLEMNNKNIKEKDI | |
| DKYLDNDAFAWKEIKDSIKIYKEHVEDAKELDENGIVKYFSSMSINEEDDEKEYSISLI | |
| KNINEKYNNVKSILEEDRTGKSDLHQDKEKVAIIKEFLDSLKQLQWFLKLLYVTVPL | |
| DEKDYEFYNELEVYYEALLPLNSLYNKVRNYMTRKPYSVEKFKLNFYSPTLLDGWD | |
| KNKETANLSIILKKNGKYYLGIMNKENNTIFENFPKSKSNDYYEKMIYKLLPGPNKM | |
| LPKVFFSKKGLEYYKPSKEILRIYEKGEFKKDKSGNFKKESLHTLIDFYKEAIAKNED | |
| WKIFKFKFKNTREYEDISQFYRDVEEQGYLIIFEKVDANYVDKLVEEGELYLFQIYNK | |
| DFSENKKSKGNPNLHTIYWESLFDNQNLKDVVYKLNGEAEVFYRKKSIDYPEEIYNN | |
| GHHKEELNGKFNYPIIKDRRYTQDKFLFHVPITMNFISKEEKRVNQLACEYLSTTKED | |
| VHIIGIDRGERHLLYLSLIDKEGNIKKQLSLNTIKNENYDKEIDYRVKLDEKEKKRDE | |
| ARKNWDVIENIKELKEGYMSQVIHIIAKMMAEEKAILIMEDLNIGFKRGRFKVEKQV | |
| YQKFEKMMIDKLNYLVFKNKEPLEPGGSLNAYQLTSKFDSFKKLGKQSGFIFYVPSA | |
| YTSKIDPTTGFYNFIRVDVPNLEKGKEFFSKFEKIIYNTKEDYFEFHCKYGKFVPEPKN | |
| KDNDRKTKESLTYYNAIKDTVWVVCSTHHERYKIVRNKAGYYESQPVDVTKNLRDI | |
| FSEANINYSDGKDIKPIIIESNNAKLLKSIAEQLKLILAMRYNNGKHDDDEKDYILSPV | |
| KNKQGKFFCSLDGDQSLPINADANGAYNIALKGLLLIEKIKKQQGKAKDLYISNLEW | |
| FMFMMSR (SEQ ID NO: 85) | |
| MRTTTSLDAFTNRYALSKTLRFELKPIGNTQMMLEQNNVFAKDRAIREKYEKTKPWI | TTTN |
| DLLHREFVAESLQNAQLGNLDDYYAALQNVQKITKDTNAEDKKRWKKGFEKQEKR | |
| LRKEVVALFDKAAHIWATQRYPQLKKKTKDFLFEEGVFEHVLFARYGSAPDTTVKI | |
| VTSNPETGEVIDEREESIFKGWKGFTGYFDKFFETRKNFYKDNGTATAIATRAINQNL | |
| RRFAENMQKLTDIKNNYPELLAHTDFGDFDIAHAQSLDFYARTCLLQEGIDAYNKKF | |
| VGVLKSAINEYQQKNKGVRISYPKTLDNQILGERERRLFDVIEDDRELHDVFRAFVD | |
| DGTVFAAEMRQLAQAFSAQNGTYDYTQIYISKKGFETISRKYTHDTRAWHDALADV | |
| FKAKAKKRIATTASGEKKFPAYIPVAYITQALTLVQESEDTECTWKERYASITENKTL | |
| EEGFFAIFADEFERLFVHMEATVQDTDYVVAEDKAKKLLSDGQITKNEQTTQIIKEY | |
| ADALLRIYQMAKYFAVEKKSMWDDAVAIDDTFYETFKEIYGNTHSTIVASYNLLRN | |
| YLTKKPWEDVQKWKLNFENPTLLDGWDKNKEAANFGVILRDGDKFYLGIMRKGHN | |
| NIFANQHHSNFEGQGLQKMVYKFFPDPKKMFPKVCFSAKGMEFFAPSEEIVRIYKNA | |
| EFKSGDTFNVESMQKLIDFYKNALQKYDGWKIYDFKHLKDTAQYTSNIGEFYDDVA | |
| KGGYQLGWQNISKEYVEEKNANGELYLFQIKNKDWNDGATGRKNLHTLYFEYLFS | |
| EKNAAADFVFRLNGGAEVFYRPAAIESKTERRGNREVAAKKRYTQDKVFLHVPITL | |
| NRTAGDVKTSAFNDAVNRFLAGNPDINIMGIDRGEKHLAYYSIIDQNGNRIVSGSFNT | |
| IGSKDYHALLTERQGAREEARKNWQRVEQIKDLKKGYISLVVREIADLAIKHNAIIVL | |
| ENLNMRFKQIRGGIEKSVYQQLEKALIEKLNFLVNKGEVDATKAGHLLRAYQLAAPF | |
| ETFEKMGNQTGIIFYTTASYTSQVDPVTGWRPHVYLKYRNAQKTKEDILRIFDDIVEN | |
| DEKQRFEFAYRHNGVSWTVCSSVERHRWNRSNNAGKGGYDVFPVEGEGSITQRLQE | |
| ACAQRGIDTTRNILAQIDELDESASATVSFLRDLCFYFRLICQIRNTDDGADDINAQDF | |
| LMSPVEPFFDTRNAQEQYPQNGDENGAYNIARKGIIILQKITAWGRSQDTQRRYPDTF | |
| VSQDEWDTFLTQHTT (SEQ ID NO: 86) | |
| MENNFIKKYSLQKTLRFELKPVGETADYIEDFKSEYLKDTVLKDEQRAKDYQEIKTLI | TTTN |
| DDYHREYIEECLREPVDKKTGEILDFTQDLEDAFSYYQKLKENPTENRVGWEKEQES | |
| LRKKLVTSFVGNDGLFKKEFITRDLPEWLQKKGLWGEYKDTVENFKKFTTYFSGFH | |
| ENRKNMYTAEAQSTAIANRLMNDNLPKFFNNYLAYQTIKEKHPDLVFRLDDALLQA | |
| AGVEHLDEAFQPRYFSRLFAQSGITAFNELIGGRTTENGEKIQGLNEQINLYRQQNPE | |
| KAKGFPRFMPLFKQILSDRETHSFLPDAFENDKELLQALRDYVDAATSEEGMISQLN | |
| KAMNQFVTADLKRVYIKSAALTSLSQELFHFFGVISDAIAWYAEKRLSPKKAQESFL | |
| KQEVYAIEELNQAVVGYIDQLEDQSELQQLLVDLPDPQKPVSSFILTHWQKSQEPLQ | |
| AVIAKVEPLFELEELSKNKRAPKHDKDQGGEGFQQVDAIKNMLDAFMEVSHAIKPL | |
| YLVKGRKAIDMPDVDTGFYADFAEAYSAYEQVTVSLYNKTRNHLSKKPFSKDKIKI | |
| NFDAPTLLNGWDLNKESDNKSIILRKDGNFYLAIMHPKHTKVFDCYSASEAAGKCYE | |
| KMNYKLLSGANKMLPKVFFSKKGIETFSPPQEILDLYKNNEHKKGATFKLESCHKLI | |
| DFFKRNIPKYKVHPTDNFGWDVFGFHFSPTSSYGDLSGFYREVEAQGYKLWFSDVSE | |
| AYINKCVEEGKLFLFQIYNKDFSPNSTGKPNLHTLYWKGLFEPENLKDVVLKLNGEA | |
| EIFYRKHSIKHEDKTIHRAKDPIANKNADNPKKQSVFDYDIIKDKRYTQDKFFFHVPIS | |
| LNFKSQGVVRFNDKINGLLAAQDDVHVIGIDRGERHLLYYTVVNGKGEVVEQGSLN | |
| QVATDQGYVVDYQQKLHAKEKERDQARKNWSTIENIKELKAGYLSQVVHKLAQLI | |
| VKHNAIVCLEDLNFGFKRGRFKVEKQVYQKFEKALIDKLNYLVFKERGATQAGGYL | |
| NAYQLAAPFESFEKLGKQTGILYYVRSDYTSKIDPATGFVDFLKPKYESMAKSKVFF | |
| ESFERIQWNQAKGYFEFEFDYKKMCPSRKFGDYRTRWVVCTFGDTRYQNRRNKSSG | |
| QWETETIDVTAQLKALFAAYGITYNQEDNIKDAIAAVKYTKFYKQLYWLLRLTLSLR | |
| HSVTGTDEDFILSPVADENGVFFDSRKATDKQPKDADANGAYHIALKGLWNLQQIR | |
| QHDWNVEKPKKLNLAMKNEEWFGFAQKKKFRA (SEQ ID NO: 53) | |
| MLKEKQFKTFGDFTNLYELSKTLRFELRPTPETKDLLDKNKIIQTDKKIAENYQEIKK | TTYN |
| YFDKLHKKFIKEALSNTQIDFSDFCKLWEQNSKDSGKIKDLSRKLRKSIKQAFDKKG | |
| ADWHKRYLEKGIKLKKKNLDILFEERVLDILKEEFKDDVDVKLFESFKGFSTYFTNF | |
| HESRKNFYKDDGTASAIATRIIDENLKRFCDNIKVKKHSKKLISELNEREAKIFEADFY | |
| NRCLLQQGIDDYNQVIGDINKKINNLRQNKIENTPTLKILYKQILGDVRRQETEQDAFI | |
| EIKNNEEVFDFLQDFIKHSDENNKYFKNLFYKFIEGKHSLDKIFLAKRFVNTISGKWF | |
| ASWEVFGAELIKKFGNKKDLPDFIPFAAVKDVLQNCNIPANELFKEKIKNDEDKNIYD | |
| IFINLWKEEFDSNLKKVEESKKEVENMIAEDKVYSNKKEKRKNDNGEEIEIEIQKEKI | |
| KNYADAAMNIFRMMKYFLLEKNGKTVEGMGEDNNFYNELNIVFKGGEIDGKVYEG | |
| VKTYLYYNEFRNYLTKKPFNEEKTKLNFDCGQILSGWDKNKESEKLGVILRKDNKY | |
| YLAIINKKHNKIFDVKKNSYAYIVGDNFYEKMEYKLFPDAKRMIPKIAFAKNNKEKF | |
| GWTDEIQKIKNEYAEFQEGKKNDKNLWKDKFNKNKMEKLITYYQNCLEKGGYKDI | |
| YNFRWKSPDKYSGIGEFNDEIDRQSYCLKFVKVDFNYVFEKVKSGELYLFQIYNKDF | |
| SDKADRAQKENIHTEYFKLLFDQRNLDNVVLKLSGGAEIFYRPKTEGLPKKKDNKG | |
| NEVVRHRRYADDKYFLHLPIQLNFGRGNLSGGEFNSKINQYLSEQREIKIIGIDRGEK | |
| HLAYYSVINQDGKIEEIESLNTVNGIDYRKKLDELEKKREQERKSWQSISKIKDLKKG | |
| YISHVIKKICDLAIEHNAIIVFEDLSGGFKNSRKKIEKQIYQNLELALATKLNYLTFKD | |
| KNFGESGHYLNAYQLAPKIDNYQDIKMQTGIVFYTPAGYTSSTCPQCGFRKTLKFDY | |
| TATISKAEDLIRGSKLNIVFEKEKNRFKINYLFNPIEKKKKKIKENELFADAGAKNEFTI | |
| YSDVKRIKWHNTGTKRLEEAEGERLLENKNSRGRDKEYDINKCLTRLFRENKIDVNG | |
| DIIGQITKIKSLKLYQDLFYYLFLATLIRNNVSGSDIDYIQCPSCHFHSDGGFQKQKFN | |
| GDANGAYNIARKGILILKKIKQFAAQDKDMKNFGWKHLTVDINEWDKFTQK (SEQ | |
| ID NO: 87) | |
| MNKNFSNFTELYTLSKTLRFELKPVAQTKENIKKGKFLESDKKKADDYKDVKKIIDN | TTTN |
| YHKFFIDDVLKNASFDWTVLEKEMSDENKSKADDSKVEAEQKKLRDQIAKKLTSDK | |
| RFKALTASTPSDLFNKDKDFIDWFTQNSTKDINKEALETFKRFSSYFKGFQENRKNVY | |
| SAEPIPTAVPYRLVNDNFPKFLQNIALFKIIQEKCPQVISDVEKELASYLGKEKLADIFT | |
| VQAFNKYLCQGGKENQRGIDFYNNILGGIAEKEGGINLRGINQFLNLYWQQHQDFA | |
| KONKRIKMIPLFKQILSDRSSLSFKIESINTDQELLTSITEYADKLETKSNDEKKSVLDI | |
| CSDLFASIKAQNLQEIYVNRKDINSISRILTGDWSWLQSRMNVYADEVFTTKAEKTR | |
| WQKSIDGDEGENKSKGVFSLAELNSVLEYSSENVSETDVRITDFFDHRNRFYYEKES | |
| GLFKQGDELVALSIRESCEDILAKRKAMDEAFANVSENNSLRDNSEDVAKIKIYLDC | |
| VQELLHRIKPLKVNGLGDPAFYAVFDTVYNSLSEVISLYNKTRNYITKKAANPEKYK | |
| LNFDNPTLADGWDLNKEQANTSVLMRKDGMYYLGIMNPKDKPKFAEKYECGNEA | |
| CYEKMIYKQFDATKQIPKCSTQVKEVKKHFQSGATDSIILNDKSKFKLDLVITKEIWF | |
| LNNHVWNGEKFVPKRESNETRPKKFQIGYYKQTGDLGGYKEALNIWISFCKTFLQSY | |
| ISSSIYDYDFKESSNYDSLDEFYNYLNATCYKLSFINIPEATISQMVSEGKLYLFQIYNK | |
| DFAPGASGMPNMHTLYWKNLFSEENLKNVVLKLNGEAELFYRPAGIKEPVIHAKGS | |
| YLVNRITKDGEPIPEKIHDEIYRNANGKLESLSKEATEYKASHKVVIKEAKHDIIKDRH | |
| YTEPKFLFHVPLTINFKASGNSYINENVRRFLKNNPDVNVIGLDRGERHLIYLSLINQK | |
| GEIIKQFTFNEVERNKNGQVIKVNYHEKLDQREKVRGAARKSWQAIGKIAELKEGYL | |
| SAVIHOLTKLMVEYNAIVVMEDLNFGFKRGRFHVEKQVYQKFEHMLIDKLNYLVFK | |
| DRGLTEAGGVLNGYQLAGQFESFQKLGKQSGMLFYVPAGYTSKIDPKTGFASMSNF | |
| KDLTNVHKKRAFFSKFDDIHFDDATGSFVFTFDYKNFDGKAKEEMKRTKWSVYSKD | |
| KRIVYLSKTKSYEDVQPTEKIKASLESVGIEYMSGNNLIDSIMVIGAELKDGAKPSKEI | |
| ADFWDRLLYNFKLILQMRNSNAKTGEDYIISPVMADDGTFFDSREEFKKGENAKMP | |
| VDADANGAYHIALKGLSLLKRFDAASENELKKFDMKISNVDWFKFAQEKSYAE | |
| (SEQ ID NO: 88) | |

In some embodiments, the system comprises a programmable nuclease, such as a programmable nuclease according to any of the various aspects or embodiments disclosed herein. In some embodiments, the programmable nuclease comprises a Type V Cas nuclease. In some embodiments, the programmable nuclease is a Cas12 protein, a Cas14 protein, or a CasΦ protein. In some embodiments, the programmable nuclease is a Cas 12 protein comprising a Cas12a, a Cas12b, a Cas12c, a Cas12d, a Cas12e, a Cas12f, a Cas12g, a Cas12h, a Cas12i, a Cas12j, or a Cas12k. In some embodiments, the programmable nuclease is a Cas12a protein. In some embodiments, the programmable nuclease comprises an amino acid sequence that is at least 80%, 90%, 95%, or 100% identical to SEQ ID NO: 43. In some embodiments, the programmable nuclease comprises SEQ ID NO: 43.

In some embodiments, the system comprises a reporter, such as a reporter according to any of the various aspects or embodiments disclosed herein. In some embodiments, each of the reporters comprises a fluorophore and a quencher, and wherein cleavage of the reporters is effective to produce a detectable loss of the quencher. In some embodiments, each of the reporters comprises a detection moiety, and cleavage of the reporters is effective to produce a detectable loss of the detection moiety. Optionally, the detection moiety comprises a fluorophore.

Also provided herein are methods of assaying for one or more target nucleic acids in a sample. In some cases, a method comprises: contacting a system described herein with the sample; cleaving the reporters in response to presence of the target nucleic acid or amplicon thereof; and detecting a change in signal resulting from cleavage of the reporters. In some cases, the detection identifies the target nucleic acid in the sample. In some cases, the method further comprises amplifying one or more of the target nucleic acids before or during said contacting

### III. Additional System Components

In some instances, additional system components include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

Additional system components may include labels listing contents and/or instructions for use, or package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Additional system components may also include the instruments described herein, such as the cartridges described herein.

### IV. Methods of Nucleic Acid Detection

Provided herein are methods of detecting target nucleic acids. Methods may comprise detecting target nucleic acids with compositions or systems described herein. Methods may also comprise amplifying target nucleic acids with compositions or systems described herein.

In some embodiments, methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some instances, methods of detecting a target nucleic acid in a sample or cell comprises contacting the sample or cell with an effector protein, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some instances, methods result in transcollateral cleavage of the reporter nucleic acid. In some instances, methods result in cis cleavage of the reporter nucleic acid. In some instances, the effector protein comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID Nos: 9-69 or 81-88.

Methods may comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample or cell with an effector protein and a guide nucleic acid at a temperature of at least about 25° C., at least about 30° C., at least about 35° C., at least about 40° C., at least about 50° C., at least about 55° C., at least about 60° C., at least about 65° C. at least about 70° C., at least about 75° C., or at least about 80° C. In some instances, the temperature is not greater than 80° C.

### Amplification of a Target Nucleic Acid

Methods may comprise amplifying a target nucleic acid for detection using any of the compositions or systems described herein.

Methods may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid. Amplifying may also comprise an amplification reagent. An amplification reagent, in some instances, comprises a primer, an activator, a deoxynucleoside triphosphate (dNTP), a ribonucleoside triphosphate (rNTP), or combinations thereof. An amplification reagent may comprise a primer. An amplification reagent may comprise an activator. An amplification reagent may comprise a dNTP. An amplification reagent may comprise an rNTP.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA). An amplification may also comprise isothermal amplification.

In some embodiments, amplification of the target nucleic acid comprises modifying the sequence of the target nucleic acid. For example, amplification may be used to insert a PAM sequence into a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid sequence.

Amplifying may take about 1, 2, 3, 4, 5, 6, 7, 8, 9, 10, 11, 12, 13, 14, 15, 16, 17, 18, 19, 20, 25, 30, 40, 50, or 60 minutes.

In some instances, methods of detecting comprise amplifying a target nucleic acid. In some instances, the all steps of the method are performed at the same temperature that amplification occurs. Amplifying may be performed at a temperature of about 20° C. to about 45° C. Amplifying may be performed at a temperature of less than about 20° C., less than about 25° C., less than about 30° C., 35° C., less than about 37° C., less than about 40° C., or less than about 45° C. Amplifying may be performed at a temperature of at least about 20° C., at least about 25° C., at least about 30° C., at least about 35° C., at least about 37° C., at least about 40° C., at least about 45° C., at least about 50° C., at least about 55° C., at least about 60° C., at least about 65° C. Amplifying may be performed at a temperature of about 20° C., about 25° C., about 30° C., about 35° C., about 37° C., about 40° C., about 45° C., about 50° C., about 55° C., about 60° C., or about 65° C.

In some cases, the method may also comprise reverse transcribing the target nucleic acid and/or amplifying the target nucleic acid before contacting the sample with the effector protein, the guide nucleic acid, and the detection reagent. In some cases, the method may also comprise reverse transcribing the target nucleic acid and/or amplifying the target nucleic acid after contacting the sample with the effector protein, the guide nucleic acid, and the detection reagent. In some cases, the contacting and the reverse transcribing are carried out at the same temperature. In some cases, the detecting and the reverse transcribing are carried out at the same temperature. In some cases, the contacting, the detecting, and the reverse transcribing are carried out at the same temperature. In some cases, the contacting and the amplifying are carried out at the same temperature. In some cases, the detecting and the amplifying are carried out at the same temperature. In some cases, the contacting, the detecting, and the amplifying are carried out at the same temperature. In some cases, the contacting, the detecting, the reverse transcribing, and the amplifying are carried out at the same temperature. The temperature may be about 25° C., about 30° C., about 35° C., about 37° C., about 40° C., about 45° C., about 50° C., about 55° C., about 60° C., about 65° C., 70° C., 75° C., or about 80° C. In some instances, the temperature may be from about 25° C. to about 80° C., from 30° C.-80° C., from 35° C.-80° C., from 37° C.-80° C., from 40° C.-80° C., from 45° C.-80° C., from 50° C.-80° C., from 55° C.-80° C., from 60° C.-80° C., from 65° C.-80° C., from 70° C.-80° C., from 75° C.-80° C., from 25° C.-30° C., from 25° C.-35° C., from 25° C.-37° C., from 25° C.-40° C., from 25° C.-45° C., from 25° C.-50° C., from 25° C.-55° C., from 25° C.-60° C., from 25° C.-65° C., from 25° C.-70° C., or from 25° C.-75° C.

In some cases, the contacting and the reverse transcribing are carried out in a single reaction chamber. In some cases, the detecting and the reverse transcribing are carried out in a single reaction chamber. In some cases, the contacting, the detecting, and the reverse transcribing are carried out in a single reaction chamber. In some cases, the contacting and the amplifying are carried out in a single reaction chamber. In some cases, the detecting and the amplifying are carried out in a single reaction chamber. In some cases, the contacting, the detecting, and the amplifying are carried out in a single reaction chamber. In some cases, the contacting, the detecting, the reverse transcribing, and the amplifying are carried out in a single reaction chamber.

A variety of suitable reverse transcriptases are available. Non-limiting examples of reverse transcriptases include avian myeloblastosis virus (AMV) reverse transcriptase, moloney murine leukemia virus (MMLV) reverse transcriptase, human immunovirus (HIV) reverse transcriptase, equine infectious anemia virus (EIAV) reverse transcriptase, Rous-associated virus-2 (RAV2) reverse transcriptase, WARMSTART reverse transcriptase (New England BioLabs, Inc. (NEB)), RAPIDXFIRE reverse transcriptase (LGC Limited, UK), ACAT138 reverse transcriptase (Watchmaker Genomics, Inc.), and ACAT141 reverse transcriptase (Watchmaker Genomics, Inc.). In some embodiments, the reverse transcriptase is selected from WARMSTART reverse transcriptase, RAPIDXFIRE reverse transcriptase, ACAT138 reverse transcriptase, and ACAT141.

In some embodiments, amplification reagents include a polymerase (e.g., a DNA-directed DNA polyemerase). A variety of suitable polymerases are available, non-limiting examples of which include Phi29 DNA polymerase, Bsm, Bst, T4, T7, DNA Pol I, Klenow fragment, and mutants, variants and derivatives thereof. Additional non-limiting examples of DNA polymerases include Taq, Tbr, Tfl, Tth, Tli, Tfi, Tne, Tma, Pfu, Pwo, and Kod DNA polymerase; VENT DNA polymerase (New England Biolabs), DEEP VENT DNA polymerase (New England Biolabs); PHUSION DNA polymerase; GOTAQ G2 Hot Start Polymerase (Promega), ONETAQ Hot Start DNA Polymerase (New England Biolabs), TAKARA TAQ DNA Polymerase Hot Start (Takara), KAPA2G Robust HotStart DNA Polymerase (KAPA), KAPA3G DNA polymerase (KAPA), FASTSTART Taq DNA Polymerase (Sigma-Aldrich), HOTSTART Taq DNA Polymerase (New England Biolabs), Q5 DNA Polymerase (New England Biolabs), KAPA HIFI DNA Polymerase (Roche), PRIMESTAR Max DNA Polymerase (Takara), PRIMESTAR GXL DNA Polymerase (Takara), RAPIDXFIRE DNA polymerase (LGC Limited, UK), and ACAT77 DNA polymerase (Watchmaker Genomics, Inc.). In some embodiments, the polymerase is selected from KAPA3G DNA polymerase, RAPIDXFIRE DNA polymerase, and ACAT77 DNA polymerase.

In some embodiments, reagents for a reverse transcription reaction (including a reverse transcriptase) are combined with reagents for nucleic acid amplification (including a polymerase). In some embodiments, the reverse transcriptase is selected from WARMSTART reverse transcriptase, RAPIDXFIRE reverse transcriptase, ACAT138 reverse transcriptase, and ACAT141, and the polymerase is selected from KAPA3G DNA polymerase, RAPIDXFIRE DNA polymerase, and ACAT77 DNA polymerase. In some embodiments a combination of reverse transcriptase and polymerase comprises WARMSTART reverse transcriptase and KAPA3G DNA polymerase. In some embodiments a combination of reverse transcriptase and polymerase comprises WARMSTART reverse transcriptase and ACAT77 DNA polymerase. In some embodiments a combination of reverse transcriptase and polymerase comprises ACAT138 reverse transcriptase and KAPA3G DNA polymerase. In some embodiments a combination of reverse transcriptase and polymerase comprises ACAT138 reverse transcriptase and ACAT77 DNA polymerase. In some embodiments a combination of reverse transcriptase and polymerase comprises ACAT141 reverse transcriptase and KAPA3G DNA polymerase. In some embodiments a combination of reverse transcriptase and polymerase comprises ACAT141 reverse transcriptase and ACAT77 DNA polymerase. In some embodiments a combination of reverse transcriptase and polymerase comprises RAPIDXFIRE reverse transcriptase and RAPIDXFIRE DNA polymerase.

In some cases, the sample may be separated into a plurality of droplets, aliquots, volumes, or subsamples. One or more targets (e.g., nucleic acids, biomolecules, etc.) may be contained within the plurality of droplets, aliquots, volumes, or subsamples. In some cases, the targets may be amplified before detection occurs. In some cases, the detection devices of the present disclosure may comprise a chamber or subsystem for amplifying the targets. In some cases, the detection devices can be configured to amplify the target sequences or target nucleic acids contained within the plurality of droplets or volumes by individually processing each (e.g., by using a thermocycling process). In some cases, the plurality of droplets or volumes can undergo separate thermocycling processes. In some cases, the thermocycling processes can occur simultaneously. In other cases, the thermocycling processes can occur at different times for each droplet or volume.

In any of the embodiments described herein, the detection device can comprise a single integrated system that is configured to perform sample collection, sample processing, droplet generation, droplet processing (e.g., amplification of target nucleic acids in droplets), droplet remixing, and/or circulation of the remixed droplets within a detection chamber so that at least a portion of the remixed droplets is placed in contact with one or more programmable nuclease probes coupled to the detection chamber. The detection devices of the present disclosure can be disposable devices configured to perform one or more rapid single reaction or multi-reaction tests to detect a presence and/or an absence of one or more target sequences or target nucleic acids.

In some embodiments, the present disclosure provides exemplary methods for programmable nuclease-based detection. The method can comprise collecting a sample. The sample can comprise any type of sample as described herein. The method can comprise preparing the sample. Sample preparation can comprise one or more sample preparation steps. The one or more sample preparation steps can be performed in any suitable order. In some cases, the one or more sample preparation steps can comprise physical filtration of non-target materials using a macro filter, nucleic acid purification, lysis, heat inactivation, or adding one or more enzymes or reagents to prepare the sample for target detection.

In some cases, the method can comprise generating one or more droplets, aliquots, or subsamples from the sample. The one or more droplets, aliquots, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, or subsamples. In some embodiments, the sample is not divided into subsamples.

In some cases, the method can comprise amplifying one or more targets within each droplet, aliquot, volume, or subsample. Amplification of the one or more targets within each droplet or volume can be performed in parallel and/or simultaneously for each droplet or volume. Dividing the sample into a plurality of droplets or volumes can enhance a speed and/or an efficiency of the amplification process (e.g., a thermocycling process) since the droplets/volumes comprise a smaller volume of material than the bulk sample introduced. Amplifying the one or more targets within each individual droplet can also permit effective amplification of various target nucleic acids that cannot be amplified as efficiently in a bulk sample containing the various target nucleic acids if the bulk sample were to undergo a singular amplification process. In some embodiments, amplification is performed on the bulk sample without first dividing the sample into subsamples. In some embodiments, amplification is performed on a crude sample (e.g., a sample that has optionally been lysed but not subjected to nucleic acid purification). In some embodiments, amplification is performed on a partially purified nucleic acid sample, in which a buffer used in a purification procedure (e.g., bead-based nucleic acid purification) is not exchanged prior to amplification.

In some embodiments, the sample can be provided manually to a detection device. For example, a swab sample can be dipped into a solution and the sample/solution can be pipetted into the device. In other embodiments, the sample can be provided via an automated syringe. The automated syringe can be configured to control a flow rate at which the sample is provided to the detection device. The automated syringe can be configured to control a volume of the sample that is provided to the detection device over a predetermined period. In some embodiments, the sample can be provided directly to the detection device. For example, a swab sample can be inserted into a sample chamber on the detection device.

The sample can be prepared before one or more targets are detected within the sample. The sample preparation steps described herein can process a crude sample to generate a pure or purer sample. Sample preparation can one or more physical or chemical processes, including, for example, nucleic acid purification, lysis, binding, washing, and/or eluting.

In some instances, a sample can be a crude sample, for example a sample that has not been purified. The methods and systems herein, in some embodiments, can be used with a crude sample. In some cases, a crude sample can be a crude lysate, such as a mechanical or chemical lysate of a biological sample. In some instances, a sample can be a bead purified nucleic acid sample. In some cases, a bead purified nucleic acid sample can be used without a buffer exchange. For example, a step for removing inhibitors in a bead purification, such as isopropanol and ethanol, may be omitted in some embodiments. In some cases, a crude sample can comprise a PCR inhibitor such as a protein, a fat, a humic acid, a phytic acid, an immunoglobulin G, a bile, a calcium chloride, an EDTA, a heparin, a ferric chloride, or any combination thereof.

In some cases, a sample can be multiplexed. For example, 2, 3, 4, 5, 6, 7, 8, 9, 10, 20 or more targets can be multiplexed on a device herein. In some cases, about: 2 targets to 20 targets can be multiplexed, 5 targets to 15 targets can be multiplexed, 4 targets to 12 targets can be multiplexed or 10 targets to 18 targets can be multiplexed. Multiplexing may facilitate detection of multiple target nucleic acids simultaneously. In some cases, the guide nucleic acids and reporter may be localized to distinct regions of a device herein to facilitate multiplexing.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). In some cases, a solution of reagents can be used to lyse the cells in the sample and release the nucleic acids so that they are accessible to the programmable nuclease (and optionally amplification reagents). Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate.2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned above. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g., a PK enzyme or a lysing reagent). In some cases, a heating element integrated into the detection device can be used for heat-inactivation. The heating element can be powered by a battery or another source of thermal or electric energy that is integrated with the detection device.

In some cases, a target nucleic acid within the sample can undergo amplification before binding to a guide nucleic acid, for example a crRNA of a CRISPR enzyme. The target nucleic acid within a purified sample can be amplified. In some instances, amplification can be accomplished using loop mediated amplification (LAMP), isothermal recombinase polymerase amplification (RPA), or polymerase chain reaction (PCR), or other amplification process described herein.

In some instances, a sample is split into several droplets, aliquots, or subsamples for performing multiple amplification reactions in parallel on the device. The sample can have a volume that ranges from about 10 μL to about 500 μL. The plurality of droplets, aliquots, or subsamples can have a volume that ranges from about 0.01 μL to about 100 μL. In some embodiments, the volume of an amplification reaction is about 5 μL to about 50 μL, about 10 μL to about 30 μL, or about 15 μL to about 25 μL. In some embodiments, the volume of an amplification reaction is about 15 μL to about 25 μL. The plurality of droplets, aliquots, or subsamples can have a same or substantially similar volume. In some cases, the plurality of droplets, aliquots, or subsamples can have different volumes. In some cases, each droplet or subsample of the sample can undergo one or more sample preparation steps (e.g., nucleic acid purification, lysis, heat inactivation, amplification, etc.) independently and/or in parallel while the droplets/subsamples are physically constrained or thermally isolated. Such nucleic acid amplification of the sample may improve at least one of a sensitivity, specificity, or accuracy of the detection of the target nucleic acid.

In some embodiments, the reagents for nucleic acid amplification can comprise a recombinase, an oligonucleotide primer, a reverse transcriptase, a single-stranded DNA binding (SSB) protein, a polymerase, or any combination thereof. In some cases, a buffer is used for nucleic acid amplification. In some cases, a dNTPs are used for nucleic acid amplification. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, 60 or 120 minutes. Sometimes, the nucleic acid amplification is performed for from 1 to 60, from 1 to 120, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes.

In some cases, a reverse transcriptase used herein can comprise a Warmstart reverse transcriptase, a RapiDxFire Thermostable reverse transcriptase, a ACAT138 reverse transcriptase, a ACAT141 reverse transcriptase, a ProtoScript II Reverse Transcriptase, a Maxima H Minus Reverse Transcriptase, a Maxima Reverse Transcriptase, a RevertAid H Minus Reverse Transcriptase, a RevertAid Reverse Transcriptase, a SuperScript III Reverse Transcriptase, a M-MuLV Reverse Transcriptase, a Induro Reverse Transcriptase, an AMV Reverse Transcriptase, a StableScript Reverse Transcriptase, a EnzScript Reverse Transcriptase, a Quantiscript Reverse Transcriptase, a QuantiNova Reverse Transcriptase, a SMART MMLV reverse transcriptase, a MMLV reverse transcriptase, a SMARTScribe reverse transcriptase, a PrimeScript reverse transcriptase, an iScript reverse transcriptase, a M-MLV reverse transcriptase, a Transcriptor reverse transcriptase, an avian reverse transcriptase, an HIV reverse transcriptase, a fragment of any of these, or any combination thereof.

In some cases, a polymerase used herein can comprise a KAPA3G polymerase, a RapiDxFire Hot Start Taq DNA Polymerase, an aCat77 polymerase, a StellarScript HT polymerase, a StellarScript HT+ polymerase, a HawkXO5 polymerase, a KAPA2G polymerase, a SpeedStar polymerase, a fragment of any of these, or any combination thereof. In some cases, an polymerase can comprise a Titanium Taq enzyme, an Advantage 2 enzyme, a PrimeSTAR GXL enzyme, a PrimeSTAR Max enzyme, a Terra PCR Direct enzyme, a Takara LA Taq enzyme, a Takara Ex Taq enzyme, a fragment of any of these, or any combination thereof. In some cases a polymerase can comprise a Q5 High-Fidelity DNA Polymerase, a Q5U Hot Start High-Fidelity DNA Polymerase, a Phusion High-Fidelity DNA Polymerase, a OneTaq DNA Polymerase, a Taq DNA Polymerase, LongAmp Taq DNA Polymerase, a Hemo KlenTaq polymerase, a Epimark Hot Start Taq DNA Polymerase, a Bst DNA Polymerase, a fragment, Bst DNA Polymerase, a Bst 2.0 DNA Polymerase, a Bst 3.0 DNA Polymerase, a T7 DNA Polymerase, a Sulfolobus DNA Polymerase IV, a Therminato DNA Polymerase, a DNA Polymerase I, a T4 DNA polymerase, a vent DNA polymerase, a deep vent DNA polymerase, a Bastaq Hotstart DNA polymerase, a fragment of any of these, or any combination thereof. In some cases, a polymerase can comprise a HGS Diamond Taq Polymerase, an EasyTaq DNA Polymerase, a TransStart Taq DNA Polymerase, a TransFast Taq DNA Polymerase, a Phusion polymerase, a Taq DNA Polymerase Enzyme, a HotStart DNA Polymerase, a Precision DNA Polymerase, a TransTaq-T DNA Polymerase, a Diamond Taq DNA Polymerase, a Red Diamond Taq DNA Polymerase, a SilverStar DNA Polymerase, a Green Taq DNA Polymerase, TaqFast DNA Polymerase, a Kodaq DNA Polymerase, a Taq DNA Polymerase Enzyme, a TransTaq DNA Polymerase High Fidelity, a TransStart a TopTaq DNA Polymerase, a Bestaq DNA Polymerase, a Hot Diamond Taq DNA Polymerase, a Taq DNA Polymerase, a Long-Range DNA Polymerase, TransStart KD Plus DNA Polymerase, a fragment of any of these, or any combination thereof.

In some cases, a polymerase used herein can comprise a KAPA3G polymerase, a RapiDxFire Hot Start Taq DNA Polymerase, an aCat77 polymerase, a StellarScript HT polymerase, a StellarScript HT+ polymerase, a HawkXO5 polymerase, a KAPA2G polymerase, a SpeedStar polymerase, a fragment of any of these, or any combination thereof and a reverse transcriptase used herein can comprise a Warmstart reverse transcriptase, a RapiDxFire Thermostable reverse transcriptase, a fragment of any of these, or any combination thereof. In a preferred example, a polymerase used herein can comprise a KAPA3G polymerase or a KAPA2G polymerase and a reverse transcriptase used herein can comprise a Warmstart reverse transcriptase or a RapiDxFire Thermostable reverse transcriptase. In some embodiments, the polymerase and/or reverse transcriptase may be tolerant of inhibitors from a bead purification, such as isopropanol and ethanol, and/or inhibitors from a manufacturing process, such as Mg²⁺ or K, and may not require a buffer exchange step prior to amplification.

In some embodiments, a buffer is used for nucleic acid amplification. In some instances, a buffer can comprise a base buffer such as a Tris buffer. In some cases, a buffer can comprise, MgCl₂, MgOAc, KCl, KOAc, Bovine Serum Albumin (BSA), Fish Gelatin, CHAPS or any combination thereof. In some cases, the Tris buffer can be 10 mM-300 mM. In some cases, the Tris buffer can be 10 mM, 20 mM, 30 mM, 40 mM, 50 mM, 60 mM, 70 mM, 80 mM, 90 mM, or 100 mM. In some cases, the Tris buffer can be 50 mM. In some cases, the pH of the buffer can be about 6.5 to 9.2. In some cases, the pH of the buffer can be about 7 pH, 7.1 pH, 7.2 pH, 7.3 pH, 7.4 pH, 7.5 pH, 7.6 pH, 7.7 pH, 7.8 pH, 7.9 pH, 8 pH, 8.1 pH, 8.2 pH, 8.3 pH, 8.4 pH, 8.5 pH, 8.6 pH, 8.7 pH, or 8.8 pH. In some embodiments, the pH is about 8.3 to about 8.8. In some cases, a buffer can comprise about: 1 mM MgCl₂, 2 mM MgCl₂, 3 mM MgCl₂, 4 mM MgCl₂. 5 mM MgCl₂, or 6 mM MgCl₂. In some cases, a buffer can comprise about: 1 mM MgCl₂, to about 10 mM MgCl₂. In some cases, a buffer can comprise about 1-3 mM of MgOAc, or about 1.75 mM of MgOAc. In some cases, a buffer can comprise about 30 mM to about 125 mM KCl. In some cases, a buffer can comprise about: 30 mM KCl, 40 mM KCl, 50 mM KCl, 60 mM KCl, 70 mM KCl, 80 mM KCl, 90 mM KCl, 100 mM KCl, or 110 mM KCl. In some embodiments, the buffer comprises about 30 mM KCl or about 75 mM KCl. In some embodiments, the buffer comprises 25 mM to 100 mM KOAc, such as about 30 mM, 40 mM, 50 mM, 60 mM, 70 mM, or 80 mM KOAc. In some embodiments, the buffer comprises 50 mM KOAc. In some cases, a buffer can comprise about 0.1 mg/mL to about 1 mg/mL of BSA. In some cases, a buffer can comprise about 0.3 mg/mL of BSA, 0.4 mg/mL of BSA, 0.5 mg/mL of BSA, 0.6 mg/mL of BSA or 0.7 mg/mL of BSA. In some embodiments, the buffer comprises about 0.25 mg/mL BSA. In some embodiments, the buffer comprises about 0.5 mg/mL BSA. In some cases, a buffer can comprise about 0.1 mg/mL to about 1 mg/mL of fish gelatin. In some cases, a buffer can comprise about 0.3 mg/mL of fish gelatin, 0.4 mg/mL of fish gelatin, 0.5 mg/mL of fish gelatin, 0.6 mg/mL of fish gelatin or 0.7 mg/mL of fish gelatin. In some embodiments, the buffer comprises about 0.25 mg/mL of fish gelatin. In some embodiments, the buffer comprises 0.5 mg/mL of fish gelatin. In some cases, a buffer can comprise about 0.1% to about 2% CHAPS detergent. In some cases, a buffer can comprise about 0.50% CHAPS detergent. In some cases, MgCl₂ can act as a divalent cation for correct function of a polymerase, as many polymerases can require a divalent cation for activity. In some cases, the addition of KCl in a buffer can improve the replication efficiency of a buffer. In some cases, KCl can act by neutralizing the charge present on the backbone of DNA. In some cases, BSA in a buffer can be replaced by a fish gelatin. In some cases, a buffer can comprise: 50 mM Tris, 3 mM of MgCl₂, 75 mM of KCL and 0.5 mg/ml of BSA and/or fish gelatin. In some embodiments, the buffer comprises: (a) 50 mM Tris, 3 mM of MgCl₂, and 0.5 mg/mL of BSA; (b) 50 mM Tris, 3 mM of MgCl₂, 75 mM of KCL and 0.5 mg/mL of BSA; (c) 50 mM Tris, 3 mM of MgCl₂, 75 mM of KCL and 0.5 mg/mL fish gelatin; (d) 50 mM Tris, 3.5 mM of MgCl₂, 30 mM of KCL and 0.5 mg/mL fish gelatin; (e) 50 mM Tris, 3.5 mM of MgCl₂, 30 mM of KCL and 0.5% CHAPS; or (f) 50 mM Tris, 1.75 mM of MgAOc, 50 mM of KOAc, and 0.5 mg/mL fish gelatin.

In some cases, a buffer can comprise a KlenTaq1 buffer. In some cases, a buffer can comprise a Phusion GB buffer, a Phire reaction buffer, a detergent free buffer, a Promega 5X PCR buffer, a Promega PCR buffer, a green GoTaq reaction buffer, a colorless GoTaq reaction buffer, Promega buffer A, Promega buffer B, Promega buffer C, a P2192 PCR buffer, a P2317 PCR buffer, or any standard buffer. In some cases, a master mix can comprise a buffer such that the master mix comprises all the reagents necessary for a PCR reaction. The reagents may be provided in a lyophilized form for reconstitution in the device (e.g., upon contact with a sample-containing fluid).

In some embodiments, a total reaction volume can comprise about 5 μL to about 50 μL, about 10 μL to about 30 μL, or about 15 μL to about 25 μL. In some instances, a total reaction volume can be about 15 μl to about 23 μl. In some instances, a total reaction volume can be about: 14 μl, 15 μl, 16 μl, 17 μl, 18 μl, 19 μl, 20 μl, 21 μl, 22 μl, 23 μl, 24 μl, or 25 μl. In some cases, a larger reaction volume is utilized to increase fluidics on a device and/or make a reaction more tolerant to variability (e.g., manufacture variations in devices and reagents, fluid loading, temperature differences within a device, etc.).

In some embodiments, amplification can comprise thermocycling of the sample. Thermocycling can be carried out in a vessel (e.g., a tube) for a single large sample, and/or independently in separate locations (e.g., multiple chambers or wells within a device). This can be accomplished by methods such as (1) by holding droplets stationary in locations where a heating element is in close proximity to the droplet on one of the droplet sides and a heat sink element is in close proximity to the other side of the droplet, (2) flowing a sample through zones in a fluid channel where heat flows across it from a heating source to a heat sink, (3) an amplification chamber of a device for thermocycling a sample before the sample is subdivided, or (4) in each of a plurality of amplification chambers of a device, each chamber containing a portion of a subdivided sample. In some cases, one or more resistive heating elements can be used to perform thermocycling.

Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., 50° C., 55° C., 60° C., 65° C. or 68° C. In some embodiments, the nucleic acid amplification reaction is performed at a temperature of at least 20° C., 25° C., 30° C., 35° C., 37° C., 40° C., 45° C., 50° C., 55° C., 60° C., 65° C. or 68° C. In some embodiments, the nucleic acid amplification reaction is performed at a temperature of about 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 68° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., 40° C. to 68° C., from 45° C. to 65° C., from 50° C. to 65° C., from 55° C. to 65° C., from 60° C. to 65° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.

In some embodiments, thermocycling comprises a plurality of cycles, wherein each cycle comprises denaturation at a first temperature and primer extension by a polymerase at a second temperature that is lower than the first temperature. In some embodiments, each cycle is about or less than about 20 seconds in duration (e.g., about or less than about 15, 14, 13, 12, 11, 10, 9, 8, 7, 6, 5, or 4 seconds in duration). In some embodiments, each cycle is less than 15 seconds in duration. In some embodiments, each cycle is less than 10 seconds in duration. In some embodiments, the plurality of cycles are about 2 seconds to about 20 seconds in duration, about 3 seconds to about 10 seconds in duration, or about 5 seconds in duration. In some embodiments, the cycles are about 4 seconds in duration. In some embodiments, the denaturation step is about 0.5 to about 5 seconds in duration, about 1 to about 3 seconds in duration, or about 1 seconds in duration. In some embodiments, the denaturation is about 2 second in duration. In some embodiments, the denaturation is about 2 seconds in duration. In some embodiments, the first temperature is about 90° C. to about 99° C., about 96° C. to about 97° C., or about 95° C. In some embodiments, the first temperature (e.g., the denaturation) is about 94° C. In some cases, a lower denaturation temperature is selected to facilitate more rapid thermocycling and reduce evaporation. Without wishing to be bound by theory, it is believed that an associated increase in non-specific amplification may be advantageously off-set by specificity of the programmable nuclease in a subsequent or concurrent programmable nuclease-based detection reaction.

In some embodiments, the primer extension step in an amplification cycle is about 1 to about 15 seconds in duration, about 2 to about 10 seconds in duration, or about 5 seconds in duration. In some embodiments, the primer extension step is about 6 seconds in duration. In some embodiments, the second temperature is about 45° C. to about 75° C., about 50° C. to about 70° C., or about 55° C. to about 65° C. In some embodiments, the second temperature is about 65° C. In some embodiments, each cycle comprises denaturation at the first temperature for about 2 second and primer extension at the second temperature for about 6 seconds. In some embodiments, each cycle comprises denaturation at the first temperature of 94° C. for about 2 second and primer extension at the second temperature of 65° C. for about 6 seconds. In some embodiments, the plurality of cycles comprises about or at least about 20 cycles (e.g., about or more than about 25, 30, 35, 40, or 45 cycles). In some embodiments, the plurality of cycles comprises about 20 cycles to about 50 cycles, or about 30 cycles to about 45 cycles. In some embodiments, the plurality of cycles comprises about 40 cycles. In some embodiments, the plurality of cycles comprises about 35 cycles. In some embodiments, the plurality of cycles comprises about: 33, 34, 35, 36, 37, 38, 39, 40, 41, 42, 43, 44, 45, 46, 47, or 48 cycles. In some embodiments, the plurality of cycles is preceded by an initial denaturation step at the first temperature that is longer in duration that the durations of the individual denaturation steps in each of the cycles. In some embodiments, the initial denaturation step is about 10 seconds to about 120 seconds in duration, about 15 seconds to about 60 seconds in duration, or about 20 seconds to about 50 seconds in duration. In some embodiments, the initial denaturation step is about 30 seconds in duration. In some embodiments, the total duration of the amplification by thermocycling is about 1 minute to about 20 minutes, about 2 minutes to about 15 minutes, or about 3 minutes to about 10 minutes. In some embodiments, the total duration of the amplification by thermocycling is less than about 10 minutes. In some embodiments, the total duration of the amplification by thermocycling is about 5 minutes.

In some non-limiting embodiments, the target nucleic acid(s) can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, a buffer, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., 65° C. or 68° C. The nucleic acid amplification reaction can be performed at a temperature of at least 20° C., 25° C., 30° C., 35° C., 37° C., 40° C., 45° C., 50° C., 55° C., 60° C., 65° C., or 68° C.

In some embodiments, Cas enzymes disclosed herein can be used with a diluted or undiluted PCR product. In some cases, a Cas enzyme can be included in a one-pot assay, for example with an undiluted PCR assay. In some instances, a single reaction volume CRISPR reactions may use a PCR assay described herein to exponentially amplify a target DNA or RNA molecule while simultaneously allowing a CRISPR/Cas nuclease to report the presence or absence of the target DNA or RNA through complementarity with a guide nucleic acid and cleavage of a reporter molecule as described herein.

Additional amplification methods are described in WO/2022/133108 which is incorporated herein by reference in its entirety.

In some instances, the effector protein, the guide nucleic acid, and the detection reagent of the method are formulated in a solution. The solution may comprise the solutions or system solutions described thereof. In some instances, amplifying a target nucleic acid may be performed using any instruments described herein. The instruments may comprise the cartridges described herein.

### Detection of a Target Nucleic Acid

In some cases, there is a threshold of detection for methods of detecting target nucleic acids. In some instances, methods are not capable of detecting target nucleic acids that are present in a sample or solution at a concentration less than or equal to 10 nM. A threshold of detection may be the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM.

In some instances, the target nucleic acid is present in a sample at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some cases, methods of detecting are performed in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, or less than about 5 minutes. In some cases, methods of detecting are performed in about 5 minutes to about 120 minutes, about 5 minutes to about 100 minutes, about 10 minutes to about 90 minutes, about 15 minutes to about 45 minutes, about 20 minutes to about 35 minutes.

In some cases, methods of detecting are performed in less than about 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the effector protein. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid

### V. Target Nucleic Acids and Samples

### A. Certain Target Nucleic Acids

Disclosed herein are compositions, systems and methods for detecting and/or modifying a target nucleic acid. In some instances, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some instances, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some cases, the target nucleic acid is from a virus, a parasite, or a bacterium described herein.

In some cases, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleosides. In some cases, the target nucleic acid comprises 10 to 90, 20 to 80, 30 to 70, or 40 to 60 linked nucleosides. In some cases, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleosides. In some instances, the target nucleic acid comprises at least 10, at least 20, at least 30, at least 40, at least 50, at least 60, at least 70, at least 80, at least 90, or at least 100 linked nucleosides.

A programmable nuclease-guide nucleic acid complex may comprise high selectivity for a target sequence. In some cases, a ribonucleoprotein may comprise a selectivity of at least 200:1, 100:1, 50:1, 20:1, 10:1, or 5:1 for a target nucleic acid over a single nucleotide variant of the target nucleic acid. In some cases, a ribonucleoprotein may comprise a selectivity of at least 5:1 for a target nucleic acid over a single nucleotide variant of the target nucleic acid. Leveraging programmable nuclease selectivity, some methods described herein may detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample comprises 1 to 10,000, 100 to 8000, 400 to 6000, 500 to 5000, 1000 to 4000, or 2000 to 3000 target nucleic acids. In some cases, the method detects target nucleic acid present at least at one copy per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids.

The target nucleic acid may be from 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. In some cases, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. The segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA. Additionally, target nucleic acid can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM.

In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the systems, devices, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some instances, compositions described herein exhibit indiscriminate trans-cleavage of ssRNA, enabling their use for detection of RNA in samples. In some cases, target ssRNA are generated from many nucleic acid templates (RNA) in order to achieve cleavage of the FQ reporter in the trans-cleavage assay platform. Certain programmable nucleases may be activated by ssRNA, upon which they may exhibit trans-cleavage of ssRNA and may, thereby, be used to cleave ssRNA FQ reporter molecules in the trans-cleavage assay system. These programmable nucleases may target ssRNA present in the sample, or generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the programmable nuclease, upon generation and amplification of ssRNA from a nucleic acid template using the methods disclosed herein, thereby generating a first detectable signal.

In some instances, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2μ, 2 μM to 3μ, 3μ M to 4μ, 4μ M to 5μ, 5μ M to 6μ, 6 μM to 7μ, 7μ M to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 M, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid sequence. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

A number of target nucleic acid populations are consistent with the methods and compositions disclosed herein. Some methods described herein may detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has 3 to 50, 5 to 40, or 10 to 25 target nucleic acid populations. In some cases, the method detects target nucleic acid populations that are present at least at one copy per 101 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids. The target nucleic acid populations may be present at different concentrations or amounts in the sample.

In some embodiments, target nucleic acids may activate a programmable nuclease to initiate sequence-independent cleavage of a nucleic acid-based reporter (e.g., a reporter comprising an RNA sequence, or a reporter comprising DNA and RNA). For example, a programmable nuclease of the present disclosure is activated by a target nucleic acid to cleave reporters having an RNA (also referred to herein as an “RNA reporter”). Alternatively, a programmable nuclease of the present disclosure is activated by a target nucleic acid to cleave reporters having an RNA. Alternatively, a programmable nuclease of the present disclosure is activated by a target RNA to cleave reporters having an RNA (also referred to herein as a “RNA reporter”). The RNA reporter may comprise a single-stranded RNA labeled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

In some embodiments, the target nucleic acid comprises a nucleic acid sequence from a pathogen responsible for a disease. Non-limiting examples of pathogens are bacteria, a virus and a fungus. The target nucleic acid, in some cases, is a portion of a nucleic acid from a sexually transmitted infection or a contagious disease. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid sequence comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. A programmable nuclease of the disclosure (e.g., Cas12) may cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

### Mutations

In some instances, target nucleic acids comprise a mutation. In some instances, a sequence comprising a mutation may be modified to a wildtype sequence with a composition, system or method described herein. In some instances, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. Non-limiting examples of mutations are insertion-deletion (indel), single nucleotide polymorphism (SNP), and frameshift mutations. In some instances, guide nucleic acids described herein hybridize to a region of the target nucleic acid comprising the mutation. The mutation may be located in a non-coding region or a coding region of a gene.

In some instances, target nucleic acids comprise a mutation, wherein the mutation is a SNP. The single nucleotide mutation or SNP may be associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. The single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a maycer cell.

In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

### B. Certain Samples

Various sample types comprising a target nucleic acid of interest are consistent with the present disclosure. These samples may comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some instances, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to the detection system of the present disclosure. Non-limiting examples of environmental samples are soil, air, or water. In some instances, an environmental sample is taken as a swab from a surface of interest or taken directly from the surface of interest.

In some instances, the sample is a raw (unprocessed, unmodified) sample. Raw samples may be applied to a system for detecting or modifying a target nucleic acid, such as those described herein. In some instances, the sample is diluted with a buffer or a fluid or concentrated prior to its application to the system. Sometimes, the sample contains no more than 20 μl of buffer or fluid. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some instances, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

In some instances, samples are used for diagnosing a disease. In some instances the disease is cancer. The sample used for cancer testing may comprise at least one target nucleic acid that may bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, system, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WTI. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some instances, samples are used to diagnose a genetic disorder, also referred to as genetic disorder testing. The sample used for genetic disorder testing may comprise at least one target nucleic acid that may bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMRI, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6VIB1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that may bind to a guide nucleic acid of the reagents described herein. The target nucleic acid, in some cases, is a nucleic acid encoding a sequence associated with a phenotypic trait.

The sample used for genotyping testing may comprise at least one target nucleic acid that may bind to a guide nucleic acid of the reagents described herein. The target nucleic acid, in some cases, is a nucleic acid encoding a sequence associated with a genotype of interest.

The sample used for ancestral testing may comprise at least one target nucleic acid that may bind to a guide nucleic acid of the reagents described herein. The target nucleic acid, in some cases, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. The disease status is prostate disease status, but the status of any disease may be assessed.

Any of the above disclosed samples are consistent with the methods, compositions, reagents, enzymes, and systems disclosed herein.

### VI. Instruments

The present disclosure provides systems and methods for nucleic acid target detection. The systems and methods of the present disclosure can be implemented using devices that are configured for programmable nuclease-based detection. In some embodiments, the devices can be configured for single reaction detection. The target can comprise a target sequence or target nucleic acid. As used herein, a target can be referred to interchangeably as a target nucleic acid. Further, a target can be referred to as a target amplicon or a target nucleic acid amplicon if such target undergoes amplification (e.g., through a thermocycling process as described elsewhere herein). The target nucleic acid can be a portion of a nucleic acid of interest, e.g., a target nucleic acid from any plant, animal, virus, or microbe of interest. The devices provided herein can be used to perform rapid tests in a single integrated system.

### D. Detection

In some embodiments, the instrument may comprise a detector/sensor system to read out the spatial multiplexing implemented in the cartridge. In some embodiments, the detector system may comprise a source and a detector. In some embodiments, the source may comprise an illumination source. In some embodiments, the source may comprise an electrical signal source.

In some embodiments, the detector comprises an optical sensor or optical detector. In some embodiments, the detector is an image sensor (e.g., a camera, photomultiplier tube, charge-coupled device, active-pixel sensor, or the like). In some embodiments, the detector may comprise an array of discrete optical detectors, one for each detection spot or chamber within the detection region of the cartridge. In some embodiments, one or more lenses and/or filters may be implemented to create an image with sufficient resolution and brightness for detection of reporter cleavage.

In some embodiments, the resolution of the detector may be about 50 micrometers. In some embodiments, the resolution of the detector may be less than about 50 micrometers. In some embodiments, the resolution of the detector may be less than about 25 micrometers. In some embodiments, the resolution of the detector may be less than about 5 micrometers.

In some embodiments, the signal is selected from the group consisting of an optical signal, a fluorescent signal, a colorimetric signal, a potentiometric signal, an amperometric signal, and a piezo-electric signal. In some embodiments, the signal is associated with a change in an index of refraction of a solid or gel volume in which said at least one programmable nuclease probe is disposed. Cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In some embodiments, the method for detection is fluorescence. In some embodiments, the detector (or plurality of detectors) may detect a change in wavelength (e.g., a change in color), intensity (e.g., brightness), or a degree of wavelength change (e.g., with appropriate dispersion elements to access wavelength space). In some embodiments, the system may be configured to detect one or more wavelengths (e.g., one for each fluorophore in a fluorescence-based system). In order to enable this fluorescence detection, optical filtering of illumination light and/or detection light may be implemented to block unwanted crosstalk (e.g., illumination light being detected in detection systems).

In some embodiments, detection mechanisms may comprise interferometry, surface plasmon resonance, electrochemical detection such as potentiometry, or other detection mechanisms.

### Detection Regions

Detection of reporter cleavage by an activated programmable nuclease may occur in a detection region of a device or surface. In some embodiments, the detection region may be downstream of an amplification region. The detection region may comprise one or more detection locations. In some embodiments, a detection location may comprise one or more detection reagents. For example, the detection location may comprise a reporter, a guide nucleic acid, and/or a programmable nuclease. The reporter may be any of the reporters, or any combination of reporters, described herein. The guide nucleic acid may be any of the guide nucleic acids, or any combination of guide nucleic acids, described herein. The programmable nuclease may be any of the programmable nucleases, or any combination of programmable nucleases, described herein. Any one of several programmable nucleases (e.g., Cas proteins) may be used individually or in combination with other programmable nucleases. In some embodiments, the programmable nuclease may be different in the different detection locations.

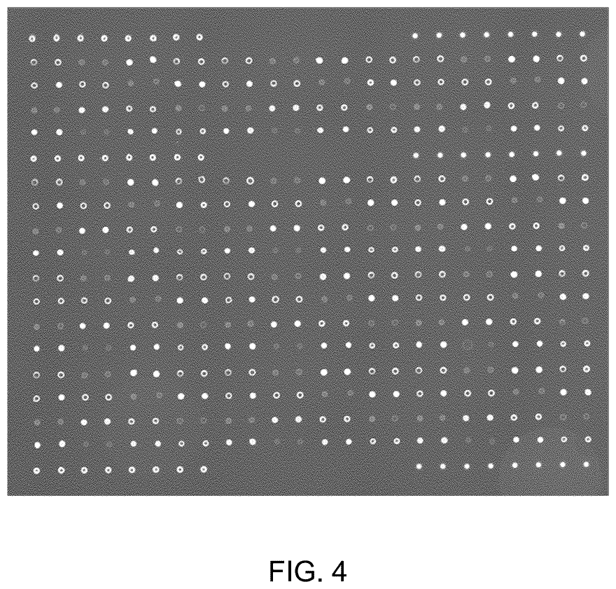

In some embodiments, the detection region may be configured to detect one or more signals from a liquid-based programmable nuclease-based detection reaction, or an immobilized array programmable nuclease-based detection reaction as described herein. For example, the detection region may comprise one or more detection channels, chambers, microwells, or the like comprising detection reagents suitable for a liquid-based assay. In another example, the detection region may comprise one or more detection locations (e.g., the detection spots or microwells of FIG. 4) comprising one or more immobilized detection reagents.

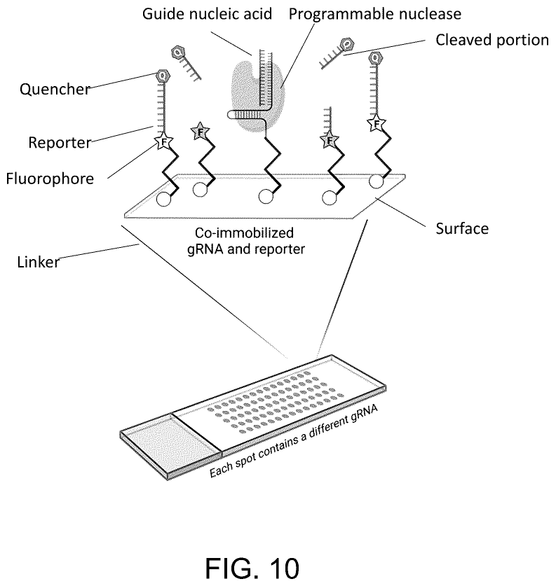

FIG. 10 depicts a surface comprising an immobilized programmable nuclease-guide nucleic acid complex and a plurality of reporters, with one reporter having been cleaved by an activated programmable nuclease. In some embodiments, one or more detection reagents may be immobilized on a surface of the detection region. For example, one or more programmable nuclease, one or more guide nucleic acid, and/or one or more reporter may be immobilized on a surface of the detection region. In some embodiments, one or more detection reagents may be immobilized on a surface of the detection region via a linker. The programmable nuclease may be complexed with a guide nucleic acid complementary to a specific target nucleic acid sequence as described herein. When a target nucleic acid is present in the sample, the programmable nuclease may be activated by binding of the guide nucleic acid (which is complexed thereto) to the target nucleic acid. Activation of the programmable nuclease may enable trans-cleavage of the reporter as described herein. In some embodiments, the reporter may comprise a detection moiety as described herein. In some embodiments, the reporter may comprise a detection moiety (e.g., a fluorophore) and a quencher moiety (e.g., a quencher) configured to generate a detectable signal when separated from one another. In some embodiments, trans-cleavage of the reporter by the activated programmable nuclease may release fluorophore of a reporter (or a quencher moiety, depending on the signal), thereby generating a signal indicative of the presence or absence of the target nucleic acid in the sample as described herein. The reporter may comprise any of the reporters described herein, and may comprise any detection moiety described herein (and/or other moieties or molecules described herein which facilitate signal detection). As shown in FIG. 10, cleavage of the reporter may release the quencher into solution, thereby allowing the fluorophore which was quenched when the reporter was intact to fluoresce. The presence of fluorescence at the detection location may therefore indicate the presence of the target nucleic acid. In another example, the reporter may not comprise a quencher and cleavage of the reporter may instead release a fluorophore into solution, thereby reducing fluorescence at the detection location. The absence of fluorescent at the detection location may therefore indicate the presence of the target nucleic acid. In some embodiments, cleavage of the reporter may result in a non-fluorescent signal as described herein. For example, cleavage of the reporter can produce a calorimetric signal, a potentiometric signal, an amperometric signal, a colorimetric signal, or a piezo-electric signal.

In some embodiments, the one or more detection reagents can be immobilized in discrete detection locations using NHS-amine chemistry as described herein. For example, a primary amine-modified guide nucleic acid and a primary amine-modified reporter may be conjugated to an NHS-coated surface of the detection region.

In some embodiments, the one or more detection reagents may be immobilized using streptavidin-biotin chemistry as described herein. For example, a biotinylated reporter and a biotinylated guide nucleic acid may be immobilized to a streptavidin-coated surface of the detection region.

In some embodiments, the one or more detection reagents may be immobilized using maleimide-thiol chemistry as described herein. For example, a thiol-modified guide nucleic acid and a thiol-modified reporter may be conjugated to a maleimide-coated surface of the detection region.

In some embodiments, the one or more detection reagents may be immobilized using epoxy-amine chemistry as described herein. For example, an amine-modified guide nucleic acid and an amine-modified reporter may be conjugated to an epoxy-coated surface of the detection region.

In some embodiments, the one or more detection reagents may be immobilized using hydrogels as described herein. For example, an acrydite-modified guide nucleic acid and an acrydite-modified reporter may be co-polymerized with an acrylate-modified oligomer (e.g., PEG-diacrylate) prior to deposition on the surface of the detection region or in situ on the surface of the detection region.

In some embodiments, the detection region may comprise one or more discrete detection locations. The detection location(s) may comprise a detection spot, a detection chamber, a detection channel, a detection well (e.g., a microwell), or the like, or any combination thereof. In some embodiments, the detection region may comprise a plurality of detection spots or microwells that are spatially separated from one another at discrete detection locations. Spatial separation of the detection locations may enable multiplexed target nucleic acid analysis of the sample within the device.

In some embodiments, one or more detection reagents may be immobilized on a surface of the detection region at one or more detection locations. For example, one or more programmable nuclease, one or more guide nucleic acid, and/or one or more reporter may be immobilized on a surface of the detection region at one or more detection locations. Alternatively, or in combination, one or more of the detection reagents at the detection location(s) may be in a dried or lyophilized form prior to mixing with the sample fluid. Each programmable nuclease may be complexed with a guide nucleic acid complementary to a specific target nucleic acid sequence. In some embodiments, each detection location may comprise a different combination of detection reagents so as to provide a plurality of spatially separated multiplex-capable detection locations. For example, one or more (e.g., each) detection location may comprise a different guide nucleic acid configured to bind to a different target nucleic acid. Alternatively, or in combination, one or more (e.g., each) detection location may comprise a different programmable nuclease. Alternatively, or in combination, one or more (e.g., each) detection location may comprise a different reporter. In some embodiments, each detection location may comprise a plurality of reporters. In some embodiments, each detection location may comprise detection reagents configured to detect a different target nucleic acid of a plurality of target nucleic acids.

In some embodiments, the detection locations may be patterned as an array. For example, an array of detection locations may comprise a two-dimensional array of detection spots, chambers, or microwells arranged in orthogonal directions. The array may be an m×n array having m columns of n detection spots, chambers, or microwells arranged in rows. In some embodiments, m and n may be different. In some embodiments, m and n may be the same. In some embodiments, an array of detection locations may be asymmetrical (e.g., detection locations may be patterned to minimize the usage of space in the detection region with regard for symmetry). It will be apparent to one of ordinary skill in the art that the detection region may comprise any suitable number of detection locations and the detection locations may be arranged in any suitable manner so as to enable multiplexed target nucleic acid analysis.

In some embodiments, the detection reagents may be provided on a surface of the detection region as an immobilized array. In some embodiments, the array may comprise a number of detection locations within a range of about 1 to about 200, within a range of about 3 to about 200, or within a range of about 10 to about 200. In some embodiments, the array may comprise at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more detection locations.

In some embodiments, one or more locations may comprise the same detection reagents (e.g., detection reagents specific to the same target nucleic acid). In some embodiments, 1 to 12 detection locations may comprise the same detection reagents. For example, an exemplary detection region may comprise anywhere from 1 to 12 replicate spots for each target nucleic acid to be detected.

In some embodiments, the detection region may comprise an array of detection spots. Each detection spot of the array may comprise a reporter and a different programmable nuclease of a plurality of programmable nucleases as described herein. In some embodiments, each of the different programmable nucleases of the plurality of programmable nucleases may comprise a different guide nucleic acid which is complementary to a different target nucleic acid of a plurality of target nucleic acids. In some embodiments, the reporter and each different programmable nuclease and/or guide nucleic acid of each detection spot of the array may be immobilized to a surface of the detection region. In some embodiments, at each detection spot of the array, each different programmable nuclease may be configured to cleave an adjacent reporter and generate a different signal of a plurality of signals. Each different signal may therefore be indicative of the presence or absence of a different target nucleic acid. In some embodiments, the reporter may comprise a fluorophore and a quencher as described herein. The guide nucleic acid and reporter may each be immobilized to a surface of a detection spot with a linker as described herein. The target nucleic acids may be freely available within the fluid volume of the detection region. In some embodiments, multiple guide nucleic acids for a single target nucleic acid may be combined within a single detection spot in order to increase a rate of reaction. Localizing the guide nucleic acids and reporter may localize the detectable signal for each target nucleic acid to the detection spot, thus enabling the spatial multiplexing. In some embodiments, the array may comprise a number of spots within a range of about 1 to about 200, within a range of about 3 to about 200, or within a range of about 10 to about 200. In some embodiments, the array may comprise at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 1000, 2000, 3000, 4000, 5000, 10000, 100000 or more spots.

In some embodiments, the detection region may comprise a plurality of chambers. In some embodiments, each chamber may comprise a reporter and a different programmable nuclease of a plurality of programmable nucleases. In some embodiments, each of the different programmable nucleases of the plurality of programmable nucleases may comprise a different guide nucleic acid which is complementary to a different target nucleic acid of a plurality of target nucleic acids. In some embodiments, one or more programmable nuclease, one or more reporter, and/or one or more guide nucleic acid may be lyophilized, vitrified, or dried as described herein. In some embodiments, each different programmable nuclease may be configured to cleave an adjacent reporter and generate a different signal of a plurality of signals. Each different signal may therefore be indicative of the presence or absence of a different target nucleic acid as described herein.

### VII. Certain Methods of Use

Any of the systems described herein (which may comprise any of the instruments and/or cartridges described herein) may be used to detect one or more target nucleic acids in a sample. In some embodiments, detecting the one or more target nucleic acids may comprise one or more of the following steps: sample collection, sample extraction, sample lysis, protein degradation, nucleic acid extraction, nucleic acid purification, nucleic acid concentration, waste removal, nucleic acid elution, nucleic acid amplification, a programmable nuclease-based detection reaction, target detection, and/or reporter detection, or any combination thereof.

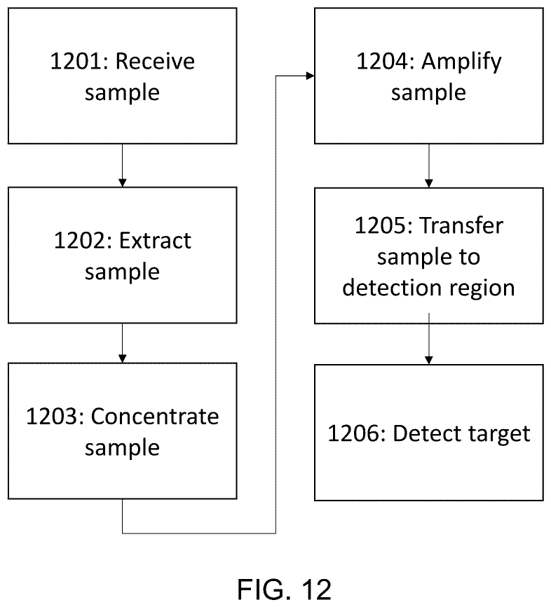

FIG. 12 shows an exemplary workflow of a method 1200 for nucleic acid analysis and detecting a target nucleic acid.

At Step 1201, a user or system may receive a sample. The sample may comprise any of the samples described herein. In some embodiments, the sample may comprise one or more different target nucleic acids. Receiving a sample containing a target nucleic acid may subsequently result in a “hit” for that target nucleic acid at Step 1208. In some embodiments, the sample may not comprise a target nucleic acid. In some embodiments, the sample may be collected with a sample collector (e.g., swab, tube, etc.) as described herein. In some embodiments, the sample collector may be received in a sample interface of a detection device as described herein. In some embodiments, the sample may be directly collected at the sample interface (e.g., without the use of a separate sample collector).

At Step 1202, In some embodiments, the sample may optionally be extracted from the sample collector. Extracting the sample may comprise eluting at least a portion of the sample from the sample collector (e.g., when the sample collector is a swab). In some embodiments, extracting the sample may comprise scraping the sample collector against a scraper as described herein. In some embodiments, extracting the sample may comprise pipetting the sample (e.g., when the sample collector is a tube). In some embodiments, extracting the sample may comprise lysing and/or heating the sample as described herein.

At Step 1203, at least a portion of the sample may optionally be concentrated as described herein. For example, the sample may comprise a plurality of nucleic acids and concentrating the sample may comprise concentrating the plurality of nucleic acids as described herein. In some embodiments, the plurality of nucleic acids may comprise one or more target nucleic acids and concentrating the sample may comprise concentrating the target nucleic acid(s).

At Step 1204, one or more nucleic acids in the sample may optionally be amplified as described herein. For example, the one or more target nucleic acids may be amplified via an isothermal or thermal amplification reaction as described herein and thereof. In some embodiments, amplifying the sample may comprise exposing the sample to one or more amplification reagents as described herein and thereof. In some embodiments, amplifying the sample may comprise raising the temperature of the fluid containing the sample to a pre-determined temperature. In some embodiments, amplifying the sample may comprise cycling the temperature of the fluid containing the sample between a plurality of pre-determined temperatures.

At Step 1206, after amplification, the sample may be transferred or applied to a detection region. The detection region may comprise one or more spatially separated detection locations (e.g., detection spots, microwells, chambers, etc.) as described elsewhere in this disclosure. In some embodiments, the detection region may be part of a detection device and transferring the sample may comprise transferring a target nucleic acid to the detection device. In some embodiments, one or more detection reagents may be immobilized on a surface of the detection region. For example, one or more programmable nuclease, one or more guide nucleic acid, and/or one or more reporter may be immobilized on a surface of the detection region as described herein and thereof. In some embodiments, one or more of the detection reagents may be in a dried or lyophilized form prior to mixing with the sample fluid. Each programmable nuclease may be complexed with a guide nucleic acid complementary to a specific target nucleic acid sequence. In some embodiments, each detection location may comprise a different combination of detection reagents so as to provide a plurality of spatially separated multiplex-capable detection locations. For example, one or more (e.g., each) detection location may comprise a different guide nucleic acid configured to bind to a different target nucleic acid. Alternatively, or in combination, one or more (e.g., each) detection location may comprise a different programmable nuclease. Alternatively, or in combination, one or more (e.g., each) detection location may comprise a different reporter. In some embodiments, each detection location may comprise detection reagents configured to detect a different target nucleic acid of a plurality of target nucleic acids.

At Step 1206, if present in the sample, the one or more target nucleic acids may be detected as described herein. Detecting the target nucleic acid may comprise detecting a signal indicative of cleavage of the reporter by the programmable nuclease at the detection location. When a target nucleic acid is present in the sample, the programmable nuclease may be activated by binding of the guide nucleic acid (which is complexed thereto) to the target nucleic acid. Activation of the programmable nuclease may enable trans-cleavage of the reporter as described herein. In some embodiments, the reporter may comprise a detection moiety as described herein. In some embodiments, trans-cleavage of the reporter by the activated programmable nuclease may release fluorophore of a reporter (or a quencher moiety, depending on the signal), thereby generating a signal indicative of the presence or absence of the target nucleic acid in the sample as described herein.

Although the steps above show a method 1200 of detecting a target nucleic acid using a programmable nuclease-based detection system in accordance with embodiments, a person of ordinary skill in the art will recognize many variations based on the teachings described herein. The steps may be completed in a different order. Steps may be added or deleted. Some of the steps may comprise sub-steps. Many of the steps may be repeated as often as necessary to perform a detection assay.

For example, in many embodiments, additional target nucleic acids are detected simultaneously (e.g., in a multiplexed fashion) as described herein. In some embodiments, the method 1200 may optionally include inactivating the sample prior to Step 1204. In some embodiments, the method 1200 may optionally include amplifying the signal produced upon cleavage of the reporter as described herein. In some embodiments, Steps 1204 and 1206 may occur in the same location and/or at the same time and step 1205 may be unnecessary. In some embodiments, Step 1204 may be optional for assays not requiring amplification.

### Illustrative Embodiments

The present disclosure provides the following illustrative embodiments.

Embodiment 1. A system for detecting a target nucleic acid, the system comprising:
a detection region comprising: i) a guide nucleic acid complementary to the target nucleic acid, or a portion thereof: ii) a reporter immobilized to a surface of the detection region, the reporter comprising a nucleic acid and a detection moiety, wherein a) the nucleic acid is at least 40 nucleotides in length; b) the nucleic acid comprises a double-stranded region; c) or a combination thereof, and wherein cleavage of the reporter by a programmable nuclease, activated upon hybridization to the target nucleic acid, releases the detection moiety from the nucleic acid, and wherein the release of the detection moiety is configured to generate a signal indicative of a presence of the target nucleic acid.

Embodiment 2. The system of embodiment 1, further comprising the programmable nuclease.

Embodiment 3. The system of embodiment 2, wherein the programmable nuclease is configured to form a complex with the guide nucleic acid and to be activated through binding of the guide nucleic acid to the target nucleic acid.

Embodiment 4. The system of embodiment 1, wherein the nucleic acid is at least 50 nucleotides in length.

Embodiment 5. The system of embodiment 4, wherein the nucleic acid comprises a single-stranded region and the double-stranded region.

Embodiment 6. The system of embodiment 5, wherein the single-stranded region is from about 5 to about 15 nucleotides in length.

Embodiment 7. The system of embodiment 5 or 6, wherein the double-stranded region is from about 45 to about 55 nucleotides in length.

Embodiment 8. The system of embodiment 5, wherein the single-stranded region is about 9 nucleotides in length, and wherein the double-stranded region is about 50 nucleotides in length.

Embodiment 9. The system of embodiment 1, wherein the nucleic acid is single-stranded.

Embodiment 10. The system of embodiment 9, wherein the single-stranded nucleic acid is at least about 50 nucleotides in length.

Embodiment 11. The system of embodiment 10, wherein the single-stranded nucleic acid is from about 55 to about 65 nucleotides in length.

Embodiment 12. The system of embodiment 11, wherein the detection moiety comprises a quencher moiety.

Embodiment 13. The system of embodiment 12, wherein the reporter comprises a fluorophore.

Embodiment 14. The system of embodiment 13, wherein the quencher moiety is configured to quench the fluorophore prior to the cleavage of the reporter.

Embodiment 15. The system of embodiment 1, wherein the detection moiety comprises a fluorophore.

Embodiment 16. The system of embodiment 1, wherein the signal is 1) a fluorescence change, 2) a color change, 3) a brightness change or 4) a combination thereof.

Embodiment 17. The system of embodiment 1, wherein the reporter comprises a nucleic acid sequence at least 80% identical to any one of the sequences set forth in Table 1.

Embodiment 18. The system of embodiment 17, Wherein the reporter comprises a nucleic acid sequence at least 90% identical to any one of the sequences set forth in Table 1.

Embodiment 19. The system of embodiment 18, wherein the reporter comprises a nucleic acid sequence at least 95% identical to any one of the sequences set forth in Table 1.

Embodiment 20. The system of embodiment 19, wherein the reporter comprises any one of the sequences set forth in Table 1.

Embodiment 21. The system of embodiment 2, wherein the programmable nuclease is a Type V Cas nuclease or a Type VI Cas nuclease.

Embodiment 22. The system of embodiment 2, wherein the programmable nuclease comprises an amino acid sequence at least 80% identical to any one of SEQ ID NOS: 26 or 43.

Embodiment 23. The system of embodiment 22, wherein the programmable nuclease comprises an amino acid sequence at least 90% identical to any one of SEQ ID NOS: 26 or 43.

Embodiment 24. The system of embodiment 23, wherein the programmable nuclease comprises an amino acid sequence at least 95% identical to any one of SEQ ID NOS: 26 or 43.

Embodiment 25. The system of embodiment 24, wherein the programmable nuclease comprises the amino acid sequence of any one of SEQ ID NOS: 26 or 43.

Embodiment 26. The system of embodiment 1, wherein the reporter is immobilized to the surface of the detection region using NHS-amine chemistry, streptavidin-biotin chemistry, epoxy-amine chemistry, maleimide-thiol chemistry, or a combination thereof.

Embodiment 27. The system of embodiment 1, wherein the reporter comprises an amine group and wherein the surface comprises an NHS coating.

Embodiment 28. The system of embodiment 1, wherein the guide nucleic acid is immobilized to the surface of the detection region.

Embodiment 29. The system of embodiment 2, wherein the programmable nuclease is immobilized to the surface of the detection region.

Embodiment 30. The system of any one of embodiments 1-29, further comprising a polymerase, a reverse transcriptase, or both; optionally wherein: (i) the polymerase is KAPA3G DNA polymerase, RAPIDXFIRE DNA polymerase, or ACAT77 DNA polymerase; and/or (ii) the reverse transcriptase is WARMSTART reverse transcriptase, RAPIDXFIRE reverse transcriptase, ACAT138 reverse transcriptase, and ACAT141.

Embodiment 31. A system for the multiplexed detection of a plurality of target nucleic acids, the system comprising: a detection region comprising a plurality of detection locations, each detection location of the plurality of detection locations comprising: i) a guide nucleic complementary to one of the plurality of target nucleic acids, or a portion thereof; ii) a reporter immobilized to a surface of the detection region, the reporter comprising a nucleic acid and a detection moiety, wherein a) the nucleic acid is at least 40 nucleotides in length; b) the nucleic acid comprises a double-stranded region; c) or a combination thereof, and wherein, at each detection location, cleavage of the reporter by a programmable nuclease activated upon hybridization to the one of the plurality of target nucleic acids releases the detection moiety from the nucleic acid, wherein the release of the detection moiety is configured to generate a signal at the detection location; and wherein the signal indicates a presence or absence of the one of the plurality of target nucleic acids at the detection location.

Embodiment 32. The system of embodiment 31, wherein the system comprises the programmable nuclease.

Embodiment 33. The system of embodiment 32, wherein the programmable nuclease is configured to form a complex with the guide nucleic acid and to be activated through binding of the guide nucleic acid to one of the plurality of target nucleic acids.

Embodiment 34. The system of embodiment 31, wherein the nucleic acid is at least 50 nucleotides in length.

Embodiment 35. The system of embodiment 34, wherein the nucleic acid comprises the single-stranded region and a double-stranded region.

Embodiment 36. The system of embodiment 35, wherein the single-stranded region is from about 5 to about 15 nucleotides in length.

Embodiment 37. The system of embodiment 35 or 36, wherein the double-stranded region is from about 45 to about 55 nucleotides in length.

Embodiment 38. The system of embodiment 35, wherein the single-stranded region is about 9 nucleotides in length, and wherein the double-stranded region is about 50 nucleotides in length.

Embodiment 39. The system of embodiment 31, wherein the nucleic acid is single-stranded.

Embodiment 40. The system of embodiment 39, wherein the single-stranded nucleic acid is at least about 50 nucleotides in length.

Embodiment 41. The system of embodiment 40, wherein the single-stranded nucleic acid is from about 55 to about 65 nucleotides in length.

Embodiment 42. The system of embodiment 31, wherein the detection moiety comprises a quencher moiety.

Embodiment 43. The system of embodiment 42, wherein the reporter comprises a fluorophore.

Embodiment 44. The system of embodiment 43, wherein the quencher moiety is configured to quench the fluorophore prior to the cleavage of the reporter.

Embodiment 45. The system of embodiment 31, wherein the detection moiety comprises a fluorophore.

Embodiment 46. The system of embodiment 31, wherein the signal is 1) a fluorescence change, 2) a color change, 3) a brightness change, 4) a wavelength change of a light, or 5) a combination thereof.

Embodiment 47. The system of embodiment 31, wherein the reporter comprises a nucleic acid sequence at least 80% identical to any one of the sequences set forth in Table 1.

Embodiment 48. The system of embodiment 47, Wherein the reporter comprises a nucleic acid sequence at least 90% identical to any one of the sequences set forth in Table 1.

Embodiment 49. The system of embodiment 48, wherein the reporter comprises a nucleic acid sequence at least 95% identical to any one of the sequences set forth in Table 1.

Embodiment 50. The system of embodiment 49, wherein the reporter comprises any one of the sequences set forth in Table 1.

Embodiment 51. The system of embodiment 2, wherein the programmable nuclease is a Type V Cas nuclease or a Type VI Cas nuclease.

Embodiment 52. The system of embodiment 2, wherein the programmable nuclease comprises an amino acid sequence at least 80% identical to any one of the sequences set forth in Table 3.

Embodiment 53. The system of embodiment 52, wherein the programmable nuclease comprises an amino acid sequence at least 90% identical to any one of the sequences set forth in Table 3.

Embodiment 54. The system of embodiment 53, wherein the programmable nuclease comprises an amino acid sequence at least 95% identical to any one of the sequences set forth in Table 3.

Embodiment 55. The system of embodiment 54, wherein the reporter comprises the amino acid sequence of any one of the sequences set forth in Table 3.

Embodiment 56. The system of embodiment 31, wherein the reporter is immobilized to the surface of the detection region using NHS-amine chemistry, streptavidin-biotin chemistry, epoxy-amine chemistry, maleimide-thiol chemistry, or a combination thereof.

Embodiment 57. The system of embodiment 31, wherein the reporter comprises an amine group and wherein the surface comprises an NHS coating.

Embodiment 58. The system of embodiment 31, wherein the guide nucleic acid is immobilized to the surface of the detection region.

Embodiment 59. The system of embodiment 32, wherein the programmable nuclease is immobilized to the surface of the detection region.

Embodiment 60. A method for detecting a target nucleic acid, the method comprising the steps of: a) applying a plurality of nucleic acids to a detection region comprising i) a programmable nuclease; ii) a guide nucleic acid complementary to the target nucleic acid, or a portion thereof; and iii) a reporter immobilized to a surface of a detection region, the reporter comprising a nucleic acid and a detection moiety, wherein 1) the nucleic acid is at least 40 nucleotides in length; 2) the nucleic acid comprises a double-stranded region; 3) or a combination thereof, and b) detecting a signal indicative of a presence or absence of a target nucleic acid in the plurality of nucleic acids, wherein the programmable nuclease is activated by binding of the target nucleic acid to the guide nucleic acid, wherein activation of the programmable nuclease cleaves the reporter, thereby releasing the detection moiety from the nucleic acid and generating the signal indicative of a presence or absence of the target nucleic acid.

Embodiment 61. The method of embodiment 60, wherein the programmable nuclease is immobilized to the detection region.

Embodiment 62. The method of embodiment 60, wherein the programmable nuclease is configured to form a complex with the guide nucleic acid and to be activated through binding of the guide nucleic acid to the target nucleic acid.

Embodiment 63. The method of embodiment 60, wherein the nucleic acid is at least 50 nucleotides in length.

Embodiment 64. The method of embodiment 63, wherein the nucleic acid comprises the single-stranded region and the double-stranded region.

Embodiment 65. The method of embodiment 64, wherein the single-stranded region is from about 5 to about 15 nucleotides in length.

Embodiment 66. The method of embodiment 64 or 65, wherein the double-stranded region is from about 45 to about 55 nucleotides in length.

Embodiment 67. The method of embodiment 64, wherein the single-stranded region is about 9 nucleotides in length, and wherein the double-stranded region is about 50 nucleotides in length.

Embodiment 68. The method of embodiment 60, wherein the nucleic acid is single-stranded.

Embodiment 69. The method of embodiment 68, wherein the single-stranded nucleic acid is at least about 50 nucleotides in length.

Embodiment 70. The method of embodiment 69, wherein the single-stranded nucleic acid is from about 55 to about 65 nucleotides in length.

Embodiment 71. The method of embodiment 70, wherein the detection moiety comprises a quencher moiety.

Embodiment 72. The method of embodiment 71, wherein the reporter comprises a fluorophore.

Embodiment 73. The method of embodiment 72, wherein the quencher moiety is configured to quench the fluorophore prior to the cleavage of the reporter.

Embodiment 74. The method of embodiment 60, wherein the detection moiety comprises a fluorophore.

Embodiment 75. The method of embodiment 60, wherein the signal is 1) a fluorescence change, 2) a color change, 3) a brightness change, or 4) a combination thereof.

Embodiment 76. The method of embodiment 60, wherein the reporter comprises a nucleic acid sequence at least 80% identical to any one of the sequences set forth in Table 1.

Embodiment 77. The method of embodiment 76, Wherein the reporter comprises a nucleic acid sequence at least 90% identical to any one of the sequences set forth in Table 1.

Embodiment 78. The method of embodiment 77, wherein the reporter comprises a nucleic acid sequence at least 95% identical to any one of the sequences set forth in Table 1.

Embodiment 79. The method of embodiment 78, wherein the reporter comprises any one of the sequences set forth in Table 1.

Embodiment 80. The method of embodiment 61, wherein the programmable nuclease is a Type V Cas nuclease or a Type VI Cas nuclease.

Embodiment 81. The method of embodiment 61, wherein the programmable nuclease comprises an amino acid sequence at least 80% identical to any one of the sequences set forth in Table 3.

Embodiment 82. The method of embodiment 81, wherein the programmable nuclease comprises an amino acid sequence at least 90% identical to any one of the sequences set forth in Table 3.

Embodiment 83. The method of embodiment 82, wherein the programmable nuclease comprises an amino acid sequence at least 95% identical to any one of the sequences set forth in Table 3.

Embodiment 84. The system of embodiment 83, wherein the programmable nuclease comprises the amino acid sequence of any one of the sequences set forth in Table 3.

Embodiment 85. The method of embodiment 60, wherein the reporter is immobilized to the surface of the detection region using NHS-amine chemistry, streptavidin-biotin chemistry, epoxy-amine chemistry, maleimide-thiol chemistry, or a combination thereof.

Embodiment 86. The method of embodiment 60, wherein the reporter comprises an amine group and wherein the surface comprises an NHS coating.

Embodiment 87. The method of embodiment 60, wherein the guide nucleic acid is immobilized to the surface of the detection region.

Embodiment 88. The method of embodiment 61, wherein the programmable nuclease is immobilized to the surface of the detection region.

Embodiment 89. The method of any one of embodiments 60-88, further comprising a polymerase, a reverse transcriptase, or both; optionally wherein: (i) the polymerase is KAPA3G DNA polymerase, RAPIDXFIRE DNA polymerase, or ACAT77 DNA polymerase; and/or (ii) the reverse transcriptase is WARMSTART reverse transcriptase, RAPIDXFIRE reverse transcriptase, ACAT138 reverse transcriptase, and ACAT141.

Embodiment 90. A method for detecting a plurality of target nucleic acids, the method comprising the steps of: a) applying a plurality of nucleic acids to the detection region comprising a plurality of detection locations, each detection location comprising i) a programmable nuclease; ii) a guide nucleic complementary to one of the plurality of target nucleic acids, or a portion thereof; iii) a reporter immobilized to a surface of a detection region, the reporter comprising a nucleic acid and a detection moiety, wherein 1) the nucleic acid is at least 40 nucleotides in length; 2) the nucleic acid comprises a double-stranded region; 3) or a combination thereof, and b) detecting, at each detection location, a signal indicative of a presence or absence of one of a plurality of target nucleic acids in the plurality of nucleic acids, wherein, at each detection location, the programmable nuclease is activated by binding of the one of the plurality of target nucleic acids to the guide nucleic acid, wherein activation of the programmable nuclease cleaves the reporter, thereby releasing the detection moiety from the nucleic acid and generating the signal indicative of a presence or absence of the one of the plurality of target nucleic acids.

Embodiment 91. The method of embodiment 90, wherein the programmable nuclease is immobilized to the detection location.

Embodiment 92. The method of embodiment 91, wherein the programmable nuclease is configured to form a complex with the guide nucleic acid and to be activated through binding of the guide nucleic acid to the target nucleic acid.

Embodiment 93. The method of embodiment 90, wherein the nucleic acid is at least 50 nucleotides in length.

Embodiment 94. The method of embodiment 93, wherein the nucleic acid comprises the single-stranded region and a double-stranded region.

Embodiment 95. The method of embodiment 94, wherein the single-stranded region is from about 5 to about 15 nucleotides in length.

Embodiment 96. The method of embodiment 94 or 95, wherein the double-stranded region is from about 45 to about 55 nucleotides in length.

Embodiment 97. The method of embodiment 94, wherein the single-stranded region is about 9 nucleotides in length, and wherein the double-stranded region is about 50 nucleotides in length.

Embodiment 98. The method of embodiment 90, wherein the nucleic acid is single-stranded.

Embodiment 99. The method of embodiment 98, wherein the single-stranded nucleic acid is at least about 50 nucleotides in length.

Embodiment 100. The method of embodiment 99, wherein the single-stranded nucleic acid is from about 55 to about 65 nucleotides in length.

Embodiment 101. The method of embodiment 100, wherein the detection moiety comprises a quencher moiety.

Embodiment 102. The method of embodiment 101, wherein the reporter comprises a fluorophore.

Embodiment 103. The method of embodiment 102, wherein the quencher moiety is configured to quench the fluorophore prior to the cleavage of the reporter.

Embodiment 104. The method of embodiment 90, wherein the detection moiety comprises a fluorophore.

Embodiment 105. The method of embodiment 90, wherein the signal is 1) a fluorescence change, 2) a color change, 3) a brightness change, or 4) a combination thereof.

Embodiment 106. The method of embodiment 90, wherein the reporter comprises a nucleic acid sequence at least 80% identical to any one of the sequences set forth in Table 1.

Embodiment 107. The method of embodiment 106, Wherein the reporter comprises a nucleic acid sequence at least 90% identical to any one of the sequences set forth in Table 1.

Embodiment 108. The method of embodiment 107, wherein the reporter comprises a nucleic acid sequence at least 95% identical to any one of the sequences set forth in Table 1.

Embodiment 109. The method of embodiment 108, wherein the reporter comprises any one of the sequences set forth in Table 1.

Embodiment 110. The method of embodiment 91, wherein the programmable nuclease is a Type V Cas nuclease or a Type VI Cas nuclease.

Embodiment 111. The method of embodiment 91, wherein the programmable nuclease comprises an amino acid sequence at least 80% identical to any one of the sequences set forth in Table 3.

Embodiment 112. The method of embodiment 111, wherein the programmable nuclease comprises an amino acid sequence at least 90% identical to any one of the sequences set forth in Table 3.

Embodiment 113. The method of embodiment 112, wherein the programmable nuclease comprises an amino acid sequence at least 95% identical to any one of the sequences set forth in Table 3.

Embodiment 114. The method of embodiment 113, wherein the programmable nuclease comprises the amino acid sequence of any one of the sequences set forth in Table 3.

Embodiment 115. The method of embodiment 90, wherein the reporter is immobilized to the surface of the detection region using NHS-amine chemistry, streptavidin-biotin chemistry, epoxy-amine chemistry, maleimide-thiol chemistry, or a combination thereof.

Embodiment 116. The method of embodiment 90, wherein the reporter comprises an amine group and wherein the surface comprises an NHS coating.

Embodiment 117. The method of embodiment 90, wherein the guide nucleic acid is immobilized to the surface of the detection region.

Embodiment 118. The method of embodiment 91, wherein the programmable nuclease is immobilized to the surface of the detection region.

Embodiment 119. A nucleic acid molecule comprising a nucleic acid sequence at least 80% identical to any one of the sequences set forth in Table 1.

Embodiment 120. The nucleic acid of embodiment 119, comprising a nucleic acid sequence at least 90% identical to any one of the sequences set forth in Table 1.

Embodiment 121. The nucleic acid of embodiment 120, comprising a nucleic acid sequence at least 95% identical to any one of the sequences set forth in Table 1.

Embodiment 122. The nucleic acid of embodiment 121, comprising a nucleic acid sequence of any one of the sequences set forth in Table 1.

Embodiment 123. A system for detecting a target nucleic acid in a sample, the system comprising one or more units, wherein a unit comprises: (a) a non-naturally occurring guide nucleic acid immobilized to a surface by a linkage; and (b) a plurality of reporters immobilized to the surface in proximity to the non-naturally occurring guide nucleic acid; wherein the non-naturally occurring guide nucleic acid comprises (i) one or more stabilized nucleotides, (ii) a repeat region comprising a first end and a second end, and (iii) a spacer region that hybridizes to a segment of the target nucleic acid or an amplicon thereof; wherein the first end of the repeat region is joined to the linkage, and the second end of the repeat region is joined to the spacer region; wherein the one or more stabilized nucleotides (i) lack a 2′—OH group, and (ii) are located within 10 nucleotides of a terminal hairpin nucleotide in the first end of the repeat region; wherein the non-naturally occurring guide nucleic acid is effective to form a complex with a programmable nuclease that is activated upon binding the target nucleic acid or amplicon thereof; wherein formation of the activated complex is effective to induce detectable transcollateral cleavage of the reporters; and wherein (i) the linkage comprises a tether having a length at least that of a 10-nucleotide DNA molecule, and/or (ii) the reporters are immobilized to the surface by attachment to the non-naturally occurring guide nucleic acid, the linkage, or both.

Embodiment 124. The system of embodiment 123, wherein the one or more stabilized nucleotides comprise DNA nucleotides, modified RNA nucleotides, or both.

Embodiment 125. The system of embodiment 123 or 124, wherein the one or more stabilized nucleotides comprise a plurality of stabilized nucleotides.

Embodiment 126. The system of any one of embodiments 123-125, wherein the one or more stabilized nucleotides are located (a) within 2 to 6 nucleotides of the terminal hairpin nucleotide, and/or (b) within the repeat region.

Embodiment 127. The system of any one of embodiment 123-126, wherein the one or more stabilized nucleotides are effective to reduce a rate of cleavage of the non-naturally occurring guide nucleic acid by the programmable nuclease, relative to that for a corresponding non-naturally occurring guide nucleic acid that consists of RNA.

Embodiment 128. The system of any one of embodiments 123-127, wherein the linkage comprises the tether, and the tether comprises a hydrocarbon chain.

Embodiment 129. The system of any one of embodiments 123-127, wherein the linkage comprises the tether, and the tether comprises a tether polynucleotide.

Embodiment 130. The system of embodiment 129, wherein the tether polynucleotide: (a) is 10 to 200 nucleotides in length, 10 to 100 nucleotides in length, 12 to 60 nucleotides in length, or 15 to 20 nucleotides in length; (b) is single stranded; and/or (c) comprises DNA.

Embodiment 131. The system of any one of embodiments 123-130, wherein the terminal hairpin nucleotide: (a) is a 5′ terminal hairpin nucleotide located 3′ relative to the stabilized nucleotides; and/or (b) is a terminal nucleotide within a sequence of nucleotides that is complementary to a sequence of nucleotides in the second end of the repeat sequence and proximal to the linkage.

Embodiment 132. The system of any one of embodiments 123-131, wherein the reporters are immobilized to the surface by attachment to the non-naturally occurring guide nucleic acid, the linkage, or both.

Embodiment 133. The system of embodiment 132, wherein at least one of the reporters is immobilized to the surface by attachment to an end of the non-naturally occurring guide nucleic acid that is distal to the linkage.

Embodiment 134. The system of embodiment 132, wherein at least one of the reporters is immobilized to the surface by attachment to the linkage.

Embodiment 135. The system of any one of embodiments 123-134, wherein each of the reporters comprises a fluorophore and a quencher, and wherein cleavage of the reporters is effective to produce a detectable loss of the quencher.

Embodiment 136. The system of any one of embodiments 123-134, wherein each of the reporters comprises a detection moiety, and wherein cleavage of the reporters is effective to produce a detectable loss of the detection moiety, optionally wherein the detection moiety comprises a fluorophore.

Embodiment 137. The system of any one of embodiments 123-136, wherein (a) the one or more units comprise a plurality of different units, and (b) each of the different units comprises a non-naturally occurring guide nucleic acid comprising a spacer region with a different sequence.

Embodiment 138. The system of any one of embodiments 123-137, further comprising the programmable nuclease complexed with the non-naturally occurring guide nucleic acid.

Embodiment 139. The system of embodiment 138, wherein the programmable nuclease comprises an RuvC catalytic domain.

Embodiment 140. The system of embodiment 139, wherein the programmable nuclease is a type V CRISPR/Cas effector protein.

Embodiment 141. The system of embodiment 140, wherein the type V CRISPR/Cas effector protein is a Cas12 protein, a Cas14 protein, or a CasΦ protein.

Embodiment 142. The system of embodiment 141, wherein the type V CRISPR/Cas effector protein is a Cas 12 protein comprising a Cas12a, a Cas12b, a Cas12c, a Cas12d, a Cas12e, a Cas12f, a Cas12g, a Cas12h, a Cas12i, a Cas12j, or a Cas12k.

Embodiment 143. The system of embodiment 141, wherein the type V CRISPR/Cas effector protein comprises an amino acid sequence that is at least 80%, 90%, 95%, or 100% identical to SEQ ID NO: 43.

Embodiment 144. The system of embodiment 139, wherein the programmable nuclease is a type VI CRISPR/Cas effector protein.

Embodiment 145. The system of embodiment 144, wherein the type VI

CRISPR/Cas effector protein is a Cas13 protein; optionally wherein the Cas13 protein comprises a a Cas13a, a Cas13b, a Cas13c, a Cas13d, a Cas13e, or a Cas13f.

Embodiment 146. A system for detecting a target nucleic acid in a sample, the system comprising one or more units, wherein a unit comprises: (a) a non-naturally occurring guide nucleic acid; (b) a plurality of reporters; and (c) a programmable nuclease; wherein the non-naturally occurring guide nucleic acid comprises a spacer region that hybridizes to a segment of the target nucleic acid or an amplicon thereof; wherein the segment hybridized by the spacer region is directly adjacent to a sequence that is complementary to a noncanonical PAM sequence; wherein the noncanonical PAM sequence differs from a naturally-occurring PAM sequence for a reference Cas nuclease; wherein the reference Cas nuclease is a Cas nuclease of the same type as the programmable nuclease; wherein the non-naturally occurring guide nucleic acid is effective to form a complex with the programmable nuclease that is activated upon binding the target nucleic acid or amplicon thereof; and wherein formation of the activated complex is effective to induce detectable transcollateral cleavage of the reporters.

Embodiment 147. The system of embodiment 146, wherein the programmable nuclease comprises a Type V Cas nuclease.

Embodiment 148. The system of embodiment 147, wherein the programmable nuclease is a Cas 12 protein, a Cas14 protein, or a CasΦ protein.

Embodiment 149. The system of embodiment 148, wherein the programmable nuclease is a Cas 12 protein comprising a Cas12a, a Cas12b, a Cas12c, a Cas12d, a Cas12e, a Cas12f, a Cas12g, a Cas12h, a Cas12i, a Cas12j, or a Cas12k.

Embodiment 150. The system of embodiment 149, wherein the programmable nuclease is a Cas 12a protein.

Embodiment 151. The system of embodiment 147, wherein the programmable nuclease comprises an amino acid sequence that is at least 80%, 90%, 95%, or 100% identical to SEQ ID NO: 43.

Embodiment 152. The system of any one of embodiments 146-151, wherein the canonical PAM sequence comprises TTTN or YYN.

Embodiment 153. The system of embodiment 152, wherein the canonical PAM sequence comprises TTTA.

Embodiment 154. The system of any one of embodiments 146-153, wherein the noncanonical PAM sequence comprises CCGT, TCCG, CCCT, TGTC, TTGT, GGGC, TGGG, TTGG, TTTG, CTTT, CCTT, TCCT, TTCC, or CTGT.

Embodiment 155. The system of any one of embodiments 146-153, wherein the noncanonical PAM sequence comprises CCG, TTC, CCC, TGT, TTG, TCTT, TTCT, TTTT, CTTT, CCTT, TCGG, CTCG, TTTG, GGGC, TGGG, TTGG, TCCT, TTCC, or CTGT.

Embodiment 156. The system of any one of embodiments 152-155, wherein the programmable nuclease is a Type V Cas nuclease comprising an amino acid sequence that is at least 80%, 90%, 95%, or 100% identical to SEQ ID NO: 43.

Embodiment 157. The system of any one of embodiments 146-156, wherein the non-naturally occurring guide nucleic acid, the reporters, or both are immobilized to a surface.

Embodiment 158. The system of embodiment 157, wherein (a) the one or more units comprise a plurality of different units, and (b) each of the different units comprises a non-naturally occurring guide nucleic acid comprising a spacer region with a different sequence.

Embodiment 159. The system of any one of embodiments 146-158, wherein each of the reporters comprises a fluorophore and a quencher, and wherein cleavage of the reporters is effective to produce a detectable loss of the quencher.

Embodiment 160. The system of any one of embodiments 146-158, wherein each of the reporters comprises a detection moiety, and wherein cleavage of the reporters is effective to produce a detectable loss of the detection moiety, optionally wherein the detection moiety comprises a fluorophore.

Embodiment 161. A method of assaying for one or more target nucleic acids in a sample, the method comprising: (a) contacting the system of any one of embodiments 123-160 with the sample; (b) cleaving the reporters in response to presence of the target nucleic acid or amplicon thereof; and (c) detecting a change in signal resulting from cleavage of the reporters; wherein the detection identifies the target nucleic acid in the sample.

Embodiment 162. The method of embodiment 161, further comprises amplifying the one or more target nucleic acids before or during said contacting.

Embodiment 163. A method of detecting a target nucleic acids in a sample, the method comprising: (a) contacting the sample with a composition comprising primers, a polymerase, a programmable nuclease, a non-naturally occurring guide nucleic acid, and a reporter; (b) amplifying the target nucleic acid with the primers and polymerase; (c) forming an activated complex comprising (i) the programmable nuclease, and (ii) the non-naturally occurring guide nucleic acid hybridized to the target nucleic acid or an amplicon thereof; (d) cleaving the reporter with the activated complex; and (e) detecting a change in signal resulting from cleavage of the reporters, thereby detecting the target nucleic acid in the sample; wherein steps (a) through (d) are performed in a single reaction volume; and wherein steps (b) through (d) comprise (i) isothermal incubation, or (ii) incubation at a first temperature followed by incubation at a reduced second temperature.

Embodiment 164. A method of detecting a first or second target nucleic acid in a sample, the method comprising: (a) contacting the sample with a composition comprising amplification reagents, a programmable nuclease, a first non-naturally occurring guide nucleic acid, a second non-naturally occurring guide nucleic acid, and reporters; (b) amplifying the first or second target nucleic acid; (c) forming an activated complex comprising the programmable nuclease and (i) the first non-naturally occurring guide nucleic acid hybridized to the first target nucleic acid or an amplicon thereof, or (ii) the second non-naturally occurring guide nucleic acid hybridized to the second target nucleic acid or an amplicon thereof; (d) cleaving the reporters with the activated complex; and (e) detecting a change in signal resulting from cleavage of the reporters at a first level or a second level, thereby detecting the first or second target nucleic acid in the sample; wherein steps (a) through (d) are performed in a single reaction volume; wherein the first level is above a first threshold and below a second threshold; wherein the second level is above the second threshold; wherein presence of the first target nucleic acid in the sample results in detection at the first level; and wherein presence of the second target nucleic acid in the sample results in detection at the second level.

### Examples

The following examples are included for illustrative purposes only and are not intended to limit the scope of the disclosure. It will be understood by those of skill in the art that numerous and various modifications can be made to yield essentially similar results without departing from the spirit of the present disclosure.

### Example 1: Designs of Improved Reporters for Immobilization on Substrates

Provided herein are methods for optimizing the cleavage of fluorescent reporters immobilized on a substrate in a system for detecting a target nucleic acid described herein. A reporter is attached to a surface via any of the chemistries described herein (e.g., amine chemistry). The immobilized reporter then can be contacted with a programmable nuclease and a guide nucleic acid in a trans cleavage reaction. FIGS. 1A-1E show the designs of various immobilized reporters. The arrow below each reporter indicates the expected signal change when the reporter is cleaved by a programmable nuclease. In some cases, cleavage of the reporter may release a fluorophore, as shown in FIGS. 1A, 1C, and 1E, thereby decreasing the fluorescence signal of the reporter. In other cases, as shown in FIGS. 1D and 1D, cleavage of the reporter may release a quencher moiety, thereby increasing the fluorescent signal of the reporter. FIGS. 1A (e.g., reporter 112) and 1B (e.g., reporter 136) depict single-stranded nucleic acid reporters that will show decreased and increased fluorescence, respectively. FIGS. 1C (e.g., reporter 204) and 1D (e.g., reporter 203) depict two exemplary optimized nucleic acid reporters that will show decreased and increased fluorescence, respectively. These reporters have increased length compared to those of FIGS. 1A and 1B and contain double-stranded regions. FIG. 1E (e.g., reporter 200) shows a single-stranded nucleic acid reporter with an increased length relative to those of FIGS. 1A-1D. Without being bound by any theory, the increased length and/or double-stranded in region in the reporters of FIGS. 1C-1E, relative to those of FIGS. 1A and 1B decrease the steric hindrance of the access of the cleavage sites on the reporter by programmable nuclease. The increased length also decreases interference of access to the cleavage site by the programmable nuclease caused, at least in part, by the surface chemistry of the substrate. FIG. 4 depicts an image of an exemplary substrate immobilized with guide nucleic acids and reporters. Each spot represents a discrete detection location on the substrate immobilized with at least one guide nucleic acid and at least one reporter.

### Example 2: Workflow for Detecting Target Nucleic Acids using Immobilized Reporters

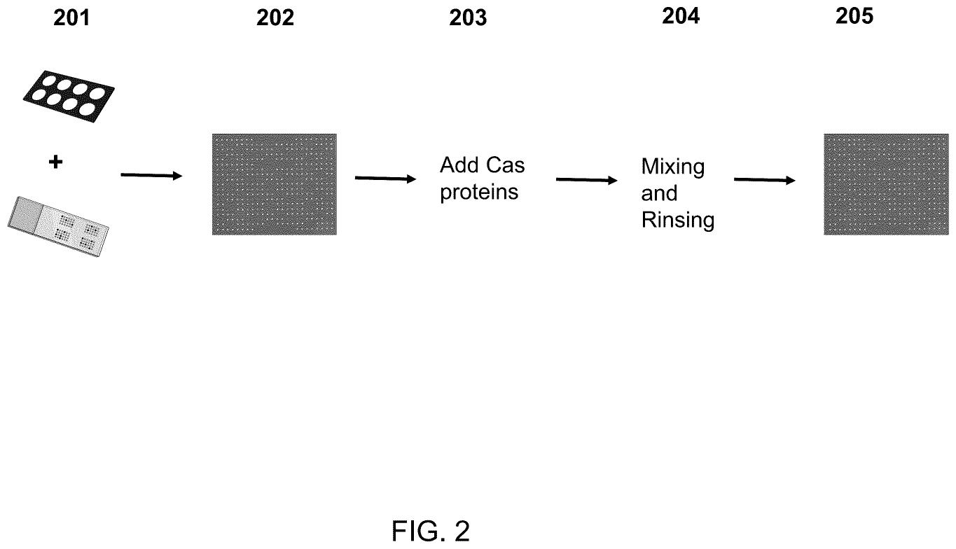

Provided herein are methods for detecting nucleic acids by cleavage of immobilized reporters and/or guide nucleic acids. FIG. 2 shows an exemplary workflow for detecting target nucleic acids using immobilized reporters. At step 201, the reporters and/or guide nucleic acids are immobilized on a glass slide, such as by NHS-amine chemistry. At step 202, the slide is imaged in an imaging buffer to identify the starting signal at each discrete detection position. Each spot represents one discrete detection position. At step 203, a programmable nuclease, such as a Cas12 protein, is added onto the slide along with target nucleic acids. The programmable nuclease may also be added onto the slide with non-target nucleic acids to produce a negative control signal. The guide nucleic acid can also be added if it has not been immobilized on the slide. At step 204, the slides are placed on a thermomixer for a period of time (e.g., 20 minutes) to allow for the programmable nuclease, the guide nucleic acid, the reporter, and the target nucleic acid to mix thoroughly. The presence of both the target nucleic acid and the guide nucleic acid that can hybridize with the target nucleic acid can trigger the trans cleavage activity of the programmable nuclease, thereby cleaving the immobilized reporter at the discrete detection position upon which a reporter is immobilized. At step 205, the slide is imaged again in the imagining buffer to identify the changes of the signal at each discrete detection position.

### Example 3: Trans-Cleavage Reactions Successfully Cleaves Immobilized Reporters

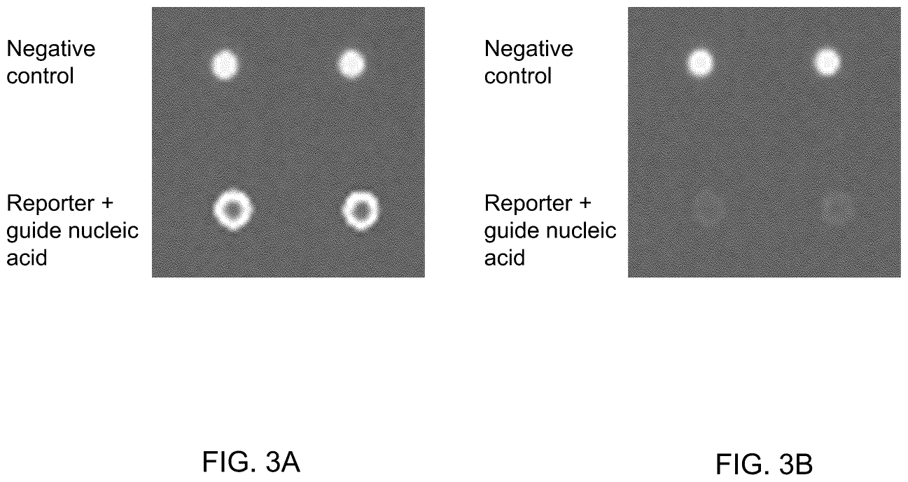

FIGS. 3A and 3B show the image of a slide on which a surface-based trans-cleavage assay was carried out thereto using the workflow of FIG. 2. FIG. 3A shows the slide before addition of the programmable nuclease and the target nucleic acid thereto. FIG. 3B shows the slide after addition of the programmable nuclease and the target nucleic acid thereto. Reporter 200 (as shown in FIG. 1C) and a guide RNA were immobilized on a glass slide (from Applied Microarrays). In the presence of a target nucleic acid and the guide nucleic acid that hybridized with the target nucleic acid, a programmable nuclease having an amino acid identical to that of SEQ ID NO: 43 was activated, triggering the trans cleavage activity and resulting in the cleavage of immobilized reporter 200, as described in Example 1. The cleavage resulted in a decrease of visible signal 20 minutes after the addition of the programmable nuclease and the target nucleic acid to the slide (the two replicates in the bottom rows). In contrast, the negative controls using uncleavable Lumiprobes did not show any changes of signals. This result suggests that the surface-based trans-cleavage assay reactions can result in visible and specific changes in the images of the array.

### Example 4: Specific and Robust Detection of Target Nucleic Acid Using Surface-Based Trans-cleavage Assays and Optimized Immobilized Reporters

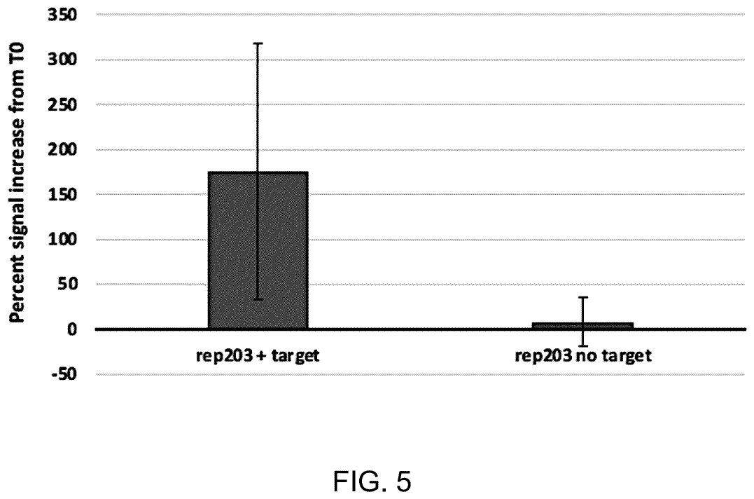

Surface-based trans-cleavage assays provided specific cleavage of immobilized reporters only in the presence of a target nucleic acid and a complementary guide nucleic acid. ˜5 μM partially double-stranded reporter 203 (as shown in FIG. 1D and described in Table 1) and ˜2.5 μM guide RNA complementary to a target nucleic acid were immobilized on a detection location of an array by NHS-amine chemistry, as described in Example 2. The array contained 10-12 spots each. FIG. 5 summarizes the results of 30 arrays. FIG. 7 shows box plots of the signal increase for each array. 20 minutes after the addition of a programmable nuclease having SEQ ID NO: 43 and the target nucleic acids, the fluorescence signal increased by 150%, relative to before the programmable nuclease and the target nucleic acids were added. The increase of the signal was specific: arrays without the addition of the target nucleic acids showed minimal increase in the fluorescence signal. Therefore, surface-based trans cleavage assays and improved reporters allowed for specific detection of the target nucleic acid.

### Example 5: Trans-Cleavage Reactions Successfully Cleavage Immobilized Reporters with Multiple gRNAs

Trans-cleavage reactions were shown to successfully cleave NHS-amine bound reporter molecules in a series of multiplex assays carried out on microarray slides. The sequences of the reporters used in each of the assays described are provided in Table 1 herein. The guides used in each of the assays are provided in Table 4.

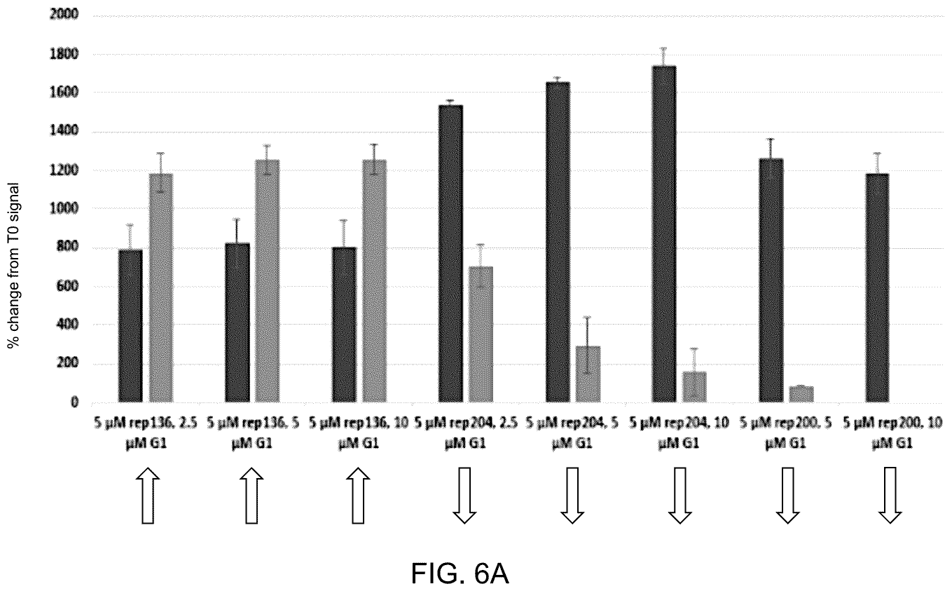

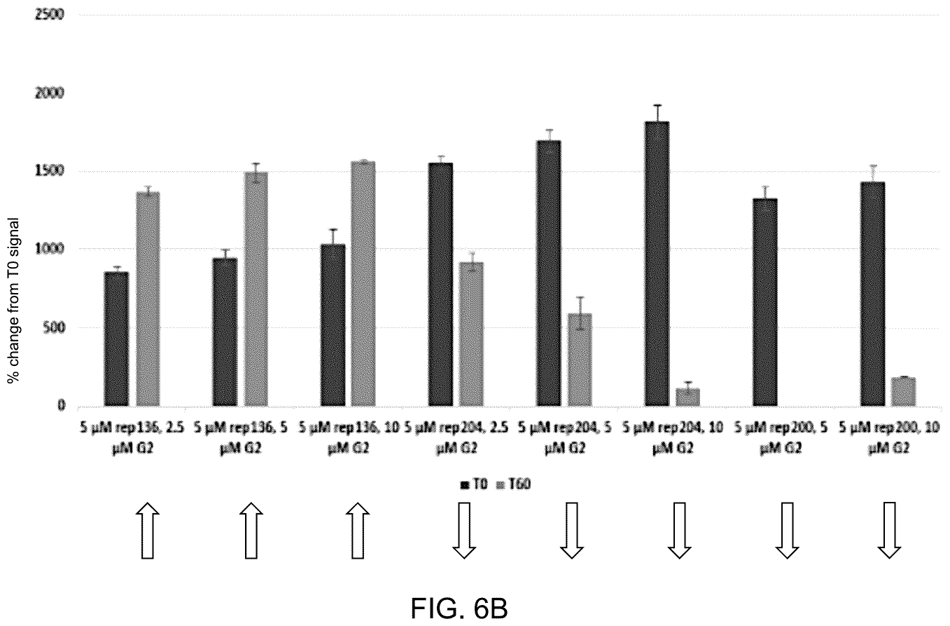

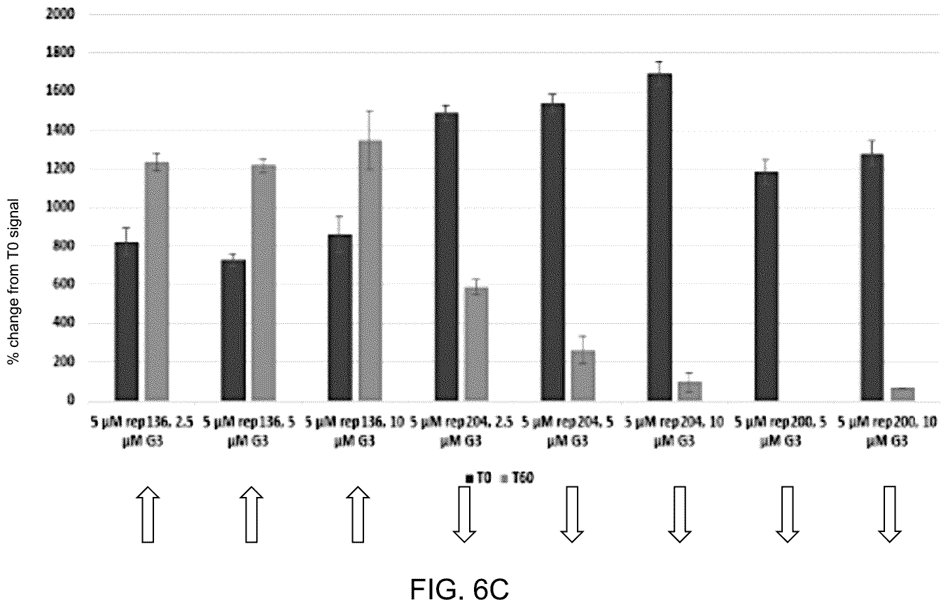

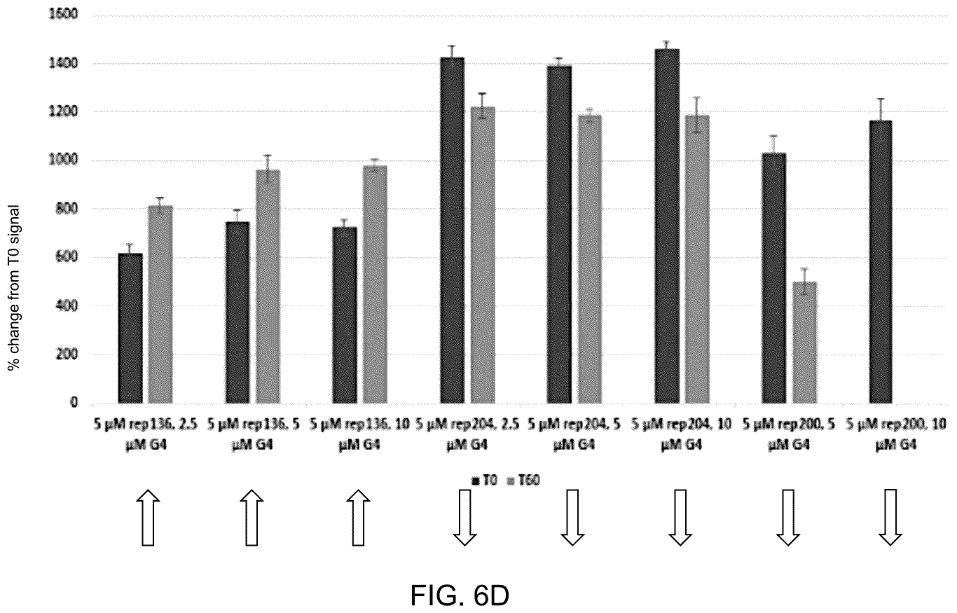

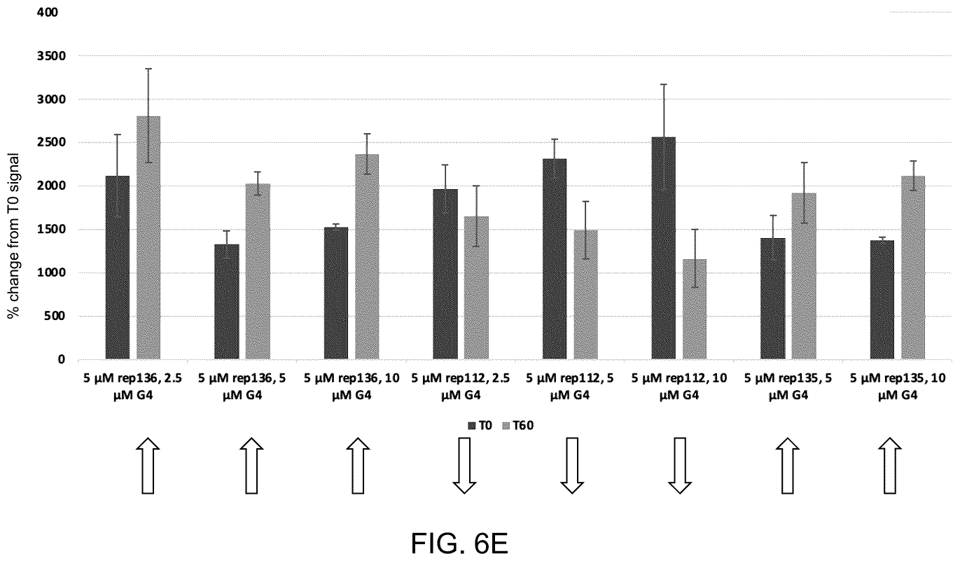

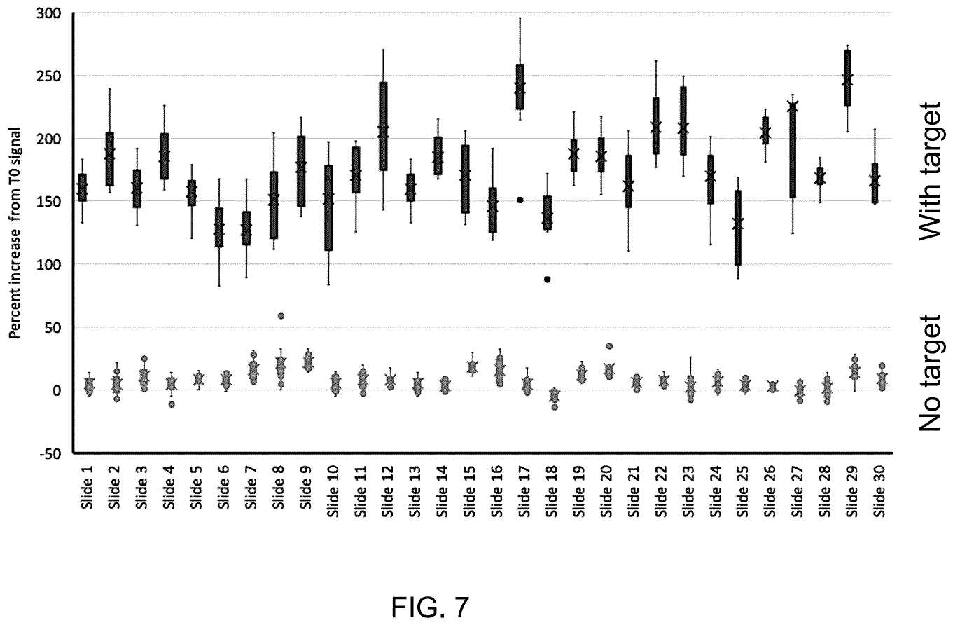

Four different assays—each assay using one of four guide nucleic acids—were carried out. For each reaction, reporters and guide nucleic acids—in a range of concentrations—were immobilized on a detection region at different discrete detection locations on a microarray slide from HD Suromodics. After immobilization, the programmable nuclease of SEQ ID NO: 43 and a buffer were added to the wells, and the plate was incubated at 55° C. on a thermomixer shaking at 500 RPM for 1 hour. The plates were imaged under the leica 100% FAM channel at 500 ms exposure before and 60 minutes after the addition of the programmable nucleases. FIGS. 6A-6D show the results from each of the four assays, with FIG. 6A showing the reactions performed with the guide G1 at varying concentrations and reporters rep 136, rep204, and rep200 at 5 μM; FIG. 6B showing the reactions performed with the guide G2 at varying concentrations and reporters rep 136, rep204, and rep200 at 5 μM; FIG. 6C showing the reactions performed with the guide G3 at varying concentrations and reporters rep 136, rep204, and rep200 at 5 μM; and FIG. 6D showing the reactions performed with the guide G4 at varying concentrations and reporters rep 136, rep204, and rep200 at 5 μM. Finally, FIG. 6E shows the reactions performed with the guide G4 at varying concentrations and reporters rep 136, rep 135, and rep 112 at 5 μM.

As shown in FIGS. 6A-6E, in the presence of the target nucleic acids and the guide RNAs, the fluorescence moiety (i.e., FAM) of reporter 200 (shown in FIG. 1E), 204 (shown in FIG. 1C), and 112 (shown in FIG. 1A) was freed by the cleavage of the programmable nuclease, resulting in a significantly decreased fluorescence signal after 60 minutes of the addition of the programmable nuclease. For example, reporter 200 showed significant fluorescence signal decrease over the course of the reaction. As shown in FIGS. 6A-6E, in the presence of the target nucleic acids and the guide RNAs, the quencher moiety of reporter 136 (shown in FIG. 1B), 203 (shown in FIG. 1D), and 135 (substantially similar to FIG. 1B) was freed by the cleavage of the programmable nuclease, resulting in a significantly increased fluorescence signal after 60 minutes of the addition of the programmable nuclease.

### Example 6: Trans-Cleavage Reactions Successfully Cleavage Immobilized Reporters on Various Substrates

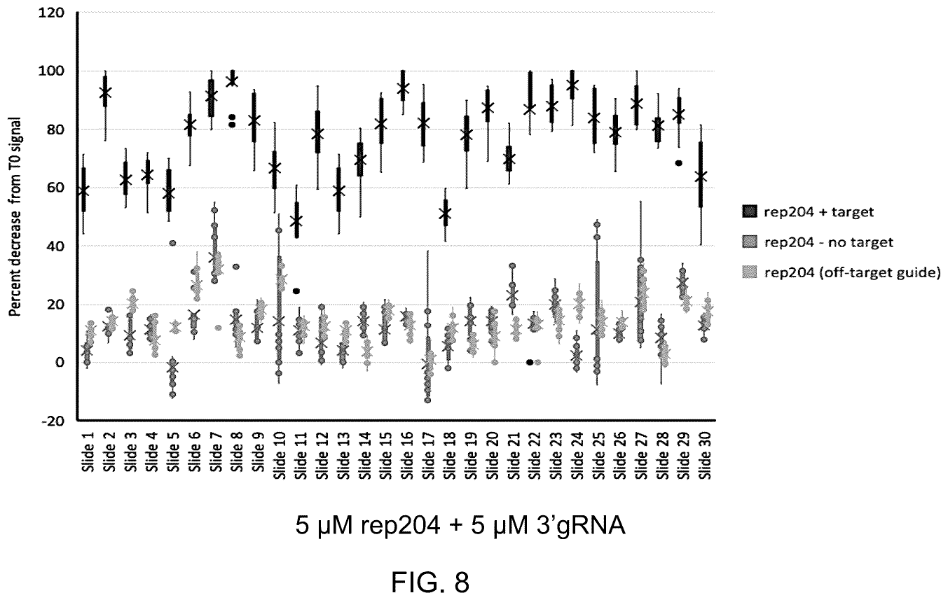

The high specificity of the immobilized reporters was reproduced in different batches of substrate. Thirty different slides, each with 10-12 spots were immobilized with reporter 203 (as shown in FIG. 1C) or reporter 204 (as shown in FIG. 1D) were constructed. ˜5 μM reporter and ˜5 μM guide nucleic acids were immobilized on each discrete detection location, respectively NO: 34, significant increases of fluorescence were observed in all slides, relative to the negative controls without any target nucleic acid, as shown in FIG. 7. Furthermore, when using reporter 204, significant decreases of fluorescence were observed in all slides, relative to the negative controls without any target nucleic acid or non-complementary guide nucleic acids, as shown in FIG. 8.

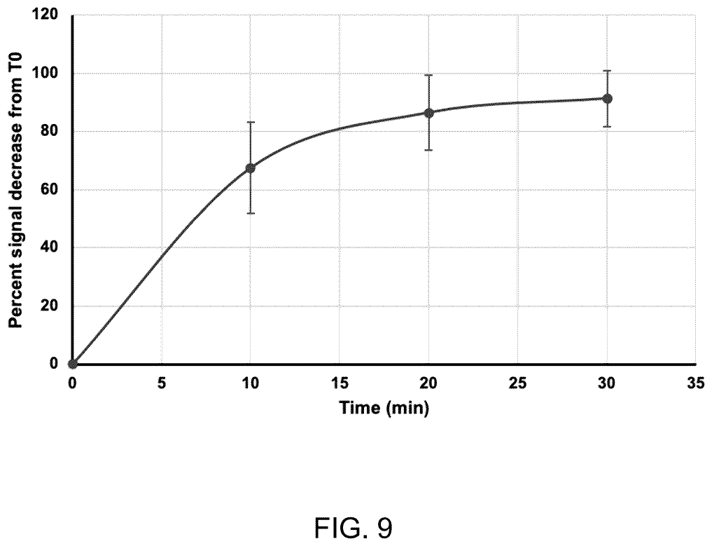

To assay for when signals of immobilized reporters become detectable, the signal of reporter 204 was assayed at multiple time points after the programmable nuclease of SEQ ID NO: 43 and the target nucleic acid were added to the immobilized reporter 204 and the guide nucleic acid complementary to the target nucleic acid. ˜5 μM reporter and ˜5 μM guide nucleic acids were immobilized on each discrete detection location. As shown in FIG. 9, percentage decrease of the reporter plateaued 20 minutes after the addition of the Cas protein and the target nucleic acid. Hence, robust signals resulted from the cleavage of the immobilized reporter can be observed soon after the activation of the Cas protein.

### Example 7: Combining Immobilized Reporters with Instruments Thereof

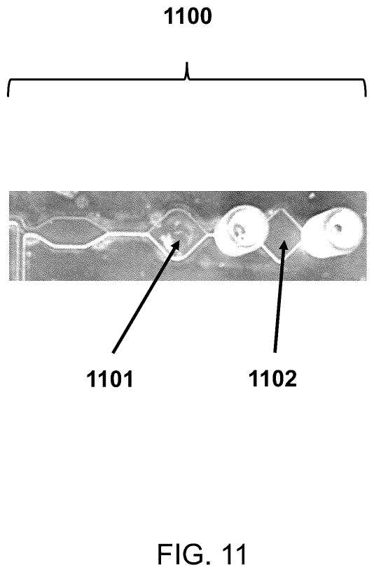

Provided herein are methods, systems, and instruments for detecting immobilized reporters in a surface-based trans-cleavage assay and optimized immobilized reporters. For example, the surface-based trans-cleavage assay using the optimized immobilized reporters can be implemented using a microfluidic device as shown in FIG. 11. FIG. 11 illustrates an exemplary microfluidic device 1100. For example, the solution comprising the target nucleic acids can be amplified in a first reaction chamber 1101 in the device. The amplified target nucleic acids can be transported to a second reaction chamber 1102 of the device comprising a plurality of immobilized guide RNAs. The reporters described in this disclosure are immobilized on the substrate surface of the second reaction chamber 1102 at discrete locations and are co-localized with the immobilized guide RNAs such that each discrete detection location comprises at least one reporter and at least one guide RNA. Improved reporters and/or guide nucleic acids described in this disclosure can be immobilized to the surface of the detection chamber 1102. Programmable nucleases can then be added to the detection chamber 1102. The binding of the target nucleic acid and the complementary guide nucleic acid can activate the programmable nuclease and trigger the cleavage of the immobilized reporters as described herein.

### Example 8: Immobilized Guide Stabilization

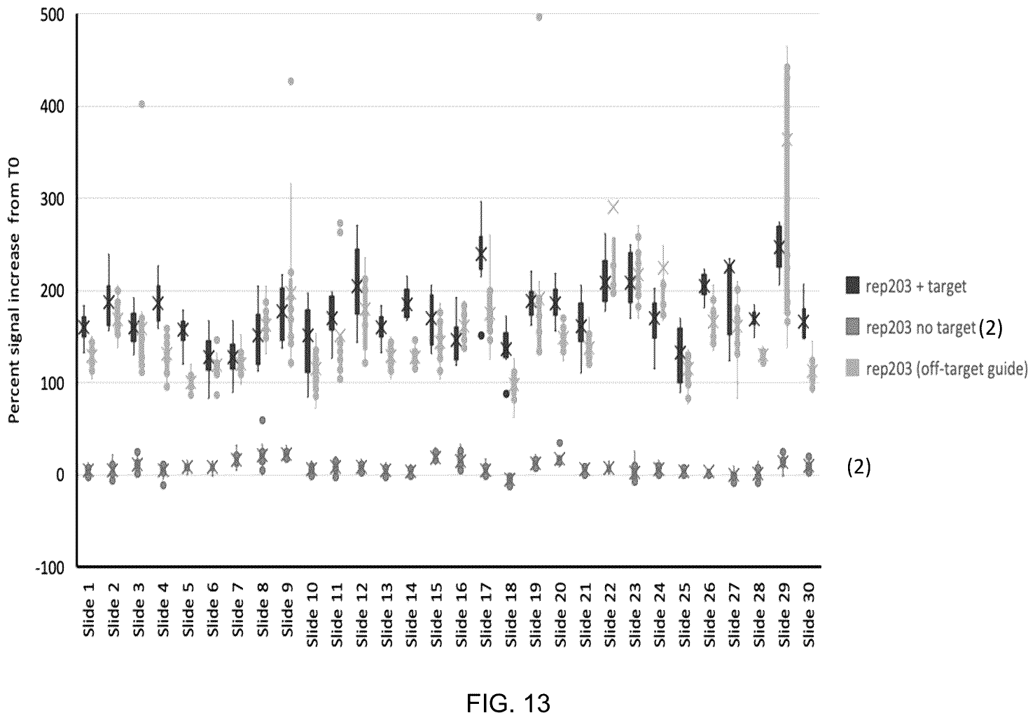

Immobilized Cas12 and Cas14 guide RNAs that are unmodified can be cleaved by a Cas protein upon complexing. Due to this ability, once complexed, the Cas protein and the guide RNA can be freed from the immobilized complex and influence nearby reporters in the same and different spots of an array. Without being bound by any particular theory, it is believed that this process occurs through RNA positioning of the guide RNA and the Cas protein. FIG. 13 shows the percent signal increase on the Y-axis and results for different array slides on the X-axis for RNA-only guides immobilized to the slides. RNA-only guides and rep203 quencher-fluorophore reporters were immobilized to a surface so as to co-localize in spots of an array as described herein. Each spot consisted of a guide RNA (on target or off target, but not both) and a reporter. Cas protein and target (or Cas protein alone, in the “no target” condition) were then added to the array and trans-cleavage activity was determined by monitoring for an increase in the fluorescence at each spot on the array (where an increase in fluorescence indicates reporter cleavage and release of the quencher moiety). The graph shows that there was a robust fold change in reporter signal when the target was added as compared to when the target was not added. However, there was no significant separation in signal for spots along the array that used an off-target guide under the conditions tested. This suggested that the guide-Cas complex with on target guides influenced co-localized reporters within their own spot but also acted on reporters in neighboring spots. When no target was present in the neighboring spot, as in the “rep203 no target” condition, where the on-target guide was present but no target was added, no signal was observed. When the target was present in the neighboring spot, as in the “rep203 (off-target guide)” condition, where the target was added to the off-target guide spot, a significant signal was observed. It was hypothesized that cleavage of the neighboring reporters was due to auto-hydrolysis of the on-target guides by the Cas protein following complexing with the target nucleic acid, which lead to release of the activated Cas complex into solution, thereby enabling the released activated Cas complex to diffuse and act on reporters located in neighboring spots.

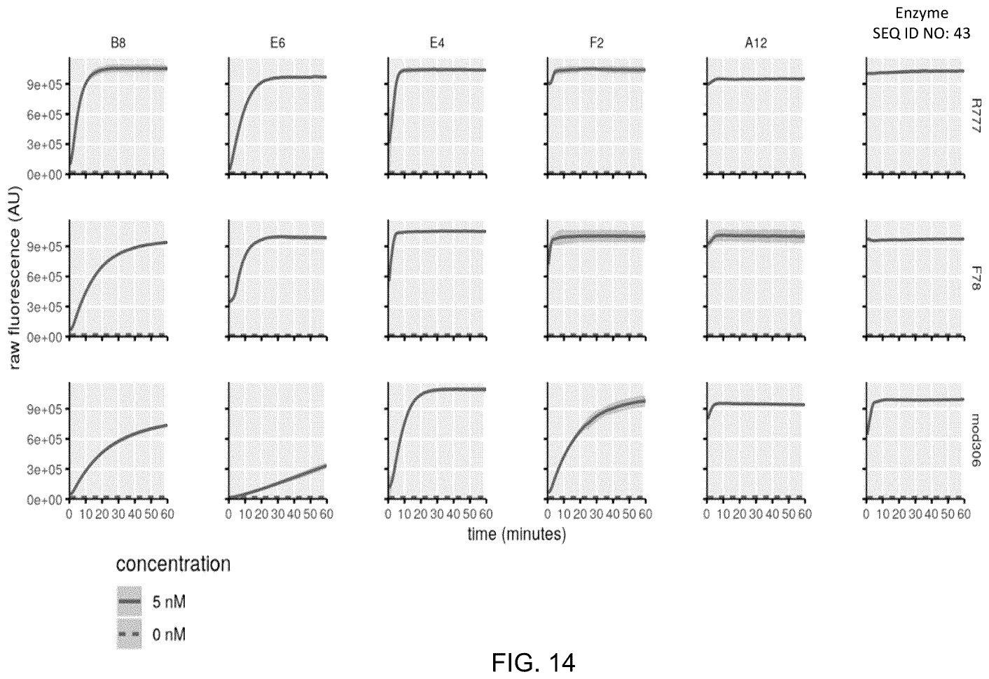

To reduce the cleavage of the guide RNA, key nucleotides from the immobilized guide RNA complex were changed to DNA bases whose linking bond was no longer cleavable in this manner. FIG. 14 shows an RNA-only guide (R777) comprising the sequence (rUrArArUrUrUrCrUrArCrUrArArGrUrGrUrArGrArUrGrCrCrGrArUrArArUrGrArUrGrUrAr GrGrGrArU SEQ ID NO: 89), a guide where the first 2 nucleotides have been converted to DNA (F78), comprising the construct (/5BiotinTEG/TTTTTTTTTTTTTTTTArArUrUrUrCrUrArCrUrArArGrUrGrUrArGrArUrGrCr CrGrArUrArArUrGrArUrGrUrArGrGrGrArU (SEQ ID NO: 90)), where the bolded TA was converted to DNA, and a guide where the first 6 nucleotides have been converted to DNA (mod306), comprising the construct (/5AmMC12/TAATTTrCrUrArCrUrArArGrUrGrUrArGrArUrGrCrCrGrArUrArArUrGrArUrG rUrArGrGrGrArU (SEQ ID NO: 91)), where the bolded TAATTT was converted to DNA.

These constructs were tested in a solution with different Cas12 orthologs for their ability to cleave a target sequence. FIG. 14 shows the modified guides could recognize their target and enable trans-cleavage of nearby reporters comprising fluorophore quencher pairs in the solution. Cas enzymes and guides were complexed for 30 minutes at 37° C. Reporters were then added and DETECTR reactions were run for 60 minutes at the temperatures indicated for the corresponding Cas proteins (B8, E6=50° C.; E4, F2, SEQ ID NO: 43=55° C.; and A12=60° C.). The Cas12 orthologs tested and shown in the graphs of FIG. 14 were “B8” (having a sequence of SEQ ID NO: 52), “E6” (having a sequence of SEQ ID NO: 87), “E4” (having a sequence of SEQ ID NO: 84), “F2” (having a sequence of SEQ ID NO: 86), “A12” (having a sequence of SEQ ID NO: 85), and Enzyme SEQ ID NO: 43.

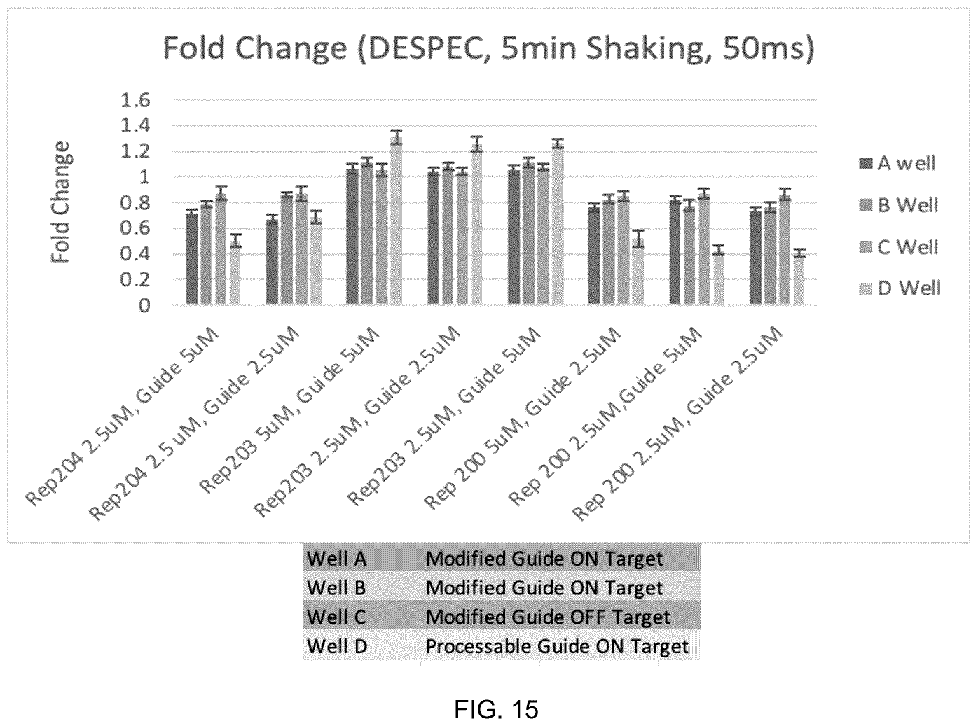

As shown in FIG. 15, upon the conversion of key nucleotides to DNA in the immobilized guide RNA sequence, there was an inability of the complex to interact with nearby oligos, which showed inhibition of guide processing and release. In FIG. 15, well A and well B show DNA modified guide sequences mixed with on target sequences; well C shows modified guides mixed with an off target sequence; and well D shows processable RNA-only guides mixed with an on target sequence. For the DNA modified guides there was little to no fold change difference in signal for spots with off target guides versus on target guides. However, the complex with the processable RNA guide was able to cleave their respective reporter. These unexpected results suggest the immobilized modified guide Cas complexes may have been sterically inhibited from reaching their targets under the conditions tested.

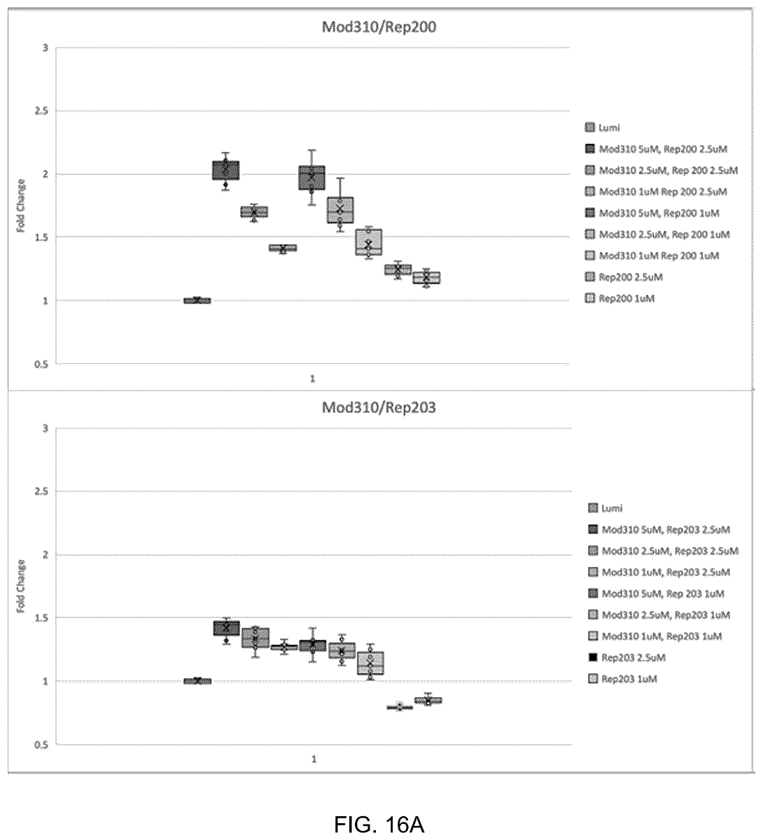

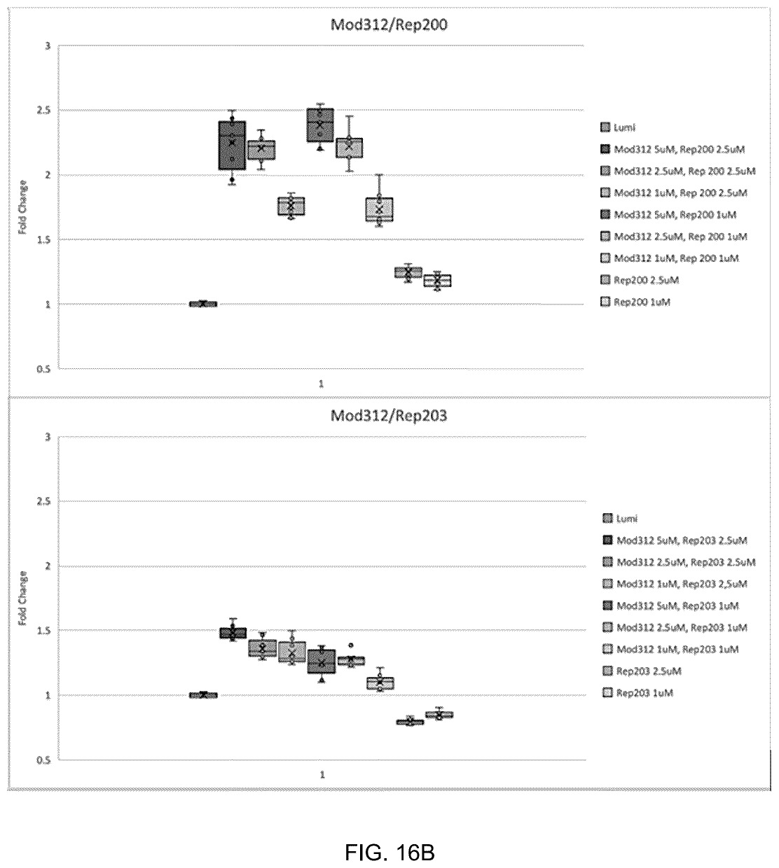

To increase the interaction of the fully bound complex with the surrounding oligos, a longer linker between the attachment chemistry and guide nucleic acid was developed to create more flexibility for the Cas protein to reach a target nucleic acid and/or the co-immobilized reporters. FIGS. 16A-16B illustrate results showing that modified guides with longer linkers (original linker of 6-12 carbon lengthened by another 18 carbons), robustly cleaved reporters in a location specific manner. For example, Mod3 10 comprised the following construct/5AmMC12//iSp18/TAATTTrCrUrArCrUrArArGrUrGrUrArGrArUrGrCrCrGrArUrArArUrGrA rUrGrUrArGrGrGrArU (SEQ ID NO: 142) and Mod3 12 comprised the following construct/5AmMC6//iSp18/TAATTTrCrUrArCrUrArArGrUrGrUrArGrArUrGrCrCrGrArUrArArUrGrAr UrGrUrArGrGrGrArU (SEQ ID NO: 143). Improvements were observed with more guide nucleic acids, whereas less impact was observed from reporter concentration under the conditions tested.

Additionally, a unique guide nucleic acid-reporter fusion molecule was developed that forced a closer relationship between the Cas and the reporter. The modified guide reporters (gREPs) enabled location specific signal production when presented with a cognate target. FIG. 17A shows the gREPs had increased signal production when mixed with their cognate target. Limited signal change was seen for reporter only molecules (e.g., Rep200), as expected if no cross-talk occurred between spots. Similarly, when modified reporters complexed with guides (mod) and reporter only molecules (rep) were mixed, increased signal production was observed for gREPs. Mod308 comprised the following construct/5AmMC12/TAATTTrCrUrArCrUrArArGrUrGrUrArGrArUrGrCrCrGrArUrArArUrGrArUrGr UrArGrGrGrArUTTTTTTTTTTTTTTTTTTT/36-FAM/(SEQ ID NO: 122). Mod309 comprised the following construct 5AmMC6/TAATTTrCrUrArCrUrArArGrUrGrUrArGrArUrGrCrCrGrArUrArArUrGrArUrGrUr ArGrGrGrArUTTTTTTTTTTTTTTTTTTT/36-FAM/(SEQ ID NO: 123). The reporter only control shows the Cas protein was not able to free itself and interact with the reporter only spots. FIG. 17B shows a representation of the composition of exemplary gREPs. Starting from the 5′ side, an attachment chemistry is provided to immobilize the gREP construct to a surface as described herein. An uncleavable carbon spacer is optionally added to give the complex room to search for its target as described herein. A chimeric (e.g., modified) guide nucleic acid follows the carbon spacer, onto which the Cas protein hybridizes to form a Cas complex. The end of the gREP may be comprised of a nucleic acid (e.g., ssDNA, ssRNA, etc.) tether holding a detection moiety (e.g., a fluorophore, quencher, etc.). In some examples, a nucleic acid tether and detection moiety can be attached to the linker before (e.g., 5′ to) the guide nucleic acid. Upon target recognition by the Cas complex, the nucleic acid tether is cleaved, allowing for the released detection moiety to diffuse away from the surface. For example, when the detection moiety comprises a fluorophore, release of the fluorophore may result in a reduction of fluorescence observed at the surface as described herein.

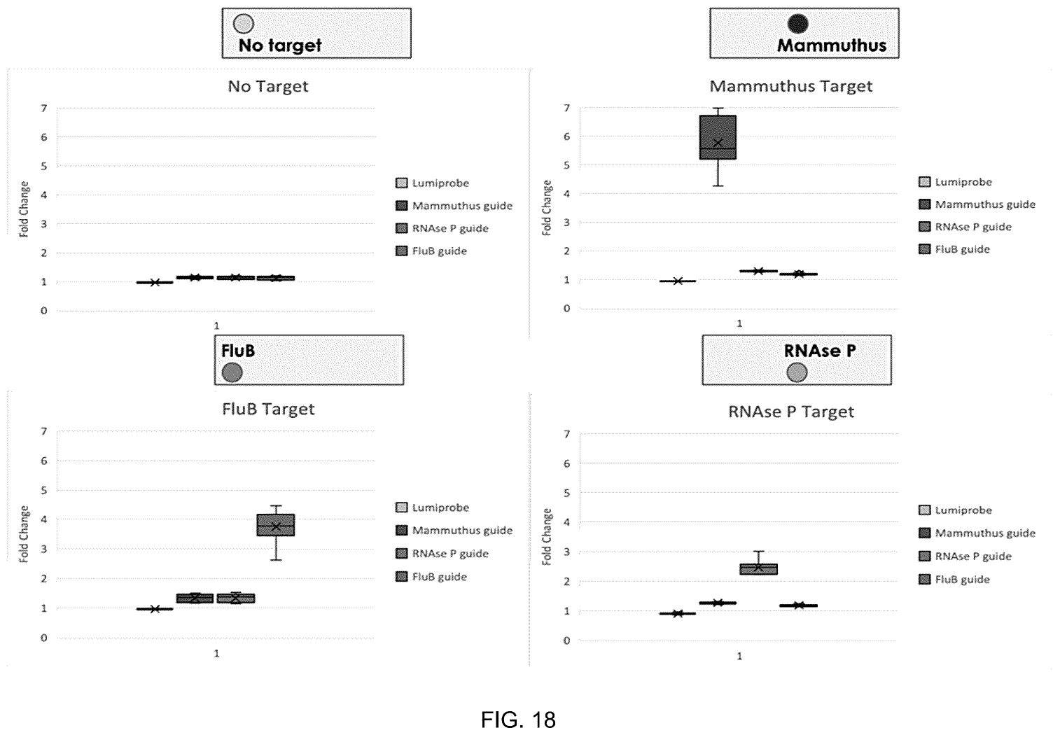

An array was developed comprising spots with unique gREPs to demonstrate multiplexing for different targets. The gREPs targeted either mammathus, RNAse P, or FluB. The array was introduced to a 5 μM amount of one of the three targets. As shown in FIG. 18, the spots containing the cognate gREP introduced to the target presented a significant fold change. When no target was added into the array, no spot presented a significant fold change. Each array contained 12 spots for each target. The results show the gREPs were target-specific and that no off target cleaving was identified.

### Example 9: Guide Nucleic Acid Design for One-Pot Assays

Single-reaction volume programmable nuclease (e.g., CRISPR/Cas) based diagnostic reactions (also referred to herein as one-pot assays) according to embodiments described herein are simpler to implement than multiple reaction assays because they require fewer steps and have a reduced chance of leading to amplicon contamination in laboratories. Single reaction volume CRISPR reactions may use an isothermal nucleic acid amplification reaction (e.g., LAMP or NEAR) to exponentially amplify a target DNA or RNA molecule while simultaneously allowing a CRISPR/Cas nuclease to report the presence or absence of the target DNA or RNA through complementarity with the gRNA and cleavage of a reporter molecule as described herein. Thermostable Cas12 or Cas13 enzymes, for example, may be used in single reaction volume programmable nuclease-based diagnostic assays. In at least some instances, it may be preferred to use Cas enzymes that have a single, short RNA component (e.g., a Cas12a) over those that have multiple and/or longer RNA components (e.g., a Cas12b). Thermostable Cas14 enzymes may be used in single reaction volume programmable nuclease-based diagnostic assays. Thermostable Cas enzymes are often advantageous for these reactions because they take place at reaction temperatures of 50-70° C. (generally 55° C. or 60° C.), thereby allowing the CRISPR/Cas and nucleic acid amplification reactions to take place essentially simultaneously if desired.

Cas12 enzymes generally prefer PAMs with specificities similar to TTTN or YYN (Y=T or C), but generally YYN PAMs are thought to perform best with T-rich versions of the YYN. However, when Cas12 gRNAs are designed for some single reaction volume reactions, the assays may not be as robust as desired in terms of sensitivity and/or speed. For example, if the canonical PAM gRNAs are slow to generate signal or fail to generate signal it can be difficult to see that a reaction was functional. This can make it challenging to use a thermostable Cas12 enzyme in single reaction volume reactions. To increase the sensitivity with Cas12 gRNAs in single reaction volume reactions, noncanonical PAMs were identified for Cas12 enzymes which improved assay performance.

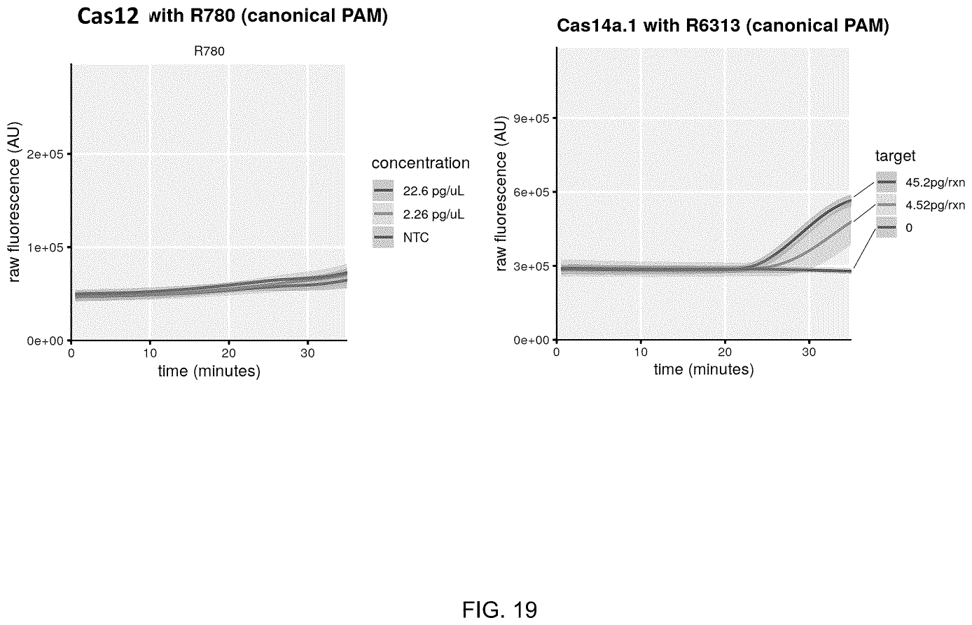

FIG. 19 shows illustrative results for single reaction volume assays with a thermostable Cas12 (SEQ ID NO: 43) enzyme with a gRNA targeting a site with a canonical PAM and a thermostable Cas14a.1 (SEQ ID NO: 11) enzyme with a sgRNA targeting a site with a canonical PAM. Both assays targeted human RNase P POP7 at the same target site and use the same TTTN PAM. To test the enzymes, 40 nM guide and 40 nM Cas (final concentration) were complexed together for 30 minutes at room temperature in buffer IB15. Reporter and target (Cas12 target=22.6 pg/μL, 2.26 pg/μL, or 0 pg/μL (“NTC”) final concentration; Cas14a. 1 target =45.2 pg/reaction, 4.52 pg/reaction, or 0 pg/reaction) were then added and the DETECTR reaction was run for 60 minutes at 55° C. Significant Cas12 signal was detectable after 60 minutes, though it remained indistinguishable from the NTC for about 30 minutes. The Cas12 enzyme does not show an increase in fluorescence as compared to the Cas14a. 1 enzyme when increasing concentrations of the target are added, under the conditions tested. The results show Cas12 with a canonical PAM crRNA (R780) was indistinguishable from the NTC, but Cas14a.1 using a similar target site with a canonical PAM clearly showed an increase in fluorescence (R6313). Without wishing to be bound by theory, the results indicate that the failure of the reaction was due to Cas 12 with canonical PAM not functioning with the primer set, even though a valid target was generated (as seen by Cas14a. 1 detection).

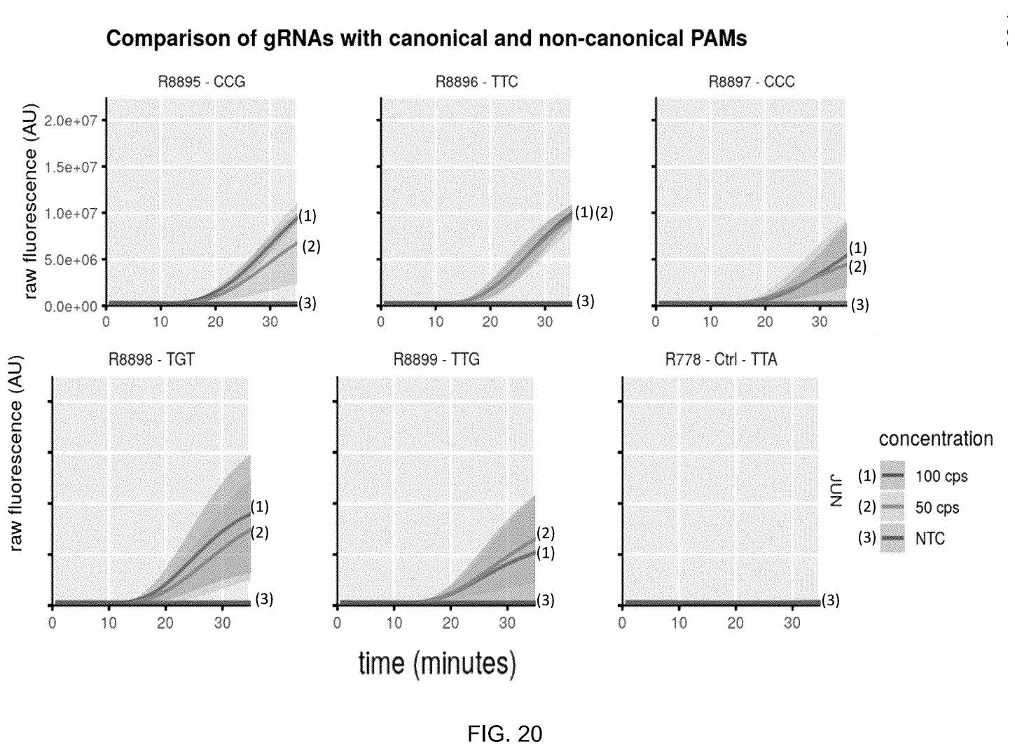

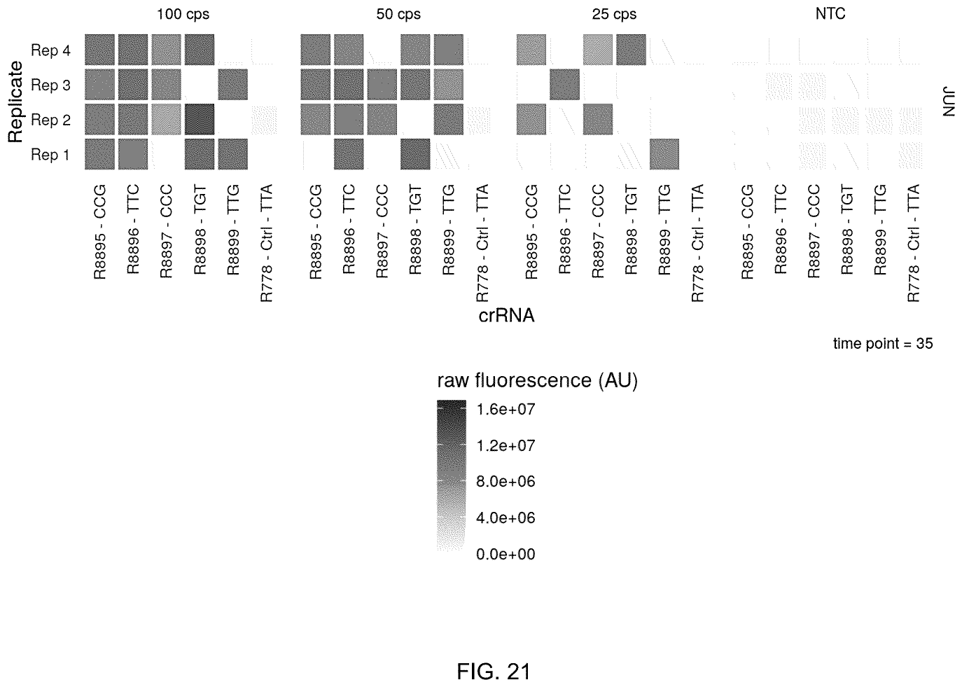

To increase the sensitivity of the one-pot assay, Cas12 gRNAs for influenza B targeting non-canonical PAMs were developed and are shown in Table 6. Table 6 shows the guide ID, the PAM sequence, the spacer sequence, the gRNA sequence, and notes related to the PAM modification relative to the canonical PAM. The spacer in Table 6 is the target site in the DNA and the gRNA sequence is the RNA sequence used to program the nuclease. The canonical PAM and several noncanonical PAMs were tested in single reaction volume assays with a thermostable Cas12 enzyme (SEQ ID NO: 43) targeting Influenza B sequences. To test the PAMs, 40 nM guide and 40 nM Cas (final concentration) were complexed together for 30 minutes at room temperature in buffer IB15. Reporter and target (100 copies per reaction, 50 copies per reaction, or 0 copies per reaction (“NTC”)) were then added and the DETECTR reaction was run for 10 minutes at 62° C. followed by 50 minutes at 55° C. As shown in FIG. 20, the noncanonical PAMs of R8895 (CCGT), R8896 (TCCG), R8897 (CCCT), R8898 (TGTC), and R8899 (TTGT) all showed an increase in fluorescence detection as compared to the canonical PAM of R778 (TTA) under the conditions tested. Similarly, as shown in FIG. 21, the noncanonical PAMs of R8895 (CCGT), R8896 (TCCG), R8897 (CCCT), R8898 (TGTC), and R8899 (TTGT) all showed an increase in fluorescence detection at two different target concentrations as compared to the canonical PAM R778 (TTA), which showed no change in fluorescence under the conditions tested (described above), even at the highest concentration of target tested. The fluorescence intensity was determined at 35 minutes after starting the reaction.

TABLE 6
Design of Cas12 gRNAs with non-canonical PAMs for influenza B.

| ID | PAM | Spacer | gRNA Sequence | Note |
|---|---|---|---|---|
| | TTTA | CTAACACTCTCAGGGACA | GAAUUUCUACUAUUGUAG | Canonical |
| | | AT (SEQ ID NO: 92) | AUCUAACACUCUCAGGGAC | |
| | | | AAU (SEQ ID NO: 98) | |
| R8895 | CCGT | TTACTAACACTCTCAGGGA | AUUUACUAACACUCUCAGG | Contains G |
| | | C (SEQ ID NO: 93) | GAAUUUCUACUAUUGUAG | |
| | | | GAC (SEQ ID NO: 99) | |
| R8896 | TCCG | TTTACTAACACTCTCAGGG | GAAUUUCUACUAUUGUAG | C rich version of YYN |
| | | A (SEQ ID NO: 94) | AUUUUACUAACACUCUCAG | |
| | | | GGA (SEQ ID NO: 100) | |
| R8897 | CCCT | GAGAGTGTTAGTAAACGG | GAAUUUCUACUAUUGUAG | C rich version of YYN |
| | | AA (SEQ ID NO: 95) | AUGAGAGUGUUAGUAAAC | |
| | | | GGAA (SEQ ID NO: 101) | |
| R8898 | TGTC | CCTGAGAGTGTTAGTAAAC | AUCCUGAGAGUGUUAGUA | Contains G |
| | | G (SEQ ID NO: 96) | GAAUUUCUACUAUUGUAG | |
| | | | AACG (SEQ ID NO: 102) | |
| R8899 | TTGT | CCCTGAGAGTGTTAGTAAA | AUCCCUGAGAGUGUUAGU | Contains G |
| | | C (SEQ ID NO: 97) | GAAUUUCUACUAUUGUAG | |
| | | | AAAC (SEQ ID NO: 103) | |

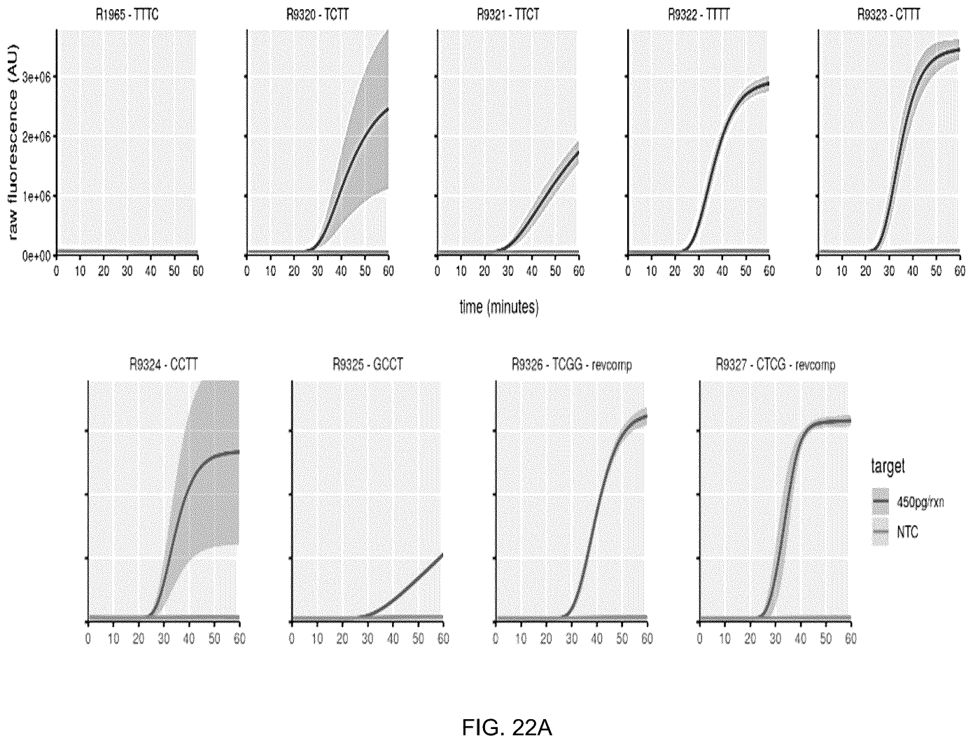

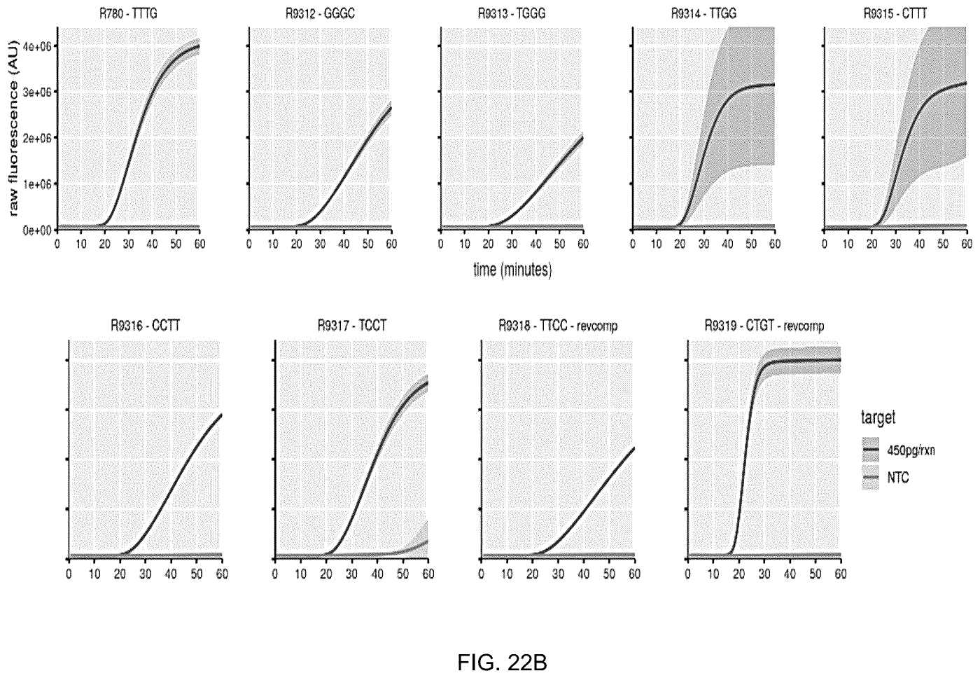

Similarly, to increase the sensitivity of assays with the Cas12 gRNAs targeting RNase P in HeLa cell RNA, non-canonical PAMs were identified and are shown in Table 7. Table 7 shows the guide ID, the PAM sequence, the spacer sequence, the gRNA sequence, and notes related to the PAM modification. Canonical PAMs and several noncanonical PAMs were tested in single reaction volume assays with a thermostable Cas12 enzyme (SEQ ID NO: 43) targeting RNase P. Canonical PAMs were TTTN or YYN. As shown in FIGS. 22A-22B, the noncanonical PAMs identified as R9312 (GGGC), R9313 (TGGG), R9314 (TTGG), R9316 (CCTT), R9317 (TCCT), R9319 (CTGT), R9320 (TCTT), R9321 (TTCT), R9323 (CTTT), R9324 (CCTT), R9325 (GCCT), R9326 (TCGG), and R9327 (CTCG) all showed fluorescence detection when 450 pg/reaction target was present compared to the no template control (NTC).

Together the data showed noncanonical PAMs enabled an increase in the detection sensitivity for the presence of target nucleic acids in single reaction volume assays.

TABLE 7
Design of Enzyme SEQ ID NO: 43 gRNAs with non-canonical PAMs for RNase P.

| ID | PAM | Spacer | gRNA Sequence | Note |
|---|---|---|---|---|
| R9312 | GGGC | TTCCAGGGAACAGGCCTTTT | GAAUUUCUACUAUUGUA | Contains GGG |
| | | (SEQ ID NO: 104) | GAUUUCCAGGGAACAGGC | |
| | | | CUUUU (SEQ ID NO: 113) | |
| R9313 | TGGG | CTTCCAGGGAACAGGCCTTT | GAAUUUCUACUAUUGUA | Contains GG |
| | | (SEQ ID NO: 105) | GAUCUUCCAGGGAACAGG | |
| | | | CCUUU (SEQ ID NO: 114) | |
| R9314 | TTGG | GCTTCCAGGGAACAGGCCTT | GAAUUUCUACUAUUGUA | Contains G |
| | | (SEQ ID NO: 106) | GAUGCUUCCAGGGAACAG | |
| | | | GCCUU (SEQ ID NO: 115) | |
| R780 | TTTG | GGCTTCCAGGGAACAGGCCT | GAAUUUCUACUAUUGUA | Canonical TTTN |
| | | (SEQ ID NO: 107) | GAUGGCUUCCAGGGAACA | |
| | | | GGCCU (SEQ ID NO: 116) | |
| R9315 | CTTT | GGGCTTCCAGGGAACAGGCC | GAAUUUCUACUAUUGUA | Canonical YYN |
| | | (SEQ ID NO: 108) | GAUGGGCUUCCAGGGAAC | |
| | | | AGGCC (SEQ ID NO: 117) | |
| R9316 | CCTT | TGGGCTTCCAGGGAACAGGC | GAAUUUCUACUAUUGUA | C rich version of YYN |
| | | (SEQ ID NO: 109) | GAUUGGGCUUCCAGGGAA | |
| | | | CAGGC (SEQ ID NO: 118) | |
| R9317 | TCCT | TTGGGCTTCCAGGGAACAGG | GAAUUUCUACUAUUGUA | C rich version of YYN |
| | | (SEQ ID NO: 110) | GAUUUGGGCUUCCAGGGA | |
| | | | ACAGG (SEQ ID NO: 119) | |
| R9318 | TTCC | CTGGAAGCCCAAAGGACTCT | GAAUUUCUACUAUUGUA | Canonical YYN |
| | | (SEQ ID NO: 111) | GAUCUGGAAGCCCAAAGG | |
| | | | ACUCU (SEQ ID NO: 120) | |
| R9319 | CTGT | TCCCTGGAAGCCCAAAGGAC | GAAUUUCUACUAUUGUA | Contains G |
| | | (SEQ ID NO: 112) | GAUUCCCUGGAAGCCCAA | |
| | | | AGGAC (SEQ ID NO: 121) | |
| R1965 | TTTC | TTACATGGCTCTGGTCCGAG | UAAUUUCUACUAAGUGU | |
| | | (SEQ ID NO: 124) | AGAUUUACAUGGCUCUGG | |
| | | | UCCGAG (SEQ ID NO: 133) | |
| R9320 | TCTT | ACATGGCTCTGGTCCGAGGT | GAAUUUCUACUAUUGUA | |
| | | (SEQ ID NO: 125) | GAUACAUGGCUCUGGUCC | |
| | | | GAGGU (SEQ ID NO: 134) | |
| R9321 | TTCT | TACATGGCTCTGGTCCGAGG | GAAUUUCUACUAUUGUA | |
| | | (SEQ ID NO: 126) | GAUUACAUGGCUCUGGUC | |
| | | | CGAGG (SEQ ID NO: 135) | |
| R9322 | TTTT | CTTACATGGCTCTGGTCCGA | GAAUUUCUACUAUUGUA | |
| | | (SEQ ID NO: 127) | GAUCUUACAUGGCUCUGG | |
| | | | UCCGA (SEQ ID NO: 136) | |
| R9323 | CTTT | TCTTACATGGCTCTGGTCCG | GAAUUUCUACUAUUGUA | |
| | | (SEQ ID NO: 128) | GAUUCUUACAUGGCUCUG | |
| | | | GUCCG (SEQ ID NO: 137) | |
| R9324 | CCTT | TTCTTACATGGCTCTGGTCC | GAAUUUCUACUAUUGUA | |
| | | (SEQ ID NO: 129) | GAUUUCUUACAUGGCUCU | |
| | | | GGUCC (SEQ ID NO: 138) | |
| R9325 | GCCT | TTTCTTACATGGCTCTGGTC | GAAUUUCUACUAUUGUA | |
| | | (SEQ ID NO: 130) | GAUUUUCUUACAUGGCUC | |
| | | | UGGUC (SEQ ID NO: 139) | |
| R9326 | TCGG | ACCAGAGCCATGTAAGAAAA | GAAUUUCUACUAUUGUA | |
| | | (SEQ ID NO: 131) | GAUACCAGAGCCAUGUAA | |
| | | | GAAAA (SEQ ID NO: 140) | |
| R9327 | CTCG | GACCAGAGCCATGTAAGAAA | GAAUUUCUACUAUUGUA | |
| | | (SEQ ID NO: 132) | GAUGACCAGAGCCAUGUA | |
| | | | AGAAA (SEQ ID NO: 141) | |

### Example 10: Optimizing Sensitivity of One-Pot Programmable Nuclease-Based Detection

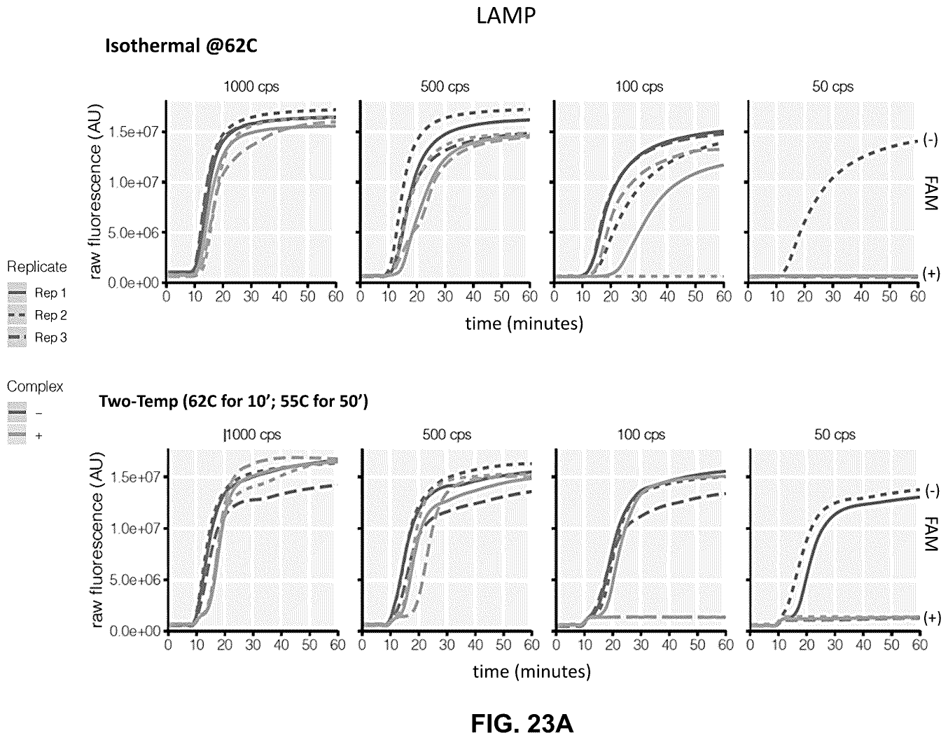

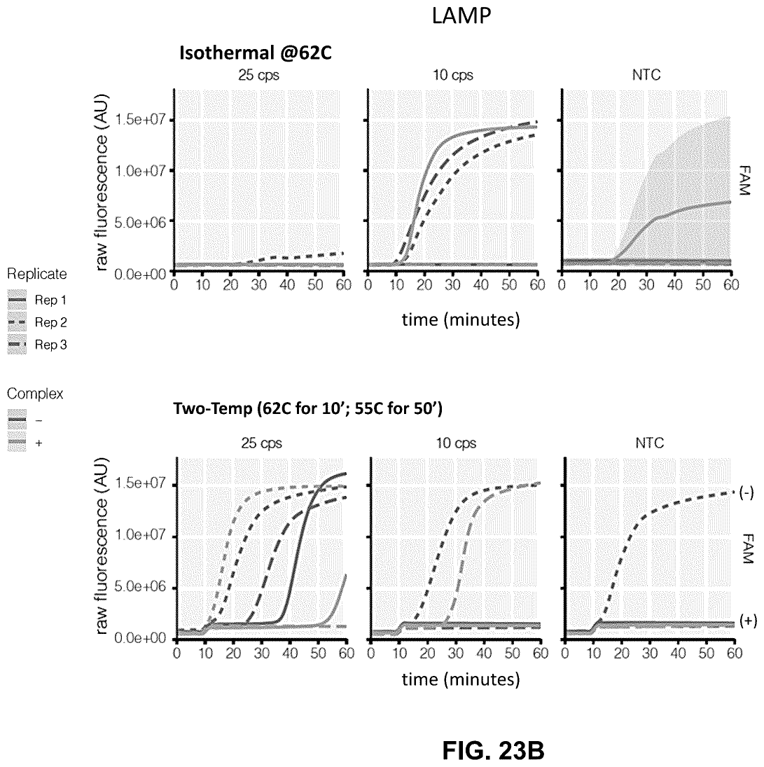

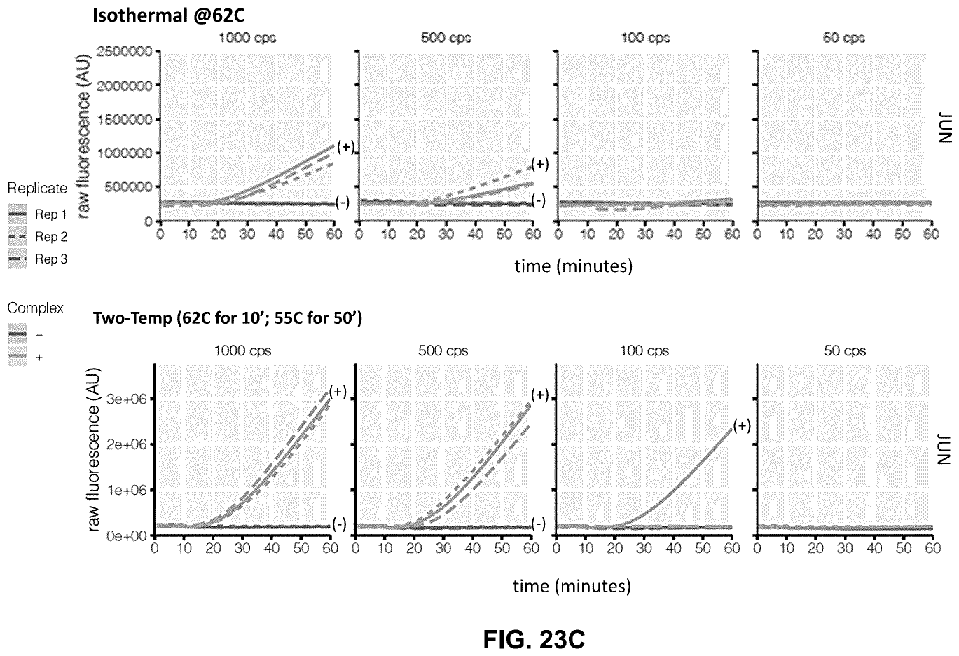

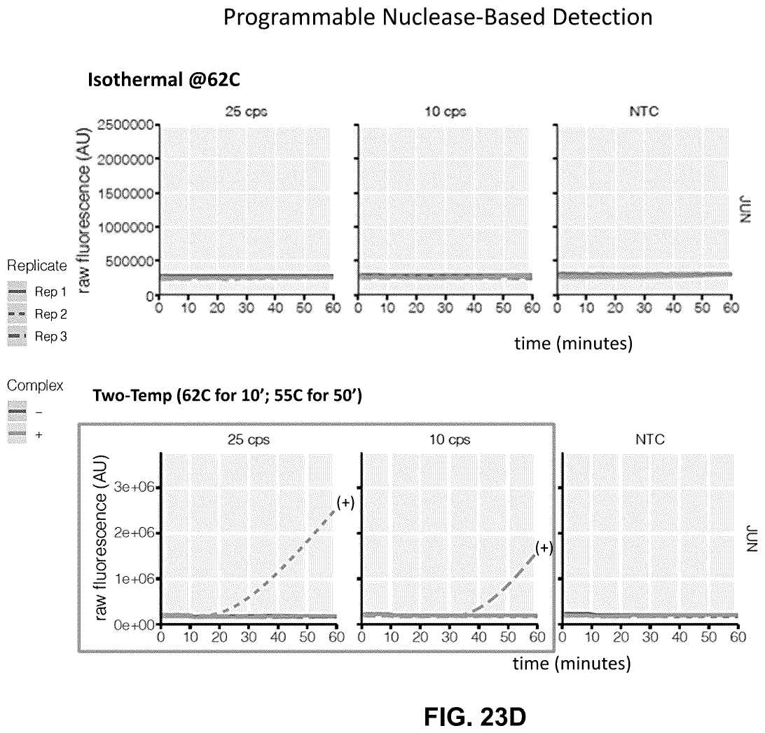

Single reaction volume LAMP-CRISPR reactions were performed at different temperatures either as isothermal reactions or a single temperature shift over the reaction time course (also referred to as “SIMMR”). Reactions included polymerase and primers for LAMP amplification of an influenza B virus (IBV) target nucleic acid sequence. Some reactions also included reagents for a programmable nuclease-based detection reaction including a thermostable Cas12 variant enzyme of SEQ ID NO: 43 complexed with a guide nucleic acid (Complex +; three replicates), while some did not (Complex −; three replicates). Reactions included different target nucleic acid concentrations to evaluate detection sensitivity under the respective conditions (10, 25, 50, 100, 500, or 1000 copies per reaction (“cps”); or a no template control (“NTC”)). Reactions also included a SYTO label to track progress of the LAMP reaction, and 1 mM of cleavable reporter, cleavage of which by the Cas enzyme indicated detection of the target nucleic acid (detected at a different wavelength from SYTO) as described herein. Cas enzymes and guide RNAs were included at a final concentration of 40 nM per reaction. Results tracking fluorescence from the SYTO label to confirm LAMP activity are shown in FIGS. 23A-23B. Results tracking fluorescence from the cleavable reporter are shown in FIGS. 23C-23D. Results also show the effect of an isothermal reaction held at 62° C. for 60 minutes (FIGS. 23A and 23C), as compared to reactions incubated at 62° C. for 10 minutes followed by incubation at 55° C. for 50 minutes. The temperature shift was designed to test whether a higher initial temperature could favor polymerase activity and a lower second temperature could favor programmable nuclease activity. As shown in FIGS. 23A-23B, LAMP successfully amplified target nucleic acid under all conditions and under both temperature profiles. FIGS. 23C-23D show that, under these conditions, the programmable nuclease-based detection reaction with the Cas 12 variant of SEQ ID NO: 43 showed stronger signal and sensitivity at lower concentrations of target nucleic acid using the two-temperature approach, as compared to the isothermal reactions (compare, e.g., results at 10 and 25 copies per reaction). Additionally, the reaction using 10 copies of the target nucleic acid produced a detectable fluorescence signal indicating the assay has substantial sensitivity.

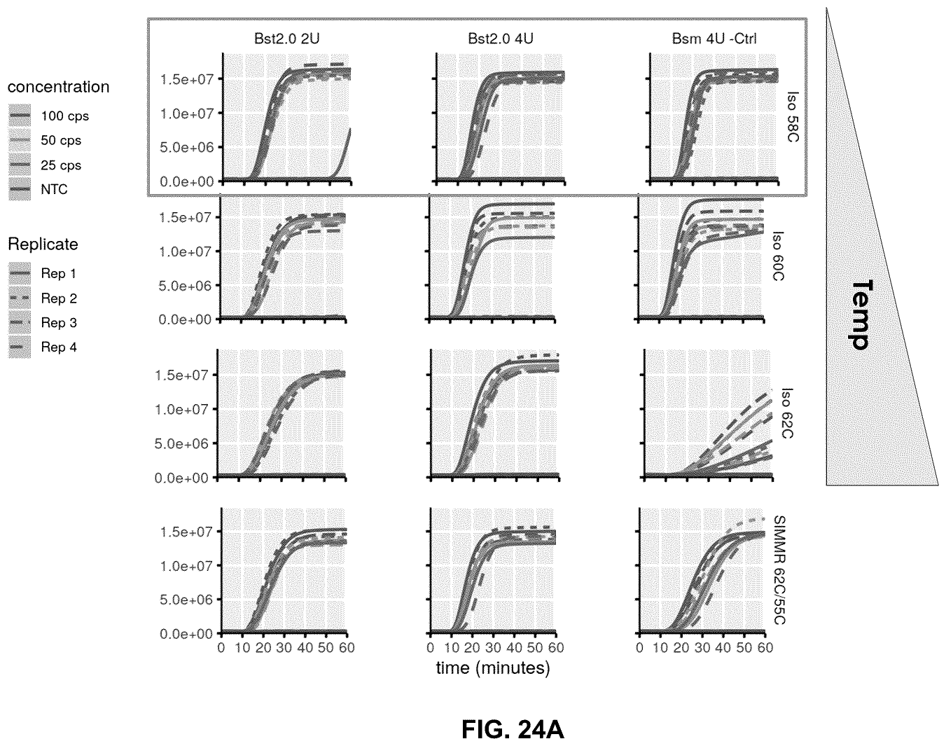

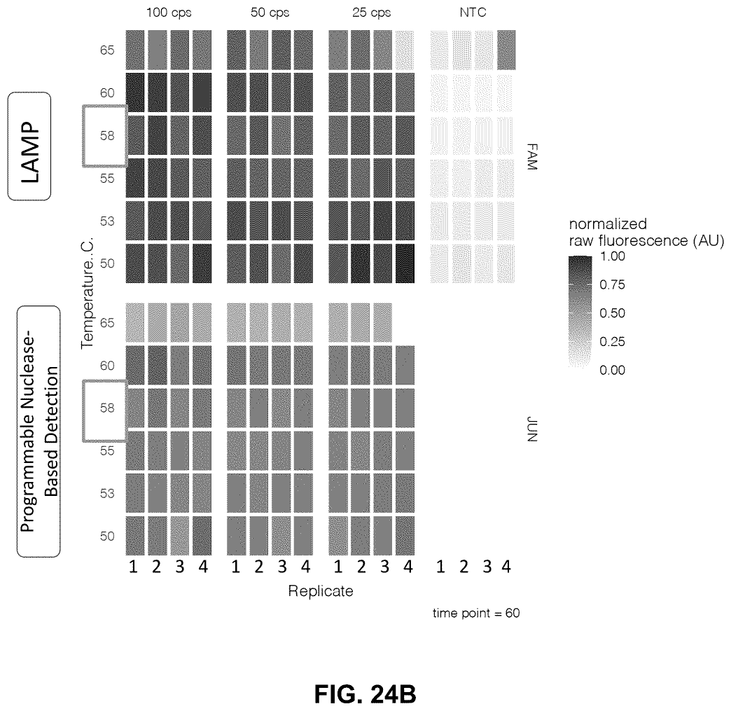

The effect of polymerase selection and concentration on detection sensitivity under isothermal or temperature-shifting incubation was also evaluated. Single reaction volume programmable nuclease-based detection reactions were performed at different temperatures, either as isothermal reactions or with a single temperature shift. Similar to the reactions described above, reactions included reagents for LAMP, reagents for programmable nuclease-based detection with a Cas12 variant of SEQ ID NO: 43, a SYTO label to monitor the LAMP reaction, and a cleavable reporter. Reactions differed in the selection of polymerase used for the LAMP reaction, either Bst2.0 polymerase (at either 2 units or 4 units per reaction; NEB), or Bsm polymerase (at 4 units per reaction; ThermoFisher). Reactions were run for 60 minutes under isothermal conditions (58° C., 60° C., or 62° C.), or at an initial temperature of 62° C. for 10 minutes followed by 55° C. for 50 minutes. Reactions included 25, 50, or 100 copies of target nucleic acid per reaction (“cps”), or lacked target nucleic acid (no template control, or “NTC”). Each reaction condition was tested in four replicates. Fluorescence detection from the cleavable reporter over the course of the reaction is illustrated in FIG. 24A. The results indicated that reactions with Bst2.0 polymerase were less sensitive to incubation temperature than those with Bsm polymerase, the isothermal reactions at 58° C. were the most sensitive, and 4 units of Bst2.0 polymerase produced a stronger and more rapid signal than 2 units under the conditions tested. The isothermal reaction at 58° C. using 4 units of Bst2.0 polymerase was also notable for its rapid and robust detection at the lowest concentration of target nucleic acid tested of 25 copies per reaction. Results for multiple replicates of reactions using 4 units of Bst2.0 polymerase at a broader range of isothermal reaction temperatures (50° C., 53° C., 55° C., 58° C., 60° C., and 65° C.) are shown in FIG. 24B, which reports normalized raw fluorescence at the end of the 60-minute reaction for both LAMP (based on SYTO signal) and programmable nuclease-based detection (based on cleavable reporter signal). Both signals indicate robust detection down to at least 25 copies per reaction with similar performance from 55° C. to 60° C. under the conditions tested.

### Example 12: Multiplex Programmable Nuclease Detection Assays

Reaction conditions were tested for multiplex detection of different target nucleic acids in a single detection reaction. For reporter molecules that are cleaved in a sequence-independent manner, any activation of the programmable nuclease in the detection reaction may lead to a positive signal. Such circumstances present a challenge to multiplexing detection of different targets in a single reaction. One solution to multiplexing is to assay for multiple different target nucleic acids using separate reaction volumes for fractions of a given sample. However, it was observed that one-pot reactions directed to amplifying and detecting different target nucleic acids surprisingly reached different plateaus of maximum fluorescence over the course of the detection reaction, even when the amount of reporter was held constant. It was hypothesized that by combining reagents that produced detection signals at different saturation endpoints, the identity of a target nucleic acid in a positive reaction could be inferred from the fluorescence level.

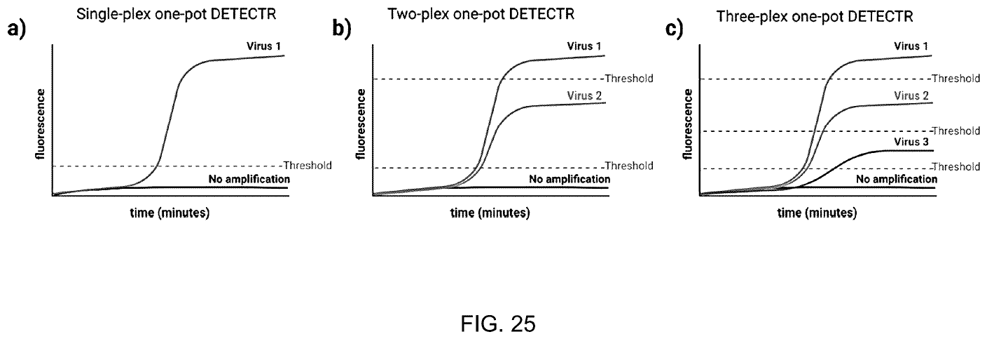

This principle is illustrated in FIG. 25, which shows idealized plots for detection reactions of hypothetical targets in single reaction volume assays performed with a programmable nuclease (e.g., Cas14a.1) to detect different target nucleic acid(s). All assays include amplification (e.g., LAMP or RT-LAMP) and programmable nuclease-based detection (e.g., DETECTR) in a single reaction volume (one-pot). Assay (a) includes reagents for the detection of a single target nucleic acid with a corresponding threshold for detection. Assays (b) and (c) include reagents for the detection of any of two (assay (b)) or three (assay (c)) different target nucleic acids in a single reaction volume. The detection reaction for each different target nucleic acid saturates at a different level when the target nucleic acid is present, and is associated with a different threshold. Thus, the level at which fluorescence plateaus is indicative of which target nucleic acid is present, allowing for the combination of reagents for detecting any of multiple distinct target nucleic acids in a single reaction, using the same reporter molecules. The graphs for assays (b) and (c) illustrate the various fluorescence plateaus and associated thresholds collapsed onto a single plot for the sake of comparison.

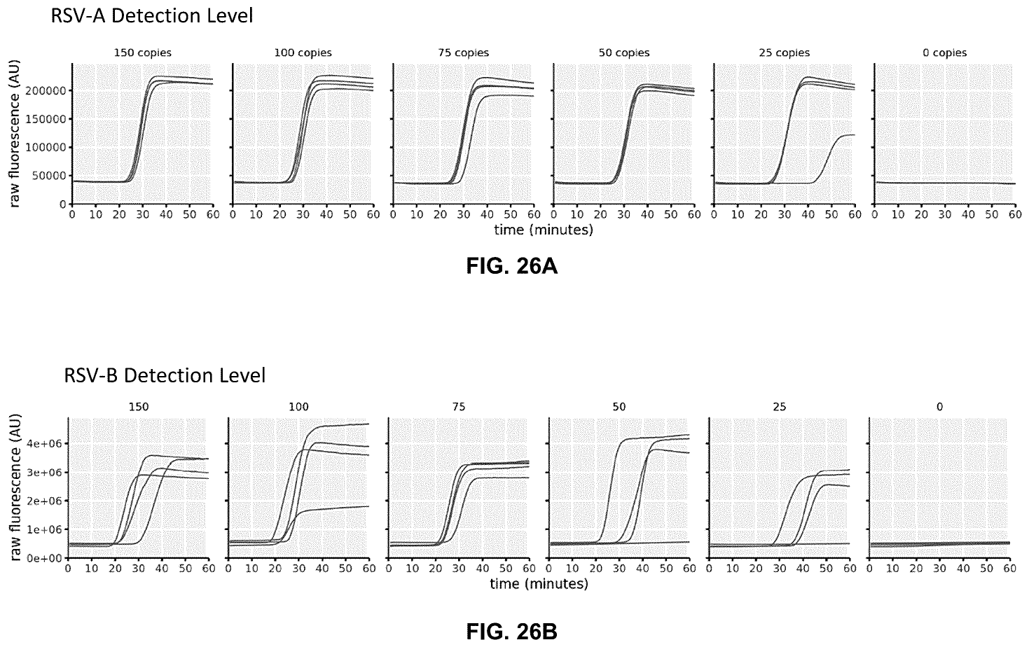

Single-plex one-pot detection reactions using reagents for reverse transcription and LAMP (RT-LAMP) and Cas14a. 1 as the programmable nuclease were first tested for detecting a target nucleic acid sequence of RSV-A or RSV-B at varying concentrations (150, 100, 75, 50, 25, or 0 copies per reaction). Briefly, Cas14a. 1 effector proteins were complexed with a crRNA for 30 minutes at 37° C. The 1× concentration of proteins was 40 nM and the final concentration of crRNAs was 40 nM. 1 μL of complexed Cas14a.1 was combined with a 9 μL mix of the following components for a total volume of ˜10 μL (listed at final concentration): IB15 one pot LAMP-trans cleavage buffer, RSV-A or RSV-B target RNA (150 to 0 copies), dNTPs (1 mM), RNAse inhibitor (NEB), Bsm DNA polymerase (ThermoFisher), Warmstart RTx reverse transcriptase (NEB), RSV-A or RSV-B primer mix, and FQ reporter (1000 nM). Reactions were carried out at 55° C. for 60 minutes. Trans cleavage activity was detected by fluorescence signal upon cleavage of a fluorophore-quencher reporter in the one-pot DETECTR reaction. Results for detection of the target nucleic acids run in the single-plex reactions are shown in FIG. 26A and FIG. 26B, in which the negative control reaction volumes with 0 copies of target nucleic acid produced no fluorescence signal, while all reactions with target nucleic acid produced a signal that eventually plateaued. FIG. 26A shows results of an assay with reagents for the detection of RSV-A where reactions with the target RSV-A produced a signal that plateaued at a first level that was substantially similar across all target concentrations. FIG. 26B shows results of an assay with reagents for the detection of RSV-B where reactions with the target RSV-B produced a signal that plateaued at a second level that was substantially similar across all target concentrations. Each reaction contained reagents for the detection of either RSV-A or RSV-B, but not both. Notably, the fluorescence level plateaus for the RSV-B target nucleic acid were significantly higher than those for the RSV-A target nucleic acid.

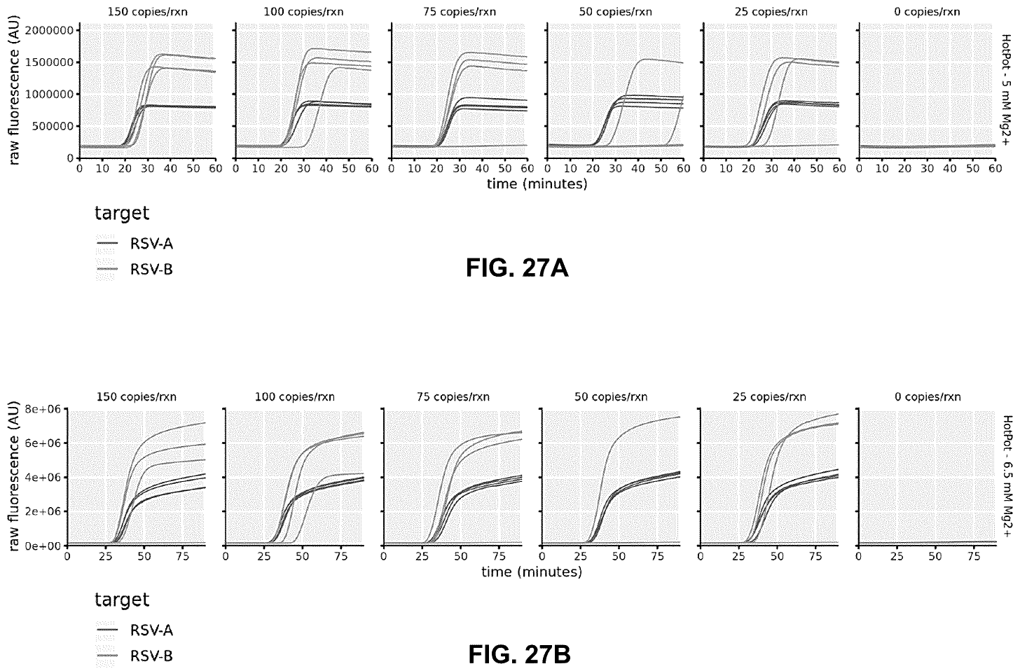

Reagents for the single-plex detection above were then combined to create a multiplex detection assay for the presence of either RSV-A or RSV-B. FIG. 27A depicts graphs showing illustrative results for the multiplex detection reactions. All reactions contained reagents for the amplification and detection of both RSV-A and RSV-B, but a target nucleic acid of only one of these was added to each reaction. Each line represents a single reaction in which either RSV-B target nucleic acids (top set of lines) or RSV-A target nucleic acids (bottom set of lines) were added to the reaction. As indicated, the maximum fluorescence of the assay for reactions with RSV-B was higher than reactions with RSV-A, despite both reactions having identical detection reagents. The maximum fluorescence level of the assay was therefore indicative of the target nucleic acid present in the reaction, thereby allowing for the distinction between RSV-A and RSV-B in a single volume with the same programmable nuclease and reporter being used to generate the signal.

The effect of magnesium concentration on the multiplex detection assay was also evaluated by repeating the assays using a higher Mg²⁺ concentration (6.5 mM instead of 5 mM). Illustrative results are depicted in FIG. 27B. Each line represents a single reaction in which either RSV-B target nucleic acids (top set of lines) or RSV-A target nucleic acids (bottom set of lines) were added to the reaction. The results showed that increased magnesium increased the overall signal of the assay, while also slightly slowing down the assay under the conditions tested. These results further demonstrate robustness of the approach across assay conditions and ability to optimize to increase signal differentiation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for detecting a target nucleic acid, the system comprising:
a detection region comprising:
i. a guide nucleic acid complementary to the target nucleic acid, or a portion thereof:
ii. a reporter immobilized to a surface of the detection region, the reporter comprising a nucleic acid and a detection moiety, wherein
a) the nucleic acid is at least 40 nucleotides in length;
b) the nucleic acid comprises a double-stranded region;
c) or a combination thereof, and
wherein cleavage of the reporter by a programmable nuclease, activated upon hybridization to the target nucleic acid, releases the detection moiety from the nucleic acid, and wherein the release of the detection moiety is configured to generate a signal indicative of a presence of the target nucleic acid.

2. The system of claim 1, further comprising the programmable nuclease.

3. The system of claim 2, wherein the programmable nuclease is configured to form a complex with the guide nucleic acid and to be activated through binding of the guide nucleic acid to the target nucleic acid.

4. The system of claim 1, wherein the nucleic acid is at least 50 nucleotides in length.

5. The system of claim 4, wherein the nucleic acid comprises a single-stranded region and the double-stranded region; and optionally wherein:
(a) the single-stranded region is from about 5 to about 15 nucleotides in length;
(b) the double-stranded region is from about 45 to about 55 nucleotides in length; or;
(c) the single-stranded region is about 9 nucleotides in length, and wherein the double-stranded region is about 50 nucleotides in length.

6. The system of claim 1, wherein the nucleic acid is single-stranded; and optionally wherein (a) the single-stranded nucleic acid is at least about 50 nucleotides in length; or (b) the single-stranded nucleic acid is from about 55 to about 65 nucleotides in length.

7. The system of claim 1, wherein:
(a) the detection moiety comprises a quencher moiety;
(b) the reporter comprises a fluorophore; or
(c) the detection moiety comprises a quencher moiety, the reporter comprises a fluorophore, and the quencher moiety is configured to quench the fluorophore prior to the cleavage of the reporter.

8. The system of claim 1, wherein:
(a) the detection moiety comprises a fluorophore; or
(b) the signal is 1) a fluorescence change, 2) a color change, 3) a brightness change or 4) a combination thereof.

9. The system of claim 1, wherein:
(a) the reporter comprises a nucleic acid sequence that is at least 80%, 90%, or 95% identical to any one of the sequences set forth in Table 1; or
(b) the reporter comprises any one of the sequences set forth in Table 1.

10. The system of claim 2, wherein the programmable nuclease is a Type V Cas nuclease or a Type VI Cas nuclease.

11. The system of claim 2, wherein:
(a) the programmable nuclease comprises an amino acid sequence at least 80%, 90%, or 95% identical to any one of SEQ ID NOS: 26 or 43; or
(b) the programmable nuclease comprises the amino acid sequence of any one of SEQ ID NOS: 26 or 43.

12. The system of claim 1, wherein:
(a) the reporter is immobilized to the surface of the detection region using NHS-amine chemistry, streptavidin-biotin chemistry, epoxy-amine chemistry, maleimide-thiol chemistry, or a combination thereof;
(b) the reporter comprises an amine group and wherein the surface comprises an NHS coating; or
(c) the guide nucleic acid is immobilized to the surface of the detection region.

13. The system of claim 2, wherein the programmable nuclease is immobilized to the surface of the detection region.

14. The system of claim 1, further comprising a polymerase, a reverse transcriptase, or both; optionally wherein:
(i) the polymerase is KAPA3G DNA polymerase, RAPIDXFIRE DNA polymerase, or ACAT77 DNA polymerase; and/or
(ii) the reverse transcriptase is WARMSTART reverse transcriptase, RAPIDXFIRE reverse transcriptase, ACAT138 reverse transcriptase, and ACAT141.

15. A system for the multiplexed detection of a plurality of target nucleic acids, the system comprising:
a detection region comprising a plurality of detection locations, each detection location of the plurality of detection locations comprising:
i. a guide nucleic complementary to one of the plurality of target nucleic acids, or a portion thereof;
ii. a reporter immobilized to a surface of the detection region, the reporter comprising a nucleic acid and a detection moiety, wherein
a) the nucleic acid is at least 40 nucleotides in length;
b) the nucleic acid comprises a double-stranded region;
c) or a combination thereof, and
wherein, at each detection location, cleavage of the reporter by a programmable nuclease activated upon hybridization to the one of the plurality of target nucleic acids releases the detection moiety from the nucleic acid,
wherein the release of the detection moiety is configured to generate a signal at the detection location;
and wherein the signal indicates a presence or absence of the one of the plurality of target nucleic acids at the detection location.

16.-27. (canceled)

28. A method for detecting a target nucleic acid, the method comprising the steps of:
a. applying a plurality of nucleic acids to a detection region comprising
i. a programmable nuclease;
ii. a guide nucleic acid complementary to the target nucleic acid, or a portion thereof; and
iii. a reporter immobilized to a surface of a detection region, the reporter comprising a nucleic acid and a detection moiety, wherein
1. the nucleic acid is at least 40 nucleotides in length;
2. the nucleic acid comprises a double-stranded region;
3, or a combination thereof, and
b. detecting a signal indicative of a presence or absence of a target nucleic acid in the plurality of nucleic acids, wherein the programmable nuclease is activated by binding of the target nucleic acid to the guide nucleic acid, wherein activation of the programmable nuclease cleaves the reporter, thereby releasing the detection moiety from the nucleic acid and generating the signal indicative of a presence or absence of the target nucleic acid.

29.-41. (canceled)

42. A method for detecting a plurality of target nucleic acids, the method comprising the steps of:
a. applying a plurality of nucleic acids to a detection region comprising a plurality of detection locations, each detection location comprising
i. a programmable nuclease;
ii. a guide nucleic complementary to one of the plurality of target nucleic acids, or a portion thereof;
iii. a reporter immobilized to a surface of a detection region, the reporter comprising a nucleic acid and a detection moiety, wherein
1. the nucleic acid is at least 40 nucleotides in length;
2. the nucleic acid comprises a double-stranded region;
3, or a combination thereof, and
b. detecting, at each detection location, a signal indicative of a presence or absence of one of a plurality of target nucleic acids in the plurality of nucleic acids, wherein, at each detection location, the programmable nuclease is activated by binding of the one of the plurality of target nucleic acids to the guide nucleic acid, wherein activation of the programmable nuclease cleaves the reporter, thereby releasing the detection moiety from the nucleic acid and generating the signal indicative of a presence or absence of the one of the plurality of target nucleic acids.

43.-54. (canceled)

55. A nucleic acid molecule comprising a nucleic acid sequence at least 80% identical to any one of the sequences set forth in Table 1.

56. (canceled)

57. A system for detecting a target nucleic acid in a sample, the system comprising one or more units, wherein a unit comprises:
(a) a non-naturally occurring guide nucleic acid immobilized to a surface by a linkage; and
(b) a plurality of reporters immobilized to the surface in proximity to the non-naturally occurring guide nucleic acid;
wherein the non-naturally occurring guide nucleic acid comprises (i) one or more stabilized nucleotides, (ii) a repeat region comprising a first end and a second end, and (iii) a spacer region that hybridizes to a segment of the target nucleic acid or an amplicon thereof;
wherein the first end of the repeat region is joined to the linkage, and the second end of the repeat region is joined to the spacer region;
wherein the one or more stabilized nucleotides (i) lack a 2′—OH group, and (ii) are located within 10 nucleotides of a terminal hairpin nucleotide in the first end of the repeat region;
wherein the non-naturally occurring guide nucleic acid is effective to form a complex with a programmable nuclease that is activated upon binding the target nucleic acid or amplicon thereof;
wherein formation of the activated complex is effective to induce detectable transcollateral cleavage of the reporters; and
wherein (i) the linkage comprises a tether having a length at least that of a 10-nucleotide DNA molecule, and/or (ii) the reporters are immobilized to the surface by attachment to the non-naturally occurring guide nucleic acid, the linkage, or both.

58.-79. (canceled)

80. A system for detecting a target nucleic acid in a sample, the system comprising one or more units, wherein a unit comprises:
(a) a non-naturally occurring guide nucleic acid;
(b) a plurality of reporters; and
(c) a programmable nuclease;
wherein the non-naturally occurring guide nucleic acid comprises a spacer region that hybridizes to a segment of the target nucleic acid or an amplicon thereof;
wherein the segment hybridized by the spacer region is directly adjacent to a sequence that is complementary to a noncanonical PAM sequence;
wherein the noncanonical PAM sequence differs from a naturally-occurring PAM sequence for a reference Cas nuclease;
wherein the reference Cas nuclease is a Cas nuclease of the same type as the programmable nuclease;
wherein the non-naturally occurring guide nucleic acid is effective to form a complex with the programmable nuclease that is activated upon binding the target nucleic acid or amplicon thereof; and
wherein formation of the activated complex is effective to induce detectable transcollateral cleavage of the reporters.

81.-94. (canceled)

95. A method of assaying for one or more target nucleic acids in a sample, the method comprising:
(a) contacting the system of claim 80 with the sample;
(b) cleaving the reporters in response to presence of the target nucleic acid or amplicon thereof; and
(c) detecting a change in signal resulting from cleavage of the reporters;
wherein the detection identifies the target nucleic acid in the sample.

96.-98. (canceled)


SEQUENCE LISTING (143 sequences)

SEQ ID NO: 1 - DNA, 50 bp, synthetic construct
  source 1..50: mol_type = other DNA; note = it consists of internal 6-Fluorescein (Azide) with dT base, 3- Iowa Black FQ, and 5-amino modifier with 6 carbons
cttacccacctctcccccaaaacaaacaccactaactcacatcacaaccc

SEQ ID NO: 2 - DNA, 59 bp, synthetic construct
  source 1..59: mol_type = other DNA; note = It consists of 5-amino modifier with 6 carbons and 3-6-Fluorescein
cttacccacctctcccccaaaacaaacaccactaactcacatcacaacccttttttttt

SEQ ID NO: 3 - DNA, 12 bp, synthetic construct
  source 1..12: mol_type = other DNA; note = It consists of 5-amino modifier with 6 carbons and 3-6-Fluorescein
tttttttttttt

SEQ ID NO: 4 - DNA, 19 bp, synthetic construct
  source 1..19: mol_type = other DNA; note = It consists of internal 6-Fluorescein (Azide) with dT base, 5-amino modifier with 6 carbons, and 3- Iowa Black FQ
ttttttttttttttttttt

SEQ ID NO: 5 - DNA, 60 bp, synthetic construct
  source 1..60: mol_type = other DNA; note = It consists of 5-amino modifier with 12 carbons and 3-6-Fluorescein
tttttttttttttttttttttttttttttttttttttttttttttttttttttttttttt

SEQ ID NO: 6 - skipped

SEQ ID NO: 7 - DNA, 50 bp, synthetic construct
  source 1..50: mol_type = other DNA; note = It consists of 5-amino modifier with 6 carbons, internal 6-Fluorescein (Azide) with dT base, and 3- Iowa Black FQ
gggttgtgatgtgagttagtggtgtttgttttgggggagaggtgggtaag

SEQ ID NO: 8 - DNA, 50 bp, synthetic construct; identical to SEQ ID NO: 7
  source 1..50: mol_type = other DNA; note = It consists of 3-6-Fluorescein and 5-amino modifier with 6 carbons

SEQ ID NO: 9 - protein, 1307 aa, synthetic construct
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 10 - protein, 492 aa, synthetic construct
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 11 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 12 - protein, 1297 aa, synthetic construct
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 13 - protein, 1145 aa, synthetic construct
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 14 - protein, 1271 aa, synthetic construct
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 15 - protein, 1229 aa, synthetic construct
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 16 - protein, 835 aa, synthetic construct
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 17 - protein, 1367 aa, synthetic construct
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 18 - protein, 1264 aa, synthetic construct
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 19 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 20 - protein, 1261 aa, synthetic construct
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 21 - protein, 478 aa, synthetic construct
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 22 - protein, 1229 aa, synthetic construct
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 23 - protein, 1242 aa, synthetic construct
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 24 - protein, 1168 aa, synthetic construct
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 25 - protein, 1168 aa, synthetic construct
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 26 - protein, 1224 aa, synthetic construct
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 27 - protein, 1225 aa, synthetic construct
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 28 - protein, 1165 aa, synthetic construct
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 29 - protein, 1194 aa, synthetic construct
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 30 - protein, 1159 aa, synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 31 - protein, 1134 aa, synthetic construct
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 32 - protein, 1282 aa, synthetic construct
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 33 - protein, 1399 aa, synthetic construct
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 34 - protein, 1313 aa, synthetic construct
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 35 - protein, 1470 aa, synthetic construct
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 36 - protein, 1259 aa, synthetic construct
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 37 - protein, 477 aa, synthetic construct
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 38 - protein, 1293 aa, synthetic construct
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 39 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 40 - protein, 1286 aa, synthetic construct
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 41 - protein, 1272 aa, synthetic construct
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 42 - protein, 477 aa, synthetic construct
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 43 - protein, 1281 aa, synthetic construct
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 44 - protein, 809 aa, synthetic construct
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 45 - protein, 839 aa, synthetic construct
LNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 46 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 47 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 48 - protein, 849 aa, synthetic construct
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 49 - protein, 822 aa, synthetic construct
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 50 - protein, 806 aa, synthetic construct
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 51 - protein, 832 aa, synthetic construct
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 52 - protein, 1281 aa, synthetic construct
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 53 - protein, 1280 aa, synthetic construct
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 54 - protein, 832 aa, synthetic construct; identical to SEQ ID NO: 51

SEQ ID NO: 55 - protein, 809 aa, synthetic construct; identical to SEQ ID NO: 44

SEQ ID NO: 56 - protein, 1285 aa, synthetic construct
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 57 - protein, 1367 aa, synthetic construct; identical to SEQ ID NO: 17

SEQ ID NO: 58 - protein, 1366 aa, synthetic construct
MNTQKKEFNPKSFKDFTNLYSLNKTLRFSLTPNKKTAEILEFNKQKEVKCFSNDRKIAGAYQEIKKYLNKLHQEFIQEAMKFFAFSEEELKGFEKEYLNLLNFTDKDNFKKKNKIRNEYEQERKILTIKIATYFSKFKSEKYQSFNLANITGKKVFSILEQKYKEDKKTLKIIHIFKYKPTKDEKKEGEAVNFSTYLTGFNENRKNFYKSEDKAGQFATRTIDNLAQFIKNKKLFEDKYQKNYSKIGILDEQIKIFNLDYFNNLFLQEGLDEYNGILGNNKGEENKSNEGINQKINIFKQKEKARLKKEKENFNKSDFPLFKELYKQIGSIRKENDVYVEIKTDKELVEELNNFPKNVENYLKDIQSFYKTFFEKLQNEEYELDKIYLPKSVGTYFSYIAFSDWNKLAFIYNKRYKNEKIKIVEGGDVNVQYRSLEVLKNRIDELKDEDNLNFNKFFIDKLKFNEAKKENNWQNFWFCIEYYINSQFIGGEKNILNKEKNEYEILPFGSLKELKEKYFEAVKKYKEKMVDTESGLTDDEEKEIKETLKNYLDRIKEIERIAKYFDLKKSFEEIKQEDLDSNFYGEYQKVVDKTNELKIYQYYSEFRNYLTQNNSVEEKIKLNFNSGLLLDGWDLNKEKVKFSIIFQENGKYYLGIINKEKDKTILDKDKHPEIFTKNSDFRKMEYKLFPSPSKMLPKISFSETAKKGDEDVGWSEEIQKIKDEFAEFQEYKKKSKDNWKDEFNRGKLNKLIDYYKQVLEKHSEGYMNTYNFELKDSSKYKNLGEFNDDIARQNYKVKFVGIDKNYIDEKVANGELFLFQIYNKDFSEDKKEGSTNNLETIYFKELFSKENLENPVFKLSGGAEMFFRNKIEKKKEKKKLDKDGKPMISKKGEKVVDKRRFSENKILFHLPIEINYGKGKMPNFNKKINEYISKNPENIKIIGIDRGEKHLLYYSIIDQNGNNIESMSLNAVDEFGNFVNPEKLEEYEIDNNGKKERRWKYIVNDKEIKVTNYQRKLDELEKERQKSRQSWQNINKIKNLKKGYISFVVKKIVDLAIENNAIIILEDLNFGFKSFRQKIEKNVYQQFEKALIDKLGFVVDKQKQNQRFAPQLSAPFESFQKIGKQTGIVYYVLANNTSKVCPSCQWIKNFYLKYEKKNTIFNLQKNQKLKVFFEQEKNRFRFEYQMSKEYISVYSDVDRQRYDKTKNQNKGGYLEYKNSNQKEIIDKDGVIQKQSITLQLKELFKENHIDLEKEILKQLDNKKEKNSGYTGVYNKFIYLFNLILQIRNAISFREKDYIQCPSCHFDTRKENYLKINDGDGNGAYNIALRGLYLLKGKNGIINNLEKIKLIFSNNDYFQWAKKLKNKK

SEQ ID NO: 59 - protein, 1275 aa, synthetic construct
MQNKQSFADFTNLYSLSKTLRFELKPIGQTQAMLDENKIFEVDENRKKAYDKTKPYFDRLHREFINESLSNAQLKGISEYFETFKQFRSNQNNKDLKELINKQQKFLRHQIVTLFDENGKHWATTKYAHLKIKKKNLDILFDEQVFYILKERYGSEKETQLVDKETGAVTSIFDNWKGFTGYFTKFFETRKNFYKSDGTSTALATRIIDQNLNRFFDNLETFHKIKDKIDVKEVEIFFKLKADNVFSIDFYNQCLLQNGIDKYNDFLGGQTLENGEKQKGINEIINKYRQDNKDQKLPFLKKLDKQILSEKDRFINEIESKEEFFQVLTEFYQSATVKVTIIKTLLNDFVHNTDKYKLEKIYLTKEAFNTIANKWTDETQIFEDNLDLVLKNKKITAKQDFIPLAYIKEALEVIEKDRKFFKDRYYNDPQIGFFPDQSYWEQFLAILNFEFMTHFQRVAKDKITGKKIELGYFVFEKRIKELLDSDPSLNSQSKIIIKEFADEVLHIFQMAKYFALEKKREWKGDYYQLDDQFYNHIDYGFKDQFYENAYEKIVQPYNKIRNYLTKKPYSDVKWKLNFGNPTLANGWDKNKEADNTAVILKKDGNYYLGVMKKGKNKIFSDQNKEKYKAYNSAYYEKLVYKLFPDPSKMFPKVCFSKKGLNFFQPSEEILRIYKNNEFKKGNTFSISSMQKLIAFYIDCLGLYEGWKHYEFKNIKDVRQYKENIGEFYADVAESGYKLWFEKISEEYITQKNQLGELFLFQIYNKDFAKKTTGRKNLHTIYFEELFSQTNIDNNFPFKLNGQAELFYRPKSLEKIEEKRNFKRSIVNKKRYTQNKIFFHVPITLNRTSENIGRFNVRVNNFLANNSNVNIVGVDRGEKNLAYYSIIKQNGEVLKSGSLNIINGVDYHALLTDRAQRREQERRNWQDVESIKDLKRGYISQVVHELVSLAIKYNAIIVMEDLNMRFKQIRGGIEKSTYQQLEKALIEKLNFLVNKEETDSNQAGNLLNAYQLTAPFKTFKDMGKQTGIIFYTQASYTSKIDPLTGWRPNIYLRYSNAKQAKADILMFTNIYFSEKKDRFEFTYDLEKIDDKRKDLPIKTEWTVCSNVERFSWEKSLNNNKGGYVHYPIQDSNGEESITSKLKKLFMDFGIDLTDIKTQIESLDTNKKDNANFFRKFIFYFQLICQIRNTQVNKSDDGNDFIFSPVEPFFDSRFADKFRKNLPKNGDENGAYNIARKGLIILHKISDYFVKEGSTDKISWKDLSISQTEWDNFTTDK

SEQ ID NO: 60 - protein, 1368 aa, synthetic construct
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 61 - protein, 1257 aa, synthetic construct
MKYTDFTGIYSVSKTLRFELIPQGSTVENMKREGILNNDMHRADSYKEMKKLIDEYHKAFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFDIVAKVPEMQEHFKTIADELRSHLQVGNDIDKMFNLQFFNKVLTQSQLDVYNAVIGGKSEGNKKIQGINEYINLYNQQHKKARLPMLKLLYKQILSDRVAISWLQDEFDNDQDMLDTIEAFYNKLNSNETGVLGEGKLKQILMGLDGYNLDGVFMRNDLQLSEVSQRLCGGWNIIKDAMTSDLKRSVQKKKKETDADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDNFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHSLLEEKEAGNKKARQDWQTIESIKELKEGYLSQVIHKISMLMQRYHAIVVLEDLNGSFMRSRQKVEKQVYQKFEHMLINKLNYLVNKQYDATEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 62 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 63 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 64 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 65 - protein, 718 aa, synthetic construct
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 66 - protein, 757 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 67 - protein, 761 aa, synthetic construct
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 68 - protein, 708 aa, synthetic construct
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 69 - protein, 661 aa, synthetic construct
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 70-71 - skipped (2 entries)

SEQ ID NO: 72 - protein, 5 aa, synthetic construct
  VARIANT 1..5: note = GSGGS repeats may vary between 1-10. up to 9 GSGGS repeats may be absent
  REGION 1..5: note = Synthetic polypetide
GSGGS

SEQ ID NO: 73 - protein, 6 aa, synthetic construct
  VARIANT 1..6: note = GGSGGS repeats may vary between 1-10. up to 9 GGSGGS repeats may be absent
  REGION 1..6: note = Synthetic polypetide
GGSGGS

SEQ ID NO: 74 - protein, 4 aa, synthetic construct
  VARIANT 1..4: note = GGGS repeats may vary between 1-10. up to 9 GGGS repeats may be absent
  REGION 1..4: note = Synthetic polypetide
GGGS

SEQ ID NO: 75 - protein, 4 aa, synthetic construct
  VARIANT 1..4: note = GGSG repeats may vary between 1-10. up to 9 GGSG repeats may be absent
  REGION 1..4: note = Synthetic polypetide
GGSG

SEQ ID NO: 76 - protein, 5 aa, synthetic construct
  VARIANT 1..5: note = GGSGG repeats may vary between 1-10. up to 9 GGSGG repeats may be absent
  REGION 1..5: note = Synthetic polypetide
GGSGG

SEQ ID NO: 77 - protein, 5 aa, synthetic construct
  VARIANT 1..5: note = GSGSG repeats may vary between 1-10. up to 9 GSGSG repeats may be absent
  REGION 1..5: note = Synthetic polypetide
GSGSG

SEQ ID NO: 78 - protein, 5 aa, synthetic construct
  VARIANT 1..5: note = GSGGG repeats may vary between 1-10. up to 9 GSGGG repeats may be absent
  REGION 1..5: note = Synthetic polypetide
GSGGG

SEQ ID NO: 79 - protein, 5 aa, synthetic construct
  VARIANT 1..5: note = GGGSG repeats may vary between 1-10. up to 9 GGGSG repeats may be absent
  REGION 1..5: note = Synthetic polypetide
GGGSG

SEQ ID NO: 80 - protein, 5 aa, synthetic construct
  VARIANT 1..5: note = GSSSG repeats may vary between 1-10. up to 9 GSSSG repeats may be absent
  REGION 1..5: note = Synthetic polypetide
GSSSG

SEQ ID NO: 81 - protein, 1166 aa, synthetic construct
MRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLL

SEQ ID NO: 82 - protein, 845 aa, synthetic construct
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 83 - protein, 1332 aa, synthetic construct
MKSIFDNFTGLYSLSKTLRFELRPVGQTLENIKNGHFLESDKKMADDYQDVKKIIDNYHKFFIDDVLKGASFDWALLEKELTDFNKNKTDDSKVEAEQKKLREQIAKTLAGDKRFKSLTASTPNDLFNKDKDFIGWLEQSSVKEIRKDALDTFKKFSSYFKGFQENRKNVYSADDIPTAVPYRIVNDNFPKFLQNISIFKTIQEKCPQVIADVENELASYLGKEKLADIFTVQAFNKYLCQGGKENQRGIDFYNQVIGGIAEKEGGVNLRGINQFLNLYWQQHPDFAKENRRIKMVPLYKQILSDRSSLSFKIETIDTDEELKTAISEYADKLESKSNDEKKSVLDVCVELFDSIKEQNLQEIYVNRKDINNISRILTGDWSWLQSRMNLYADEVFTTKAEKTRWQKSVDGDEGENKSKGAYSLAELNRVLEYASENVAETDIRITDYFCHRNRFYYEKESGLFKQGEELVALSIKESCEDILSKRKAMNEAFANISESNSLRDNSEDIAKTKTYLDSVQDLLHRIKPLKVNGLGDPSFYAVFDSIYSALSEVISIYNKTRNYITRKAESPEKYKLNFDNPTLANGWDLNKEKDNTCVLLRKNGMYYLGIMNPKDKPKFAEKYDCGTESCYEKMIYKLLPGPNKMLPKVFFSTKGKKQYNPPENILHGYEQGKHKKGVAFDINFCHELIDWFKSAINQHEDWKKFGFKFSDTKSYKDISDFYREVTEQGYKLTFINIPESEISKMVSEGKLYLFQIYNMDFAPGANGMPNMHTLYWKNLFSEENLKDVVLKLNGEAELFYRPAGIKEPVVHAKGSYLVNRITKDGEPIPEKIHDEIYRNANGNLNNLSKEASEYKESHKVVIKQASHEIIKDRHYTEPKFLFHIPLTINFKAPSMKPTVCTAIQNENVRRFLKNNPDVNIIGLDRGERNLIYLSLINQKGEIIKQFSFNDVEREQNGQTVKVNYHEKLDQREKERDAARKSWQAIGKIAELKEGYLSAVIHQLTKLMVEYNAIVVMEDLNFGFKRGRFHVEKQVYQKFEHMLIDKLNYLVFKDRGLNEPGGALNGYQLAGQFESFQKLGKQSGMLFYVPAAYTSKIDPKTGFVSMMNFKDLTNVHKKRDFFSKFEDIHFDEATCSFVFTFDYKNFNGKAKEEMKQTKWAVYSREKRIVYFSKTKSSEDIMPTEKLRALFESDGIEYKSGNNIHDSVMAVGADLKEGAKPSKEIADFWDGLLYNFKLILQMRNSNAKTGEDYIISPVMASDGTFFDSRVEAKKGKDAKLPLDADANGAYHIALKGLSLINKINLAGEDELKKFDMKISNEDWFNFAQEKKYAE

SEQ ID NO: 84 - protein, 1345 aa, synthetic construct
MTKQNKSVTQNTKRKNFGEFTNLYSLSKTLRFELKPVKATKTILEVERKDRENFKKDRKIAKNYQKLKGILNELHQEFIQDVMREFSFQEKEIKEFEERYLEALNFKEKDNYKKRTQLKNAYEKVAKKLAGKIATAFGKYNQEKYGVKFTKKNLTGENVFDILEGKYKGDKKILGIIHTFKFKPTKEEKKQGKEAVNFSTYLGGFNQNRENFYKGEMKAGQFATRTIENLIQFLKNKKLFIDKYKDNYQKIGFSQEQVEIFNLNYFNNLFLQEGLDVYNGILGAKKGEKNTENDGLNQKINLFKQKEKTRCKANGEKFNKSDYPIFKELYKQIGSIKKDNDVYVEIKSDEELVNVLQSLPEKTANTLREVQKFYENFFDKIFNDEFDLDKIYLPKSVGTHFSHLAFSDWSKLAFVFNKRWRNEKVKIKEGEDVNVQSRSLADIKKRMEEILEMDGGVSFGKTYCQKVGLEKEARTIEDVWSGFWKIIQYHINSQFIGGEKEVFDKEKKDDKTEKIQTIDDLQEEYLQATEMYRERMVESEEGLNDGEEKEIKTKLKNYLDRIKDIERIARYFDLRKHFDDIDEASKDGDFYFIYQELLQDISEAKINDHYNEIRNYLTKANVVDDKFKLNFNDGQTLSGWDLNKETEKFSLIFKRKVDGGVEYYLGIINKEKNKTIFDKKKHPEIFTENSEFEKMEYKLFPSPSKMLPKIAFTKNKEGERIKPVFLDENAGKEIAQIKKEFALFQDAKKEDKNKWSDEFDRKKLNKLIDYYKLVLEKHPEKYMQTFNFVFKSSAKYKNLGEFNDDVARQNYVTKFVSVDKDYIDQKVESGELYLFKIHNKDWNLTKAGDTKKQSKKNLHTIYFEELFSEKNIAEPVFKLSGGAEVFFRDAIEKKKQKKKKDKKGKEILEKFRFTKNKILFHVPITINYGKPSINQGQFNQKINEFIADNSRSVNILGIDRGEKHLLYYSLVDNNGKIIKSGSLNEINGVDYHEKLDKAEKERQEARKSWQKINQIKNLKAGYISQVIKKIVDLAIENNAIIVLEDLNFGFKSFRQKIEKNVYQQFEKALIDKLGFVTDKEKLNHRQAPQLSAPFESFEKMGKQTGIVFYVLATNTSKVCPQCQWKKNIFPHYSTKKSIAENLQKQYKMKMYWRENENRFEFEYKGDGDKEFSSIFSNVDRVRYDKRANNNQGGYVIYQIDSTTKEKDGRNIKEKSITNLLKELLLEKFEIDNLEGELLVKLSEKSPDVSKETIKDFFGLLNSILNIRNSMTDTEEDYIQCPACGFDTRKENKIGIKNGDDNGAYNIALRGRFLIERIKKAKKEDKKPNLTFSNNDYFQWVREFVK

SEQ ID NO: 85 - protein, 1281 aa, synthetic construct
MIKSYDDFTKLYSLQKTLRFELKPIGKTLEHIKKSEIIESDETLEEQYAIVKNIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELYLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITMNFISKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 86 - protein, 1265 aa, synthetic construct
MRTTTSLDAFTNRYALSKTLRFELKPIGNTQMMLEQNNVFAKDRAIREKYEKTKPWIDLLHREFVAESLQNAQLGNLDDYYAALQNVQKITKDTNAEDKKRWKKGFEKQEKRLRKEVVALFDKAAHIWATQRYPQLKKKTKDFLFEEGVFEHVLFARYGSAPDTTVKIVTSNPETGEVIDEREESIFKGWKGFTGYFDKFFETRKNFYKDNGTATAIATRAINQNLRRFAENMQKLTDIKNNYPELLAHTDFGDFDIAHAQSLDFYARTCLLQEGIDAYNKKFVGVLKSAINEYQQKNKGVRISYPKTLDNQILGERERRLFDVIEDDRELHDVFRAFVDDGTVFAAEMRQLAQAFSAQNGTYDYTQIYISKKGFETISRKYTHDTRAWHDALADVFKAKAKKRIATTASGEKKFPAYIPVAYITQALTLVQESEDTECTWKERYASITENKTLEEGFFAIFADEFERLFVHMEATVQDTDYVVAEDKAKKLLSDGQITKNEQTTQIIKEYADALLRIYQMAKYFAVEKKSMWDDAVAIDDTFYETFKEIYGNTHSTIVASYNLLRNYLTKKPWEDVQKWKLNFENPTLLDGWDKNKEAANFGVILRDGDKFYLGIMRKGHNNIFANQHHSNFEGQGLQKMVYKFFPDPKKMFPKVCFSAKGMEFFAPSEEIVRIYKNAEFKSGDTFNVESMQKLIDFYKNALQKYDGWKIYDFKHLKDTAQYTSNIGEFYDDVAKGGYQLGWQNISKEYVEEKNANGELYLFQIKNKDWNDGATGRKNLHTLYFEYLFSEKNAAADFVFRLNGGAEVFYRPAAIESKTERRGNREVAAKKRYTQDKVFLHVPITLNRTAGDVKTSAFNDAVNRFLAGNPDINIMGIDRGEKHLAYYSIIDQNGNRIVSGSFNTIGSKDYHALLTERQGAREEARKNWQRVEQIKDLKKGYISLVVREIADLAIKHNAIIVLENLNMRFKQIRGGIEKSVYQQLEKALIEKLNFLVNKGEVDATKAGHLLRAYQLAAPFETFEKMGNQTGIIFYTTASYTSQVDPVTGWRPHVYLKYRNAQKTKEDILRIFDDIVFNDEKQRFEFAYRHNGVSWTVCSSVERHRWNRSNNAGKGGYDVFPVEGEGSITQRLQEACAQRGIDTTRNILAQIDELDESASATVSFLRDLCFYFRLICQIRNTDDGADDINAQDFLMSPVEPFFDTRNAQEQYPQNGDENGAYNIARKGIIILQKITAWGRSQDTQRRYPDTFVSQDEWDTFLTQHTT

SEQ ID NO: 87 - protein, 1259 aa, synthetic construct
MLKEKQFKTFGDFTNLYELSKTLRFELRPTPETKDLLDKNKIIQTDKKIAENYQEIKKYFDKLHKKFIKEALSNTQIDFSDFCKLWEQNSKDSGKIKDLSRKLRKSIKQAFDKKGADWHKRYLEKGIKLKKKNLDILFEERVLDILKEEFKDDVDVKLFESFKGFSTYFTNFHESRKNFYKDDGTASAIATRIIDENLKRFCDNIKVKKHSKKLISELNEREAKIFEADFYNRCLLQQGIDDYNQVIGDINKKINNLRQNKIENTPTLKILYKQILGDVRRQETEQDAFIEIKNNEEVFDFLQDFIKHSDENNKYFKNLFYKFIEGKHSLDKIFLAKRFVNTISGKWFASWEVFGAELIKKFGNKKDLPDFIPFAAVKDVLQNCNIPANELFKEKIKNDEDKNIYDIFINLWKEEFDSNLKKVEESKKEVENMIAEDKVYSNKKEKRKNDNGEEIEIEIQKEKIKNYADAAMNIFRMMKYFLLEKNGKTVEGMGEDNNFYNELNIVFKGGEIDGKVYEGVKTYLYYNEFRNYLTKKPFNEEKTKLNFDCGQILSGWDKNKESEKLGVILRKDNKYYLAIINKKHNKIFDVKKNSYAYIVGDNFYEKMEYKLFPDAKRMIPKIAFAKNNKEKFGWTDEIQKIKNEYAEFQEGKKNDKNLWKDKFNKNKMEKLITYYQNCLEKGGYKDIYNFRWKSPDKYSGIGEFNDEIDRQSYCLKFVKVDFNYVFEKVKSGELYLFQIYNKDFSDKADRAQKENIHTEYFKLLFDQRNLDNVVLKLSGGAEIFYRPKTEGLPKKKDNKGNEVVRHRRYADDKYFLHLPIQLNFGRGNLSGGEFNSKINQYLSEQREIKIIGIDRGEKHLAYYSVINQDGKIEEIESLNTVNGIDYRKKLDELEKKREQERKSWQSISKIKDLKKGYISHVIKKICDLAIEHNAIIVFEDLSGGFKNSRKKIEKQIYQNLELALATKLNYLTFKDKNFGESGHYLNAYQLAPKIDNYQDIKMQTGIVFYTPAGYTSSTCPQCGFRKTLKFDYTATISKAEDLIRGSKLNIVFEKEKNRFKINYLFNPIEKKKKKIKENELFADAGAKNEFTIYSDVKRIKWHNTGTKRLEEAEGERLLENKNSRGRDKEYDINKCLTRLFRENKIDVNGDIIGQITKIKSLKLYQDLFYYLFLATLIRNNVSGSDIDYIQCPSCHFHSDGGFQKQKFNGDANGAYNIARKGILILKKIKQFAAQDKDMKNFGWKHLTVDINEWDKFTQK

SEQ ID NO: 88 - protein, 1371 aa, synthetic construct
MNKNFSNFTELYTLSKTLRFELKPVAQTKENIKKGKFLESDKKKADDYKDVKKIIDNYHKFFIDDVLKNASFDWTVLEKEMSDFNKSKADDSKVEAEQKKLRDQIAKKLTSDKRFKALTASTPSDLFNKDKDFIDWFTQNSTKDINKEALETFKRFSSYFKGFQENRKNVYSAEPIPTAVPYRLVNDNFPKFLQNIALFKIIQEKCPQVISDVEKELASYLGKEKLADIFTVQAFNKYLCQGGKENQRGIDFYNNILGGIAEKEGGINLRGINQFLNLYWQQHQDFAKQNKRIKMIPLFKQILSDRSSLSFKIESINTDQELLTSITEYADKLETKSNDEKKSVLDICSDLFASIKAQNLQEIYVNRKDINSISRILTGDWSWLQSRMNVYADEVFTTKAEKTRWQKSIDGDEGENKSKGVFSLAELNSVLEYSSENVSETDVRITDFFDHRNRFYYEKESGLFKQGDELVALSIRESCEDILAKRKAMDEAFANVSENNSLRDNSEDVAKIKIYLDCVQELLHRIKPLKVNGLGDPAFYAVFDTVYNSLSEVISLYNKTRNYITKKAANPEKYKLNFDNPTLADGWDLNKEQANTSVLMRKDGMYYLGIMNPKDKPKFAEKYECGNEACYEKMIYKQFDATKQIPKCSTQVKEVKKHFQSGATDSIILNDKSKFKLDLVITKEIWFLNNHVWNGEKFVPKRESNETRPKKFQIGYYKQTGDLGGYKEALNIWISFCKTFLQSYISSSIYDYDFKESSNYDSLDEFYNYLNATCYKLSFINIPEATISQMVSEGKLYLFQIYNKDFAPGASGMPNMHTLYWKNLFSEENLKNVVLKLNGEAELFYRPAGIKEPVIHAKGSYLVNRITKDGEPIPEKIHDEIYRNANGKLESLSKEATEYKASHKVVIKEAKHDIIKDRHYTEPKFLFHVPLTINFKASGNSYINENVRRFLKNNPDVNVIGLDRGERHLIYLSLINQKGEIIKQFTFNEVERNKNGQVIKVNYHEKLDQREKVRGAARKSWQAIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMSNFKDLTNVHKKRAFFSKFDDIHFDDATGSFVFTFDYKNFDGKAKEEMKRTKWSVYSKDKRIVYLSKTKSYEDVQPTEKIKASLESVGIEYMSGNNLIDSIMVIGAELKDGAKPSKEIADFWDRLLYNFKLILQMRNSNAKTGEDYIISPVMADDGTFFDSREEFKKGENAKMPVDADANGAYHIALKGLSLLKRFDAASENELKKFDMKISNVDWFKFAQEKSYAE

SEQ ID NO: 89 - RNA, 41 nt, synthetic construct
  source 1..41: mol_type = other RNA
taatttctactaagtgtagatgccgataatgatgtagggat

SEQ ID NO: 90 - DNA, 56 bp, synthetic construct
  source 1..56: mol_type = other DNA; note = It consists of 5BiotinTEG
  misc_structure 1..17: note = DNA
  misc_feature 18..56: note = RNA
ttttttttttttttttaatttctactaagtgtagatgccgataatgatgtagggat

SEQ ID NO: 91 - DNA, 41 bp, synthetic construct
  source 1..41: mol_type = other DNA; note = it consists of 5-amino modifier with 12 carbons
  misc_difference 1..6: note = DNA
  misc_feature 7..41: note = RNA
taatttctactaagtgtagatgccgataatgatgtagggat

SEQ ID NO: 92 - DNA, 20 bp, synthetic construct
  source 1..20: mol_type = other DNA
ctaacactctcagggacaat

SEQ ID NO: 93 - DNA, 20 bp, synthetic construct
  source 1..20: mol_type = other DNA
ttactaacactctcagggac

SEQ ID NO: 94 - DNA, 20 bp, synthetic construct
  source 1..20: mol_type = other DNA
tttactaacactctcaggga

SEQ ID NO: 95 - DNA, 20 bp, synthetic construct
  source 1..20: mol_type = other DNA
gagagtgttagtaaacggaa

SEQ ID NO: 96 - DNA, 20 bp, synthetic construct
  source 1..20: mol_type = other DNA
cctgagagtgttagtaaacg

SEQ ID NO: 97 - DNA, 20 bp, synthetic construct
  source 1..20: mol_type = other DNA
ccctgagagtgttagtaaac

SEQ ID NO: 98 - RNA, 40 nt, synthetic construct
  source 1..40: mol_type = other RNA
gaatttctactattgtagatctaacactctcagggacaat

SEQ ID NO: 99 - RNA, 40 nt, synthetic construct
  source 1..40: mol_type = other RNA
gaatttctactattgtagatttactaacactctcagggac

SEQ ID NO: 100 - RNA, 40 nt, synthetic construct
  source 1..40: mol_type = other RNA
gaatttctactattgtagattttactaacactctcaggga

SEQ ID NO: 101 - RNA, 40 nt, synthetic construct
  source 1..40: mol_type = other RNA
gaatttctactattgtagatgagagtgttagtaaacggaa

SEQ ID NO: 102 - RNA, 40 nt, synthetic construct
  source 1..40: mol_type = other RNA
gaatttctactattgtagatcctgagagtgttagtaaacg

SEQ ID NO: 103 - RNA, 40 nt, synthetic construct
  source 1..40: mol_type = other RNA
gaatttctactattgtagatccctgagagtgttagtaaac

SEQ ID NO: 104 - DNA, 20 bp, synthetic construct
  source 1..20: mol_type = other DNA
ttccagggaacaggcctttt

SEQ ID NO: 105 - DNA, 20 bp, synthetic construct
  source 1..20: mol_type = other DNA
cttccagggaacaggccttt

SEQ ID NO: 106 - DNA, 20 bp, synthetic construct
  source 1..20: mol_type = other DNA
gcttccagggaacaggcctt

SEQ ID NO: 107 - DNA, 20 bp, synthetic construct
  source 1..20: mol_type = other DNA
ggcttccagggaacaggcct

SEQ ID NO: 108 - DNA, 20 bp, synthetic construct
  source 1..20: mol_type = other DNA
gggcttccagggaacaggcc

SEQ ID NO: 109 - DNA, 20 bp, synthetic construct
  source 1..20: mol_type = other DNA
tgggcttccagggaacaggc

SEQ ID NO: 110 - DNA, 20 bp, synthetic construct
  source 1..20: mol_type = other DNA
ttgggcttccagggaacagg

SEQ ID NO: 111 - DNA, 20 bp, synthetic construct
  source 1..20: mol_type = other DNA
ctggaagcccaaaggactct

SEQ ID NO: 112 - DNA, 20 bp, synthetic construct
  source 1..20: mol_type = other DNA
tccctggaagcccaaaggac

SEQ ID NO: 113 - RNA, 40 nt, synthetic construct
  source 1..40: mol_type = other RNA
gaatttctactattgtagatttccagggaacaggcctttt

SEQ ID NO: 114 - RNA, 40 nt, synthetic construct
  source 1..40: mol_type = other RNA
gaatttctactattgtagatcttccagggaacaggccttt

SEQ ID NO: 115 - RNA, 40 nt, synthetic construct
  source 1..40: mol_type = other RNA
gaatttctactattgtagatgcttccagggaacaggcctt

SEQ ID NO: 116 - RNA, 40 nt, synthetic construct
  source 1..40: mol_type = other RNA
gaatttctactattgtagatggcttccagggaacaggcct

SEQ ID NO: 117 - RNA, 40 nt, synthetic construct
  source 1..40: mol_type = other RNA
gaatttctactattgtagatgggcttccagggaacaggcc

SEQ ID NO: 118 - RNA, 40 nt, synthetic construct
  source 1..40: mol_type = other RNA
gaatttctactattgtagattgggcttccagggaacaggc

SEQ ID NO: 119 - RNA, 40 nt, synthetic construct
  source 1..40: mol_type = other RNA
gaatttctactattgtagatttgggcttccagggaacagg

SEQ ID NO: 120 - RNA, 40 nt, synthetic construct
  source 1..40: mol_type = other RNA
gaatttctactattgtagatctggaagcccaaaggactct

SEQ ID NO: 121 - RNA, 40 nt, synthetic construct
  source 1..40: mol_type = other RNA
gaatttctactattgtagattccctggaagcccaaaggac

SEQ ID NO: 122 - DNA, 60 bp, synthetic construct
  source 1..60: mol_type = other DNA; note = It consists of 5-amino modifier with 12 carbons and 3-6-Fluorescein
  misc_feature 1..6: note = DNA
  misc_feature 7..41: note = RNA
  misc_feature 42..60: note = DNA
taatttctactaagtgtagatgccgataatgatgtagggatttttttttttttttttttt

SEQ ID NO: 123 - DNA, 60 bp, synthetic construct; identical to SEQ ID NO: 122
  source 1..60: mol_type = other DNA; note = It consists of 5-amino modifier with 6 carbons and 3-6-Fluorescein
  misc_feature 1..6: note = DNA
  misc_feature 7..41: note = RNA
  misc_feature 42..60: note = DNA

SEQ ID NO: 124 - DNA, 20 bp, synthetic construct
  source 1..20: mol_type = other DNA
ttacatggctctggtccgag

SEQ ID NO: 125 - DNA, 20 bp, synthetic construct
  source 1..20: mol_type = other DNA
acatggctctggtccgaggt

SEQ ID NO: 126 - DNA, 20 bp, synthetic construct
  source 1..20: mol_type = other DNA
tacatggctctggtccgagg

SEQ ID NO: 127 - DNA, 20 bp, synthetic construct
  source 1..20: mol_type = other DNA
cttacatggctctggtccga

SEQ ID NO: 128 - DNA, 20 bp, synthetic construct
  source 1..20: mol_type = other DNA
tcttacatggctctggtccg

SEQ ID NO: 129 - DNA, 20 bp, synthetic construct
  source 1..20: mol_type = other DNA
ttcttacatggctctggtcc

SEQ ID NO: 130 - DNA, 20 bp, synthetic construct
  source 1..20: mol_type = other DNA
tttcttacatggctctggtc

SEQ ID NO: 131 - DNA, 20 bp, synthetic construct
  source 1..20: mol_type = other DNA
accagagccatgtaagaaaa

SEQ ID NO: 132 - DNA, 20 bp, synthetic construct
  source 1..20: mol_type = other DNA
gaccagagccatgtaagaaa

SEQ ID NO: 133 - RNA, 41 nt, synthetic construct
  source 1..41: mol_type = other RNA
taatttctactaagtgtagatttacatggctctggtccgag

SEQ ID NO: 134 - RNA, 40 nt, synthetic construct
  source 1..40: mol_type = other RNA
gaatttctactattgtagatacatggctctggtccgaggt

SEQ ID NO: 135 - RNA, 40 nt, synthetic construct
  source 1..40: mol_type = other RNA
gaatttctactattgtagattacatggctctggtccgagg

SEQ ID NO: 136 - RNA, 40 nt, synthetic construct
  source 1..40: mol_type = other RNA
gaatttctactattgtagatcttacatggctctggtccga

SEQ ID NO: 137 - RNA, 40 nt, synthetic construct
  source 1..40: mol_type = other RNA
gaatttctactattgtagattcttacatggctctggtccg

SEQ ID NO: 138 - RNA, 40 nt, synthetic construct
  source 1..40: mol_type = other RNA
gaatttctactattgtagatttcttacatggctctggtcc

SEQ ID NO: 139 - RNA, 40 nt, synthetic construct
  source 1..40: mol_type = other RNA
gaatttctactattgtagattttcttacatggctctggtc

SEQ ID NO: 140 - RNA, 40 nt, synthetic construct
  source 1..40: mol_type = other RNA
gaatttctactattgtagataccagagccatgtaagaaaa

SEQ ID NO: 141 - RNA, 40 nt, synthetic construct
  source 1..40: mol_type = other RNA
gaatttctactattgtagatgaccagagccatgtaagaaa

SEQ ID NO: 142 - DNA, 41 bp, synthetic construct; identical to SEQ ID NO: 91
  source 1..41: mol_type = other DNA; note = It consists of 5-amino modifier with 12 carbons and 18-atom hexa-ethyleneglycol spacer
  misc_feature 1..6: note = DNA
  misc_feature 7..41: note = RNA

SEQ ID NO: 143 - DNA, 41 bp, synthetic construct; identical to SEQ ID NO: 91
  source 1..41: mol_type = other DNA; note = It consists of 5-amino modifier with 6 carbons and 18-atom hexa-ethyleneglycol spacer
  misc_feature 1..6: note = DNA
  misc_feature 7..41: note = RNA